(19) United States
(12) Patent Application Publication
(10) Pub. No.: US 2022/0227889 A1
(43) Pub. Date: Jul. 21, 2022

(54) METHODS AND REAGENTS FOR CLEAVAGE OF THE N-TERMINAL AMINO ACID FROM A POLYPEPTIDE

(71) Applicant: Encodia, Inc., San Diego, CA (US)

(72) Inventors: Kevin L. GUNDERSON, San Diego, CA (US); Fei HUANG, San Diego, CA (US); Robert C. JAMES, San Diego, CA (US); Luca MONFREGOLA, San Diego, CA (US); Stephen VERESPY, III, San Diego, CA (US); Eric Cunyu ZHOU, San Diego, CA (US)

(73) Assignee: Encodia, Inc., San Diego, CA (US)

(21) Appl. No.: 17/606,759

(22) PCT Filed: Apr. 24, 2020
(86) PCT No.: PCT/US20/29969

§ 371 Date: Oct. 26, 2021

Related U.S. Application Data
(60) Provisional application No. 62/841,171, filed on Apr. 30, 2019.

(51) Int. Cl. C07K 17/00; C07D 231/12; C07K 14/195; C12N 9/48

(52) U.S. Cl. CPC C07K 17/00; C07D 231/12; C12Y 304/19003; C12N 9/485; C07K 14/195

(57) ABSTRACT

The present invention relates to methods of cleaving the N-terminal amino acid from a polypeptide, which may be in free form or conjugated to a carrier or surface, such as a bead. It provides methods to activate the N-terminal amine of a polypeptide to promote formation of a cyclic adduct of the N-terminal amino acid, resulting in cleavage of the N-terminal amino acid from the polypeptide. The method can be used to sequence and/or analyze a polypeptide. For example, the methods can be combined with methods described herein for sequencing and/or analysis that employ barcoding and nucleic acid encoding of molecular recognition events, and/or detectable labels. The invention also provides compounds and kits useful for practicing these methods.

### RELATED APPLICATIONS

The present application claims priority to U.S. provisional patent application No. 62/841,171, filed on Apr. 30, 2019, the disclosures and contents of which are incorporated by reference in their entireties for all purposes.

### SEQUENCE LISTING ON ASCII TEXT

This patent or application file contains a Sequence Listing submitted in computer readable ASCII text format (file name: 4614-2001440_20200422_SeqList_ST25.txt, recorded: Apr. 22, 2020, size: 54,3804 bytes). The content of the Sequence Listing file is incorporated herein by reference in its entirety.

### TECHNICAL FIELD

The present disclosure relates to methods, reagents and kits for analysis of polypeptides. In some embodiments, the present methods, reagents and kits employ mild conditions for removal of the N-terminal amino acid of a polypeptide and may be used to modify and remove one or more N-terminal amino acids from a polypeptide, and they may be readily applied to polypeptide analysis and/or sequence determinations.

### BACKGROUND

Proteins play an integral role in cell biology and physiology, performing and facilitating many different biological functions. The repertoire of different protein molecules is extensive, much more complex than the transcriptome, due to additional diversity introduced by post-translational modifications (PTMs). Additionally, proteins within a cell dynamically change (in expression level and modification state) in response to the environment, physiological state, and disease state. Thus, proteins contain a vast amount of relevant information that is largely unexplored, especially relative to genomic information. In general, innovation has been lagging in proteomics analysis relative to genomics analysis. In the field of genomics, next-generation sequencing (NGS) has transformed the field by enabling analysis of billions of DNA sequences in a single instrument run, whereas in protein analysis and peptide sequencing, throughput is still limited.

Yet this protein information is direly needed for a better understanding of proteome dynamics in health and disease and to help enable precision medicine. As such, there is great interest in developing “next-generation” tools to miniaturize and highly-parallelize collection of this proteomic information.

Highly-parallel macromolecular characterization and recognition of proteins is challenging for several reasons. The use of affinity-based assays is often difficult due to several key challenges. One significant challenge is multiplexing the readout of a collection of affinity agents to a collection of cognate macromolecules; another challenge is minimizing cross-reactivity between the affinity agents and off-target macromolecules; a third challenge is developing an efficient high-throughput read out platform. An example of this problem occurs in proteomics in which one goal is to identify and quantitate most or all the proteins in a sample. Additionally, it is desirable to characterize various post-translational modifications (PTMs) on the proteins at a single molecule level. Currently this is a formidable task to accomplish in a high-throughput way. Direct protein characterization via peptide sequencing (Edman degradation or Mass Spectroscopy) provide useful approaches. However, neither of these approaches is very parallel or high-throughput.

Peptide sequencing based on Edman degradation was first proposed by Pehr Edman in 1950; namely, stepwise removal of the N-terminal amino acid on a peptide through a series of chemical modifications and downstream HPLC analysis (later replaced by mass spectrometry analysis). In a first step, the N-terminal amino acid is modified with phenyl isothiocyanate (PITC) under mildly basic conditions (NMP/methanol/H₂O) to form a phenylthiocarbamoyl (PTC) derivative. In a second step, the PTC-modified amino group is treated with acid (anhydrous TFA) to create a cleaved cyclic ATZ (2-anilino-5(4)-thiozolinone) modified amino acid, leaving a new N-terminus on the peptide. The cleaved cyclic ATZ-amino acid is converted to a phenylthiohydantoin (PTH) amino acid derivative and analyzed by reverse phase HPLC. This process is continued in an iterative fashion until some or all of the amino acids comprising a peptide sequence have been removed from the N-terminal end and identified. In general, Edman degradation peptide sequencing is slow and has a limited throughput of only a few peptides per day. Moreover, because the cleavage step uses a very strong acid (typically anhydrous TFA), this method is incompatible with samples containing acid-sensitive moieties such as oligonucleotides or polynucleotides. Thus improved methods are needed for sequencing of polypeptides.

Accordingly, there remains a need in the art for improved techniques relating to macromolecule sequencing and/or analysis, with applications to protein sequencing and/or analysis, as well as to products, methods and kits for accomplishing the same. There is furthermore a need for protein sequencing methods that are highly-parallelized, accurate, sensitive, and high-throughput, while also being mild enough to avoid degrading other materials commonly found in protein samples to be analyzed, such as oligonucleotides or polynucleotides. The present invention addresses this and related need and provides a milder, more flexible alternative to Edman degradation for cleaving or selectively cleaving the N-terminal amino acid from a polypeptide and identifying the amino acid that was removed.

These and other aspects of the invention will be apparent upon reference to the following detailed description. To this end, various references are set forth herein which describe in more detail certain background information, procedures, compounds and/or compositions, and are each hereby incorporated by reference in their entirety

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, the invention provides a method to cleave or selectively cleave the N-terminal amino acid (NTAA) from a polypeptide of any length. In particular, it provides methods to cleave an N-terminal amino acid residue from a peptidic compound of Formula (I)

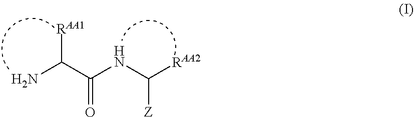

wherein the method comprises:
(1) Converting the peptidic compound to a guanidinyl derivative of Formula (II):

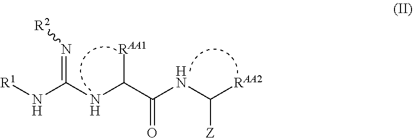

or a tautomer thereof; and
(2) contacting the guanidinyl derivative with a suitable medium to produce a compound of Formula (III)

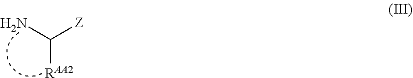

wherein:
R¹ is R³, NHR³, —NHC(O)—R³, or —NH—SO₂—R³
R² is H, R⁴, OH, OR⁴, NH₂, or —NHR⁴;
R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;

and wherein two R′ or two R″ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
R^{AA1} and R^{AA2} are each independently selected amino acid side chains;
   and the dashed semi-circle connecting R^{AA1} and/or R^{AA2} to the nearest N atom indicates that R^{AA1} and/or R^{AA2} can optionally cyclize onto the designated N atom; and
Z is —COOH, CONH₂, or an amino acid or a polypeptide that is optionally attached to a carrier or solid support.

Provided herein are different methods to convert the peptidic compound to a compound of Formula (II) as well as novel reagents for these methods. It can be used on any suitable polypeptide comprised of alpha-amino acids, which may be natural, synthetic, or post-translationally modified. In general, the descriptions and methods provided herein may apply to modification, cleavage, treatment, and/or contact of beta amino acids. For example, isoaspartic acid is a biologically relevant beta amino acid that may be modified, cleaved, treated, and/or contacted as described herein.

In another aspect, the invention provides compounds useful in the methods disclosed herein. For example, the invention provides compounds of the Formula (AB)

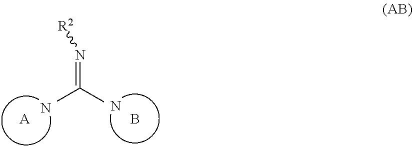

wherein:
R² is H, R⁴, OH, OR⁴, NH₂, or —NHR⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;

ring A and ring B are each independently a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;

wherein each R is independently selected from H and C₁₋₃ alkyl optionally substituted with OH, OR*, —NH₂, —NHR*, or —NR*₂; and

each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

with the proviso that Ring A and Ring B are not both unsubstituted imidazole and that Ring A and Ring B are not both unsubstituted benzotriazole;

or a salt thereof.

These compounds are useful for activing an NTAA for further modification or for cleavage from a polypeptide, and for methods disclosed herein for using this cleavage method to analyze a polypeptide, including providing information about the amino acid sequence of the polypeptide.

In another aspect, the invention provides compounds of Formula (II), which are polypeptides in which the NTAA has been activated for further modification and/or cleavage. These compounds are useful as intermediates in certain of the methods disclosed herein for analyzing or sequencing a polypeptide, as they can be induced to undergo cleavage of the NTAA residue under mild conditions that permit NTAA cleavage without damaging acid-sensitive substances such as polynucleotides that may be present in the sample, and may be conjugated to the polypeptide and used, as described herein, to capture information about the sequence of the polypeptide. For example, the invention provides compounds of Formula (II):

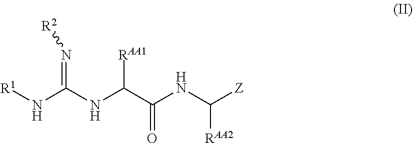

or a tautomer thereof,
wherein:
R¹ is R³, NHR³, —NHC(O)—R³, or —NH—SO₂—R³;
R² is H, R⁴, OH, OR⁴, NH₂, or —NHR⁴;
R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
wherein two R′ or two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C1-2 alkoxy, or CN;
R^{AA1} and R^{AA2} are each independently selected from H and C₁₋₆ alkyl optionally substituted with one or two groups independently selected from —OR⁵, —N(R⁵)₂, —SR⁵, —SeR⁵, —COOR⁵, CON(R⁵)₂, —NR⁵—C(═NR⁵)—N(R⁵)₂, phenyl, imidazolyl, and indolyl, where phenyl, imidazolyl and indolyl are each optionally substituted with halo, C₁₋₃ alkyl, C₁₋₃ haloalkyl, —OH, C₁₋₃ alkoxy, CN, COOR⁵, or CON(R⁵)₂;
   each R⁵ is independently selected from H and C₁₋₂ alkyl;
and Z is —COOH, CONH₂, or an amino acid or polypeptide that is optionally attached to a carrier or surface; or a salt thereof.

The compounds of Formula (II) are especially useful intermediates in the methods described herein, because they readily undergo an internal cyclization at the functionalized N-terminal amino acid (NTAA) under mild conditions at pH about 5-10, which results in cleavage of the NTAA. The invention further provides two ways to make these compounds under mild conditions: both the formation of compounds of Formula (II) and the elimination of the NTAA from compounds of Formula (II) occur under mild conditions that do not cause degradation of a nucleic acid in the same medium with the polypeptide. This is important for some of the methods described herein, where the polypeptide of interest may be mixed with or conjugated to a nucleic acid that serves as a recording tag to capture information about the NTAA being removed at each step.

The invention further provides polypeptide compounds of Formula (IV) as further described herein, which are useful activated forms of a polypeptide that can be prepared under very mild and selective conditions, and can be further modified to undergo NTAA elimination or cleavage under mild conditions. For example, the invention provides compounds of Formula (IV)

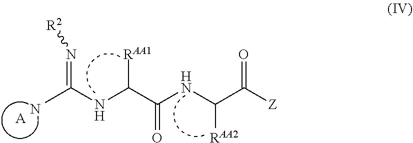

wherein:
R² is H, R⁴, OH, OR⁴, NH₂, or —NHR⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
wherein two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;

wherein each R is independently selected from H and C₁₋₃ alkyl optionally substituted with OH, OR*, —NH₂, —NHR*, or —NR*₂; and

each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
R^{AA1} and R^{AA2} are each independently selected amino acid side chains;
   and the dashed semi-circle connecting R^{AA1} and/or R^{AA2} to the nearest N atom indicates that R^{AA1} and/or R^{AA2} can optionally cyclize onto the designated N atom; and
Z is —COOH, CONH₂, or an amino acid or a polypeptide that is optionally attached to a carrier or solid support;
   or a salt thereof.

In another aspect, the invention provides a method to identify the N-terminal amino acid of a polypeptide by cleaving or selectively cleaving the NTAA from the polypeptide. This can be done using the methods herein under surprisingly mild conditions, which are compatible with the presence of acid-sensitive materials such as polynucleotides. This feature is especially valuable because, as further disclosed herein, polynucleotides may be present in samples of polypeptides of interest, and may even be conjugated to the polypeptide for various purposes. For example, the invention provides a method to identify the N-terminal amino acid residue of a peptidic compound of the Formula (I):

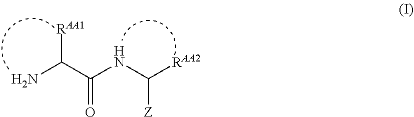

wherein the method comprises:
(1) converting the compound of Formula (I) to a guanidinyl derivative of Formula (II) or a tautomer thereof:

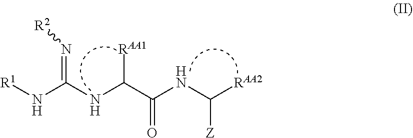

wherein:
R¹, NHR³, —NHC(O)—R³, or —NH—SO₂—R³
R² is H, R⁴, OH, OR⁴, NH₂, or NHR⁴;
R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
      where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
wherein two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
R^{AA1} and R^{AA2} are each independently selected amino acid side chains;
   and the dashed semi-circle connecting R^{AA1} and/or R^{AA2} to the nearest N atom indicates that R^{AA1} and/or R^{AA2} can optionally cyclize onto the designated N atom; and
and Z is —COOH, CONH₂, or an amino acid or polypeptide that is optionally attached to a carrier or surface;
(2) contacting the guanidinyl derivative with a suitable medium to induce elimination of the modified N-terminal amino acid and produce at least one cleavage product selected from:

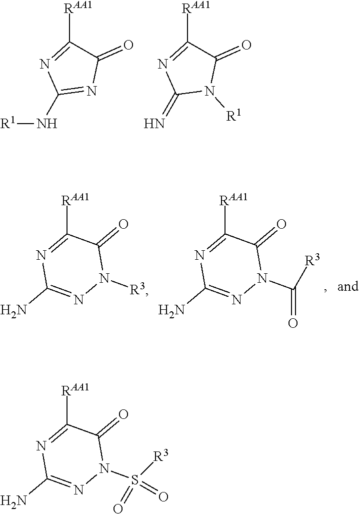

(when R¹ is NHR³, —NHC(O)—R³, or —NH—SO₂—R³, respectively) or a tautomer thereof; and
(3) determining the structure or identity of the at least one cleavage product to identify the N-terminal amino acid of the compound of Formula (I).

Provided in some aspects are methods for analyzing a polypeptide, comprising the steps of: (a) providing the polypeptide optionally associated directly or indirectly with a recording tag; (b) functionalizing the N-terminal amino acid (NTAA) of the polypeptide with a chemical reagent as further described herein; (c) contacting the polypeptide with a first binding agent comprising a first binding portion capable of binding to the functionalized NTAA and (c1) a first coding tag with identifying information regarding the first binding agent, or (c2) a first detectable label; and (d) (d1) transferring the information of the first coding tag to the recording tag to generate an extended recording tag and analyzing the extended recording tag, or (d2) detecting the first detectable label. In some embodiments, step (a) comprises providing the polypeptide and an associated recording tag joined to a support (e.g., a solid support).

For example, the invention provides a method for analyzing a polypeptide, comprising the steps of:

(a) providing the polypeptide optionally associated directly or indirectly with a recording tag;

(b) functionalizing the N-terminal amino acid (NTAA) of the polypeptide with a chemical reagent, wherein the chemical reagent is selected from:
(b1) a compound of Formula (AA):

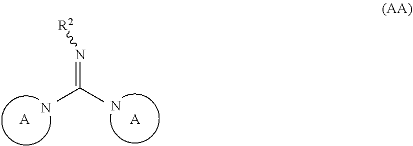

wherein:

R² is H or R⁴;

R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
wherein two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;

or ring A a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, B(OR)₂, Bpin (boranyl pinacolate), phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN; or
   (b2) a compound of the formula R³—NCS;

wherein R³ is H or an optionally substituted group selected from phenyl, 5-membered heteroaryl, 6-membered heteroaryl, C₁₋₃ haloalkyl, and C₁₋₆ alkyl,
wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;

wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

to provide an initial NTAA functionalized polypeptide;

optionally treating the initial NTAA functionalized polypeptide with an amine of Formula R²—NH₂ or with a diheteronucleophile to form a secondary NTAA functionalized polypeptide;

and optionally treating the initial NTAA functionalized polypeptide or the secondary NTAA functionalized polypeptide with a suitable medium to eliminate the NTAA and form an N-terminally truncated polypeptide;

(c) contacting the polypeptide with a first binding agent comprising a first binding portion capable of binding to the polypeptide, or to the initial NTAA functionalized polypeptide, or to the secondary NTAA functionalized polypeptide, or to the N-terminally truncated polypeptide; and either
(c1) a first coding tag with identifying information regarding the first binding agent, or
(c2) a first detectable label;

(d) (d1) transferring the information of the first coding tag, if present, to the recording tag to generate an extended recording tag and analyzing the extended recording tag, or
(d2) detecting the first detectable label, if present.

In some embodiments, step (a) comprises providing the polypeptide joined to an associated recording tag in a solution. In some embodiments, step (a) comprises providing the polypeptide associated indirectly with a recording tag. In some embodiments, the polypeptide is not associated with a recording tag in step (a). In one embodiment, the recording tag and/or the polypeptide are configured to be immobilized directly or indirectly to a support. In a further embodiment, the recording tag is configured to be immobilized to the support, thereby immobilizing the polypeptide associated with the recording tag. In another embodiment, the polypeptide is configured to be immobilized to the support, thereby immobilizing the recording tag associated with the polypeptide. In yet another embodiment, each of the recording tag and the polypeptide is configured to be immobilized to the support. In still another embodiment, the recording tag and the polypeptide are configured to co-localize when both are immobilized to the support. In some embodiments, the distance between (i) a polypeptide and (ii) a recording tag for information transfer between the recording tag and the coding tag of a binding agent bound to the polypeptide, is less than about 10⁻⁶ nm, about 10⁻⁶ nm, about 10⁻⁵ nm, about 10⁻⁴ nm, about 0.001 nm, about 0.01 nm, about 0.1 nm, about 0.5 nm, about 1 nm, about 2 nm, about 5 nm, or more than about 5 nm, or of any value in between the above ranges.

In another aspect, the invention provides kits for practicing the methods described herein. For example, the invention provides a kit for analyzing a polypeptide, which includes determining the NTAA of the polypeptide or determining at least a part of the amino acid sequence of the polypeptide, starting with the N-terminal amino acid. In one aspect, the invention provides such a kit comprising:

(a) a reagent for functionalizing the N-terminal amino acid (NTAA) of the polypeptide, wherein the reagent comprises a compound of the formula (AA):

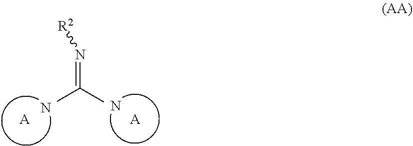

wherein Ring A is selected from:

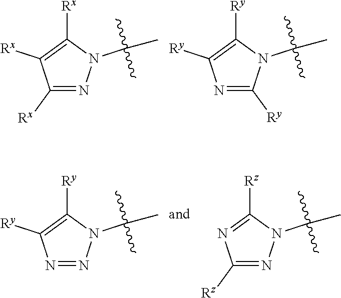

wherein:

each R^{x}, R^{y} and R^{z} is independently selected from H, halo, C₁₋₂ alkyl, C₁₋₂ haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂ haloalkyl, NO₂, SO₂(C₁₋₂ alkyl), COOR^{#}, and C(O)N(R^{#})₂,

and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;

wherein each R^{#} is independently H or C₁₋₂ alkyl; and wherein two R# on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;

(b) a plurality of binding agents, each comprising a binding portion capable of binding to the NTAA of a polypeptide either before or after the NTAA is functionalized by reaction with the compound of Formula (AA); and
(b1) a coding tag with identifying information regarding the binding agent, or
(b2) a detectable label; and

(c) a reagent for transferring the information of the first coding tag to the recording tag to generate an extended recording tag; and optionally

(d) a reagent for analyzing the extended recording tag or a reagent for detecting the first detectable label.

Provided herein are binding agents comprising a binding portion capable of binding to the N-terminal portion of a modified polypeptide, e.g., a polypeptide treated with any of the reagents provided for functionalizing the N-terminal amino acid (NTAA) of the polypeptide. In some aspects, a kit comprising a plurality of binding agents are provided.

Further aspects and embodiments of the invention are described in the detailed description and Examples that follow.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1A illustrates key for functional elements shown in the figures. Thus in one embodiment, provided herein is a recording tag or an extended recording tag, comprising one or more universal primer sequences (or one or more pairs of universal primer sequences, for example, one universal prime of the pair at the 5′ end and the other of the pair at the 3′ end of the recording tag or extended recording tag), one or more barcode sequences that can identify the recording tag or extended recording tag among a plurality of recording tags or extended recording tags, one or more UMI sequences, one or more spacer sequences, and/or one or more encoder sequences (also referred to as the coding sequence, e.g., of a coding tag). In certain embodiments, the extended recording tag comprises (i) one universal primer sequence, one barcode sequence, one UMI sequence, and one spacer (all from the unextended recording tag), (ii) one or more “cassettes” arranged in tandem, each cassette comprising an encoder sequence for a binding agent, a UMI sequence, and a spacer, and each cassette comprises sequence information from a coding tag, and (iii) another universal primer sequence, which may be provided by the coding tag of the coding agent in the n^{th} binding cycle, where n is an integer representing the number of binding cycle after which assay read out is desired. In one embodiment, after a universal primer sequence is introduced into an extended recoding tag, the binding cycles may continue, the extended recording tag may be further extended, and one or more additional universal primer sequences may be introduced. In that case, amplification and/or sequencing of the extended recording tag may be done using any combination of the universal primer sequences. FIG. 1B illustrates a general overview of transducing or converting a protein code to a nucleic acid (e.g., DNA) code where a plurality of proteins or polypeptides are fragmented into a plurality of peptides, which are then converted into a library of extended recording tags, representing the plurality of peptides. The extended recording tags constitute a DNA Encoded Library (DEL) representing the peptide sequences. The library can be appropriately modified to sequence on any Next Generation Sequencing (NGS) platform.

FIGS. 1C-1D illustrate examples of methods for recording tag encoded polypeptide analysis. FIG. 1C illustrates a method wherein (i) the nucleotide-peptide conjugate is captured on a solid surface; (ii) the NTAA is functionalized with a chemical reagent such as a compound of Formula (AA) or R³—NCS as described herein; (iii) a recognition element with a coding tag anchors to the substrate; (iv) the coding tag information is transferred to the recording tag using extension; and (v) the NTAA is eliminated. Cycles of steps (ii)-(v) can be repeated for multiple amino acids in the polypeptide. FIG. 1D illustrates a method wherein (i) the nucleotide-peptide conjugate is captured on a solid surface; (ii) a recognition element with a coding tag anchors to the substrate; (iii) the coding tag information is transferred to the recording tag using extension; (iv) the NTAA is functionalized with a chemical reagent such as a compound of Formula (AA) or R³—NCS as described herein; and (v) the NTAA is eliminated. Cycles of steps (ii)-(v) can be repeated for multiple amino acids in the polypeptide.

FIGS. 1E-1F illustrate examples of methods of polypeptide analysis using an alternative detection method. In the method described in FIG. 1E, (i) the peptide is captured on a solid surface; (ii) the NTAA is functionalized with a chemical reagent such as a compound of Formula (AA) or R³—NCS as described herein; (iii) a recognition element with detection element, such as a fluorophore, anchors to the substrate; (iv) the detection element is detected; and (v) the NTAA is eliminated. Cycles of steps (ii)-(v) can be repeated for multiple amino acids in the polypeptide. FIG. 1F shows a method in which (i) the peptide is captured on a solid surface; (ii) a recognition element with detection element, such as a fluorophore, anchors to the substrate; (iii) the detection element is detected; (iv) the NTAA is functionalized with reagents akin to Formulas I-VII; and (v) the NTAA is eliminated. Cycles of steps (ii)-(v) can be repeated for multiple amino acids in the polypeptide.

FIG. 1G illustrates methods used for nucleic acid screening. (A) shows an example of the solid phase screening for nucleotide reactivity detailed herein. A surface anchored oligonucleotide is treated with a chemical reagent such as a compound of Formula (AA) or R³—NCS as described herein. After which the oligonucleotide is cleaved and subjected to mass analysis. (B) shows drawings of “no reaction” (left) and “reaction detected” (right).

FIG. 1H illustrates an example of a method of a single cycle of recording tag encoded polypeptide analysis using ligation elements detailed herein. In this method, (i) the nucleotide-peptide conjugate is captured on a solid surface; (ii) the NTAA is functionalized with a chemical reagent which comprises a ligand that is capable of forming a covalent bond such as a compound of Formula (AA)-Q as described herein, wherein Q is a ligand that is capable of forming a covalent bond (e.g., with a binding agent); (iii) a recognition element with a coding tag anchors to the substrate; (iv) a reaction, spontaneous or stimulated, is initiated ligating the recognition element to the polypeptide; (v) the coding tag information is transferred to the recording tag using extension; and (vi) the NTAA-Recognition element complex is eliminated.

FIGS. 2A-2D illustrate an example of polypeptide analysis according to the methods disclosed herein, using multiple cycles of binding agents (e.g., antibodies, anticalins, N-recognins proteins (e.g., ATP-dependent Clp protease adaptor protein (ClpS)), aptamers, etc. and variants/homologues thereof) comprising coding tags interacting with an immobilized protein that is co-localized or co-labeled with a single or multiple recording tags. In this example, the recording tag is comprised of a universal priming site, a barcode (e.g., partition barcode, compartment barcode, and/or fraction barcode), an optional unique molecular identifier (UMI) sequence, and optionally a spacer sequence (Sp) used in information transfer between the coding tag and the recording tag (or an extended recording tag). The spacer sequence (Sp) can be constant across all binding cycles, be binding agent specific, and/or be binding cycle number specific (e.g., used for “clocking” the binding cycles). In this example, the coding tag comprises an encoder sequence providing identifying information for the binding agent (or a class of binding agents, for example, a class of binders that all specifically bind to a terminal amino acid, such as a modified N-terminal Q as shown in FIG. 3), an optional UMI, and a spacer sequence that hybridizes to the complementary spacer sequence on the recording tag, facilitating transfer of coding tag information to the recording tag (e.g., by primer extension, also referred to herein as polymerase extension). Ligation may also be used to transfer sequence information and in that case, a spacer sequence may be used but is not necessary.

FIGS. 2A-2D illustrate an example of polypeptide analysis according to the methods disclosed herein, using multiple cycles of binding agents (e.g., antibodies, anticalins, N-recognins proteins (e.g., ATP-dependent Clp protease adaptor protein (ClpS)), aptamers, etc. and variants/homologues thereof) comprising coding tags interacting with an immobilized protein that is co-localized or co-labeled with a single or multiple recording tags. In this example, the recording tag is comprised of a universal priming site, a barcode (e.g., partition barcode, compartment barcode, and/or fraction barcode), an optional unique molecular identifier (UMI) sequence, and optionally a spacer sequence (Sp) used in information transfer between the coding tag and the recording tag (or an extended recording tag). The spacer sequence (Sp) can be constant across all binding cycles, be binding agent specific, and/or be binding cycle number specific (e.g., used for “clocking” the binding cycles). In this example, the coding tag comprises an encoder sequence providing identifying information for the binding agent (or a class of binding agents, for example, a class of binders that all specifically bind to a terminal amino acid, such as a modified N-terminal Q as shown in FIG. 3), an optional UMI, and a spacer sequence that hybridizes to the complementary spacer sequence on the recording tag, facilitating transfer of coding tag information to the recording tag (e.g., by primer extension, also referred to herein as polymerase extension). Ligation may also be used to transfer sequence information and in that case, a spacer sequence may be used but is not necessary.

FIG. 2A illustrates a process of creating an extended recording tag through the cyclic binding of cognate binding agents to a polypeptide (such as a protein or protein complex), and corresponding information transfer from the binding agent's coding tag to the polypeptide's recording tag. After a series of sequential binding and coding tag information transfer steps, the final extended recording tag is produced, containing binding agent coding tag information including encoder sequences from “n” binding cycles providing identifying information for the binding agents (e.g., antibody 1 (Ab1), antibody 2 (Ab2), antibody 3 (Ab3), . . . antibody “n” (Abn)), a barcode/optional UMI sequence from the recording tag, an optional UMI sequence from the binding agent's coding tag, and flanking universal priming sequences at each end of the library construct to facilitate amplification and/or analysis by digital next-generation sequencing.

FIG. 2B illustrates an example of a scheme for labeling a protein with DNA barcoded recording tags. In the top panel, N-hydroxysuccinimide (NHS) is an amine reactive functional group, and Dibenzocyclooctyl (DBCO) is a strained alkyne useful in “click” coupling to the surface of a solid substrate. In this scheme, the recording tags are coupled to ε amines of lysine (K) residues (and optionally N-terminal amino acids) of the protein via NHS moieties. In the bottom panel, a heterobifunctional linker, NHS-alkyne, is used to label the c amines of lysine (K) residues to create an alkyne “click” moiety. Azide-labeled DNA recording tags can then easily be attached to these reactive alkyne groups via standard click chemistry. Moreover, the DNA recording tag can also be designed with an orthogonal methyltetrazine (e.g., mTet or pTet) moiety for downstream coupling to a trans-cyclooctene (TCO)-derivatized sequencing substrate via an inverse Electron Demand Diels-Alder (iEDDA) reaction.

FIG. 2C illustrates two examples of the protein analysis methods using recording tags. In the top panel, polypeptides are immobilized on a solid support via a capture agent and optionally cross-linked. Either the protein or capture agent may co-localize or be labeled with a recording tag. In the bottom panel, proteins with associated recording tags are directly immobilized on a solid support.

FIG. 2D illustrates an example of an overall workflow for a simple protein immunoassay using DNA encoding of cognate binders and sequencing of the resultant extended recording tag. The proteins can be sample barcoded (i.e., indexed) via recording tags and pooled prior to cyclic binding analysis, greatly increasing sample throughput and economizing on binding reagents. This approach is effectively a digital, simpler, and more scalable approach to performing reverse phase protein assays (RPPA), allowing measurement of protein levels (such as expression levels) in a large number of biological samples simultaneously in a quantitative manner.

FIGS. 3A-D illustrate a process for a degradation-based polypeptide sequencing assay by construction of an extended recording tag (e.g., DNA sequence) representing the polypeptide sequence. This is accomplished through an Edman degradation-like approach using a cyclic process such as terminal amino acid functionalization (e.g., N-terminal amino acid (NTAA) functionalization), coding tag information transfer to a recording tag attached to the polypeptide, terminal amino acid elimination (e.g., NTAA elimination), and repeating the process in a cyclic manner, for example, all on a solid support. Provided is an overview of an exemplary construction of an extended recording tag from N-terminal degradation of a peptide: (A) N-terminal amino acid of a polypeptide is functionalized (e.g., with a phenylthiocarbamoyl (PTC), dinitrophenyl (DNP), sulfonyl nitrophenyl (SNP), acetyl, or guanidinyl moiety); (B) shows a binding agent and an associated coding tag bound to the functionalized NTAA; (C) shows the polypeptide bound to a solid support (e.g., bead) and associated with a recording tag (e.g., via a trifunctional linker), wherein upon binding of the binding agent to the NTAA of the polypeptide, information of the coding tag is transferred to the recording tag (e.g., via primer extension) to generate an extended recording tag; (D) the functionalized NTAA is eliminated via chemical or biological (e.g., enzymatic) means to expose a new NTAA. As illustrated by the arrows, the cycle is repeated “n” times to generate a final extended recording tag. The final extended recording tag is optionally flanked by universal priming sites to facilitate downstream amplification and/or DNA sequencing. The forward universal priming site (e.g., Illumina's P5-S1 sequence) can be part of the original recording tag design and the reverse universal priming site (e.g., Illumina's P7-S2′ sequence) can be added as a final step in the extension of the recording tag. This final step may be done independently of a binding agent. In some embodiments, the order in the steps in the process for a degradation-based peptide polypeptide sequencing assay can be reversed or moved around. For example, in some embodiments, the terminal amino acid functionalization of step (A) can be conducted after the polypeptide is bound to the binding agent and/or associated coding tag (step (B)). In some embodiments, the terminal amino acid functionalization of step (A) can be conducted after the polypeptide is bound a support (step (C)).

FIGS. 4A-B illustrate exemplary protein sequencing workflows according to the methods disclosed herein. FIG. 4A illustrates exemplary work flows with alternative modes outlined in light grey dashed lines, with a particular embodiment shown in boxes linked by arrows. Alternative modes for each step of the workflow are shown in boxes below the arrows. FIG. 4B illustrates options in conducting a cyclic binding and coding tag information transfer step to improve the efficiency of information transfer. Multiple recording tags per molecule can be employed. Moreover, for a given binding event, the transfer of coding tag information to the recording tag can be conducted multiples times, or alternatively, a surface amplification step can be employed to create copies of the extended recording tag library, etc.

FIGS. 5A-B illustrate an overview of an exemplary construction of an extended recording tag using primer extension to transfer identifying information of a coding tag of a binding agent to a recording tag associated with a polypeptide to generate an extended recording tag. A coding tag comprising a unique encoder sequence with identifying information regarding the binding agent is optionally flanked on each end by a common spacer sequence (Sp′). FIG. 5A illustrates an NTAA binding agent comprising a coding tag binding to an NTAA of a polypeptide which is labeled with a recording-tag and linked to a bead. The recording tag anneals to the coding tag via complementary spacer sequences (Sp anneals to Sp′), and a primer extension reaction mediates transfer of coding tag information to the recording tag using the spacer (Sp) as a priming site. The coding tag is illustrated as a duplex with a single stranded spacer (Sp′) sequence at the terminus distal to the binding agent. This configuration minimizes hybridization of the coding tag to internal sites in the recording tag and favors hybridization of the recording tag's terminal spacer (Sp) sequence with the single stranded spacer overhang (Sp′) of the coding tag. Moreover, the extended recording tag may be pre-annealed with one or more oligonucleotides (e.g., complementary to an encoder and/or spacer sequence) to block hybridization of the coding tag to internal recording tag sequence elements. FIG. 5B shows a final extended recording tag produced after “n” cycles of binding (“***” represents intervening binding cycles not shown in the extended recording tag) and transfer of coding tag information and the addition of a universal priming site at the 3′-end.

FIG. 6 illustrates coding tag information being transferred to an extended recording tag via enzymatic ligation. Two different polypeptides are shown with their respective recording tags, with recording tag extension proceeding in parallel. Ligation can be facilitated by designing the double stranded coding tags so that the spacer sequences (Sp′) have a “sticky end” overhang on one strand that anneals with a complementary spacer (Sp) on the recording tag. The complementary strand of the double stranded coding tag, after being ligated to the recording tag, transfers information to the recording tag. The complementary strand may comprise another spacer sequence, which may be the same as or different from the Sp of the recording tag before the ligation. When ligation is used to extend the recording tag, the direction of extension can be 5′ to 3′ as illustrated, or optionally 3′ to 5′.

FIG. 7 illustrates a “spacer-less” approach of transferring coding tag information to a recording tag via chemical ligation to link the 3′ nucleotide of a recording tag or extended recording tag to the 5′ nucleotide of the coding tag (or its complement) without inserting a spacer sequence into the extended recording tag. The orientation of the extended recording tag and coding tag could also be inverted such that the 5′ end of the recording tag is ligated to the 3′ end of the coding tag (or complement). In the example shown, hybridization between complementary “helper” oligonucleotide sequences on the recording tag (“recording helper”) and the coding tag are used to stabilize the complex to enable specific chemical ligation of the recording tag to coding tag complementary strand. The resulting extended recording tag is devoid of spacer sequences. Also illustrated is a “click chemistry” version of chemical ligation (e.g., using azide and alkyne moieties (shown as a triple line symbol)) which can employ DNA, PNA, or similar nucleic acid polymers.

FIGS. 8A-B illustrate an exemplary method of writing of post-translational modification (PTM) information of a peptide into an extended recording tag prior to N-terminal amino acid degradation. FIG. 8A: A binding agent comprising a coding tag with identifying information regarding the binding agent (e.g., a phosphotyrosine antibody comprising a coding tag with identifying information for phosphotyrosine antibody) is capable of binding to the peptide. If phosphotyrosine is present in the recording tag-labeled peptide, as illustrated, upon binding of the phosphotyrosine antibody to phosphotyrosine, the coding tag and recording tag anneal via complementary spacer sequences and the coding tag information is transferred to the recording tag to generate an extended recording tag. FIG. 8B: An extended recording tag may comprise coding tag information for both primary amino acid sequence (e.g., “aa₁”, “aa₂”, “aa₃”, . . . , “aa_{N}”) and post-translational modifications (e.g., “PTM₁”, “PTM₂”) of the peptide.

FIGS. 9A-B illustrate a process of multiple cycles of binding of a binding agent to a polypeptide and transferring information of a coding tag that is attached to a binding agent to an individual recording tag among a plurality of recording tags, for example, which are co-localized at a site of a single polypeptide attached to a solid support (e.g., a bead), thereby generating multiple extended recording tags that collectively represent the polypeptide information (e.g., presence or absence, level, or amount in a sample, binding profile to a library of binders, activity or reactivity, amino acid sequence, post-translational modification, sample origin, or any combination thereof). In this figure, for purposes of example only, each cycle involves binding a binding agent to an N-terminal amino acid (NTAA) of the polypeptide, recording the binding event by transferring coding tag information to a recording tag, followed by removal of the NTAA to expose a new NTAA. FIG. 9A illustrates on a solid support a plurality of recording tags (e.g., comprising universal forward priming sequence and a UMI) which are available to a binding agent bound to the polypeptide. Individual recording tags possess a common spacer sequence (Sp) complementary to a common spacer sequence within coding tags of binding agents, which can be used to prime an extension reaction to transfer coding tag information to a recording tag. For example, the plurality of recording tags may co-localize with the polypeptide on the support, and some of the recording tags may be closer to the analyte than others. In one aspect, the density of recording tags relative to the polypeptide density on the support may be controlled, so that statistically each polypeptide will have a plurality of recording tags (e.g., at least about two, about five, about ten, about 20, about 50, about 100, about 200, about 500, about 1000, about 2000, about 5000, or more) available to a binding agent bound to that polypeptide. This mode may be particularly useful for analyzing low abundance proteins or polypeptides in a sample. Although FIG. 9A shows a different recording tag is extended in each of Cycles 1-3 (e.g., a cycle-specific barcode in the binding agent or separately added in each binding/reaction cycle may be used to “clock” the binding/reactions), it is envisaged that an extended recording tag may be further extended in any one or more of subsequent binding cycles, and the resultant pool of extended recording tags may be a mix of recording tags that are extended only once, twice, three times, or more.

FIG. 9B illustrates different pools of cycle-specific NTAA binding agents that are used for each successive cycle of binding, each pool having a cycle specific sequence, such as a cycle specific spacer sequence. Alternatively, the cycle specific sequence may be provided in a reagent separate from the binding agents.

FIGS. 10A-C illustrate an exemplary mode comprising multiple cycles of transferring information of a coding tag that is attached to a binding agent to a recording tag among a plurality of recording tags co-localized at a site of a single polypeptide attached to a solid support (e.g., a bead), thereby generating multiple extended recording tags that collectively represent the polypeptide. In this figure, for purposes of example only, the polypeptide is a peptide and each round of processing involves binding to an NTAA, recording the binding event, followed by removal of the NTAA to expose a new NTAA. FIG. 10A illustrates a plurality of recording tags (comprising a universal forward priming sequence and a UMI) co-localized on a solid support with the polypeptide, preferably a single molecule per bead. Individual recording tags possess different spacer sequences at their 3′-end with different “cycle specific” sequences (e.g., C₁, C₂, C₃, . . . Cₙ). Preferably, the recording tags on each bead share the same UMI sequence. In a first cycle of binding (Cycle 1), a plurality of NTAA binding agents is contacted with the polypeptide. The binding agents used in Cycle 1 possess a common 5′-spacer sequence (C′1) that is complementary to the Cycle 1 C₁ spacer sequence of the recording tag. The binding agents used in Cycle 1 also possess a 3′-spacer sequence (C′2) that is complementary to the Cycle 2 spacer C₂. During binding Cycle 1, a first NTAA binding agent binds to the free N-terminus of the polypeptide, and the information of a first coding tag is transferred to a cognate recording tag via primer extension from the C₁ sequence hybridized to the complementary C′₁ spacer sequence. Following removal of the NTAA to expose a new NTAA, binding Cycle 2 contacts a plurality of NTAA binding agents that possess a Cycle 2 5′-spacer sequence (C′₂) that is identical to the 3′-spacer sequence of the Cycle 1 binding agents and a common Cycle 3 3′-spacer sequence (C′₃), with the polypeptide. A second NTAA binding agent binds to the NTAA of the polypeptide, and the information of a second coding tag is transferred to a cognate recording tag via primer extension from the complementary C₂ and C′₂ spacer sequences. These cycles are repeated up to “n” binding cycles, wherein the last extended recording tag is capped with a universal reverse priming sequence, generating a plurality of extended recording tags co-localized with the single polypeptide, wherein each extended recording tag possesses coding tag information from one binding cycle. Because each set of binding agents used in each successive binding cycle possess cycle specific spacer sequences in the coding tags, binding cycle information can be associated with binding agent information in the resulting extended recording tags. FIG. 10B illustrates different pools of cycle-specific binding agents that are used for each successive cycle of binding, each pool having cycle specific spacer sequences. FIG. 10C illustrates how the collection of extended recording tags (e.g., that are co-localized at the site of the polypeptide) can be assembled in a sequential order based on PCR assembly of the extended recording tags using cycle specific spacer sequences, thereby providing an ordered sequence of the polypeptide. In some embodiments, multiple copies of each extended recording tag are generated via amplification prior to concatenation.

FIGS. 11A-B illustrate information transfer from recording tag to a coding tag or di-tag construct. Two methods of recording binding information are illustrated in (A) and (B). A binding agent may be any type of binding agent as described herein; an anti-phosphotyrosine binding agent is shown for illustration purposes only. For extended coding tag or di-tag construction, rather than transferring binding information from the coding tag to the recording tag, information is either transferred from the recording tag to the coding tag to generate an extended coding tag (FIG. 11A), or information is transferred from both the recording tag and coding tag to a third di-tag-forming construct (FIG. 11B). The di-tag and extended coding tag comprise the information of the recording tag (containing a barcode, an optional UMI sequence, and an optional compartment tag (CT) sequence (not illustrated)) and the coding tag. The di-tag and extended coding tag can be eluted from the recording tag, collected, and optionally amplified and read out on a next generation sequencer.

FIGS. 12A-D illustrate design of PNA combinatorial barcode/UMI recording tag and di-tag detection of binding events. In FIG. 12A, the construction of a combinatorial PNA barcode/UMI via chemical ligation of four elementary PNA word sequences (A, A′-B, B′-C, and C′) is illustrated. Hybridizing DNA arms are included to create a spacer-less combinatorial template for combinatorial assembly of a PNA barcode/UMI. Chemical ligation is used to stitch the annealed PNA “words” together. FIG. 12B shows a method to transfer the PNA information of the recording tag to a DNA intermediate. The DNA intermediate is capable of transferring information to the coding tag. Namely, complementary DNA word sequences are annealed to the PNA and chemically ligated (optionally enzymatically ligated if a ligase is discovered that uses a PNA template). In FIG. 12C, the DNA intermediate is designed to interact with the coding tag via a spacer sequence, Sp. A strand-displacing primer extension step displaces the ligated DNA and transfers the recording tag information from the DNA intermediate to the coding tag to generate an extended coding tag. A terminator nucleotide may be incorporated into the end of the DNA intermediate to prevent transfer of coding tag information to the DNA intermediate via primer extension. FIG. 12D: Alternatively, information can be transferred from coding tag to the DNA intermediate to generate a di-tag construct. A terminator nucleotide may be incorporated into the end of the coding tag to prevent transfer of recording tag information from the DNA intermediate to the coding tag.

FIGS. 13A-E illustrate proteome partitioning on a compartment barcoded bead, and subsequent di-tag assembly via emulsion fusion PCR to generate a library of elements representing peptide sequence composition. The amino acid content of the peptide can be subsequently characterized through N-terminal sequencing or alternatively through attachment (covalent or non-covalent) of amino acid specific chemical labels or binding agents associated with a coding tag. The coding tag comprises a universal priming sequence, as well as an encoder sequence for the amino acid identity, a compartment tag, and an amino acid UMI. After information transfer, the di-tags are mapped back to the originating molecule via the recording tag UMI. In FIG. 13A, the proteome is compartmentalized into droplets with barcoded beads. Peptides with associated recording tags (comprising compartment barcode information) are attached to the bead surface. The droplet emulsion is broken releasing barcoded beads with partitioned peptides. In FIG. 13B, specific amino acid residues on the peptides are chemically labeled with DNA coding tags that are conjugated to site-specific labeling moieties. The DNA coding tags comprise amino acid barcode information and optionally an amino acid UMI. FIG. 13C: Labeled peptide-recording tag complexes are released from the beads. FIG. 13D: The labeled peptide-recording tag complexes are emulsified into nano or microemulsions such that there is, on average, less than one peptide-recording tag complex per compartment. FIG. 13E: An emulsion fusion PCR transfers recording tag information (e.g., compartment barcode) to all of the DNA coding tags attached to the amino acid residues.

FIG. 14 illustrates generation of extended coding tags from emulsified peptide recording tag-coding tags complex. The peptide complexes from FIG. 13C are co-emulsified with PCR reagents into droplets with on average a single peptide complex per droplet. A three-primer fusion PCR approach is used to amplify the recording tag associated with the peptide, fuse the amplified recording tags to multiple binding agent coding tags or coding tags of covalently labeled amino acids, extend the coding tags via primer extension to transfer peptide UMI and compartment tag information from the recording tag to the coding tag, and amplify the resultant extended coding tags. There are multiple extended coding tag species per droplet, with a different species for each amino acid encoder sequence-UMI coding tag present. In this way, both the identity and count of amino acids within the peptide can be determined. The U1 universal primer and Sp primer are designed to have a higher melting Tm than the U2ₜᵣ universal primer. This enables a two-step PCR in which the first few cycles are performed at a higher annealing temperature to amplify the recording tag, and then stepped to a lower Tm so that the recording tags and coding tags prime on each other during PCR to produce an extended coding tag, and the U1 and U2ₜᵣ universal primers are used to prime amplification of the resultant extended coding tag product. In certain embodiments, premature polymerase extension from the U2ₜᵣ primer can be prevented by using a photo-labile 3′ blocking group (Young et al., 2008, Chem. Commun. (Camb) 4:462-464). After the first round of PCR amplifying the recording tags, and a second-round fusion PCR step in which the coding tag Spₜᵣ primes extension of the coding tag on the amplified Sp′ sequences of the recording tag, the 3′ blocking group of U2ₜᵣ is removed, and a higher temperature PCR is initiated for amplifying the extended coding tags with U1 and U2ₜᵣ primers.

FIG. 15 illustrates use of proteome partitioning and barcoding facilitating enhanced mappability and phasing of proteins. In polypeptide sequencing, proteins are typically digested into peptides. In this process, information about the relationship between individual polypeptides that originated from a parent protein molecule, and their relationship to the parent protein molecule is lost. In order to reconstruct this information, individual peptide sequences are mapped back to a collection of protein sequences from which they may have derived. The task of finding a unique match in such a set is rendered more difficult with short and/or partial peptide sequences, and as the size and complexity of the collection (e.g., proteome sequence complexity) increases. The partitioning of the proteome into barcoded (e.g., compartment tagged) compartments or partitions, subsequent digestion of the protein into peptides, and the joining of the compartment tags to the peptides reduces the “protein” space to which a peptide sequence needs to be mapped to, greatly simplifying the task in the case of complex protein samples. Labeling of a protein with unique molecular identifier (UMI) prior to digestion into peptides facilitates mapping of peptides back to the originating protein molecule and allows annotation of phasing information between post-translational modified (PTM) variants derived from the same protein molecule and identification of individual proteoforms. FIG. 15A shows an example of proteome partitioning comprising labeling proteins with recording tags comprising a partition barcode and subsequent fragmentation into recording-tag labeled peptides. FIG. 15B: For partial peptide sequence information or even just composition information, this mapping is highly-degenerate. However, partial peptide sequence or composition information coupled with information from multiple peptides from the same protein, allow unique identification of the originating protein molecule.

FIG. 16 illustrates exemplary modes of compartment tagged bead sequence design. The compartment tags comprise a barcode of X₅₋₂₀ to identify an individual compartment and a unique molecular identifier (UMI) of N₅₋₁₀ to identify the peptide to which the compartment tag is joined, where X and N represent degenerate nucleobases or nucleobase words (e.g., SEQ ID NO: 137). Compartment tags can be single stranded (upper depictions) or double stranded (lower depictions). Optionally, compartment tags can be a chimeric molecule comprising a peptide sequence with a recognition sequence for a protein ligase (e.g., butelase I; CGSNVH; SEQ ID NO: 138) for joining to a peptide of interest (left depictions). Alternatively, a chemical moiety can be included on the compartment tag for coupling to a peptide of interest (e.g., azide as shown in right depictions).

FIGS. 17A-B illustrate: (A) a plurality of extended recording tags representing a plurality of peptides; and (B) an exemplary method of target peptide enrichment via standard hybrid capture techniques. For example, hybrid capture enrichment may use one or more biotinylated “bait” oligonucleotides that hybridize to extended recording tags representing one or more peptides of interest (“target peptides”) from a library of extended recording tags representing a library of peptides. The bait oligonucleotide:target extended recording tag hybridization pairs are pulled down from solution via the biotin tag after hybridization to generate an enriched fraction of extended recording tags representing the peptide or peptides of interest. The separation (“pull down”) of extended recording tags can be accomplished, for example, using streptavidin-coated magnetic beads. The biotin moieties bind to streptavidin on the beads, and separation is accomplished by localizing the beads using a magnet while solution is removed or exchanged. A non-biotinylated competitor enrichment oligonucleotide that competitively hybridizes to extended recording tags representing undesirable or over-abundant peptides can optionally be included in the hybridization step of a hybrid capture assay to modulate the amount of the enriched target peptide. The non-biotinylated competitor oligonucleotide competes for hybridization to the target peptide, but the hybridization duplex is not captured during the capture step due to the absence of a biotin moiety. Therefore, the enriched extended recording tag fraction can be modulated by adjusting the ratio of the competitor oligonucleotide to the biotinylated “bait” oligonucleotide over a large dynamic range. This step will be important to address the dynamic range issue of protein abundance within the sample.

FIGS. 18A-B illustrate exemplary methods of single cell and bulk proteome partitioning into individual droplets, each droplet comprising a bead having a plurality of compartment tags attached thereto to correlate peptides to their originating protein complex, or to proteins originating from a single cell. The compartment tags comprise barcodes. Manipulation of droplet constituents after droplet formation: (A) Single cell partitioning into an individual droplet followed by cell lysis to release the cell proteome, and proteolysis to digest the cell proteome into peptides, and inactivation of the protease following sufficient proteolysis; (B) Bulk proteome partitioning into a plurality of droplets wherein an individual droplet comprises a protein complex followed by proteolysis to digest the protein complex into peptides, and inactivation of the protease following sufficient proteolysis. A heat labile metallo-protease can be used to digest the encapsulated proteins into peptides after photo-release of photo-caged divalent cations to activate the protease. The protease can be heat inactivated following sufficient proteolysis, or the divalent cations may be chelated. Droplets contain hybridized or releasable compartment tags comprising nucleic acid barcodes (separate from recording tag) capable of being ligated to either an N- or C-terminal amino acid of a peptide.

FIGS. 19A-B illustrate exemplary methods of single cell and bulk proteome partitioning into individual droplets, each droplet comprising a bead having a plurality of bifunctional recording tags with compartment tags attached thereto to correlate peptides to their originating protein or protein complex, or proteins to originating single cell. Manipulation of droplet constituents after post droplet formation: (A) Single cell partitioning into an individual droplet followed by cell lysis to release the cell proteome, and proteolysis to digest the cell proteome into peptides, and inactivation of the protease following sufficient proteolysis; (B) Bulk proteome partitioning into a plurality of droplets wherein an individual droplet comprises a protein complex followed by proteolysis to digest the protein complex into peptides, and inactivation of the protease following sufficient proteolysis. A heat labile metallo-protease can be used to digest the encapsulated proteins into peptides after photo-release of photo-caged divalent cations (e.g., Zn2+). The protease can be heat inactivated following sufficient proteolysis or the divalent cations may be chelated. Droplets contain hybridized or releasable compartment tags comprising nucleic acid barcodes (separate from recording tag) capable of being ligated to either an N- or C-terminal amino acid of a peptide.

FIGS. 20A-L illustrate generation of compartment barcoded recording tags attached to peptides. Compartment barcoding technology (e.g., barcoded beads in microfluidic droplets, etc.) can be used to transfer a compartment-specific barcode to molecular contents encapsulated within a particular compartment. (A) In a particular embodiment, the protein molecule is denatured, and the ε-amine group of lysine residues (K) is chemically conjugated to an activated universal DNA tag molecule (comprising a universal priming sequence (U1)), shown with NHS moiety at the 5′ end). After conjugation of universal DNA tags to the polypeptide, excess universal DNA tags are removed. (B) The universal DNA tagged-polypeptides are hybridized to nucleic acid molecules bound to beads, wherein the nucleic acid molecules bound to an individual bead comprise a unique population of compartment tag (barcode) sequences. The compartmentalization can occur by separating the sample into different physical compartments, such as droplets (illustrated by the dashed oval). Alternatively, compartmentalization can be directly accomplished by the immobilization of the labeled polypeptides on the bead surface, e.g., via annealing of the universal DNA tags on the polypeptide to the compartment DNA tags on the bead, without the need for additional physical separation. A single polypeptide molecule interacts with only a single bead (e.g., a single polypeptide does not span multiple beads). Multiple polypeptides, however, may interact with the same bead. In addition to the compartment barcode sequence (BC), the nucleic acid molecules bound to the bead may be comprised of a common Sp (spacer) sequence, a unique molecular identifier (UMI), and a sequence complementary to the polypeptide DNA tag, U1′. (C) After annealing of the universal DNA tagged polypeptides to the compartment tags bound to the bead, the compartment tags are released from the beads via cleavage of the attachment linkers. (D) The annealed U1 DNA tag primers are extended via polymerase-based primer extension using the compartment tag nucleic acid molecule originating from the bead as template. The primer extension step may be carried out after release of the compartment tags from the bead as shown in (C) or, optionally, while the compartment tags are still attached to the bead (not shown). This effectively writes the barcode sequence from the compartment tags on the bead onto the U1 DNA-tag sequence on the polypeptide. This new sequence constitutes a recording tag. After primer extension, a protease, e.g., Lys-C (cleaves on C-terminal side of lysine residues), Glu-C (cleaves on C-terminal side of glutamic acid residues and to a lower extent glutamic acid residues), or random protease such as Proteinase K, is used to cleave the polypeptide into peptide fragments. (E) Each peptide fragment is labeled with an extended DNA tag sequence constituting a recording tag on its C-terminal lysine for downstream peptide sequencing as disclosed herein. (F) The recording tagged peptides are coupled to azide beads through a strained alkyne label, DBCO. The azide beads optionally also contain a capture sequence complementary to the recording tag to facilitate the efficiency of DBCO-azide immobilization. It should be noted that removing the peptides from the original beads and re-immobilizing to a new solid support (e.g., beads) permits optimal intermolecular spacing between peptides to facilitate peptide sequencing methods as disclosed herein. FIG. 20G-L illustrates a similar concept as illustrated in FIGS. 20A-F except using click chemistry conjugation of DNA tags to an alkyne pre-labeled polypeptide (as described in FIG. 2B). The Azide and mTet chemistries are orthogonal allowing click conjugation to DNA tags and click iEDDA conjugation (mTet and TCO) to the sequencing substrate.

FIG. 21 illustrates an exemplary method using flow-focusing T-junction for single cell and compartment tagged (e.g., barcode) compartmentalization with beads. With two aqueous flows, cell lysis and protease activation (Zn²⁺ mixing) can easily be initiated upon droplet formation.

FIGS. 22A-B illustrate exemplary tagging details. (A) A compartment tag (DNA-peptide chimera) is attached onto the peptide using peptide ligation with Butelase I. (B) Compartment tag information is transferred to an associated recording tag prior to commencement of peptide sequencing. Optionally, an endopeptidase AspN, which selectively cleaves peptide bonds N-terminal to aspartic acid residues, can be used to cleave the compartment tag after information transfer to the recording tag.

FIGS. 23A-C: Array-based barcodes for a spatial proteomics-based analysis of a tissue slice. (A) An array of spatially-encoded DNA barcodes (feature barcodes denoted by BCᵢⱼ), is combined with a tissue slice (FFPE or frozen). In one embodiment, the tissue slice is fixed and permeabilized. In some embodiments, the array feature size is smaller than the cell size (˜10 μm for human cells). (B) The array-mounted tissue slice is treated with reagents to reverse cross-linking (e.g., antigen retrieval protocol w/citraconic anhydride (Namimatsu, Ghazizadeh et al. 2005), and then the proteins therein are labeled with site-reactive DNA labels, that effectively label all protein molecules with DNA recording tags (e.g., lysine labeling, liberated after antigen retrieval). After labeling and washing, the array bound DNA barcode sequences are cleaved and allowed to diffuse into the mounted tissue slice and hybridize to DNA recording tags attached to the proteins therein. (C) The array-mounted tissue is now subjected to polymerase extension to transfer information of the hybridized barcodes to the DNA recording tags labeling the proteins. After transfer of the barcode information, the array-mounted tissue is scraped from the slides, optionally digested with a protease, and the proteins or peptides extracted into solution.

FIGS. 24A-B illustrate two different exemplary DNA target polypeptides (AB and CD) that are immobilized on beads and assayed by binding agents attached to coding tags. This model system serves to illustrate the single molecule behavior of coding tag transfer from a bound agent to a proximal reporting tag. In some embodiments, the coding tags are incorporated into an extended recoding tag via primer extension. FIG. 24A illustrates the interaction of an AB polypeptide with an A-specific binding agent (“A′”, an oligonucleotide sequence complementary to the “A” component of the AB polypeptide) and transfer of information of an associated coding tag to a recording tag via primer extension, and a B-specific binding agent (“B′”, an oligonucleotide sequence complementary to the “B” component of the AB polypeptide) and transfer of information of an associated coding tag to a recoding tag via primer extension. Coding tags A and B are of different sequence, and for ease of identification in this illustration, are also of different length. The different lengths facilitate analysis of coding tag transfer by gel electrophoresis, but are not required for analysis by next generation sequencing. The binding of A′ and B′ binding agents are illustrated as alternative possibilities for a single binding cycle. If a second cycle is added, the extended recording tag would be further extended. Depending on which of A′ or B′ binding agents are added in the first and second cycles, the extended recording tags can contain coding tag information of the form AA, AB, BA, and BB. Thus, the extended recording tag contains information on the order of binding events as well as the identity of binders. Similarly, FIG. 24B illustrates the interaction of a CD polypeptide with a C-specific binding agent (“C′”, an oligonucleotide sequence complementary to the “C” component of the CD polypeptide) and transfer of information of an associated coding tag to a recording tag via primer extension, and a D-specific binding agent (“D′”, an oligonucleotide sequence complementary to the “D” component of the CD polypeptide) and transfer of information of an associated coding tag to a recording tag via primer extension. Coding tags C and D are of different sequence and for ease of identification in this illustration are also of different length. The different lengths facilitate analysis of coding tag transfer by gel electrophoresis, but are not required for analysis by next generation sequencing. The binding of C′ and D′ binding agents are illustrated as alternative possibilities for a single binding cycle. If a second cycle is added, the extended recording tag would be further extended. Depending on which of C′ or D′ binding agents are added in the first and second cycles, the extended recording tags can contain coding tag information of the form CC, CD, DC, and DD. Coding tags may optionally comprise a UMI. The inclusion of UMIs in coding tags allows additional information to be recorded about a binding event; it allows binding events to be distinguished at the level of individual binding agents. This can be useful if an individual binding agent can participate in more than one binding event (e.g. its binding affinity is such that it can disengage and re-bind sufficiently frequently to participate in more than one event). It can also be useful for error-correction. For example, under some circumstances a coding tag might transfer information to the recording tag twice or more in the same binding cycle. The use of a UMI would reveal that these were likely repeated information transfer events all linked to a single binding event.

FIG. 25 illustrates exemplary DNA target polypeptides (AB) and immobilized on beads and assayed by binding agents attached to coding tags. An A-specific binding agent (“A′”, oligonucleotide complementary to A component of AB polypeptide) interacts with an AB polypeptide and information of an associated coding tag is transferred to a recording tag by ligation. A B-specific binding agent (“B′”, an oligonucleotide complementary to B component of AB polypeptide) interacts with an AB polypeptide and information of an associated coding tag is transferred to a recording tag by ligation. Coding tags A and B are of different sequence and for ease of identification in this illustration are also of different length. The different lengths facilitate analysis of coding tag transfer by gel electrophoresis, but are not required for analysis by next generation sequencing.

FIGS. 26A-B illustrate exemplary DNA-peptide polypeptides for binding/coding tag transfer via primer extension. FIG. 26A illustrates an exemplary oligonucleotide-peptide target polypeptide (“A” oligonucleotide-cMyc peptide) immobilized on beads. A cMyc-specific binding agent (e.g. antibody) interacts with the cMyc peptide portion of the polypeptide and information of an associated coding tag is transferred to a recording tag. The transfer of information of the cMyc coding tag to a recording tag may be analyzed by gel electrophoresis. FIG. 26B illustrates an exemplary oligonucleotide-peptide target polypeptide (“C” oligonucleotide-hemagglutinin (HA) peptide) immobilized on beads. An HA-specific binding agent (e.g., antibody) interacts with the HA peptide portion of the polypeptide and information of an associated coding tag is transferred to a recording tag. The transfer of information of the coding tag to a recording tag may be analyzed by gel electrophoresis. The binding of cMyc antibody-coding tag and HA antibody-coding tag are illustrated as alternative possibilities for a single binding cycle. If a second binding cycle is performed, the extended recording tag would be further extended. Depending on which of cMyc antibody-coding tag or HA antibody-coding tag are added in the first and second binding cycles, the extended recording tags can contain coding tag information of the form cMyc-HA, HA-cMyc, cMyc-cMyc, and HA-HA. Although not illustrated, additional binding agents can also be introduced to enable detection of the A and C oligonucleotide components of the polypeptides. Thus, hybrid polypeptides comprising different types of backbone can be analyzed via transfer of information to a recording tag and readout of the extended recording tag, which contains information on the order of binding events as well as the identity of the binding agents.

FIGS. 27A-B illustrate examples for the generation of Error-Correcting Barcodes. (A) A subset of 65 error-correcting barcodes (SEQ ID NOs:1-65, Table 1) were selected from a set of 77 barcodes derived from the R software package ‘DNABarcodes’ (https://bioconductor.rikenjp/packages/3.3/bioc/manuals/DNABarcodes/man/DNABarcodes. pdf) using the command parameters [create.dnabarcodes(n=15,dist=10)]. This algorithm generates 15-mer “Hamming” barcodes that can correct substitution errors out to a distance of four substitutions, and detect errors out to nine substitutions. The subset of 65 barcodes was created by filtering out barcodes that didn't exhibit a variety of nanopore current levels (for nanopore-based sequencing) or that were too correlated with other members of the set. (B) A plot of the predicted nanopore current levels for the 15-mer barcodes passing through the pore. The predicted currents were computed by splitting each 15-mer barcode word into composite sets of 11 overlapping 5-mer words, and using a 5-mer R9 nanopore current level look-up table (template_median68 pA.5mers.model (https://github.com/jts/nanopolish/tree/master/etc/r9-models) to predict the corresponding current level as the barcode passes through the nanopore, one base at a time. As can be appreciated from (B), this set of 65 barcodes exhibit unique current signatures for each of its members.

TABLE 1
Exemplary Barcodes

| | | | | | |
|---|---|---|---|---|---|
| SEQ ID NO: 1 | SEQ ID NO: 12 | SEQ ID NO: 23 | SEQ ID NO: 34 | SEQ ID NO: 45 | SEQ ID NO: 56 |
| atgtctagcatgccg | gagtactagagccaa | cctatagcacaatcc | gcaacgtgaattgag | ctgatgtagtcgaag | ccacgaggcttagtt |
| SEQ ID NO: 2 | SEQ ID NO: 13 | SEQ ID NO: 24 | SEQ ID NO: 35 | SEQ ID NO: 46 | SEQ ID NO: 57 |
| ccgtgtcatgtggaa | gagcgtcaataacgg | atcaccgaggttgga | ctaagtagagccaca | gtcggttgcggatag | ggccaactaaggtgc |
| SEQ ID NO: 3 | SEQ ID NO: 14 | SEQ ID NO: 25 | SEQ ID NO: 36 | SEQ ID NO: 47 | SEQ ID NO: 58 |
| taagccggtatatca | gcggtatctacactg | gattcaacggagaag | tgtctgttggaagcg | tcctcctcctaagaa | gcacctattcgacaa |
| SEQ ID NO: 4 | SEQ ID NO: 15 | SEQ ID NO: 26 | SEQ ID NO: 37 | SEQ ID NO: 48 | SEQ ID NO: 59 |
| ttcgatatgacggaa | cttctccgaagagaa | acgaacctcgcacca | ttaatagacagcgcg | attcggtccacttca | tggacacgatcggct |
| SEQ ID NO: 5 | SEQ ID NO: 16 | SEQ ID NO: 27 | SEQ ID NO: 38 | SEQ ID NO: 49 | SEQ ID NO: 60 |
| cgtatacgcgttagg | tgaagcctgtgttaa | aggacttcaagaaga | cgacgctctaacaag | ccttacaggtctgcg | ctataattccaacgg |
| SEQ ID NO: 6 | SEQ ID NO: 17 | SEQ ID NO: 28 | SEQ ID NO: 39 | SEQ ID NO: 50 | SEQ ID NO: 61 |
| aactgccgagattcc | ctggatggttgtcga | ggttgaatcctcgca | catggcttattgaga | gatcattggccaatt | aacgtggttagtaag |
| SEQ ID NO: 7 | SEQ ID NO: 18 | SEQ ID NO: 29 | SEQ ID NO: 40 | SEQ ID NO: 51 | SEQ ID NO: 62 |
| tgatcttagctgtgc | actgcacggttccaa | aaccaacctctagcg | actaggtatggccgg | ttcaaggctgagttg | caaggaacgagtggc |
| SEQ ID NO: 8 | SEQ ID NO: 19 | SEQ ID NO: 30 | SEQ ID NO: 41 | SEQ ID NO: 52 | SEQ ID NO: 63 |
| gagtcggtaccttga | cgagagatggtcctt | acgcgaatatctaac | gtcctcgtctatcct | tggctcgattgaatc | caccagaacggaaga |
| SEQ ID NO: 9 | SEQ ID NO: 20 | SEQ ID NO: 31 | SEQ ID NO: 42 | SEQ ID NO: 53 | SEQ ID NO: 64 |
| ccgcttgtgatctgg | tcttgagagacaaga | gttgagaattacacc | taggattccgttacc | gtaagccatccgctc | cgtacggtcaagcaa |
| SEQ ID NO: 10 | SEQ ID NO: 21 | SEQ ID NO: 32 | SEQ ID NO: 43 | SEQ ID NO: 54 | SEQ ID NO: 65 |
| agatagcgtaccgga | aattcgcactgtgtt | ctctctctgtgaacc | tctgaccaccggaag | acacatgcgtagaca | tcggtgacaggctaa |
| SEQ ID NO: 11 | SEQ ID NO: 22 | SEQ ID NO: 33 | SEQ ID NO: 44 | SEQ ID NO: 55 | |
| tccaggctcatcatc | gtagtgccgctaaga | gccatcagtaagaga | agagtcacctcgtgg | tgctatggattcaag | |

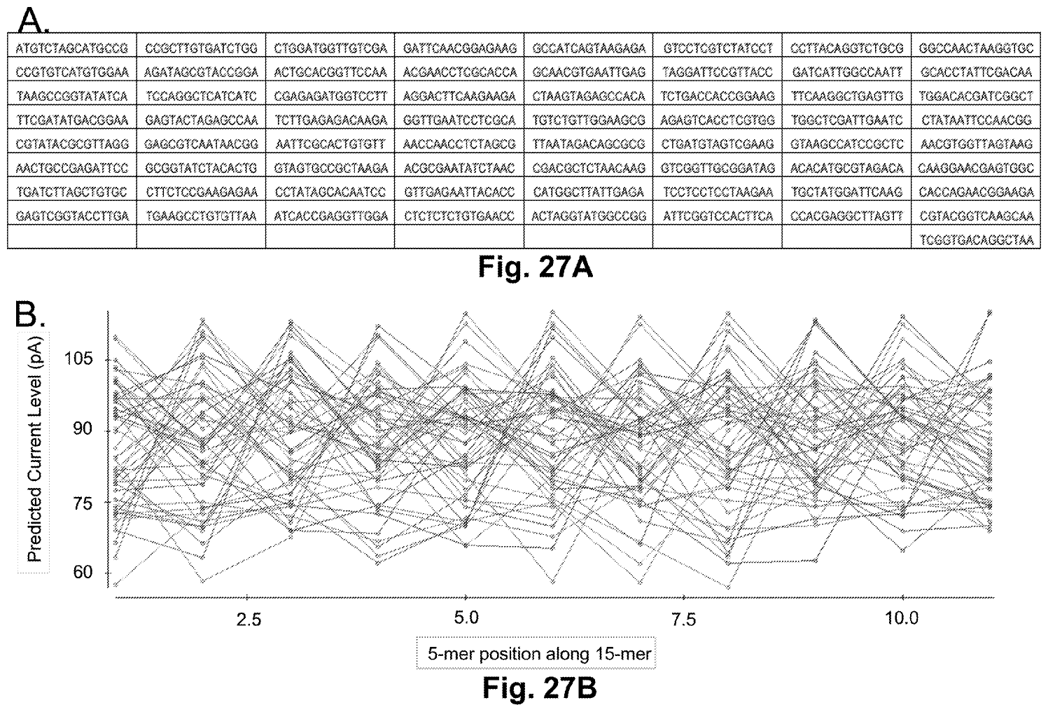

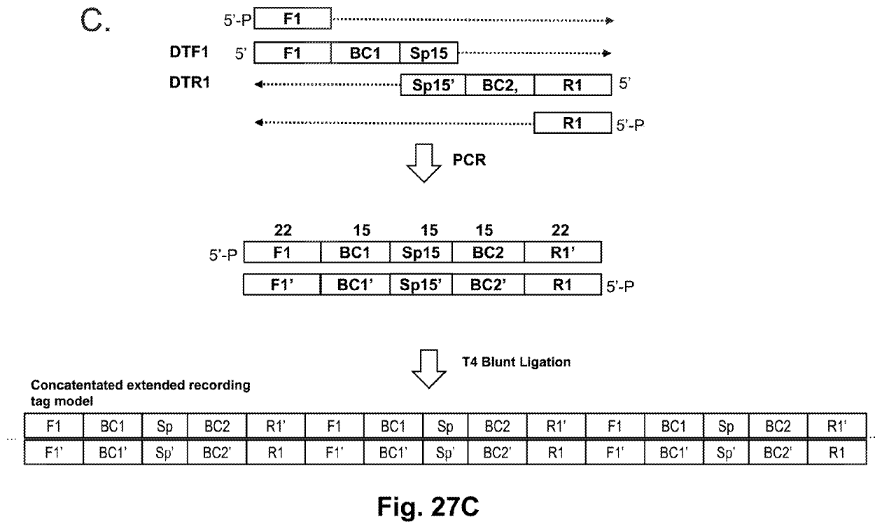

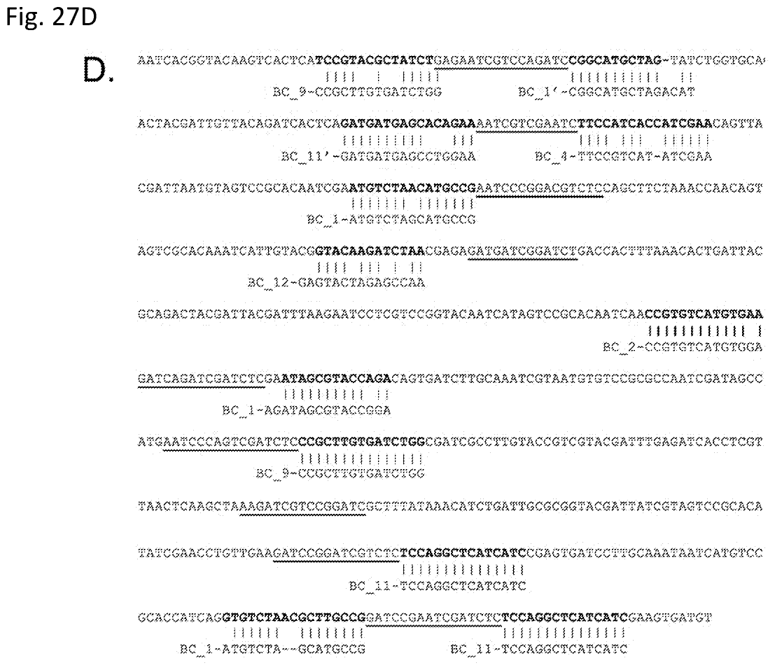

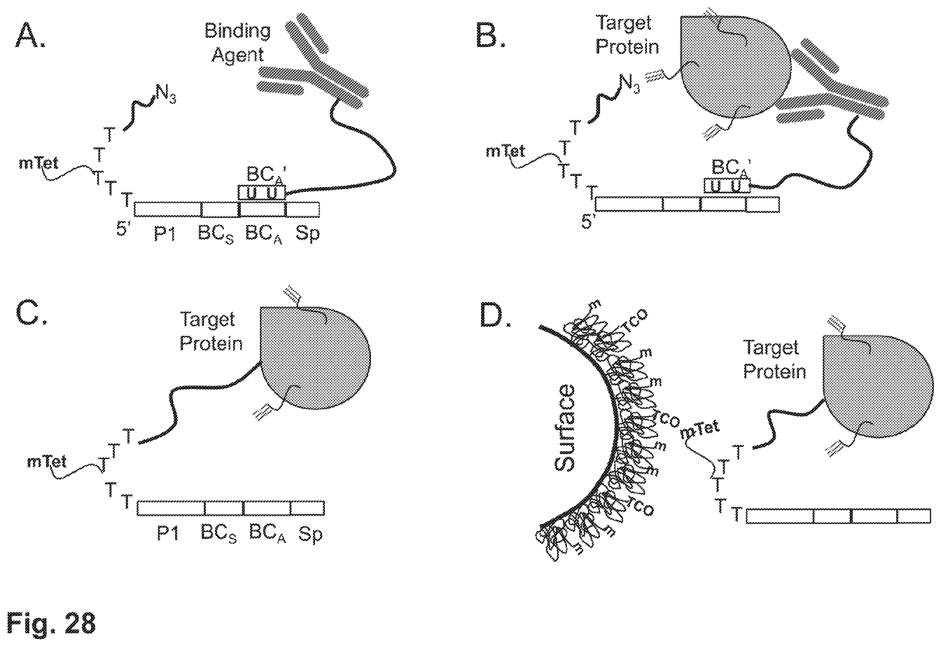

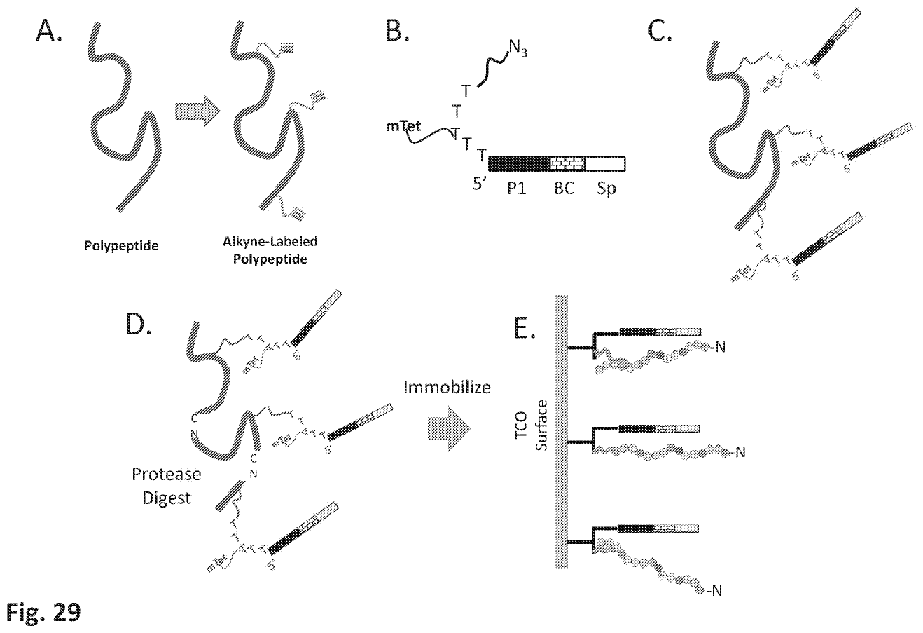

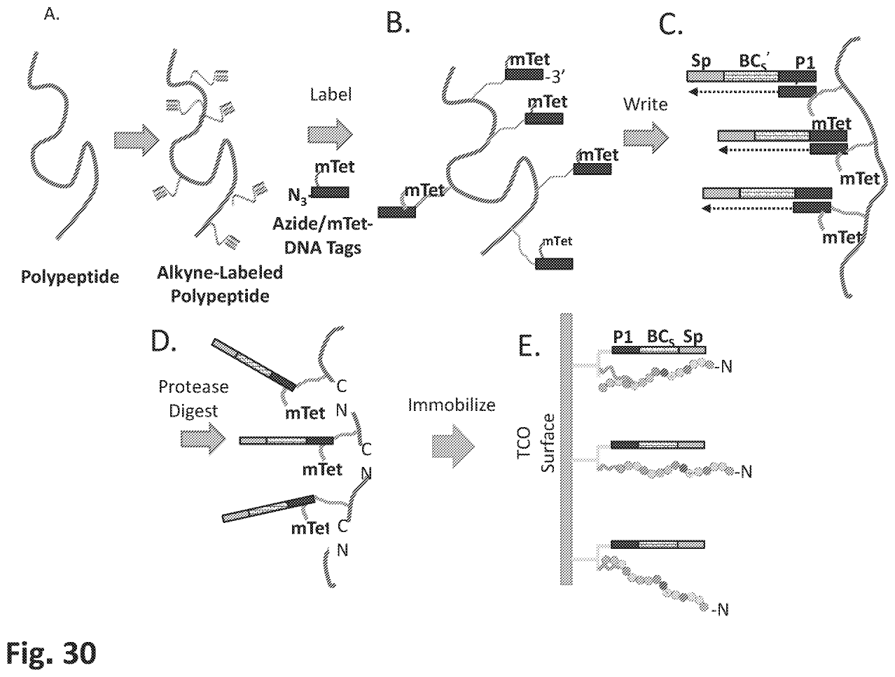

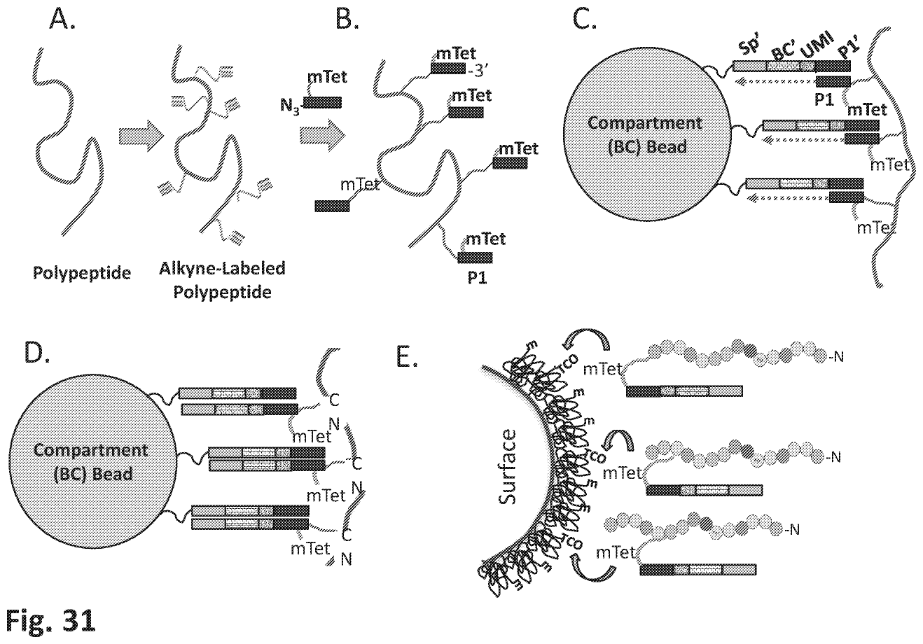

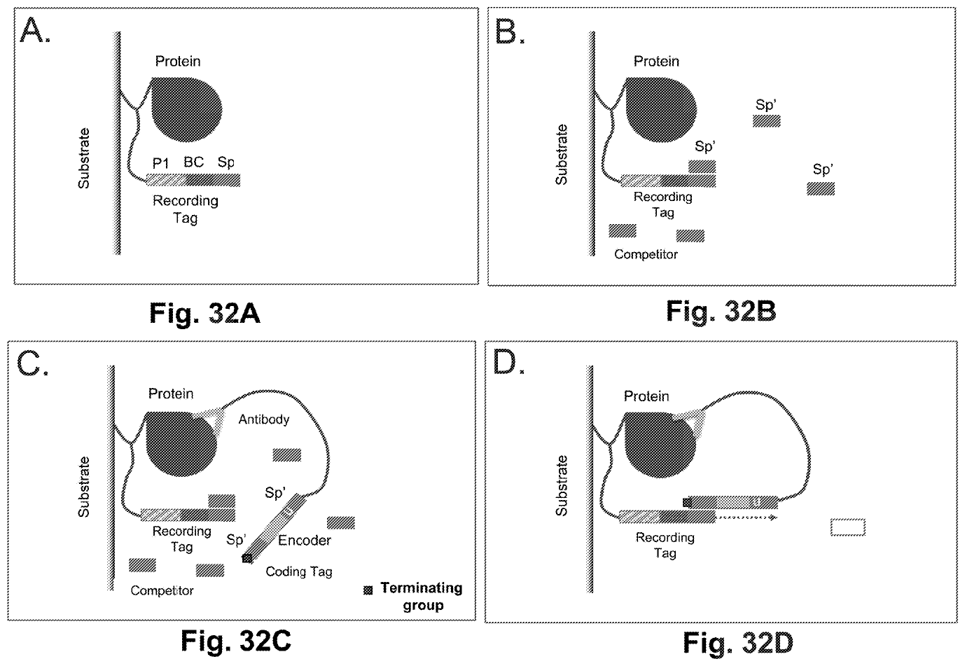

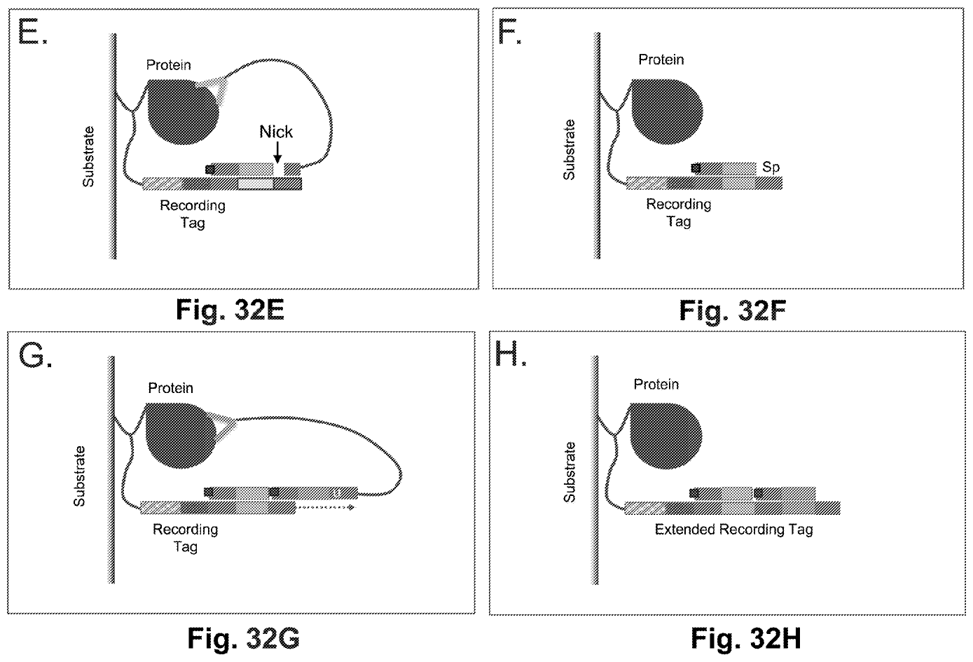

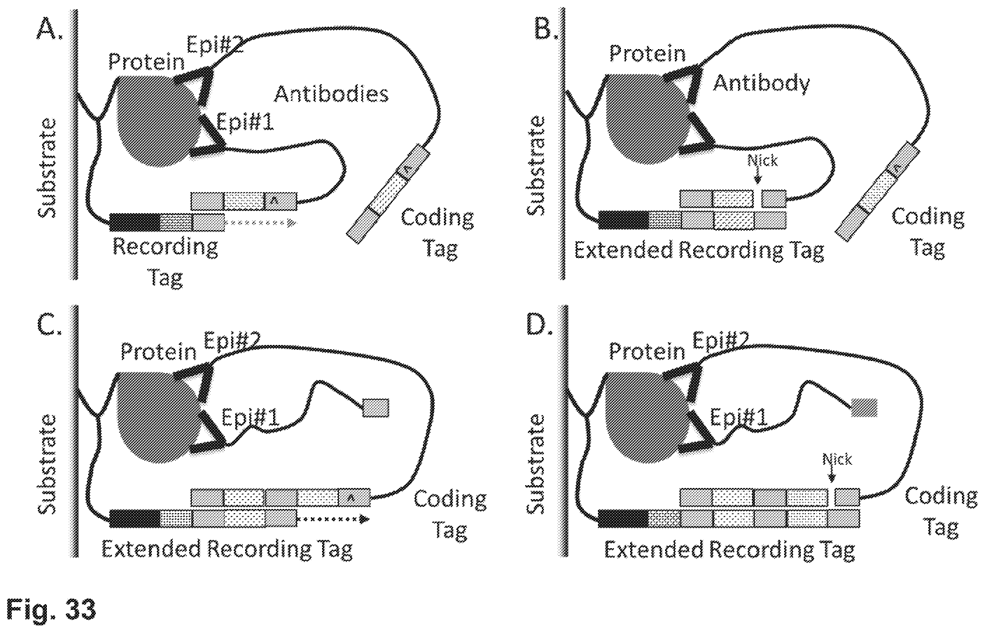

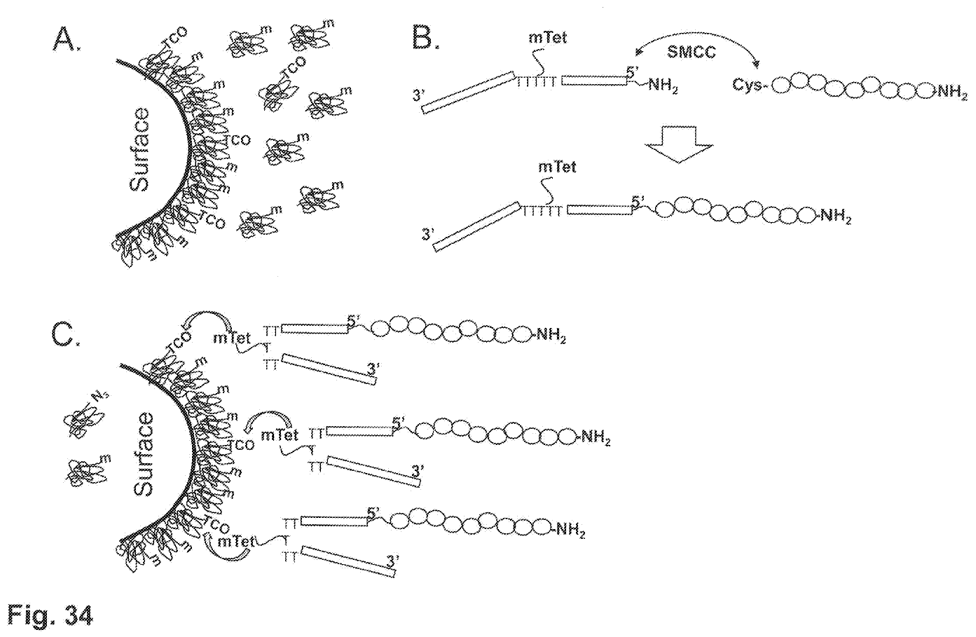

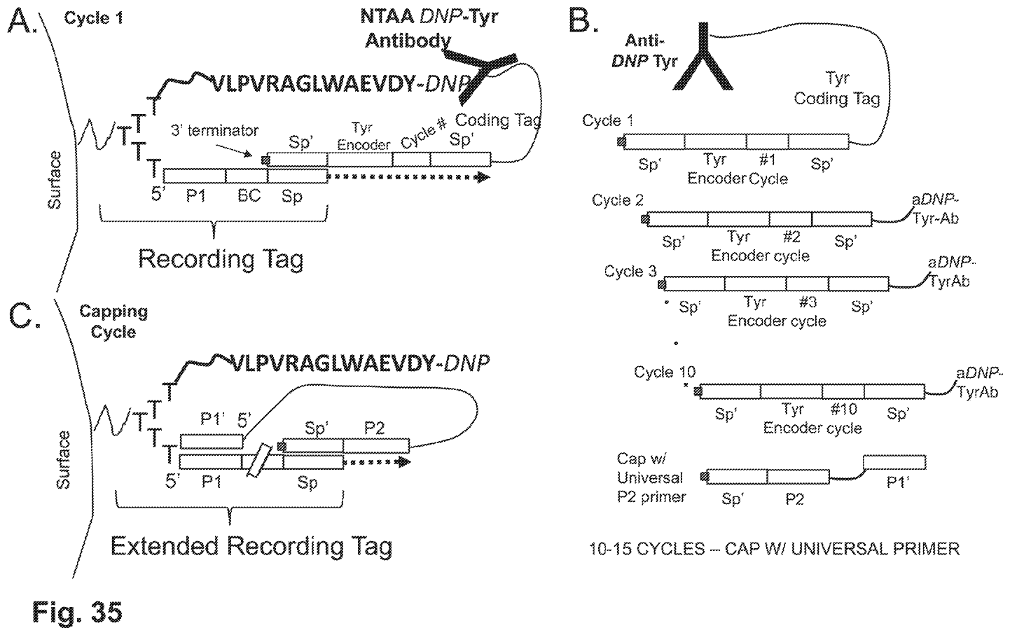

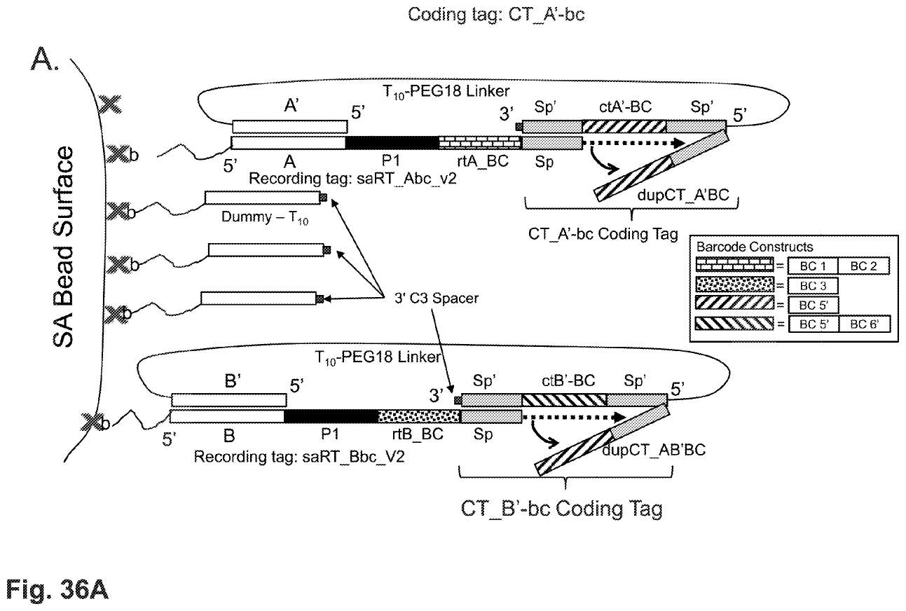

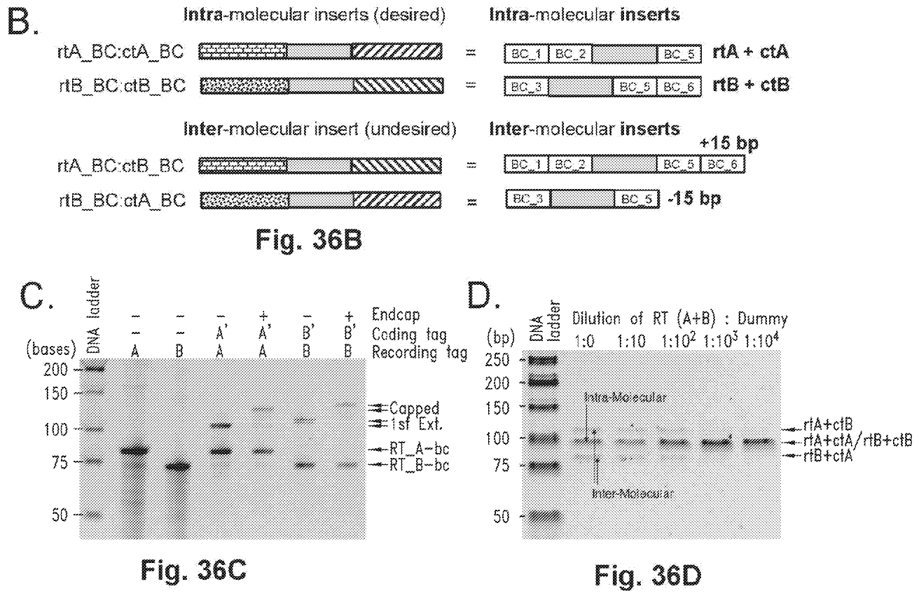

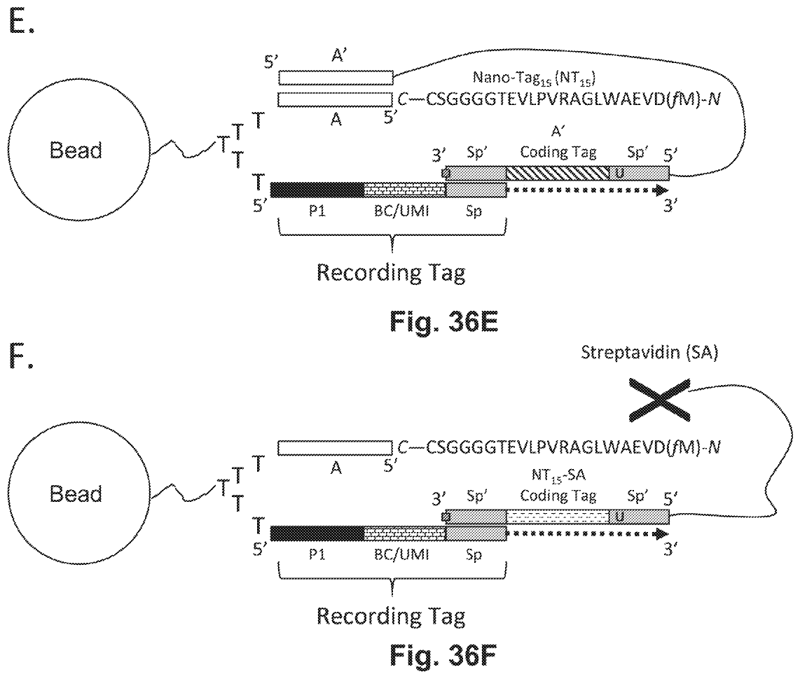

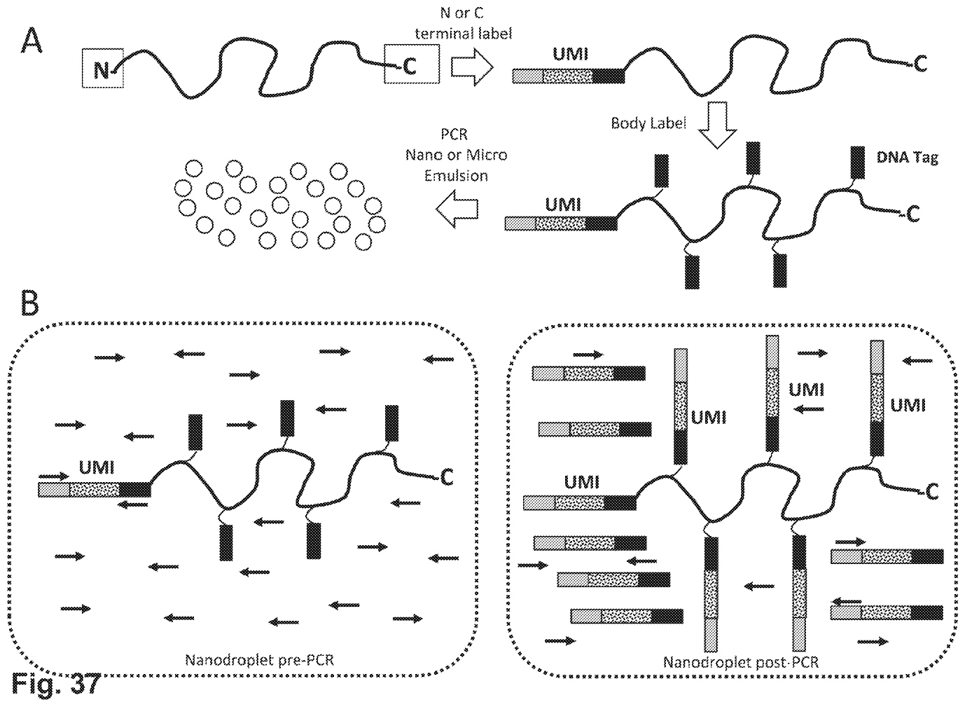

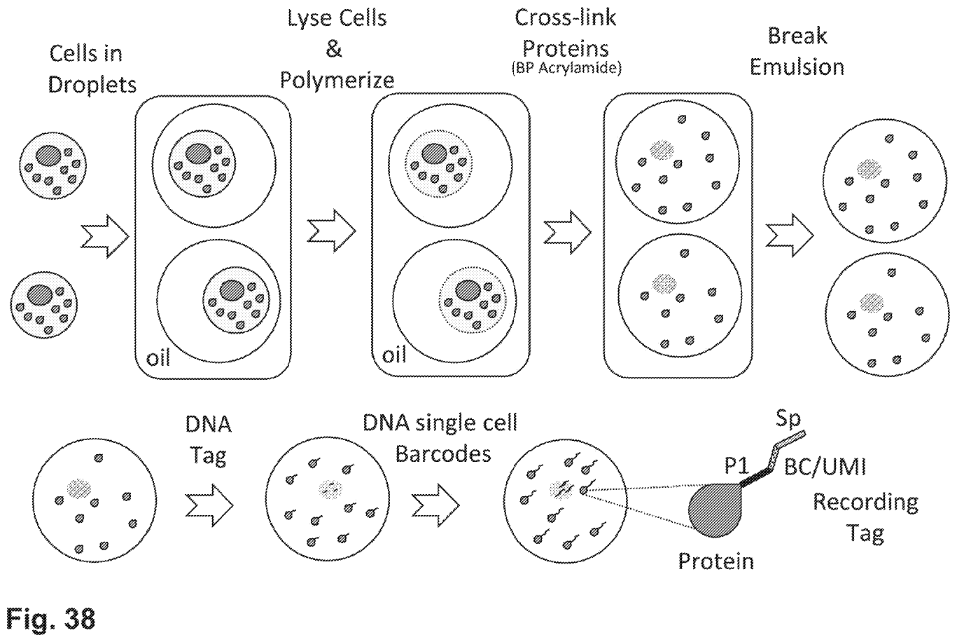

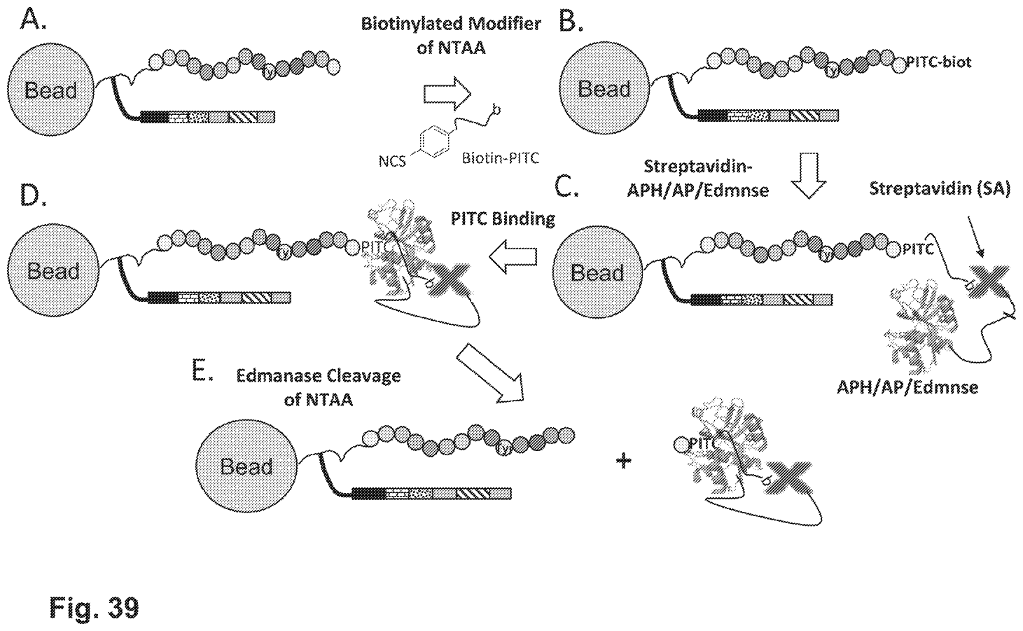

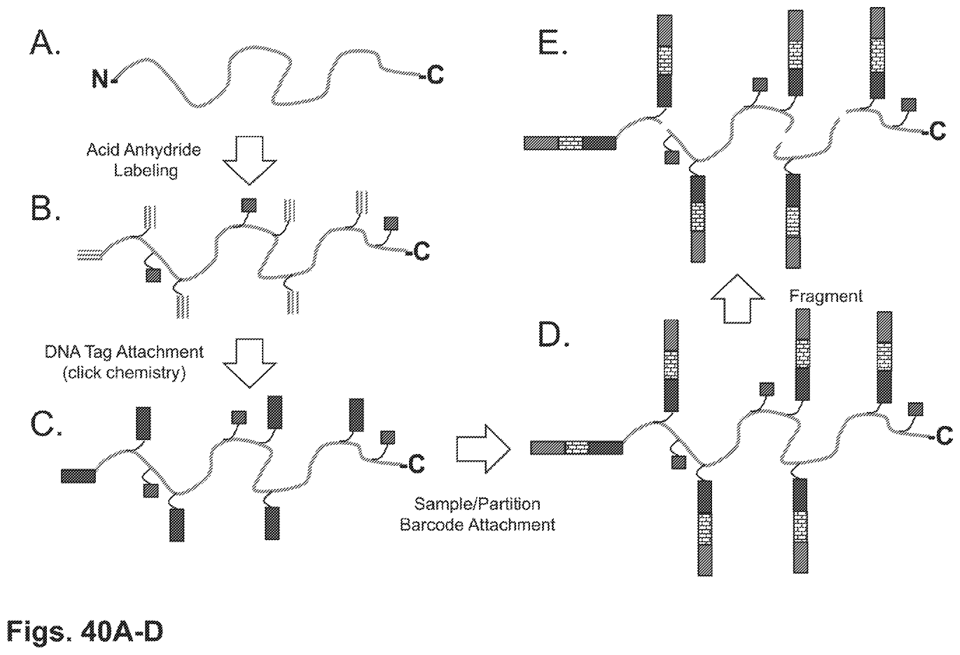

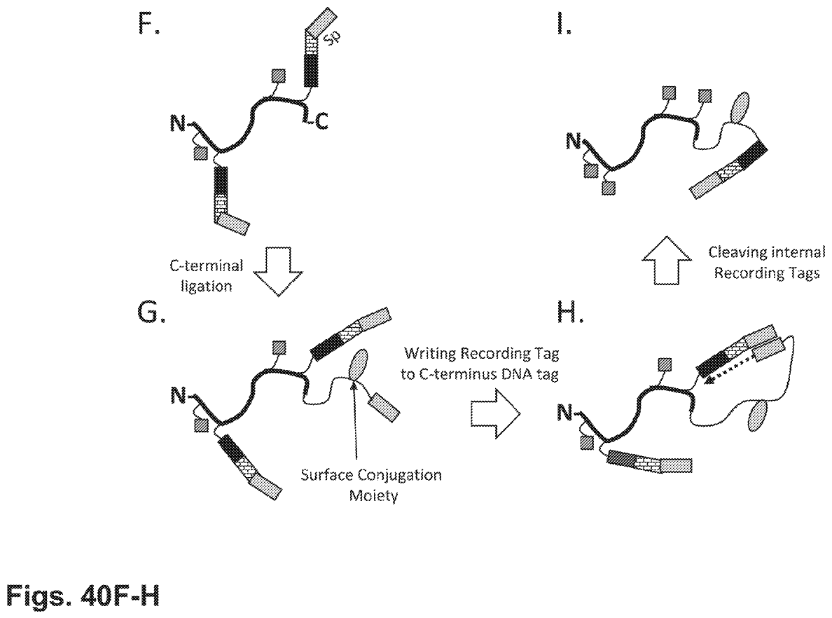

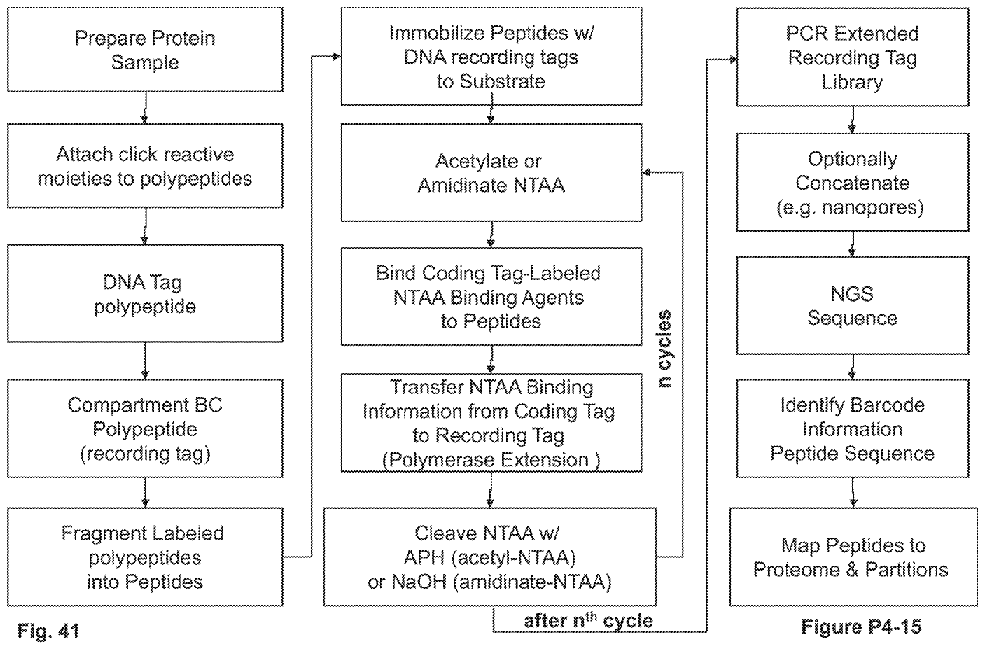

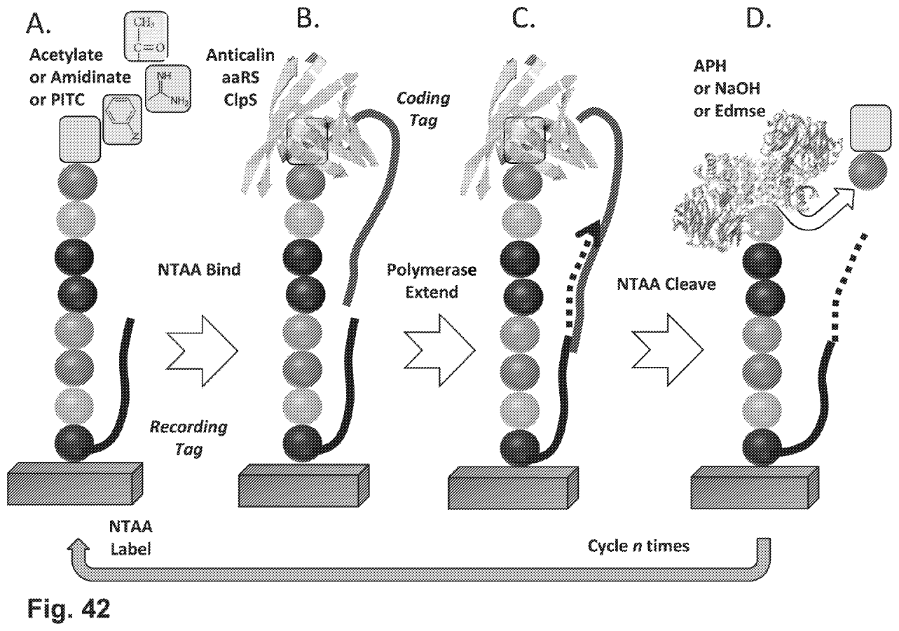

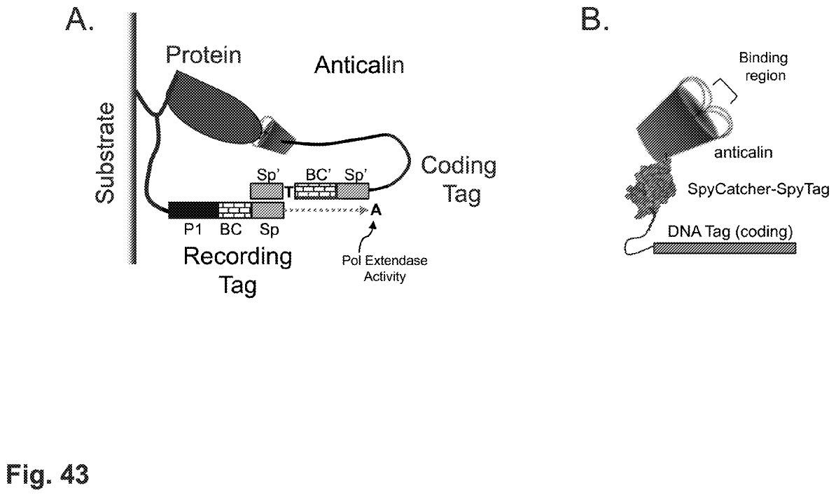

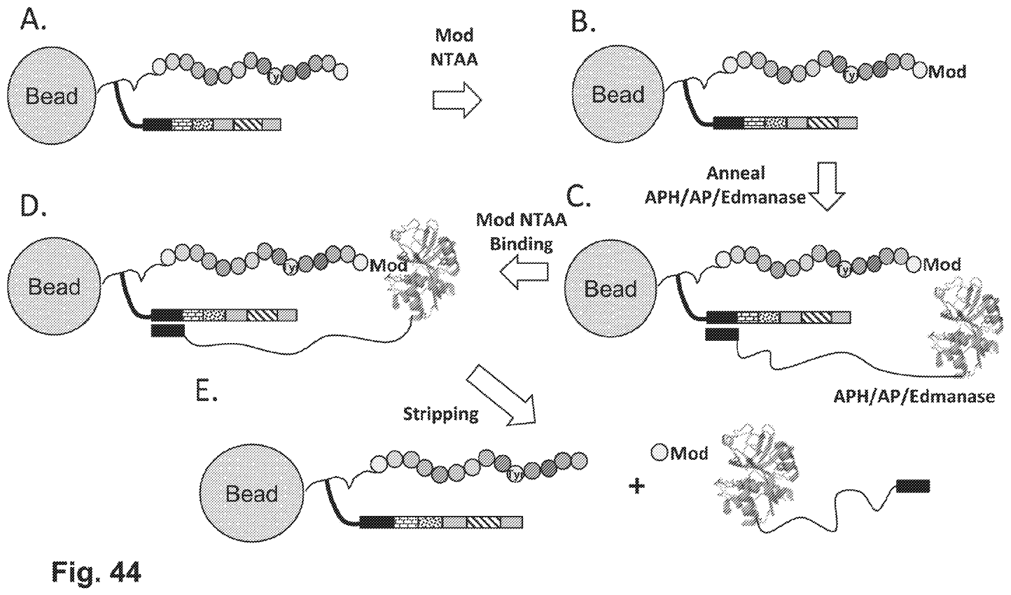

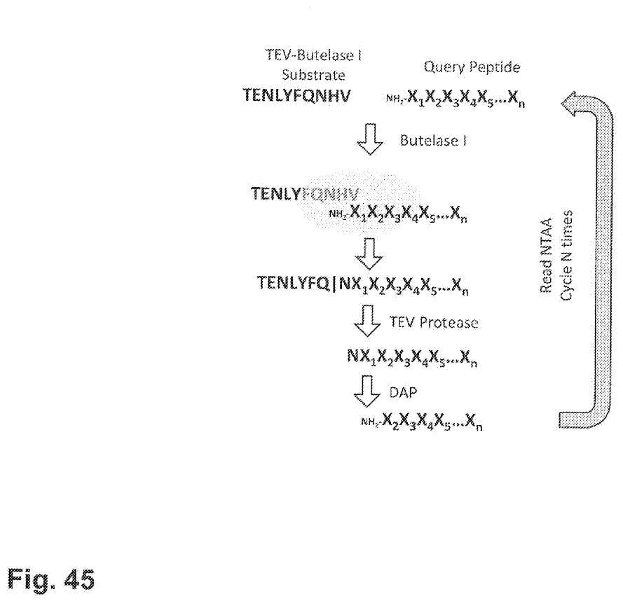

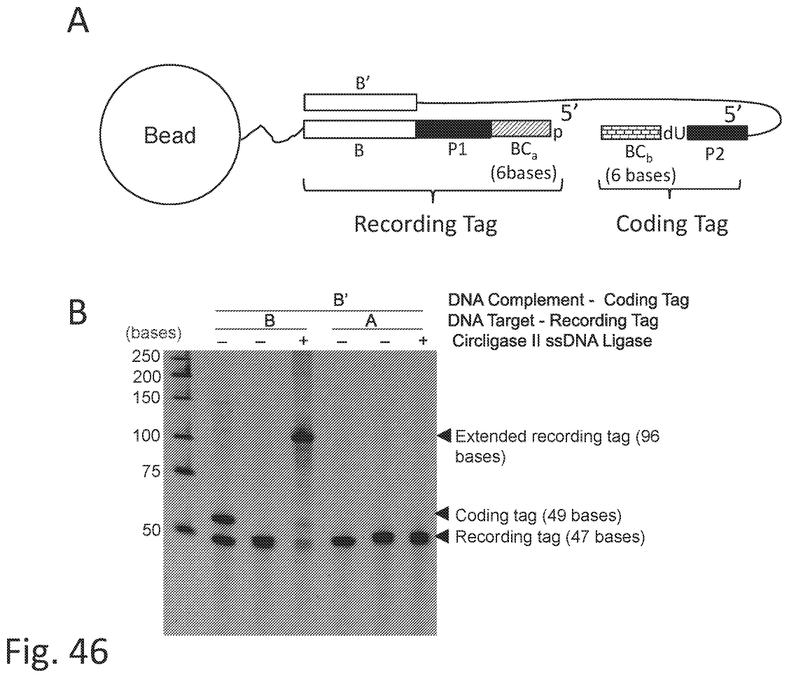

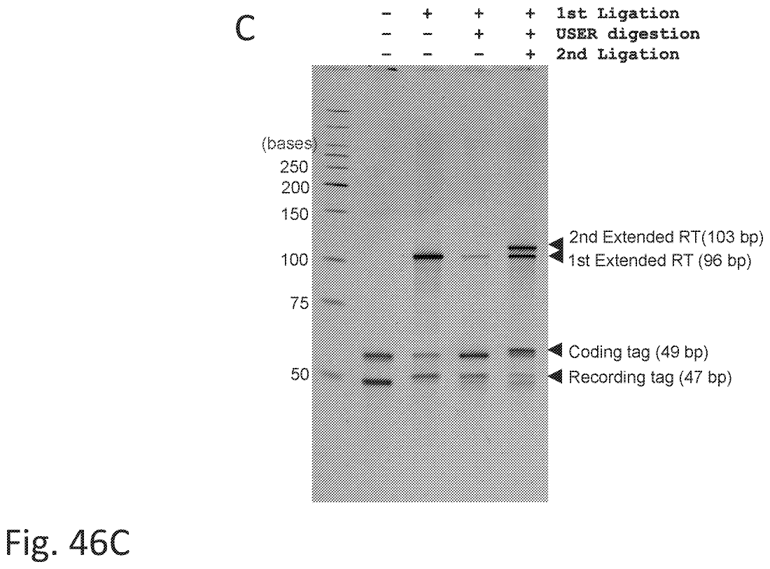

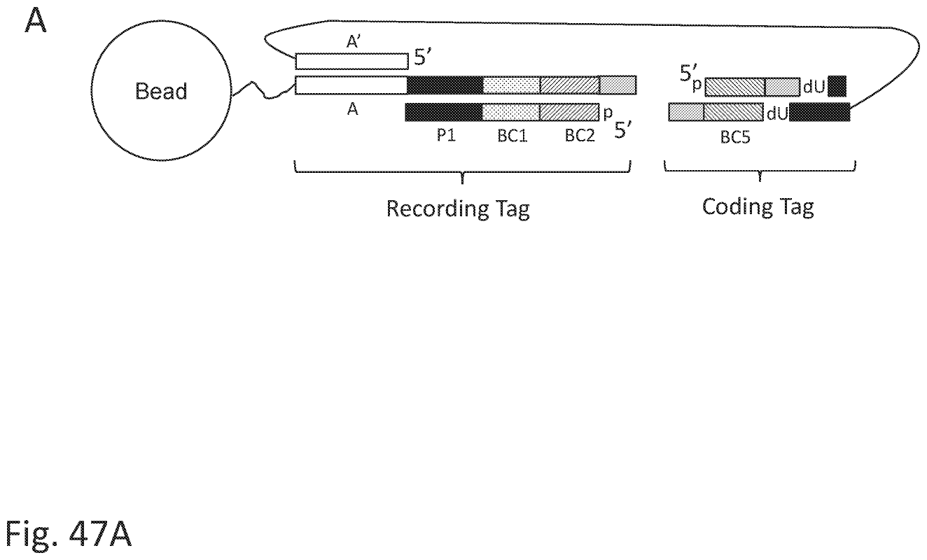

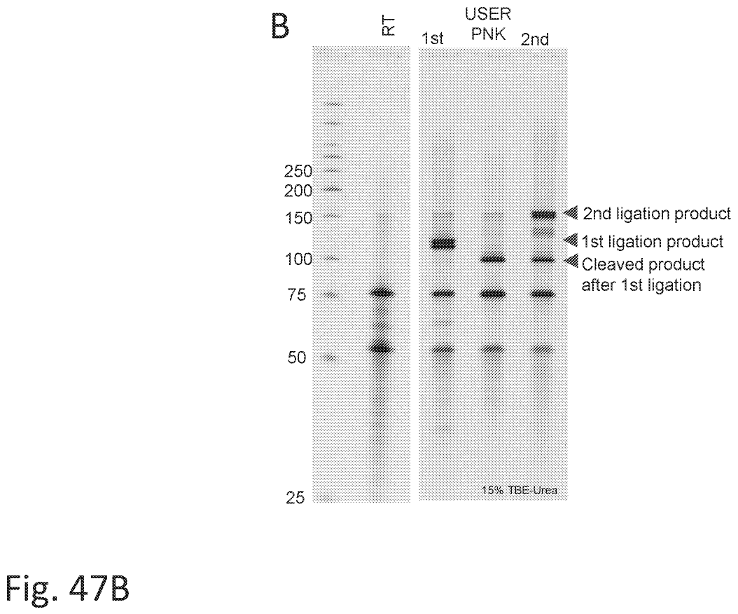

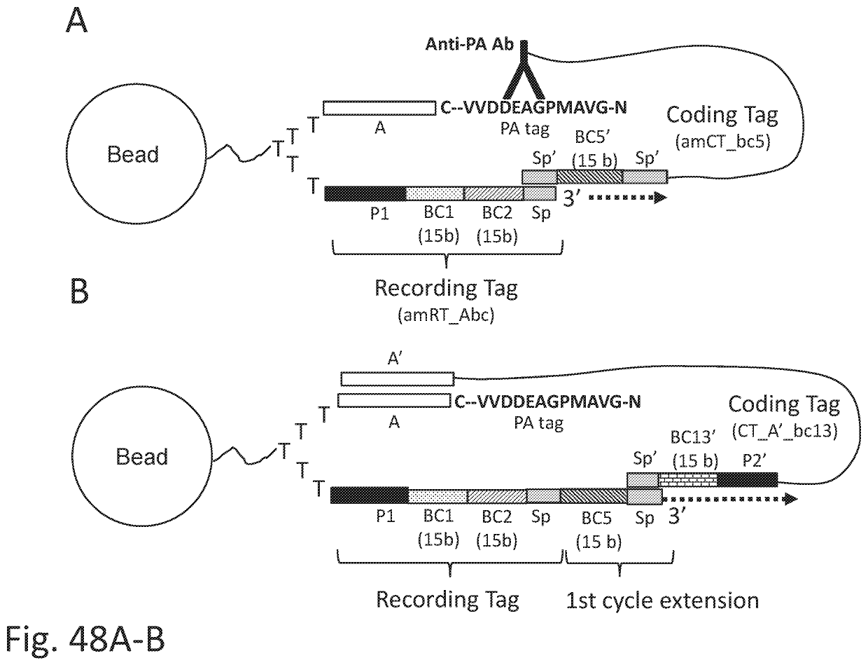

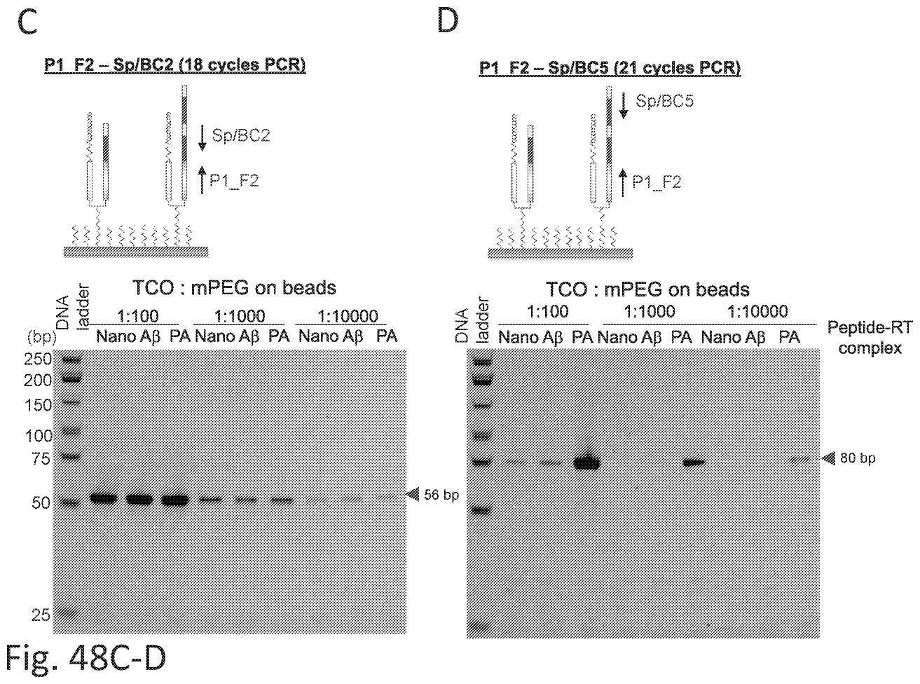

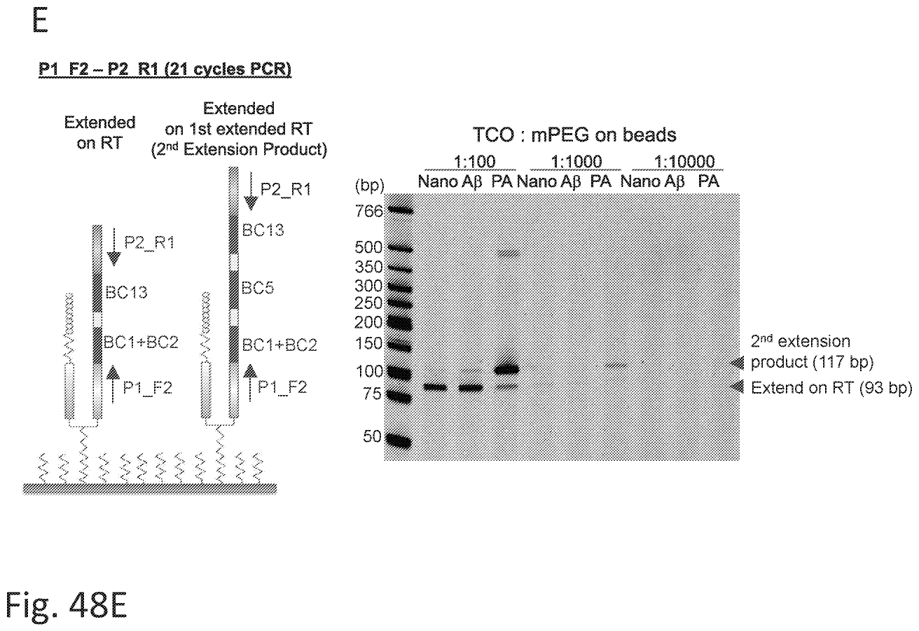

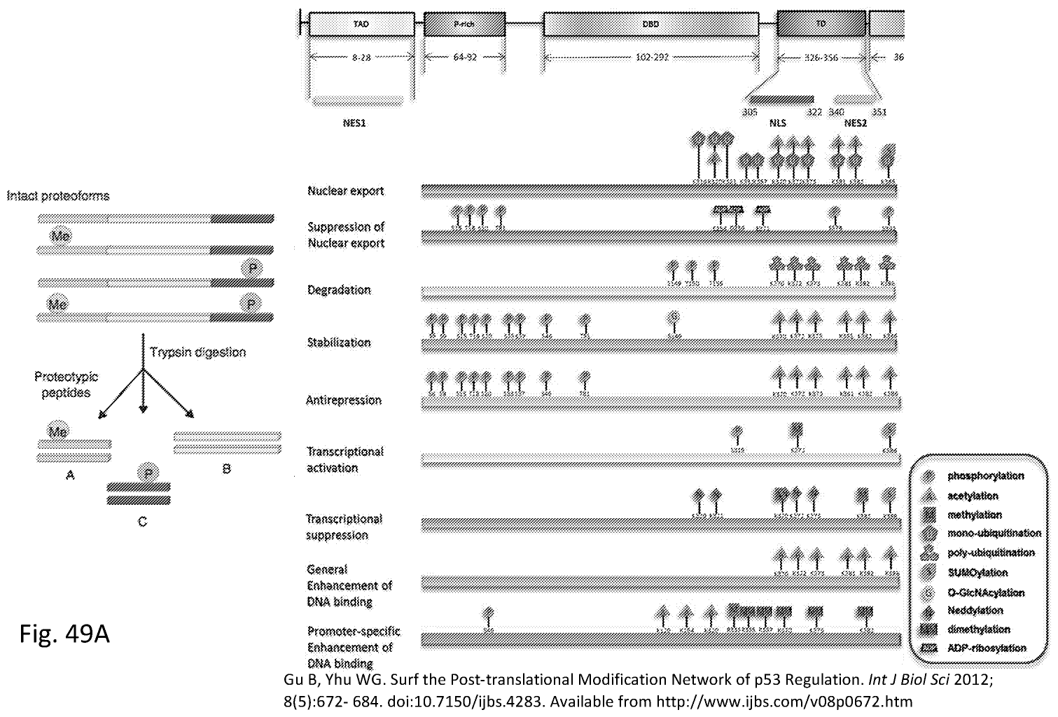

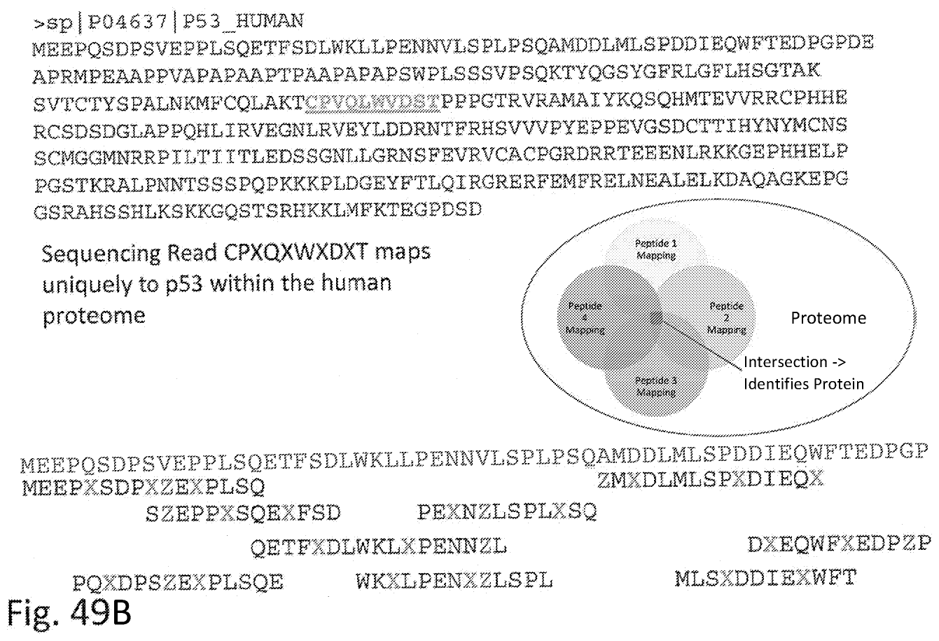

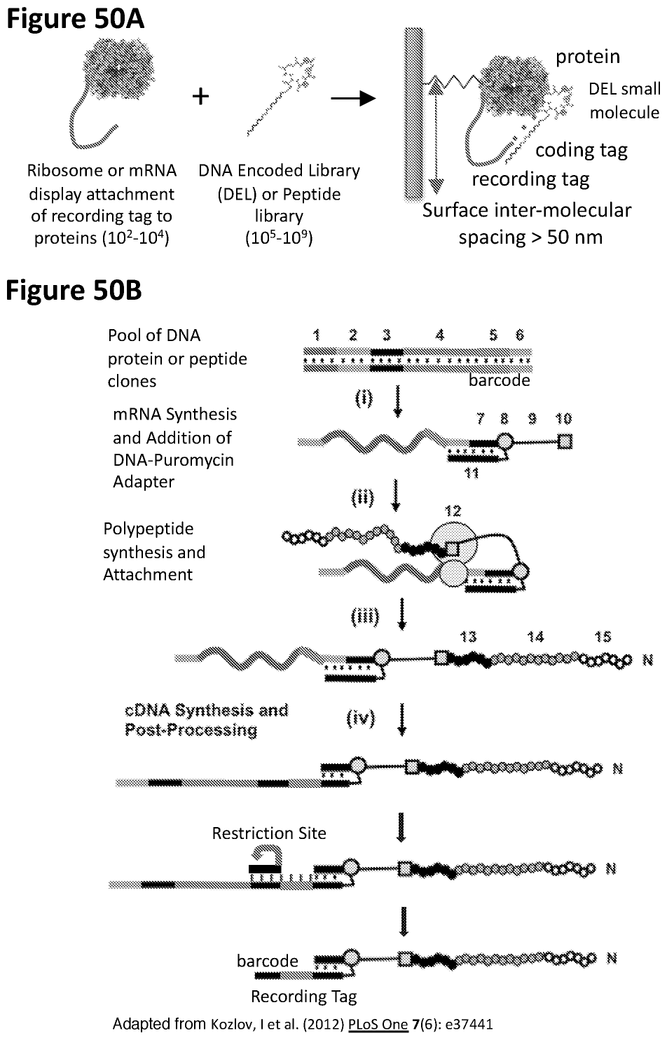

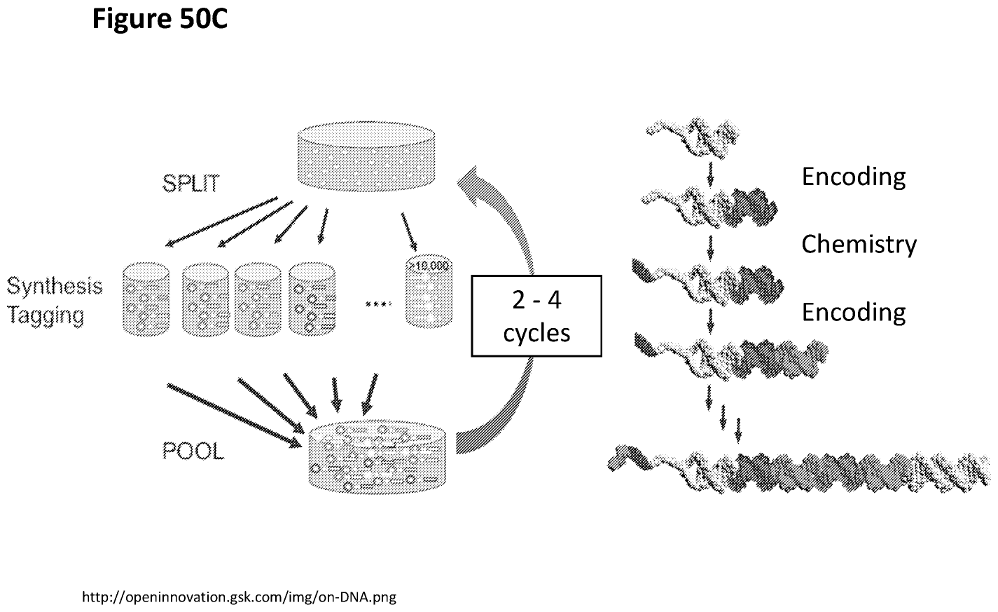

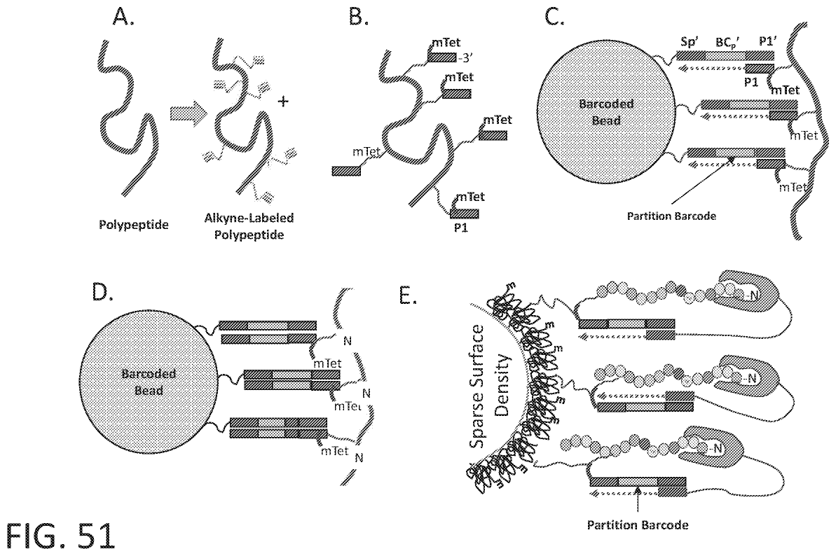

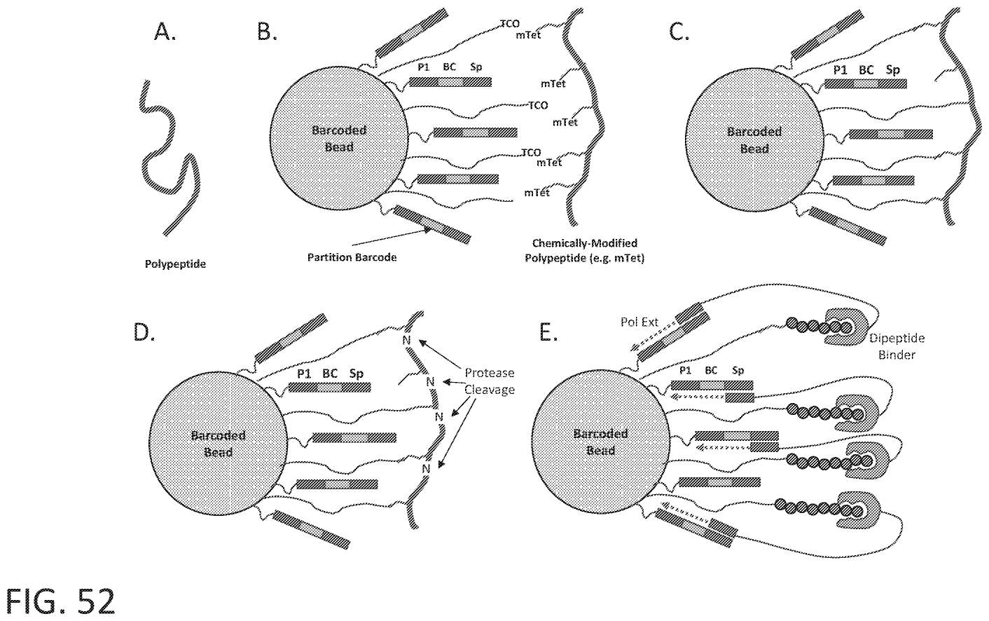

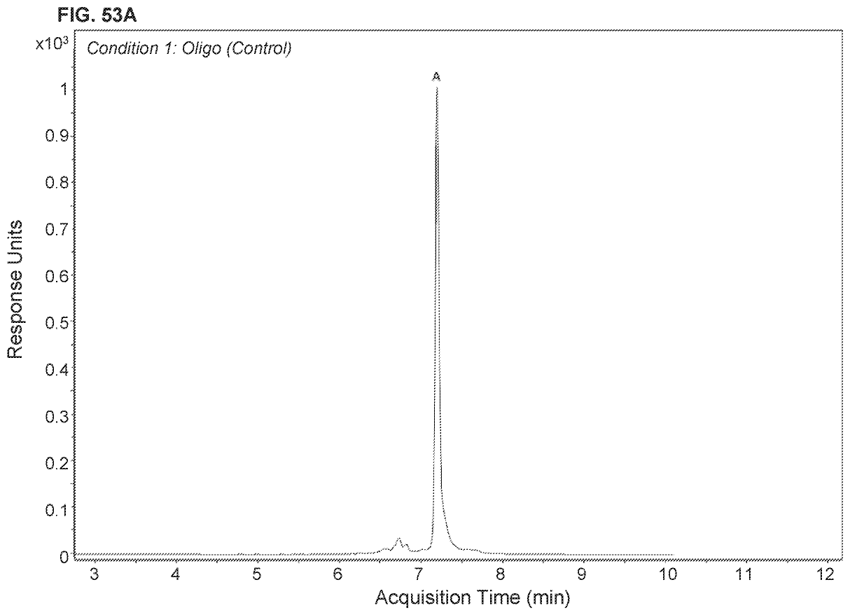

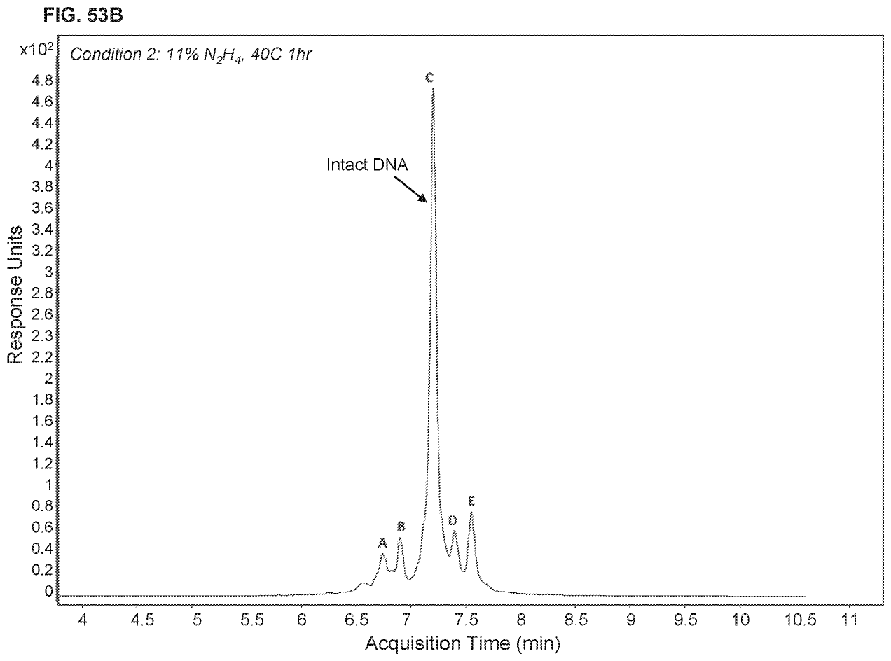

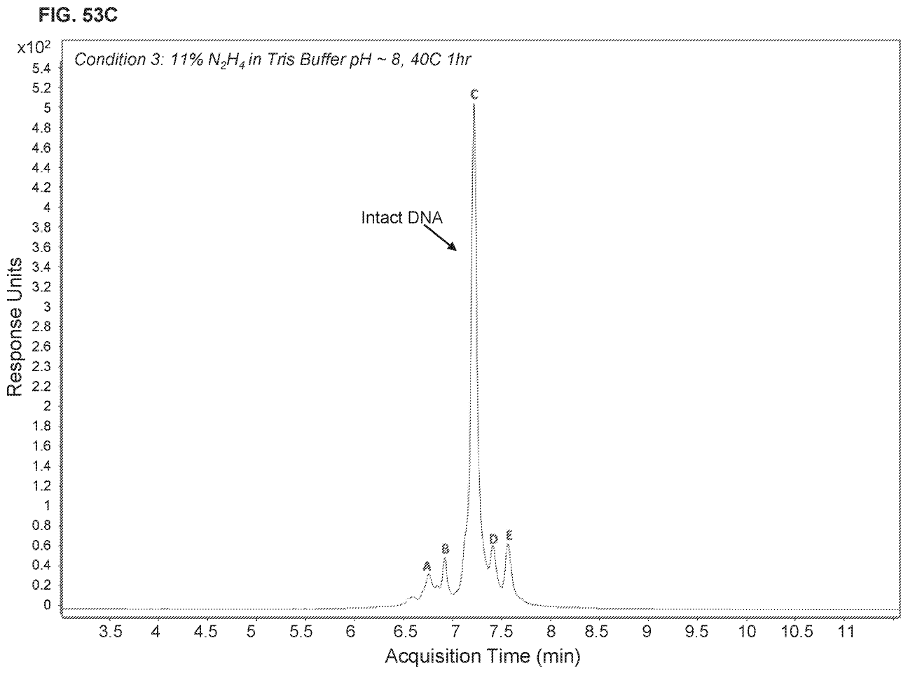

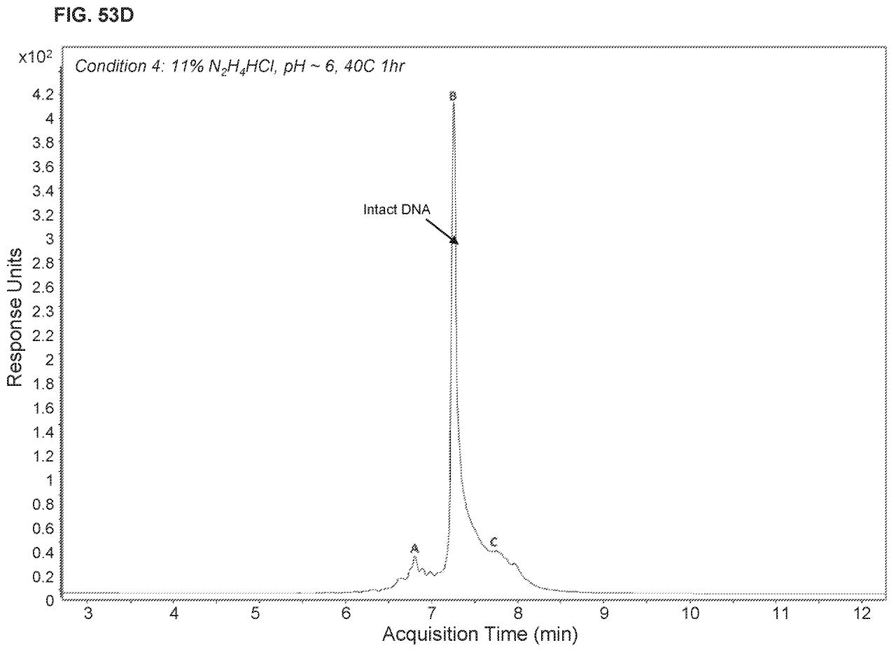

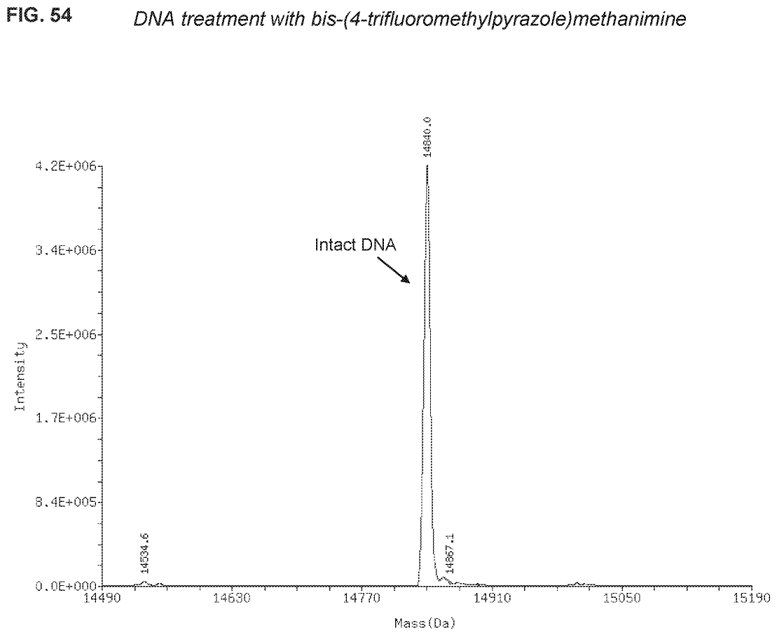

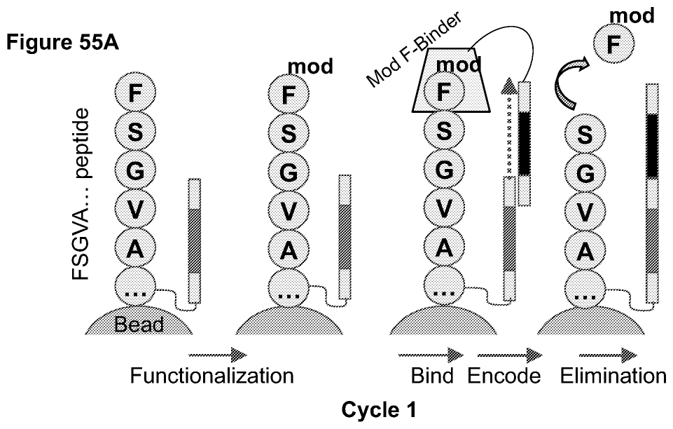

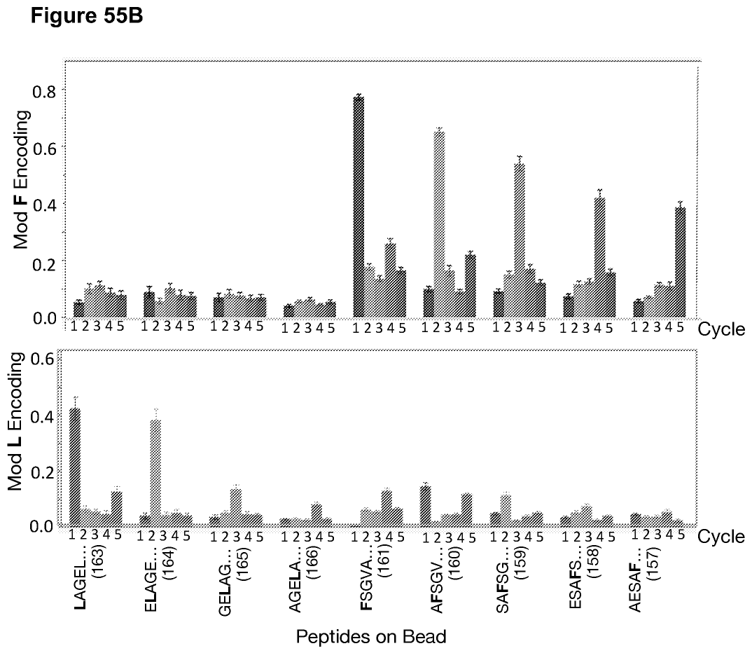

FIG. 27C: Generation of PCR products as model extended recording tags for nanopore sequencing is shown using overlapping sets of DTR and DTR primers. PCR amplicons are then ligated to form a concatenated extended recording tag model. FIG. 27D: Nanopore sequencing read of exemplary “extended recording tag” model (read length 734 bases; SEQ ID NO: 168) generated as shown in FIG. 27C. The MinIon R9.4 Read has a quality score of 7.2 (poor read quality). However, barcode sequences can easily be identified using lalign even with a poor quality read (Qscore=7.2). A 15-mer spacer element is underlined. Barcodes can align in either forward or reverse orientation, denoted by BC or BC′ designation (BC 9—SEQ ID NO: 9; BC 1′—SEQ ID NO: 66; BC 11′—SEQ ID NO: 76; BC 4—SEQ ID NO: 4; BC 1—SEQ ID NO: 1; BC 12—SEQ ID NO: 12; BC 2—SEQ ID NO: 2; BC 11—SEQ ID NO: 11).

FIGS. 28A-D illustrate examples for the analyte-specific labeling of proteins with recording tags. (A) A binding agent targeting a protein analyte of interest in its native conformation comprises an analyte-specific barcode (BC_{A}′) that hybridizes to a complementary analyte-specific barcode (BC_{A}) on a DNA recording tag. Alternatively, the DNA recording tag could be attached to the binding agent via a cleavable linker, and the DNA recording tag is “clicked” to the protein directly and is subsequently cleaved from the binding agent (via the cleavable linker). The DNA recording tag comprises a reactive coupling moiety (such as a click chemistry reagent (e.g., azide, mTet, etc.) for coupling to the protein of interest, and other functional components (e.g., universal priming sequence (P1), sample barcode (BCs), analyte specific barcode (BC_{A}), and spacer sequence (Sp)). A sample barcode (BCs) can also be used to label and distinguish proteins from different samples. The DNA recording tag may also comprise an orthogonal coupling moiety (e.g., mTet) for subsequent coupling to a substrate surface. For click chemistry coupling of the recording tag to the protein of interest, the protein is pre-labeled with a click chemistry coupling moiety cognate for the click chemistry coupling moiety on the DNA recording tag (e.g., alkyne moiety on protein is cognate for azide moiety on DNA recording tag). Examples of reagents for labeling the DNA recording tag with coupling moieties for click chemistry coupling include alkyne-NHS reagents for lysine labeling, alkyne-benzophenone reagents for photoaffinity labeling, etc. (B) After the binding agent binds to a proximal target protein, the reactive coupling moiety on the recording tag (e.g., azide) covalently attaches to the cognate click chemistry coupling moiety (shown as a triple line symbol) on the proximal protein. (C) After the target protein analyte is labeled with the recording tag, the attached binding agent is removed by digestion of uracils (U) using a uracil-specific excision reagent (e.g., USER™). (D) The DNA recording tag labeled target protein analyte is immobilized to a substrate surface using a suitable bioconjugate chemistry reaction, such as click chemistry (alkyne-azide binding pair, methyl tetrazine (mTET)—trans-cyclooctene (TCO) binding pair, etc.). In certain embodiments, the entire target protein-recording tag labeling assay is performed in a single tube comprising many different target protein analytes using a pool of binding agents and a pool of recording tags. After targeted labeling of protein analytes within a sample with recording tags comprising a sample barcode (BCs), multiple protein analyte samples can be pooled before the immobilization step in (D). Accordingly, in certain embodiments, up to thousands of protein analytes across hundreds of samples can be labeled and immobilized in a single tube next generation protein assay (NGPA), greatly economizing on expensive affinity reagents (e.g., antibodies).

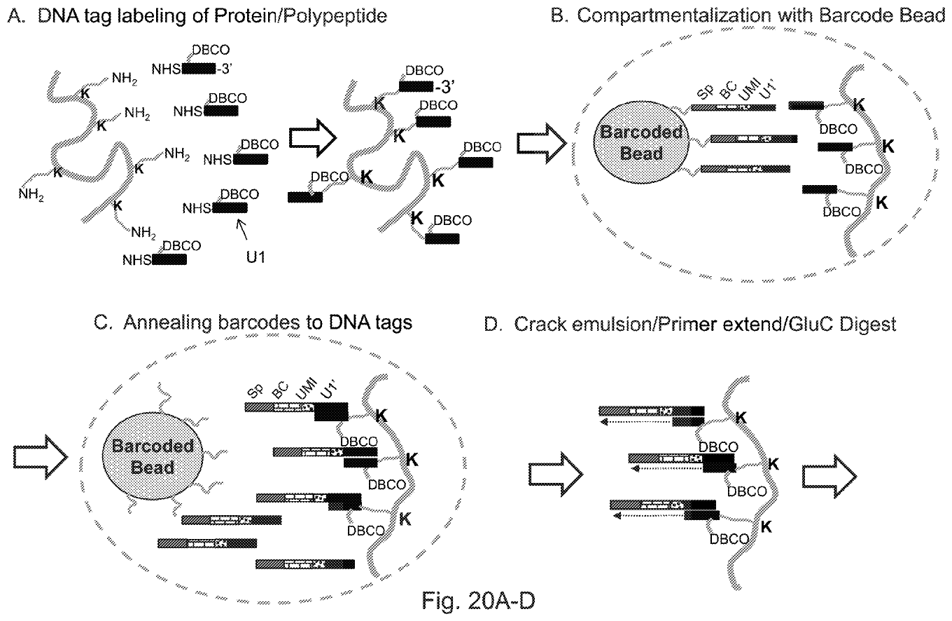

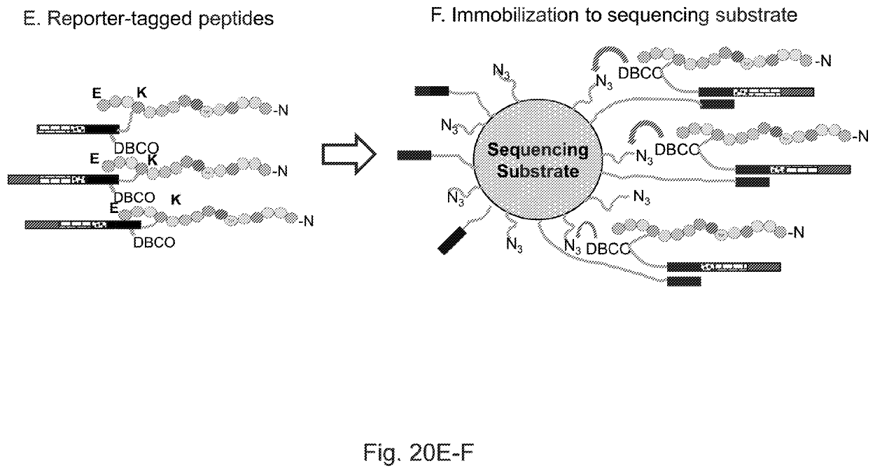

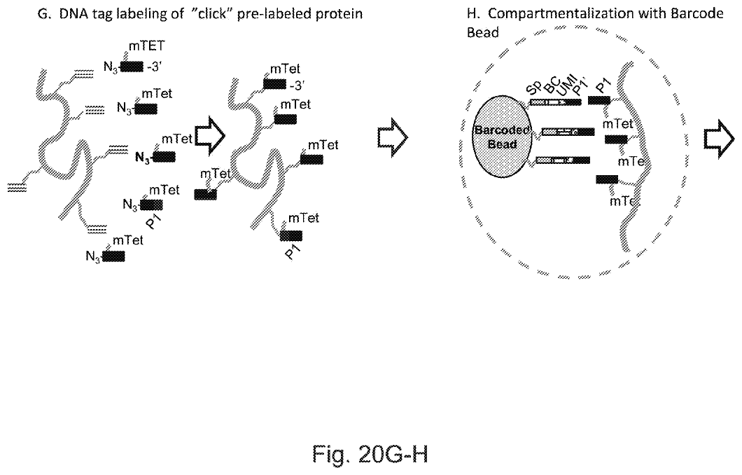

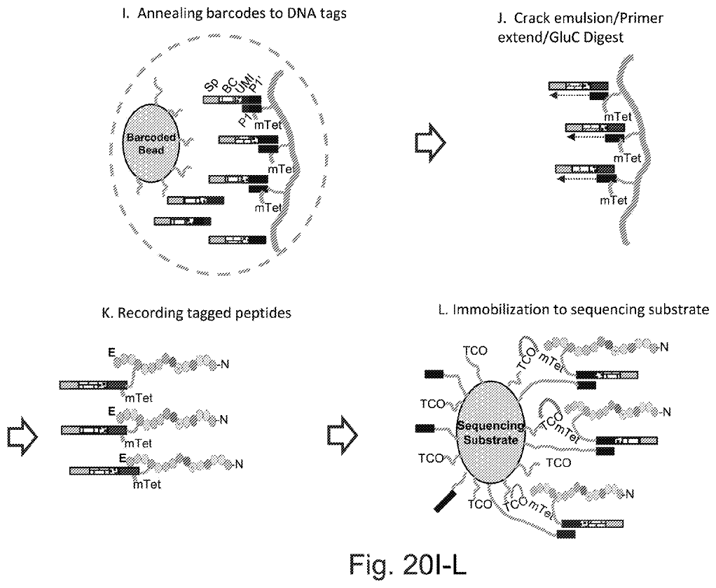

FIGS. 29A-E illustrate examples for the conjugation of DNA recording tags to polypeptides. (A) A denatured polypeptide is labeled with a bifunctional click chemistry reagent, such as alkyne-NHS ester (acetylene-PEG-NETS ester) reagent or alkyne-benzophenone to generate an alkyne-labeled (triple line symbol) polypeptide. An alkyne can also be a strained alkyne, such as cyclooctynes including Dibenzocyclooctyl (DBCO), etc. (B) An example of a DNA recording tag design that is chemically coupled to the alkyne-labeled polypeptide is shown. The recording tag comprises a universal priming sequence (P1), a barcode (BC), and a spacer sequence (Sp). The recording tag is labeled with a mTet moiety for coupling to a substrate surface and an azide moiety for coupling with the alkyne moiety of the labeled polypeptide. (C) A denatured, alkyne-labeled protein or polypeptide is labeled with a recording tag via the alkyne and azide moieties. Optionally, the recording tag-labeled polypeptide can be further labeled with a compartment barcode, e.g., via annealing to complementary sequences attached to a compartment bead and primer extension (also referred to as polymerase extension), or a shown in FIGS. 20H-J. (D) Protease digestion of the recording tag-labeled polypeptide creates a population of recording tag-labeled peptides. In some embodiments, some peptides will not be labeled with any recording tags. In other embodiments, some peptides may have one or more recording tags attached. (E) Recording tag-labeled peptides are immobilized onto a substrate surface using an inverse electron demand Diels-Alder (iEDDA) click chemistry reaction between the substrate surface functionalized with TCO groups and the mTet moieties of the recording tags attached to the peptides. In certain embodiments, clean-up steps may be employed between the different stages shown. The use of orthogonal click chemistries (e.g., azide-alkyne and mTet-TCO) allows both click chemistry labeling of the polypeptides with recording tags, and click chemistry immobilization of the recording tag-labeled peptides onto a substrate surface (see, McKay et al., 2014, Chem. Biol. 21:1075-1101, incorporated by reference in its entirety).

FIGS. 30A-E illustrate an exemplary process of writing sample barcodes into recording tags after initial DNA tag labeling of polypeptides. (A) A denatured polypeptide is labeled with a bifunctional click chemistry reagent such as an alkyne-NHS reagent or alkyne-benzophenone to generate an alkyne-labeled polypeptide. (B) After alkyne (or alternative click chemistry moiety) labeling of the polypeptide, DNA tags comprising a universal priming sequence (P1) and labeled with an azide moiety and an mTet moiety are coupled to the polypeptide via the azide-alkyne interaction. It is understood that other click chemistry interactions may be employed. (C) A recording tag DNA construct comprising a sample barcode information (BCs′) and other recording tag functional components (e.g., universal priming sequence (P1′), spacer sequence (Sp′)) anneals to the DNA tag-labeled polypeptide via complementary universal priming sequences (P1-P1′). Recording tag information is transferred to the DNA tag by polymerase extension. (D) Protease digestion of the recording tag-labeled polypeptide creates a population of recording tag-labeled peptides. (E) Recording tag-labeled peptides are immobilized onto a substrate surface using an inverse electron demand Diels-Alder (iEDDA) click chemistry reaction between a surface functionalized with TCO groups and the mTet moieties of the recording tags attached to the peptides. In certain embodiments, clean-up steps may be employed between the different stages shown. The use of orthogonal click chemistries (e.g., azide-alkyne and mTet-TCO) allows both click chemistry labeling of the polypeptides with recording tags, and click chemistry immobilization of the recording tag-labeled polypeptides onto a substrate surface (see, McKay et al., 2014, Chem. Biol. 21:1075-1101, incorporated by reference in its entirety).

FIGS. 31A-E illustrate examples for bead compartmentalization for barcoding polypeptides. (A) A polypeptide is labeled in solution with a heterobifunctional click chemistry reagent using standard bioconjugation or photoaffinity labeling techniques. Possible labeling sites include ε-amine of lysine residues (e.g., with NHS-alkyne as shown) or the carbon backbone of the peptide (e.g., with benzophenone-alkyne). (B) Azide-labeled DNA tags comprising a universal priming sequence (P1) are coupled to the alkyne moieties of the labeled polypeptide. (C) The DNA tag-labeled polypeptide is annealed to DNA recording tag labeled beads via complementary DNA sequences (P1 and P1′). The DNA recording tags on the bead comprises a spacer sequence (Sp′), a compartment barcode sequence (BC_{P}′), an optional unique molecular identifier (UMI), and a universal sequence (P1′). The DNA recording tag information is transferred to the DNA tags on the polypeptide via polymerase extension (alternatively, ligation could be employed). After information transfer, the resulting polypeptide comprises multiple recording tags containing several functional elements including compartment barcodes. (D) Protease digestion of the recording tag-labeled polypeptide creates a population of recording tag-labeled peptides. The recording tag-labeled peptides are dissociated from the beads, and (E) re-immobilized onto a sequencing substrate (e.g., using iEDDA click chemistry between mTet and TCO moieties as shown).

FIGS. 32A-H illustrate examples for the workflow for Next Generation Protein Assay (NGPA). A protein sample is labeled with a DNA recording tag comprised of several functional units, e.g., a universal priming sequence (P1), a barcode sequence (BC), an optional UMI sequence, and a spacer sequence (Sp) (enables information transfer with a binding agent coding tag). (A) The labeled proteins are immobilized (passively or covalently) to a substrate (e.g., bead, porous bead or porous matrix). (B) The substrate is blocked with protein and, optionally, competitor oligonucleotides (Sp′) complementary to the spacer sequence are added to minimize non-specific interaction of the analyte recording tag sequence. (C) Analyte-specific antibodies (with associated coding tags) are incubated with substrate-bound protein. The coding tag may comprise a uracil base for subsequent uracil specific cleavage. (D) After antibody binding, excess competitor oligonucleotides (Sp′), if added, are washed away. The coding tag transiently anneals to the recording tag via complementary spacer sequences, and the coding tag information is transferred to the recording tag in a primer extension reaction to generate an extended recording tag. If the immobilized protein is denatured, the bound antibody and annealed coding tag can be removed under alkaline wash conditions such as with 0.1N NaOH. If the immobilized protein is in a native conformation, then milder conditions may be needed to remove the bound antibody and coding tag. An example of milder antibody removal conditions is outlined in panels E-H. (E) After information transfer from the coding tag to the recording tag, the coding tag is nicked (cleaved) at its uracil site using a uracil-specific excision reagent (e.g., USER™) enzyme mix. (F) The bound antibody is removed from the protein using a high-salt, low/high pH wash. The truncated DNA coding tag remaining attached to the antibody is short and rapidly elutes off as well. The longer DNA coding tag fragment may or may not remain annealed to the recording tag. (G) A second binding cycle commences as in steps (B)-(D) and a second primer extension step transfers the coding tag information from the second antibody to the extended recording tag via primer extension. (H) The result of two binding cycles is a concatenate of binding information from the first antibody and second antibody attached to the recording tag.

FIGS. 33A-D illustrate Single-step Next Generation Protein Assay (NGPA) using multiple binding agents and enzymatically-mediated sequential information transfer. NGPA assay with immobilized protein molecule simultaneously bound by two cognate binding agents (e.g., antibodies). After multiple cognate antibody binding events, a combined primer extension and DNA nicking step is used to transfer information from the coding tags of bound antibodies to the recording tag. The caret symbol (\hat{ }) in the coding tags represents a double stranded DNA nicking endonuclease site. In FIG. 33A, the coding tag of the antibody bound to epitope 1 (Epi #1) of a protein transfers coding tag information (e.g., encoder sequence) to the recording tag in a primer extension step following hybridization of complementary spacer sequences. In FIG. 33B, once the double stranded DNA duplex between the extended recording tag and coding tag is formed, a nicking endonuclease that cleaves only one strand of DNA on a double-stranded DNA substrate, such as Nt.BsmAI, which is active at 37° C., is used to cleave the coding tag. Following the nicking step, the duplex formed from the truncated coding tag-binding agent and extended recording tag is thermodynamically unstable and dissociates. The longer coding tag fragment may or may not remain annealed to the recording tag. In FIG. 33C, this allows the coding tag from the antibody bound to epitope #2 (Epi #2) of the protein to anneal to the extended recording tag via complementary spacer sequences, and the extended recording tag to be further extended by transferring information from the coding tag of Epi #2 antibody to the extended recording tag via primer extension. In FIG. 33D, once again, after a double stranded DNA duplex is formed between the extended recording tag and coding tag of Epi #2 antibody, the coding tag is nicked by a nicking endonuclease, such Nb.BssSI. In certain embodiments, use of a non-strand displacing polymerase during primer extension (also referred to as polymerase extension) is preferred. A non-strand displacing polymerase prevents extension of the cleaved coding tag stub that remains annealed to the recording tag by more than a single base. The process of Figures A-D can repeat itself until all the coding tags of proximal bound binding agents are “consumed” by the hybridization, information transfer to the extended recording tag, and nicking steps. The coding tag can comprise an encoder sequence identical for all binding agents (e.g., antibodies) specific for a given analyte (e.g., cognate protein), can comprise an epitope-specific encoder sequence, or can comprise a unique molecular identifier (UMI) to distinguish between different molecular events.

FIGS. 34A-C illustrate examples for controlled density of recording tag-peptide immobilization using titration of reactive moieties on substrate surface. In FIG. 34A, peptide density on a substrate surface may be titrated by controlling the density of functional coupling moieties on the surface of the substrate. This can be accomplished by derivatizing the surface of the substrate with an appropriate ratio of active coupling molecules to “dummy” coupling molecules. In the example shown, NHS—PEG-TCO reagent (active coupling molecule) is combined with NHS-mPEG (dummy molecule) in a defined ratio to derivitize an amine surface with TCO. Functionalized PEGs come in various molecular weights from 300 to over 40,000. In FIG. 34B, a bifunctional 5′ amine DNA recording tag (mTet is other functional moiety) is coupled to a N-terminal Cys residue of a peptide using a succinimidyl 4-(N-maleimidomethyl)cyclohexane-1 (SMCC) bifunctional cross-linker. The internal mTet-dT group on the recording tag is created from an azide-dT group using mTetrazine-Azide. In FIG. 34C, the recording tag labeled peptides are immobilized to the activated substrate surface from FIG. 34A using the iEDDA click chemistry reaction with mTet and TCO. The mTet-TCO iEDDA coupling reaction is extremely fast, efficient, and stable (mTet-TCO is more stable than Tet-TCO).

FIGS. 35A-C illustrate examples for Next Generation Protein Sequencing (NGPS) Binding Cycle-Specific Coding Tags. (A) Design of NGPS assay with a cycle-specific N-terminal amino acid (NTAA) binding agent coding tags. An NTAA binding agent (e.g., antibody specific for N-terminal DNP-labeled tyrosine) binds to a DNP-labeled NTAA of a peptide associated with a recording tag comprising a universal priming sequence (P1), barcode (BC) and spacer sequence (Sp). When the binding agent binds to a cognate NTAA of the peptide, the coding tag associated with the NTAA binding agent comes into proximity of the recording tag and anneals to the recording tag via complementary spacer sequences. Coding tag information is transferred to the recording tag via primer extension. To keep track of which binding cycle a coding tag represents, the coding tag can comprise of a cycle-specific barcode. In certain embodiments, coding tags of binding agents that bind to an analyte have the same encoder barcode independent of cycle number, which is combined with a unique binding cycle-specific barcode. In other embodiments, a coding tag for a binding agent to an analyte comprises a unique encoder barcode for the combined analyte-binding cycle information. In either approach, a common spacer sequence can be used for binding agents' coding tags in each binding cycle. (B) In this example, binding agents from each binding cycle have a short binding cycle-specific barcode to identify the binding cycle, which together with the encoder barcode that identifies the binding agent, provides a unique combination barcode that identifies a particular binding agent-binding cycle combination. (C) After completion of the binding cycles, the extended recording tag can be converted into an amplifiable library using a capping cycle step where, for example, a cap comprising a universal priming sequence P1′ linked to a universal priming sequence P2 and spacer sequence Sp′ initially anneals to the extended recording tag via complementary P1 and P1′ sequences to bring the cap in proximity to the extended recording tag. The complementary Sp and Sp′ sequences in the extended recording tag and cap anneal and primer extension adds the second universal primer sequence (P2) to the extended recording tag.

FIGS. 36A-E illustrate examples for DNA based model system for demonstrating information transfer from coding tags to recording tags. Exemplary binding and intra-molecular writing was demonstrated by an oligonucleotide model system. The targeting agent A′ and B′ in coding tags were designed to hybridize to target binding regions A and B in recording tags. Recording tag (RT) mix was prepared by pooling two recoding tags, saRT_Abc_v2 (A target) and saRT_Bbc_V2 (B target), at equal concentrations. Recording tags are biotinylated at their 5′ end and contain a unique target binding region, a universal forward primer sequence, a unique DNA barcode, and an 8 base common spacer sequence (Sp). The coding tags contain unique encoder barcodes base flanked by 8 base common spacer sequences (Sp′), one of which is covalently linked to A or B target agents via polyethylene glycol linker. In FIG. 36A, biotinylated recording tag oligonucleotides (saRT_Abc_v2 and saRT_Bbc_V2) along with a biotinylated Dummy-T10 oligonucleotide were immobilized to streptavidin beads. The recording tags were designed with A or B capture sequences (recognized by cognate binding agents—A′ and B′, respectively), and corresponding barcodes (rtA_BC and rtB_BC) to identify the binding target. All barcodes in this model system were chosen from the set of 65 15-mer barcodes (SEQ ID NOs:1-65). In some cases, 15-mer barcodes were combined to constitute a longer barcode for ease of gel analysis. In particular, rtA_BC=BC_1+BC_2; rtB_BC=BC_3. Two coding tags for binding agents cognate to the A and B sequences of the recording tags, namely CT_A′-bc (encoder barcode=BC_5) and CT_B′-bc (encoder barcode=BC_5+BC_6) were also synthesized. Complementary blocking oligonucleotides (DupCT_A′BC and DupCT_AB′BC) to a portion of the coding tag sequence (leaving a single stranded Sp′ sequence) were optionally pre-annealed to the coding tags prior to annealing of coding tags to the bead-immobilized recording tags. A strand displacing polymerase removes the blocking oligonucleotide during polymerase extension. A barcode key (inset) indicates the assignment of 15-mer barcodes to the functional barcodes in the recording tags and coding tags. In FIG. 36B, the recording tag barcode design and coding tag encoder barcode design provide an easy gel analysis of “intra-molecular” vs. “inter-molecular” interactions between recording tags and coding tags. In this design, undesired “inter-molecular” interactions (A recording tag with B′ coding tag, and B recording tag with A′ coding tag) generate gel products that are wither 15 bases longer or shorter than the desired “intra-molecular” (A recording tag with A′ coding tag; B recording tag with B′ coding tag) interaction products. The primer extension step changes the A′ and B′ coding tag barcodes (ctA′_BC, ctB′_BC) to the reverse complement barcodes (ctA_BC and ctB_BC). In FIG. 36C, a primer extension assay demonstrated information transfer from coding tags to recording tags, and addition of adapter sequences via primer extension on annealed EndCap oligonucleotide for PCR analysis. FIG. 36D shows optimization of “intra-molecular” information transfer via titration of surface density of recording tags via use of Dummy-T20 oligo. Biotinylated recording tag oligonucleotides were mixed with biotinylated Dummy-T20 oligonucleotide at various ratios from 1:0, 1:10, all the way down to 1:10000. At reduced recording tag density (1:10³ and 1:10⁴), “intra-molecular” interactions predominate over “inter-molecular” interactions. In FIG. 36E, as a simple extension of the DNA model system, a simple protein binding system comprising Nano-Tag₁₅ peptide-Streptavidin binding pair is illustrated (K_{D} ˜4 nM) (Perbandt et al., 2007, Proteins 67:1147-1153), but any number of peptide-binding agent model systems can be employed. Nano-Tag₁₅ peptide sequence is (fM)DVEAWLGARVPLVET (SEQ ID NO:131) (fM=formyl-Met). Nano-Tag₁₅ peptide further comprises a short, flexible linker peptide (GGGGS; SEQ ID NO: 140) and a cysteine residue for coupling to the DNA recording tag. Other examples peptide tag-cognate binding agent pairs include: calmodulin binding peptide (CBP)-calmodulin (K_{D} ˜2 pM) (Mukherjee et al., 2015, J. Mol. Biol. 427: 2707-2725), amyloid-beta (Aβ16-27) peptide-US7/Lcn2 anticalin (0.2 nM) (Rauth et al., 2016, Biochem. J. 473: 1563-1578), PA tag/NZ-1 antibody (K_{D} ˜400 pM), FLAG-M2 Ab (28 nM), HA-4B2 Ab (1.6 nM), and Myc-9E10 Ab (2.2 nM) (Fujii et al., 2014, Protein Expr. Purif. 95:240-247). As a test of intra-molecular information transfer from the binding agent's coding tag to the recording tag via primer extension, an oligonucleotide “binding agent” that binds to complementary DNA sequence “A” can be used in testing and development. This hybridization event has essentially greater than fM affinity. Streptavidin may be used as a test binding agent for the Nano-tag₁₅ peptide epitope. The peptide tag-binding agent interaction is high affinity, but can easily be disrupted with an acidic and/or high salt washes (Perbandt et al., supra).

FIGS. 37A-B illustrate examples for use of nano- or micro-emulsion PCR to transfer information from UMI-labeled N or C terminus to DNA tags labeling body of polypeptide. In FIG. 37A, a polypeptide is labeled, at its N- or C-terminus with a nucleic acid molecule comprising a unique molecular identifier (UMI). The UMI may be flanked by sequences that are used to prime subsequent PCR. The polypeptide is then “body labeled” at internal sites with a separate DNA tag comprising sequence complementary to a priming sequence flanking the UMI. In FIG. 37B, the resultant labeled polypeptides are emulsified and undergo an emulsion PCR (ePCR) (alternatively, an emulsion in vitro transcription-RT-PCR (IVT-RT-PCR) reaction or other suitable amplification reaction can be performed) to amplify the N- or C-terminal UMI. A microemulsion or nanoemulsion is formed such that the average droplet diameter is 50-1000 nm, and that on average there is fewer than one polypeptide per droplet. A snapshot of a droplet content pre- and post PCR is shown in the left panel and right panel, respectively. The UMI amplicons hybridize to the internal polypeptide body DNA tags via complementary priming sequences and the UMI information is transferred from the amplicons to the internal polypeptide body DNA tags via primer extension.

FIG. 38 illustrates examples for single cell proteomics. Cells are encapsulated and lysed in droplets containing polymer-forming subunits (e.g., acrylamide). The polymer-forming subunits are polymerized (e.g., polyacrylamide), and proteins are cross-linked to the polymer matrix. The emulsion droplets are broken and polymerized gel beads that contain a single cell protein lysate attached to the permeable polymer matrix are released. The proteins are cross-linked to the polymer matrix in either their native conformation or in a denatured state by including a denaturant such as urea in the lysis and encapsulation buffer. Recording tags comprising a compartment barcode and other recording tag components (e.g., universal priming sequence (P1), spacer sequence (Sp), optional unique molecular identifier (UMI)) are attached to the proteins using a number of methods known in the art and disclosed herein, including emulsification with barcoded beads, or combinatorial indexing. The polymerized gel bead containing the single cell protein can also be subjected to proteinase digest after addition of the recording tag to generate recording tag labeled peptides suitable for peptide sequencing. In certain embodiments, the polymer matrix can be designed such that is dissolves in the appropriate additive such as disulfide cross-linked polymer that break upon exposure to a reducing agent such as tris(2-carboxyethyl)phosphine (TCEP) or dithiothreitol (DTT).

FIGS. 39A-E illustrate examples for enhancement of amino acid elimination reaction using a bifunctional N-terminal amino acid (NTAA) modifier and a chimeric elimination reagent. (A) and (B) A peptide attached to a solid-phase substrate is modified with a bifunctional NTAA modifier, such as biotin-phenyl isothiocyanate (PITC). (C) A low affinity Edmanase (>μM Kd) is recruited to biotin-PITC labeled NTAAs using a streptavidin-Edmanase chimeric protein. (D) The efficiency of Edmanase elimination is greatly improved due to the increase in effective local concentration as a result of the biotin-strepavidin interaction. (E) The cleaved biotin-PITC labeled NTAA and associated streptavidin-Edmanase chimeric protein diffuse away after elimination. A number of other bioconjugation recruitment strategies can also be employed. An azide modified PITC is commercially available (4-Azidophenyl isothiocyanate, Sigma), allowing a number of simple transformations of azide-PITC into other bioconjugates of PITC, such as biotin-PITC via a click chemistry reaction with alkyne-biotin.

FIGS. 40A-I illustrate examples for generation of C-terminal recording tag-labeled peptides from protein lysate (may be encapsulated in a gel bead). (A) A denatured polypeptide is reacted with an acid anhydride to label lysine residues. In one embodiment, a mix of alkyne (mTet)-substituted citraconic anhydride+proprionic anhydride is used to label the lysines with mTet. (shown as striped rectangles). (B) The result is an alkyne (mTet)-labeled polypeptide, with a fraction of lysines blocked with a proprionic group (shown as squares on the polypeptide chain). The alkyne (mTet) moiety is useful in click-chemistry based DNA labeling. (C) DNA tags (shown as solid rectangles) are attached by click chemistry using azide or trans-cyclooctene (TCO) labels for alkyne or mTet moieties, respectively. (D) Barcodes and functional elements such as a spacer (Sp) sequence and universal priming sequence are appended to the DNA tags using a primer extension step as shown in FIG. 31 to produce recording tag-labeled polypeptide. The barcodes may be a sample barcode, a partition barcode, a compartment barcode, a spatial location barcode, etc., or any combination thereof. (E) The resulting recording tag-labeled polypeptide is fragmented into recording tag-labeled peptides with a protease or chemically. (F) For illustration, a peptide fragment labeled with two recording tags is shown. (G) A DNA tag comprising universal priming sequence that is complementary to the universal priming sequence in the recording tag is ligated to the C-terminal end of the peptide. The C-terminal DNA tag also comprises a moiety for conjugating the peptide to a surface. (H) The complementary universal priming sequences in the C-terminal DNA tag and a stochastically selected recording tag anneal. An intra-molecular primer extension reaction is used to transfer information from the recording tag to the C-terminal DNA tag. (I) The internal recording tags on the peptide are coupled to lysine residues via maleic anhydride, which coupling is reversible at acidic pH. The internal recording tags are cleaved from the peptide's lysine residues at acidic pH, leaving the C-terminal recording tag. The newly exposed lysine residues can optionally be blocked with a non-hydrolyzable anhydride, such as proprionic anhydride.

FIG. 41 illustrates an exemplary workflow for an embodiment of the NGPS assay.

FIGS. 42A-D illustrate exemplary steps of Next-Gen Protein Sequencing (NGPS or ProteoCode) sequencing assay. An N-terminal amino acid (NTAA) acetylation or amidination step on a recording tag-labeled, surface bound peptide can occur before or after binding by an NTAA binding agent, depending on whether NTAA binding agents have been engineered to bind to acetylated NTAAs or native NTAAs. In the first case, (A) the peptide is initially acetylated at the NTAA by chemical means using acetic anhydride or enzymatically with an N-terminal acetyltransferase (NAT). (B) The NTAA is recognized by an NTAA binding agent, such as an engineered anticalin, aminoacyl tRNA synthetase (aaRS), ClpS, etc. A DNA coding tag is attached to the binding agent and comprises a barcode encoder sequence that identifies the particular NTAA binding agent. (C) After binding of the acetylated NTAA by the NTAA binding agent, the DNA coding tag transiently anneals to the recording tag via complementary sequences and the coding tag information is transferred to the recording tag via polymerase extension. In an alternative embodiment, the recording tag information is transferred to the coding tag via polymerase extension. (D) The acetylated NTAA is cleaved from the peptide by an engineered acylpeptide hydrolase (APH), which catalyzes the hydrolysis of terminal acetylated amino acid from acetylated peptides. After elimination of the acetylated NTAA, the cycle repeats itself starting with acetylation of the newly exposed NTAA.N-terminal acetylation is used as an exemplary mode of NTAA modification/elimination, but other N-terminal moieties, such as a guanidinyl moiety can be substituted with a concomitant change in elimination chemistry. If guanidinylation is employed, the guanidinylated NTAA can be cleaved under mild conditions using 0.5-2% NaOH solution (see Hamada, 2016, incorporated by reference in its entirety). APH is a serine peptidase able to catalyse the removal of Nα-acetylated amino acids from blocked peptides and it belongs to the prolyl oligopeptidase (POP) family (clan SC, family S9). It is a crucial regulator of N-terminally acetylated proteins in eukaryal, bacterial and archaeal cells.

FIGS. 43A-B illustrate exemplary recording tag-coding tag design features. (A) Structure of an exemplary recording tag associated protein (or peptide) and bound binding agent (e.g., anticalin) with associated coding tag. A thymidine (T) base is inserted between the spacer (Sp′) and barcode (BC′) sequence on the coding tag to accommodate a stochastic non-templated 3′ terminal adenosine (A) addition in the primer extension reaction. (B) DNA coding tag is attached to a binding agent (e.g., anticalin) via SpyCatcher-SpyTag protein-peptide interaction.

FIGS. 44A-E illustrate examples for enhancement of NTAA cleavage reaction using hybridization of cleavage agent to recording tag. In FIGS. 44A-B, a recording tag-labeled peptide attached to a solid-phase substrate (e.g., bead) is modified or labeled at the NTAA (Mod). In FIG. 44C, a cleavage enzyme for the elimination of the NTAA (e.g., acylpeptide hydrolase (APH), amino peptidase (AP), Edmanase, etc.) is attached to a DNA tag comprising a universal priming sequence complementary to the universal priming sequence on the recording tag. The cleavage enzyme is recruited to the functionalized NTAA via hybridization of complementary universal priming sequences on the elimination enzyme's DNA tag and the recording tag. In FIG. 44D, the hybridization step greatly improves the effective affinity of the cleavage enzyme for the NTAA. (E) The eliminated NTAA diffuses away and associated cleavage enzyme can be removed by stripping the hybridized DNA tag.

FIG. 45 illustrates an exemplary cyclic degradation peptide sequencing using peptide ligase+protease+diaminopeptidase. Butelase I ligates the TEV-Butelase I peptide substrate (TENLYFQNHV, SEQ ID NO:132) to the NTAA of the query peptide. Butelase requires an NHV motif at the C-terminus of the peptide substrate. After ligation, Tobacco Etch Virus (TEV) protease is used to cleave the chimeric peptide substrate after the glutamine (Q) residue, leaving a chimeric peptide having an asparagine (N) residue attached to the N-terminus of the query peptide. Diaminopeptidase (DAP) or Dipeptidyl-peptidase, which cleaves two amino acid residues from the N-terminus, shortens the N-added query peptide by two amino acids effectively removing the asparagine residue (N) and the original NTAA on the query peptide. The newly exposed NTAA is read using binding agents as provided herein, and then the entire cycle is repeated “n” times for “n” amino acids sequenced. The use of a streptavidin-DAP metalloenzyme chimeric protein and tethering a biotin moiety to the N-terminal asparagine residue may allow control of DAP processivity.

FIGS. 46A-C illustrate an exemplary “spacer-less” coding tag transfer via ligation of single strand DNA coding tag to single strand DNA recording tag. A single strand DNA coding tag is transferred directly by ligating the coding tag to a recording tag to generate an extended recording tag. (A) Overview of DNA based model system via single strand DNA ligation. The targeting agent B′ sequence conjugated to a coding tag was designed for detecting the B DNA target in the recording tag. The ssDNA recording tag, saRT_Bbca_ssLig is 5′ phosphorylated and 3′ biotinylated, and comprised of a 6 base DNA barcode BCa, a universal forward primer sequence, and a target DNA B sequence. The coding tag, CT_B′bcb_ssLig contains a universal reverse primer sequence, a uracil base, and a unique 6 bases encoder barcode BCb. The coding tag is covalently liked to B′DNA sequence via polyethylene glycol linker. Hybridization of the B′ sequence attached to the coding tag to the B sequence attached to the recording tag brings the 5′ phosphate group of the recording tag and 3′ hydroxyl group of the coding tag into close proximity on the solid surface, resulting in the information transfer via single strand DNA ligation with a ligase, such as CircLigase II. (B) Gel analysis to confirm single strand DNA ligation. Single strand DNA ligation assay demonstrated binding information transfer from coding tags to recording tags. The size of ligated products of 47 bases recording tags with 49 bases coding tag is 96 bases. Specificity is demonstrated given that a ligated product band was observed in the presence of the cognate saRT_Bbca_ssLig recording tag, while no product bands were observed in the presence of the non-cognate saRT_Abcb_ssLig recording tag. (C) Multiple cycles information transfer of coding tag. The first cycle ligated product was treated with USER enzyme to generate a free 5′ phosphorylated terminus for use in the second cycle of information transfer.

FIGS. 47A-B illustrate an exemplary coding tag transfer via ligation of double strand DNA coding tag to double strand DNA recording tag. Multiple information transfer of coding tag via double strand DNA ligation was demonstrated by DNA based model system. (A) Overview of DNA based model system via double strand DNA ligation. The targeting agent A′ sequence conjugated to coding tag was prepared for detection of target binding agent A in recording tag. Both of recording tag and coding tag are composed of two strands with 4 bases overhangs. The proximity overhang ends of both tags hybridize when targeting agent A′ in coding tag hybridizes to target binding agent A in recording tag immobilized on solid surface, resulting in the information transfer via double strand DNA ligation by a ligase, such as a T4 DNA ligase. (B) Gel analysis to confirm double strand DNA ligation. Double strand DNA ligation assay demonstrated A/A′ binding information transfer from coding tags to recording tags. The size of ligated products of 76 and 54 bases recording tags with double strand coding tag is 116 and 111 bases, respectively. The first cycle ligated products were digested by USER Enzyme (NEB), and used in the second cycle assay. The second cycle ligated product bands were observed at around 150 bases.

FIGS. 48A-E illustrate an exemplary peptide-based and DNA-based model system for demonstrating information transfer from coding tags to recording tags with multiple cycles. Multiple information transfer was demonstrated by sequential peptide and DNA model systems. (A) Overview of the first cycle in the peptide based model system. The targeting agent anti-PA antibody conjugated to coding tag was prepared for detecting the PA-peptide tag in recording tag at the first cycle information transfer. In addition, peptide-recording tag complex negative controls were also generated, using a Nanotag peptide or an amyloid beta (Aβ) peptide. Recording tag, amRT_Abc that contains A sequence target agents, poly-dT, a universal forward primer sequence, unique DNA barcodes BC1 and BC2, and an 8 bases common spacer sequence (Sp) is covalently attached to peptide and solid support via amine group at 5′ end and internal alkyne group, respectively. The coding tag, amCT_bc5 that contains unique encoder barcode BC5′ flanked by 8 base common spacer sequences (Sp′) is covalently liked to antibody and C3 linker at the 5′ end and 3′ end, respectively. The information transfer from coding tags to recording tags is done by polymerase extension when anti-PA antibody binds to PA-tag peptide-recording tag (RT) complex. (B) Overview of the second cycle in the DNA based model assay. The targeting agent A′ sequence linked to coding tag was prepared for detecting the A sequence target agent in recording tag. The coding tag, CT_A′_bc13 that contains an 8 bases common spacer sequence (Sp′), a unique encoder barcode BC13′, a universal reverse primer sequence. The information transfer from coding tags to recording tags are done by polymerase extension when A′ sequence hybridizes to A sequence. (C) Recording tag amplification for PCR analysis. The immobilized recording tags were amplified by 18 cycles PCR using P1_F2 and Sp/BC2 primer sets. The recording tag density dependent PCR products were observed at around 56 bp. (D) PCR analysis to confirm the first cycle extension assay. The first cycle extended recording tags were amplified by 21 cycles PCR using P1_F2 and Sp/BC5 primer sets. The strong bands of PCR products from the first cycle extended products were observed at around 80 bp for the PA-peptide RT complex across the different density titration of the complexes. A small background band is observed at the highest complex density for Nano and Aβ peptide complexes as well, ostensibly due to non-specific binding. (E) PCR analysis to confirm the second cycle extension assay. The second extended recording tags were amplified by 21 cycles PCR using P1_F2 and P2_R1 primer sets. Relatively strong bands of PCR products were observed at 117 base pairs for all peptides immobilized beads, which correspond to only the second cycle extended products on original recording tags (BC1+BC2+BC13). The bands corresponding to the second cycle extended products on the first cycle extended recording tags (BC1+BC2+BC5+BC13) were observed at 93 base pairs only when PA-tag immobilized beads were used in the assay.

FIGS. 49A-B use p53 protein sequencing as an example to illustrate the importance of proteoform and the robust mappability of the sequencing reads, e.g., those obtained using a single molecule approach. FIG. 49A at the left panel shows the intact proteoform may be digested to fragments, each of which may comprise one or more methylated amino acids, one or more phosphorylated amino acids, or no post-translational modification. The post-translational modification information may be analyzed together with sequencing reads. The right panel shows various post-translational modifications along the protein. FIG. 49B shows mapping reads using partitions, for example, the read “CPXQXWXDXT” (SEQ ID NO: 170, where X=any amino acid) maps uniquely back to p53 (at the CPVQLWVDST sequence, SEQ ID NO: 169) after blasting the entire human proteome. The sequencing reads do not have to be long—for example, about 10-15 amino acid sequences may give sufficient information to identify the protein within the proteome. The sequencing reads may overlap and the redundancy of sequence information at the overlapping sequences may be used to deduce and/or validate the entire polypeptide sequence.

FIGS. 50A-C illustrate labeling a protein or peptide with a DNA recording Tag using mRNA Display.

FIGS. 51A-E illustrate a single cycle protein identification via N-terminal dipeptide binding to partition barcode-labeled peptides.

FIGS. 52A-E illustrate a single cycle protein identification via N-terminal dipeptide binders to peptides immobilized partition barcoded beads.

FIGS. 53A-D show mass spectrometry analysis of the DNA with the sequence in SEQ ID NO:171 (TTT/i5OCTdU/TTUCGTAGTCCGCGACACTAGTAAGCCGGTATATCAACTGAGTG) that was subjected to water (FIG. 53A), hydrazine hydrate (FIG. 53B), hydrazine hydrate in Tris buffer (FIG. 53C), and hydrazine hydrochloride (FIG. 53D): the Figures show that a nucleic acid is stable to conditions used herein for elimination of a functionalized NTAA from a polypeptide.

FIG. 54 shows mass spectrometry analysis of the DNA with the sequence in SEQ ID NO:171 (TTT/i5OCTdU/TTUCGTAGTCCGCGACACTAGTAAGCCGGTATATCAACTGAGTG) after it was subjected to bis-(4-trifluoromethylpyrazole)methanimine and N-ethylmorpholine buffer, and illustrates that a nucleic acid is stable under conditions useful to form a compound of Formula (II).

FIG. 55A depicts an exemplary assay including modification (e.g., functionalization) and elimination of the N-terminal amino acid (NTAA) of peptides treated with an exemplary chemical reagent, binding of an exemplary binding agent to the modified NTAA and encoding by transferring information from a coding tag associated with the binding agent to a recording tag associated with the peptide. FIG. 55B is a summary of encoding for various peptides (SEQ ID NO: 157-161, 162-166) assessed in a peptide analysis assay using a F-binding agent (top) or L-binding agent (bottom).

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ some materials, steps, terms, and techniques that are conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide and/or oligonucleotide synthesis and modification, polymer array synthesis, hybridization and ligation of polynucleotides and/or oligonucleotides, detection of hybridization, and nucleotide sequencing. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y., all of which are herein incorporated in their entireties by reference for all purposes.

### INTRODUCTION AND OVERVIEW

Molecular recognition and characterization of a protein or polypeptide analyte is typically performed using an immunoassay. There are many different immunoassay formats including ELISA, multiplex ELISA (e.g., spotted antibody arrays, liquid particle ELISA arrays), digital ELISA (e.g., Quanterix, Singulex), reverse phase protein arrays (RPPA), and many others. These different immunoassay platforms all face similar challenges including the development of high affinity and highly-specific (or selective) antibodies (binding agents), limited ability to multiplex at both the sample level and the analyte level, limited sensitivity and dynamic range, and cross-reactivity and background signals.

Binding agent agnostic approaches such as direct protein characterization via peptide sequencing (Edman degradation or Mass Spectroscopy) provide useful alternative approaches. However, neither of these approaches is very parallel or high-throughput. In general, the Edman degradation peptide sequencing method is slow and has a limited throughput of only a few peptides per day. It also employs a strongly acidic reaction step that is incompatible with oligonucleotides, as they are known to degrade under such strongly acidic conditions.

Accordingly, there remains a need in the art for improved techniques relating to macromolecule (e.g., polypeptide or polynucleotide) sequencing and/or analysis, with applications to protein sequencing and/or analysis, as well as to products, methods and kits for accomplishing the same. There is a need for proteomics technology that is highly-parallelized, accurate, sensitive, and high-throughput. These and other aspects of the invention will be apparent upon reference to the following detailed description. To this end, various references are set forth herein which describe in more detail certain background information, procedures, compounds and/or compositions, and are each hereby incorporated by reference in their entirety.

The present disclosure provides methods for modification and removal of the N-terminal amino acid from a peptidic molecule. Because the methods are mild and selective, they can be used for proteins that are conjugated to other materials, e.g. a proteinaceous or oligosaccharide carrier, and they can be applied in the presence of acid-sensitive materials such as oligosaccharides and oligonucleotides. Also, because the methods form an activated intermediate that is reasonably stable, and then apply a second set of conditions to cause cleavage of the N-terminal amino acid, the methods can be used iteratively to remove two, three, ten, or more amino acids from the N-terminal end of the polypeptide. Accordingly, the methods are useful for selectively modifying a polypeptide by removing one or more amino acid residues from the N-terminal end of the polypeptide.

The methods disclosed herein, like Edman degradation, cleave the N-terminal amino acid to leave a truncated polypeptide lacking the N-terminal amino acid residue of the starting polypeptide. They also form a cleavage product, like Edman degradation, that can be characterized to identify the N-terminal amino acid that was removed. Especially for polypeptides from natural origins, which are typically composed mainly or entirely of the 21 commonly known proteinogenic amino acids, there are convenient methods to identify the cleavage products that predictably form when applying the methods herein to a polypeptide. Thus, by sequentially applying the N-terminal cleavage method to a polypeptide, the sequence of amino acids in the polypeptide can be determined by identifying the cleavage product released in each iteration.

In some embodiments, the methods for treating a polypeptide and cleaving the N-terminal amino acid are used for determining the sequence of at least a portion of the polypeptide. In some aspects, the provided methods can be used in the context of a degradation-based polypeptide sequencing assay. In some embodiments, determining the sequence of at least a portion of the polypeptide includes performing any of the methods as described in International Patent Publication Nos. WO 2017/192633, WO 2019/089836, WO 2019/089851. In some cases, the sequence of the polypeptide is analyzed by construction of an extended recording tag (e.g., DNA sequence) representing the polypeptide sequence, such as an extended recording tag. In some embodiments, the assay includes a cyclic including NTAA functionalization and NTAA removal. In some embodiments, the assay includes transfer of coding tag information (e.g., joined to a binding agent) to a recording tag attached to the polypeptide. In some embodiments, one or more steps of the polypeptide analysis assay is repeated in a cyclic manner. For example, the methods for analyzing a polypeptide provided in the present disclosure comprise multiple binding cycles, where the polypeptide is contacted with a plurality of binding agents, and successive binding of binding agents transfers historical binding information in the form of a nucleic acid based coding tag to at least one recording tag associated with the polypeptide. In this way, a historical record containing information about multiple binding events is generated in a nucleic acid format.

Accordingly, the invention provides methods for sequencing a polypeptide by sequentially removing the N-terminal amino acid, and analyzing the cleavage product released with each step to determine which amino acid was cleaved in that step. In some embodiments, the invention provides methods for sequencing a polypeptide by sequentially removing the N-terminal amino acid in a nucleic acid encoding based analysis method that includes binding of the NTAA.

The invention also provides reagents useful for removal of the N-terminal amino acid of a polypeptide, methods of making these reagents, and kits comprising suitable reagents for performing the methods of the invention.

Because the methods for cleaving the N-terminal amino acid employ mild reagents and conditions, they can be applied in samples that also contain acid-sensitive materials. For example, a sample containing the polypeptide of interest might also contain oligonucleotides, which could be used to encode information about the sample for automated processing: while typical Edman conditions, employing a strong acid to cleave the NTAA, are expected to degrade such oligonucleotides, the present methods can be used on such samples without degrading oligonucleotides.

Other aspects and advantages of the invention will be appreciated from the detailed description and examples below.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 1000 amino acids, e.g., having more than 20-30 amino acids. However, it will be appreciated that the step-wise N-terminal amino acid cleavage, when applied to a polypeptide many times, can eventually result in smaller oligopeptides and ultimately tri- and di-peptides and finally a single remaining amino acid. For simplicity, when the methods are described as being applied to a polypeptide, the methods are intended to include smaller oligopeptides, down to a dipeptide. In some embodiments, a polypeptide does not comprise a secondary, tertiary, or higher structure. In some embodiments, the polypeptide is a protein; in other embodiments, it may be a cleavage product from a protein, or it may be a shorter chain of amino acids. In some embodiments, a protein comprises 30 or more amino acids, e.g. having more than 50 amino acids. In some embodiments, in addition to a primary structure, a protein comprises a secondary, tertiary, or higher structure.

The amino acids of the polypeptides are most typically L-amino acids when the polypeptides are of natural origin, since the proteinogenic amino acids are all of the L-configuration. However, the methods work equally well to cleave an N-terminal amino acid of D-configuration, so the residues of a polypeptide to be used in the methods may also be D-amino acids, mixtures of D- and L-amino acids, modified amino acids, amino acid analogs, amino acid mimetics, or any combination thereof, that have the alpha-amino acid backbone. In general, the descriptions and methods provided herein may apply to modification, cleavage, treatment, and/or contact of at least some beta amino acids. For example, isoaspartic acid is a biologically relevant beta amino acid that may be modified, cleaved, treated, and/or contacted as described herein.

Polypeptides may be naturally occurring, synthetically produced, or recombinantly expressed. Polypeptides may be synthetically produced, isolated, recombinantly expressed, or they may be produced by a combination of methodologies as described above. Polypeptides may also comprise additional groups modifying the amino acid chain, for example, functional groups added via post-translational modification to the side chain groups of the amino acid residues. The polymer may be linear or branched, it may comprise modified amino acids, and it may be interrupted by non-amino acids, though the method may not cleave amino acids that do not have the alpha-amino core structure. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group at the alpha position of an acetic acid group, and the acetic acid moiety may contain a side-chain also at the alpha carbon. As used herein, unless otherwise limited, it includes natural and unnatural compounds having the alpha-amino acid core structure and zero, one or two hydrocarbon groups on the alpha carbon along with the amino group. These hydrocarbon groups can vary widely without interfering with the methods described herein. Typically, the common natural amino acids comprise a side chain that is specific to each amino acid, and the amino group plus acetic acid moiety and optional side chain taken together serve as a monomeric subunit of a peptide, commonly referred to as an amino acid residue. The term also includes amino acids having a side chain that forms a 5-6 membered ring by connecting to the amino group; proline is an example of this type of amino acid. An amino acid particularly includes the 20 standard, naturally occurring or canonical amino acids plus selenocysteine, which, while less common, is one of the natural proteinogenic amino acids, and the term also includes non-standard amino acids and modified amino acids. The standard, naturally-occurring proteinogenic amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Selenocysteine (Sec), Serine (S or Ser), Threonine (T or Thr), Valine (V or Val), Tryptophan (W or Trp), and Tyrosine (Y or Tyr).

An amino acid in polypeptides used in the methods herein may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, pyrrolysine, and N-formylmethionine, Proline and Pyruvic acid derivatives such as hydroxyprolines, 3-substituted alanine derivatives, glycine derivatives, ring-substituted phenylalanine and tyrosine derivatives, linear core amino acids, N-methyl amino acids. In a preferred embodiment, the polypeptides of the invention are comprised of the proteinogenic amino acids, and optionally include naturally occurring post-translational modifications of these amino acids.

While the methods of the invention can generally be used on any polypeptide, it is sometimes advantageous to prepare a polypeptide to enhance reliability and efficiency of the methods described herein. For example, as the methods of the invention operate by functionalizing the N-terminal amine group of a polypeptide, they may also modify certain functional groups that may be present elsewhere on the polypeptide. One example is lysine, which may be present in a polypeptide and possesses a free —NH₂ group. In some embodiments, it may be useful to modify any lysine —NH₂ that may be present, which can be done using methods known in the art. Also, while the methods of the invention are capable of modifying and eliminating proline when it is the NTAA, in the interest of efficiency it is sometimes helpful to treat the polypeptide with an enzyme (e.g., proline aminopeptidase or proline iminopeptidase (PIP)) before or during the process of modifying the NTAA for cleavage. Thus methods of the invention may include an optional step of treating a polypeptide with one or more enzymes to remove the N-terminal amino acid of the polypeptide (e.g., proline aminopeptidase, proline iminopeptidase (PIP), pyroglutamate aminopeptidase (pGAP), asparagine amidohydrolase, peptidoglutaminase asparaginase, protein glutaminase, or a homolog thereof); and kits for practicing methods of the invention may optionally include one or more enzymes to remove the N-terminal amino acid of the polypeptide (e.g., proline aminopeptidase, proline iminopeptidase (PIP), pyroglutamate aminopeptidase (pGAP), asparagine amidohydrolase, peptidoglutaminase asparaginase, protein glutaminase, or a homolog thereof) for use in this fashion.

As used herein, the term “post-translational modification” and variations thereof refers to modifications that occur on a peptide after its translation by ribosomes is complete. A post-translational modification may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels. In some embodiments, the term excludes modifications of the amino group of the N-terminal amino acid of a polypeptide.

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is the study of the proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof. As used herein, the term “proteomics” refers to quantitative analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

As used herein, the term “binding agent” refers to a nucleic acid molecule, a peptide, a polypeptide, a protein, carbohydrate, or a small molecule that binds to, associates, unites with, recognizes, or combines with a polypeptide or a component or feature of a polypeptide. A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a polypeptide (e.g., a single amino acid of a polypeptide) or bind to a plurality of linked subunits of a polypeptide (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid (e.g., an amino acid that has been functionalized by a reagent such as a compound of Formula (AA) as described herein) over a non-modified or unlabeled amino acid. For example, a binding agent may preferably bind to an amino acid that has been functionalized with an acetyl moiety, guanyl moiety, dansyl moiety, PTC moiety, DNP moiety, SNP moiety, etc., over an amino acid that does not possess said moiety. A binding agent may bind to a post-translational modification of a peptide molecule. A binding agent may exhibit selective binding to a component or feature of a polypeptide (e.g., a binding agent may selectively bind to one of the 20 possible natural amino acid residues and with bind with very low affinity or not at all to the other 19 natural amino acid residues). A binding agent may exhibit less selective binding, where the binding agent is capable of binding a plurality of components or features of a polypeptide (e.g., a binding agent may bind with similar affinity to two or more different amino acid residues). A binding agent comprises a coding tag, which may be joined to the binding agent by a linker.

As used herein, the term “fluorophore” refers to a molecule which absorbs electromagnetic energy at one wavelength and re-emits energy at another wavelength. A fluorophore may be a molecule or part of a molecule including fluorescent dyes and proteins. Additionally, a fluorophore may be chemically, genetically, or otherwise connected or fused to another molecule to produce a molecule that has been “tagged” with the fluorophore.

As used herein, the term “linker” refers to one or more of a nucleotide, a nucleotide analog, an amino acid, a peptide, a polypeptide, or a non-nucleotide chemical moiety that is used to join two molecules. A linker may be used to join a binding agent with a coding tag, a recording tag with a polypeptide, a polypeptide with a solid support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

As used herein, the term “non-cognate binding agent” refers to a binding agent that is not capable of binding or binds with low affinity to a polypeptide feature, component, or subunit being interrogated in a particular binding cycle reaction as compared to a “cognate binding agent”, which binds with high affinity to the corresponding polypeptide feature, component, or subunit. For example, if a tyrosine residue of a peptide molecule is being interrogated in a binding reaction, non-cognate binding agents are those that bind with low affinity or not at all to the tyrosine residue, such that the non-cognate binding agent does not efficiently transfer coding tag information to the recording tag under conditions that are suitable for transferring coding tag information from cognate binding agents to the recording tag. Alternatively, if a tyrosine residue of a peptide molecule is being interrogated in a binding reaction, non-cognate binding agents are those that bind with low affinity or not at all to the tyrosine residue, such that recording tag information does not efficiently transfer to the coding tag under suitable conditions for those embodiments involving extended coding tags rather than extended recording tags.

The terminal amino acid at one end of the peptide chain that has a free amino group is referred to herein as the “N-terminal amino acid” (NTAA). Note that, as depicted in some of the structures herein, the side chain of an amino acid, including the NTAA, can optionally cyclize onto the amine; so the free amino group may not be —NH₂ if the side chain (like that of proline) cyclizes onto the amine. It is nevertheless an accessible and nucleophilic amine, subject to functionalization according to the methods described herein, and the functionalized NTAA is still subject to elimination under the cleavage conditions of the methods.

The terminal amino acid at the other end of the chain typically has a free carboxyl group and is referred to herein as the “C-terminal amino acid” (CTAA). It is common for a polypeptide to be attached to a carrier or surface via the carboxyl of the C-terminal amino acid; for example, the CTAA is commonly used to attach or conjugate the polypeptide to a particle for solid phase peptide synthesis. The methods of the invention are useful to cleave N-terminal amino acid residues from such C-terminal conjugated polypeptides attached to a solid surface such as a particle or bead or glass slide, and to polypeptides attached to a carrier such as an oligosaccharide or other carrier, as well as free polypeptides.

The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be functionalized with a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error correcting barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

A “sample barcode”, also referred to as “sample tag” identifies from which sample a polypeptide derives.

A “spatial barcode” identifies which region of a 2-D or 3-D tissue section from which a polypeptide derives. Spatial barcodes may be used for molecular pathology on tissue sections. A spatial barcode allows for multiplex sequencing of a plurality of samples or libraries from tissue section(s).

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence, which is optionally flanked by one spacer on one side or flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “encoder sequence” or “encoder barcode” refers to a nucleic acid molecule of about 2 bases to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) in length that provides identifying information for its associated binding agent. The encoder sequence may uniquely identify its associated binding agent. In certain embodiments, an encoder sequence provides identifying information for its associated binding agent and for the binding cycle in which the binding agent is used. In other embodiments, an encoder sequence is combined with a separate binding cycle-specific barcode within a coding tag. Alternatively, the encoder sequence may identify its associated binding agent as belonging to a member of a set of two or more different binding agents. In some embodiments, this level of identification is sufficient for the purposes of analysis. For example, in some embodiments involving a binding agent that binds to an amino acid, it may be sufficient to know that a peptide comprises one of two possible amino acids at a particular position, rather than definitively identify the amino acid residue at that position. In another example, a common encoder sequence is used for polyclonal antibodies, which comprises a mixture of antibodies that recognize more than one epitope of a protein target, and have varying specificities. In other embodiments, where an encoder sequence identifies a set of possible binding agents, a sequential decoding approach can be used to produce unique identification of each binding agent. This is accomplished by varying encoder sequences for a given binding agent in repeated cycles of binding (see, Gunderson, et al., 2004, Genome Res. 14:870-7). The partially identifying coding tag information from each binding cycle, when combined with coding information from other cycles, produces a unique identifier for the binding agent, e.g., the particular combination of coding tags rather than an individual coding tag (or encoder sequence) provides the uniquely identifying information for the binding agent. Preferably, the encoder sequences within a library of binding agents possess the same or a similar number of bases.

As used herein the term “binding cycle specific tag”, “binding cycle specific barcode”, or “binding cycle specific sequence” refers to a unique sequence used to identify a library of binding agents used within a particular binding cycle. A binding cycle specific tag may comprise about 2 bases to about 8 bases (e.g., 2, 3, 4, 5, 6, 7, or 8 bases) in length. A binding cycle specific tag may be incorporated within a binding agent's coding tag as part of a spacer sequence, part of an encoder sequence, part of a UMI, or as a separate component within the coding tag.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the macromolecule (e.g., UMI information) associated with the recording tag can be transferred to the coding tag. Identifying information can comprise any information characterizing a molecule such as information pertaining to identity, sample, fraction, partition, spatial location, interacting neighboring molecule(s), cycle number, etc. Additionally, the presence of UMI information can also be classified as identifying information. In certain embodiments, after a binding agent binds a polypeptide, information from a coding tag linked to a binding agent can be transferred to the recording tag associated with the polypeptide while the binding agent is bound to the polypeptide. In other embodiments, after a binding agent binds a polypeptide, information from a recording tag associated with the polypeptide can be transferred to the coding tag linked to the binding agent while the binding agent is bound to the polypeptide. A recoding tag may be directly linked to a polypeptide, linked to a polypeptide via a multifunctional linker, or associated with a polypeptide by virtue of its proximity (or co-localization) on a solid support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length providing a unique identifier tag for each polypeptide or binding agent to which the UMI is linked. A polypeptide UMI can be used to computationally deconvolute sequencing data from a plurality of extended recording tags to identify extended recording tags that originated from an individual polypeptide. A binding agent UMI can be used to identify each individual binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5” or “sense”. The term “reverse” when used in context with a “universal priming site” or “universal primer” may also be referred to as “3′” or “antisense”.

As used herein, the term “extended recording tag” refers to a recording tag to which information of at least one binding agent's coding tag (or its complementary sequence) has been transferred following binding of the binding agent to a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

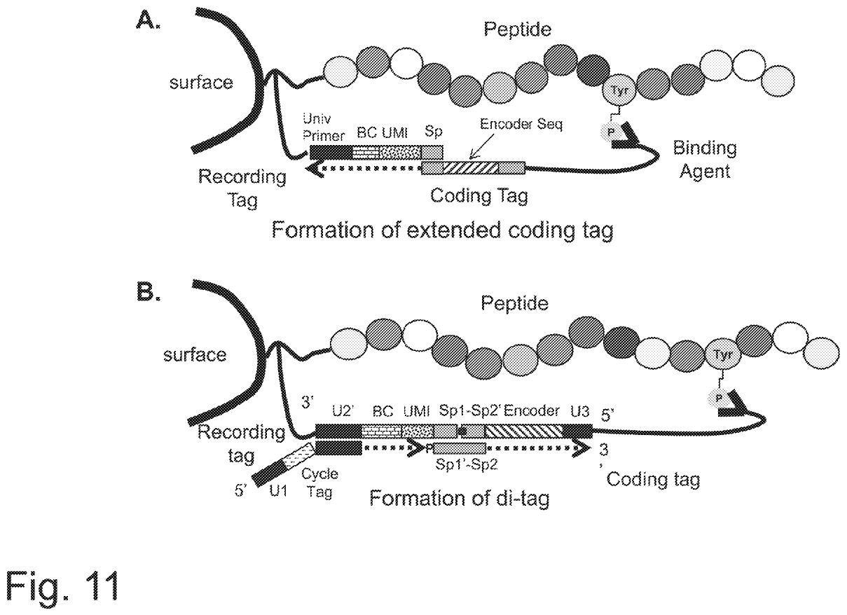

As used herein, the term “extended coding tag” refers to a coding tag to which information of at least one recording tag (or its complementary sequence) has been transferred following binding of a binding agent, to which the coding tag is joined, to a polypeptide, to which the recording tag is associated. Information of a recording tag may be transferred to the coding tag directly (e.g., ligation), or indirectly (e.g., primer extension). Information of a recording tag may be transferred enzymatically or chemically. In certain embodiments, an extended coding tag comprises information of one recording tag, reflecting one binding event. As used herein, the term “di-tag” or “di-tag construct” or “di-tag molecule” refers to a nucleic acid molecule to which information of at least one recording tag (or its complementary sequence) and at least one coding tag (or its complementary sequence) has been transferred following binding of a binding agent, to which the coding tag is joined, to a polypeptide, to which the recording tag is associated (see, e.g., FIG. 11B). Information of a recording tag and coding tag may be transferred to the di-tag indirectly (e.g., primer extension). Information of a recording tag may be transferred enzymatically or chemically. In certain embodiments, a di-tag comprises a UMI of a recording tag, a compartment tag of a recording tag, a universal priming site of a recording tag, a UMI of a coding tag, an encoder sequence of a coding tag, a binding cycle specific barcode, a universal priming site of a coding tag, or any combination thereof.

As used herein, the term “solid support”, “solid surface”, or “solid substrate” or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a PTFE membrane, a nitrocellulose membrane, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polyester, polyacrylate, polystyrene, polyethylene vinyl acetate, polypropylene, polymethacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

The compounds described herein are in many cases capable of forming salts with an acid or base, and the invention is intended to include stable salts of the compounds. Indeed, in some instances it is advantageous to use or isolate a salt rather than the neutral compound for reasons of stability or solubility, for example; and in some cases, compounds are prepared in a medium that produces them as a salt, or they are used in a medium that produces a salt. Moreover, compounds comprising a polypeptide or amino acid typically include one or more ionizable groups that are suitable for salt formation. The invention thus includes acid addition salts of compounds that accept an acidic proton, and base addition salts of compounds that readily donate a proton, as well as zwitterionic forms of compounds having both acidic and basic properties, which is the case with many polypeptides.

For a compound of the invention that contains a basic nitrogen, a suitable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

Examples of suitable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

Compounds of the invention having an acidic moiety may be treated with a base to produce a salt having a positively charged counterion, and these salts are also suitable for use in the compounds and methods of the invention. They include salts such as sodium, lithium, potassium, calcium, magnesium, ammonium, alkylated ammoniums, quaternary ammoniums, and the like. In addition to these, the base can be a cyclic amine such as piperidine, piperazine, morpholine, DBU, DABCO, N-methyl morpholine, pyridine, DMAP, and similar proton-accepting compounds, including diheteronucleophiles such as hydrazine that may be present in excess in a reaction mixture forming a compound of the invention, and thus may form a salt with the compound at least in the reaction mixture. The term ‘salt’ or ‘salts’ as used herein is intended to include all of these types of salts.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” a polypeptide means to identify, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n−1, n−2, n−3, and so forth). This is accomplished by elimination of the n NTAA, thereby converting the n−1 amino acid of the peptide to an N-terminal amino acid (referred to herein as the “n−1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “compartment” refers to a physical area or volume that separates or isolates a subset of polypeptides from a sample of polypeptides. For example, a compartment may separate an individual cell from other cells, or a subset of a sample's proteome from the rest of the sample's proteome. A compartment may be an aqueous compartment (e.g., microfluidic droplet), a solid compartment (e.g., picotiter well or microtiter well on a plate, tube, vial, gel bead), or a separated region on a surface. A compartment may comprise one or more beads to which polypeptides may be immobilized.

As used herein, the term “compartment tag” or “compartment barcode” refers to a single or double stranded nucleic acid molecule of about 4 bases to about 100 bases (including 4 bases, 100 bases, and any integer between) that comprises identifying information for the constituents (e.g., a single cell's proteome), within one or more compartments (e.g., microfluidic droplet). A compartment barcode identifies a subset of polypeptides in a sample that have been separated into the same physical compartment or group of compartments from a plurality (e.g., millions to billions) of compartments. Thus, a compartment tag can be used to distinguish constituents derived from one or more compartments having the same compartment tag from those in another compartment having a different compartment tag, even after the constituents are pooled together. By labeling the proteins and/or peptides within each compartment or within a group of two or more compartments with a unique compartment tag, peptides derived from the same protein, protein complex, or cell within an individual compartment or group of compartments can be identified. A compartment tag comprises a barcode, which is optionally flanked by a spacer sequence on one or both sides, and an optional universal primer. The spacer sequence can be complementary to the spacer sequence of a recording tag, enabling transfer of compartment tag information to the recording tag. A compartment tag may also comprise a universal priming site, a unique molecular identifier (for providing identifying information for the peptide attached thereto), or both, particularly for embodiments where a compartment tag comprises a recording tag to be used in downstream peptide analysis methods described herein. A compartment tag can comprise a functional moiety (e.g., aldehyde, NHS, mTet, alkyne, etc.) for coupling to a peptide. Alternatively, a compartment tag can comprise a peptide comprising a recognition sequence for a protein ligase to allow ligation of the compartment tag to a peptide of interest. A compartment can comprise a single compartment tag, a plurality of identical compartment tags save for an optional UMI sequence, or two or more different compartment tags. In certain embodiments each compartment comprises a unique compartment tag (one-to-one mapping). In other embodiments, multiple compartments from a larger population of compartments comprise the same compartment tag (many-to-one mapping). A compartment tag may be joined to a solid support within a compartment (e.g., bead) or joined to the surface of the compartment itself (e.g., surface of a picotiter well). Alternatively, a compartment tag may be free in solution within a compartment.

As used herein, the term “partition” refers to an assignment (e.g., random assignment) of a unique barcode to a subpopulation of polypeptides from a population of polypeptides within a sample. In certain embodiments, partitioning may be achieved by distributing polypeptides into compartments. A partition may be comprised of the polypeptides within a single compartment or the polypeptides within multiple compartments from a population of compartments.

As used herein, a “partition tag” or “partition barcode” refers to a single or double stranded nucleic acid molecule of about 4 bases to about 100 bases (including 4 bases, 100 bases, and any integer between) that comprises identifying information for a partition. In certain embodiments, a partition tag for a polypeptide refers to identical compartment tags arising from the partitioning of polypeptides into compartment(s) labeled with the same barcode.

As used herein, the term “fraction” refers to a subset of polypeptides within a sample that have been sorted from the rest of the sample or organelles using physical or chemical separation methods, such as fractionating by size, hydrophobicity, isoelectric point, affinity, and so on. Separation methods include HPLC separation, gel separation, affinity separation, cellular fractionation, cellular organelle fractionation, tissue fractionation, etc. Physical properties such as fluid flow, magnetism, electrical current, mass, density, or the like can also be used for separation.

As used herein, the term “fraction barcode” refers to a single or double stranded nucleic acid molecule of about 4 bases to about 100 bases (including 4 bases, 100 bases, and any integer therebetween) that comprises identifying information for the polypeptides within a fraction.

As used herein, the term ‘proline aminopeptidase’ refers to an enzyme that is capable of specifically cleaving an N-terminal proline from a polypeptide. Enzymes with this activity are well known in the art, and may also be referred to as proline iminopeptidases or as PAPs. Known monomeric PAPs include family members from B. coagulans, L. delbrueckii, N. gonorrhoeae, F. meningosepticum, S. marcescens, T. acidophilum, L. plantarum (MEROPS S33.001) (Nakajima, Ito et al. 2006) (Kitazono, Yoshimoto et al. 1992). Known multimeric PAPs including D. hansenii (Bolumar, Sanz et al. 2003) and similar homologues from other species (Basten, Moers et al. 2005). Either native or engineered variants/mutants of PAPs may be employed.

As used herein, the term “alkyl” refers to and includes saturated linear and branched univalent hydrocarbon structures and combination thereof, having the number of carbon atoms designated (i.e., C₁-C₁₀ or C₁₋₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”), unless otherwise specified Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

As used herein, “alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

The term “aminoalkyl” refers to an alkyl group that is substituted with one or more —NH₂ groups. In certain embodiments, an aminoalkyl group is substituted with one, two, three, four, five or more —NH₂ groups. An aminoalkyl group may optionally be substituted with one or more additional substituents as described herein.

As used herein, “aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. In some embodiments, phenyl is a preferred aryl group.

As used herein, the term “arylalkyl” refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

As used herein, the term “cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. In some embodiments, the cycloalkyl is a cyclic hydrocarbon having from 3 to 13 annular carbon atoms. In some embodiments, the cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

As used herein, the “halogen” represents chlorine, fluorine, bromine, or iodine. The term “halo” represents chloro, fluoro, bromo, or iodo.

The term “haloalkyl” refers to an alkyl group as described above, wherein one or more hydrogen atoms on the alkyl group have been replaced by a halo group. Examples of such groups include, without limitation, fluoroalkyl groups, such as fluoroethyl, trifluoromethyl, difluoromethyl, trifluoroethyl and the like.

As used herein, the term “heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. It is understood that the selection and order of heteroatoms in a heteroaryl ring must conform to standard valence requirements and provide an aromatic ring character, and also must provide a ring that is sufficiently stable for use in the reactions described herein. Typically, a heteroaryl ring has 5-6 ring atoms and 1-4 heteroatoms, which are selected from N, O and S unless otherwise specified; and a bicyclic heteroaryl group contains two 5-6 membered rings that share one bond and contain at least one heteroatom and up to 5 heteroatoms selected from N, O and S as ring members. A heteroaryl group can be attached to the remainder of the molecule at an annular carbon or at an annular heteroatom, in which case the heteroatom is typically nitrogen. Heteroaryl groups may contain additional fused rings (e.g., from 1 to 3 rings), including additionally fused aryl, heteroaryl, cycloalkyl, and/or heterocyclyl rings. Examples of heteroaryl groups include, but are not limited to, pyrazolyl, imidazolyl, triazolyl, pyrrolyl, pyridyl, pyrimidyl, pyrazinyl, pyridazinyl, triazinyl, thiophenyl, furanyl, thiazolyl, and the like.

As used herein, the term “heterocycle”, “heterocyclic”, or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

As used herein, the term “side product” refers to a by-product formed during the generation or subsequent reaction of a polypeptide having a functionalized NTAA, such as a thiourea of Formula

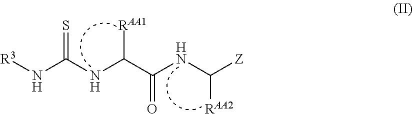

or of a compound of Formula (II) or Formula (IV) as described herein, wherein the side product arises by hydrolysis, intramolecular cyclization, or oxidation of the functionalized polypeptide before the functionalized polypeptide undergoes a reaction progressing toward NTAA cleavage, such as those depicted in Scheme I. Examples of side products are described herein. In some embodiments, side products can retain the NTAA in modified form after a sequence of steps designed to cleave the NTAA from the polypeptide. In some of the methods herein, an optional step of identifying or detecting one or more of said side products may be included in the NTAA cleavage method.

The term “substituted” means that the specified group or moiety bears one or more substituents in place of a hydrogen atom of the unsubstituted group, including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents and thus includes both substituted and unsubstituted versions of the group. Where the term “substituted” is used to describe a structural system, the substitution is meant to occur at any valency-allowed position on the system.

The term ‘diheteronucleophile’ as used herein refers to a compound having nucleophilic character at a heteroatom, usually nitrogen, that is directly bonded to another heteroatom. Typical examples include amine compounds having a nitrogen that is attached via a single bond to another heteroatom, typically selected from N, O and S. Common examples are hydrazine and hydroxylamine compounds. The amine nitrogen may be substituted provided it retains nucleophilic character, and the attached N, O or S may also be substituted. Some suitable diheteronucleophiles for use in the methods and kits of the invention include:

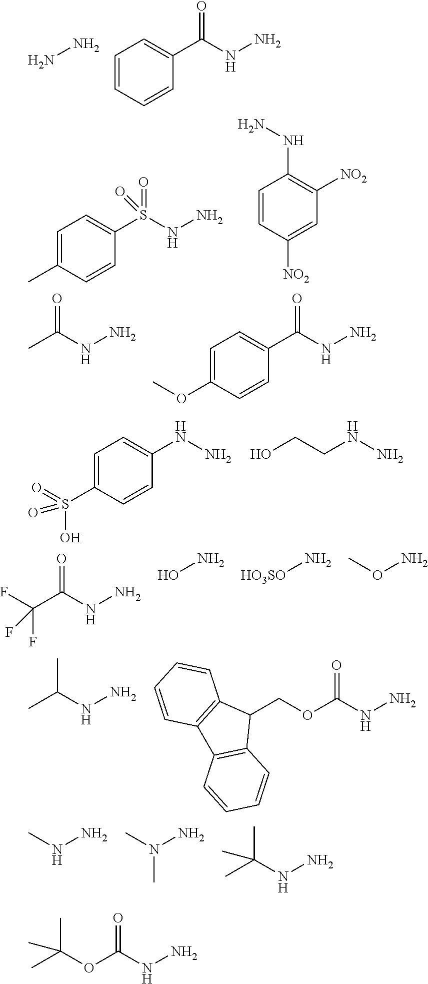

Structures described or depicted herein may be capable of forming multiple tautomers, as is well understood in the art. The particular tautomer or tautomers present often depend on solvent, pH, and other environmental factors as well as the structure itself. An example of tautomerism is shown here, where at least three different tautomers could be drawn to represent one compound:

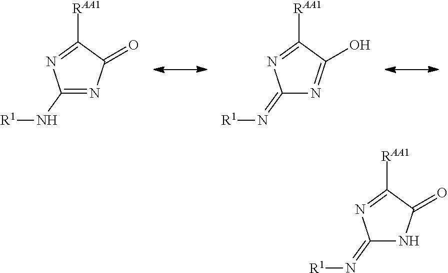

Where a compound can exist in more than one tautomeric form, typically one tautomer is depicted or described, and the structure is understood to represent each stable tautomer as well as mixtures of the tautomers. In particular, guanidine groups and heteroaryl groups substituted by hydroxyl or amine groups are often able to exist in multiple tautomers, and the description or depiction of one tautomer is understood to include the other tautomers of the same compound.

Methods of the invention utilize novel ways to functionalize an N-terminal amino acid to form compounds of Formula (II) as described herein, and to induce elimination of the functionalized NTAA of these compounds under mild conditions at around pH 5-10, as shown in Scheme I.

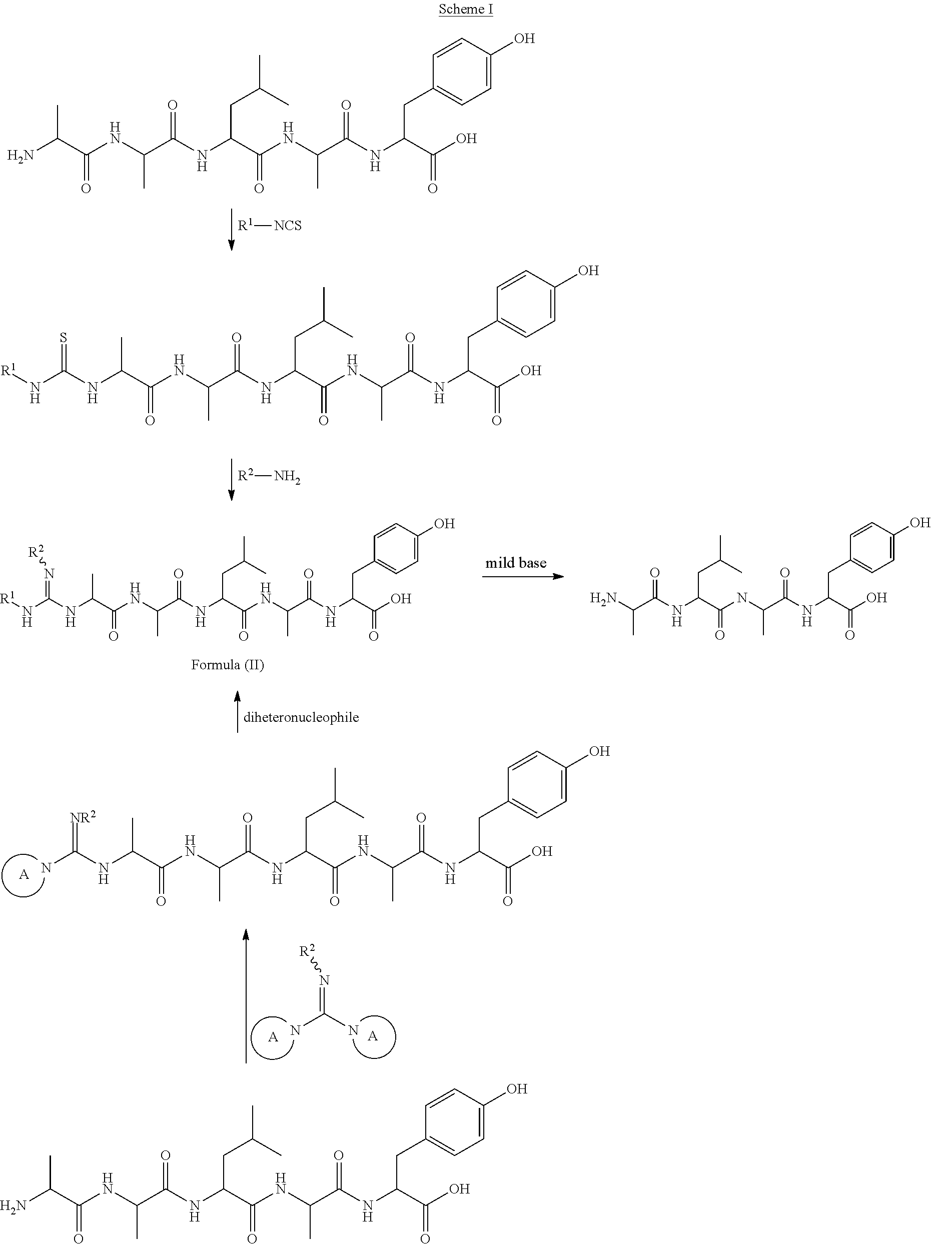

These reactions, as shown in Scheme I, result in cleavage of the NTAA from a polypeptide under mild conditions, and thus enable a novel method for removal of the NTAA from a polypeptide. Like Edman degradation, the cleavage of each NTAA produces a by-product that is determined by and therefore indicative of the structure of the NTAA that was removed. Because the method can be used repeatedly, to remove one NTAA at a time from a polypeptide, the invention includes a method to use these reactions and intermediates for sequencing a polypeptide, starting at the N-terminal end and removing the NTAAs one at a time, and identifying each cleavage by-product to identify the NTAA just removed.

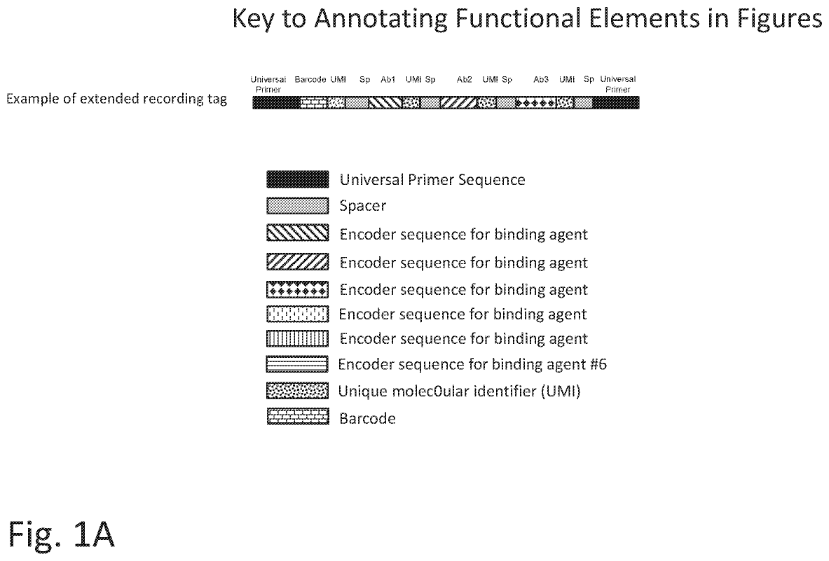

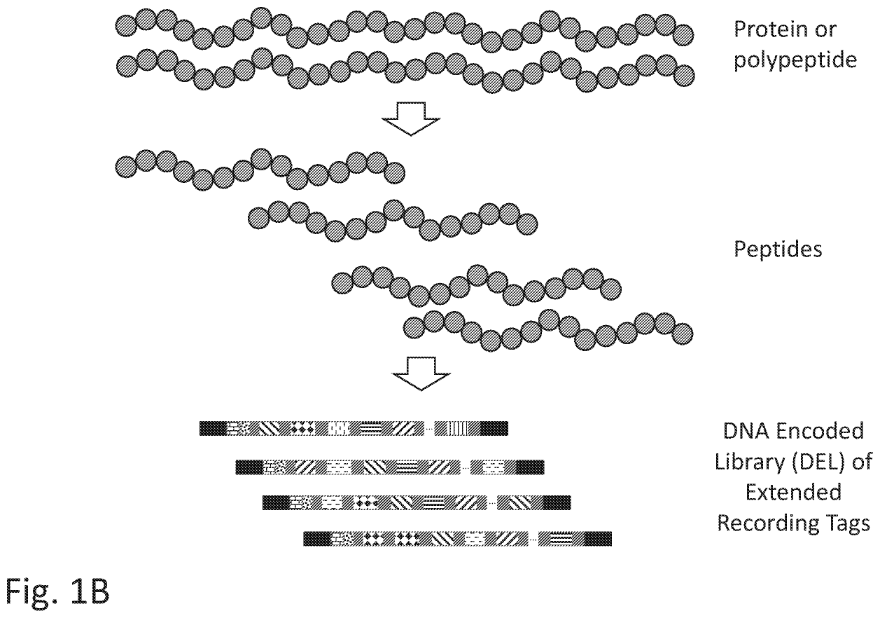

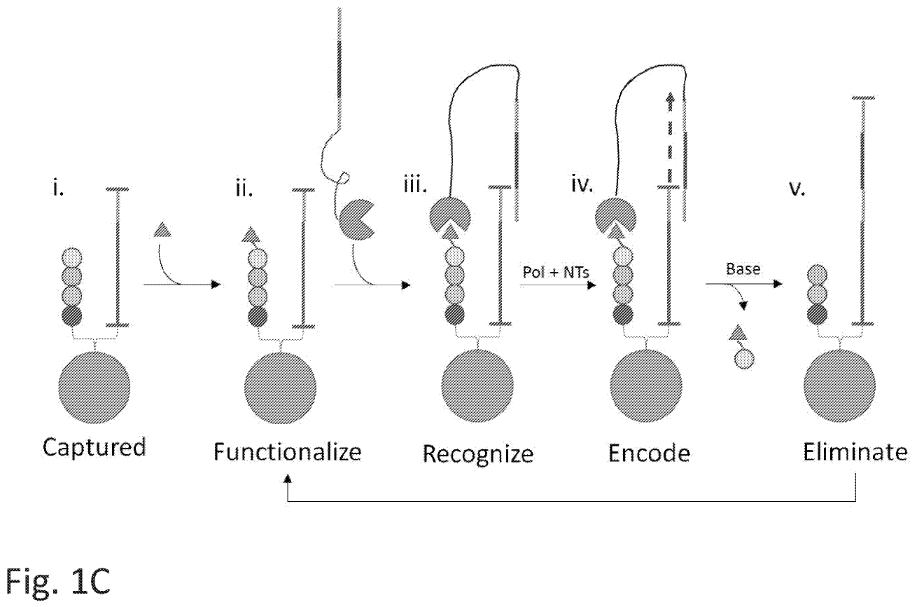

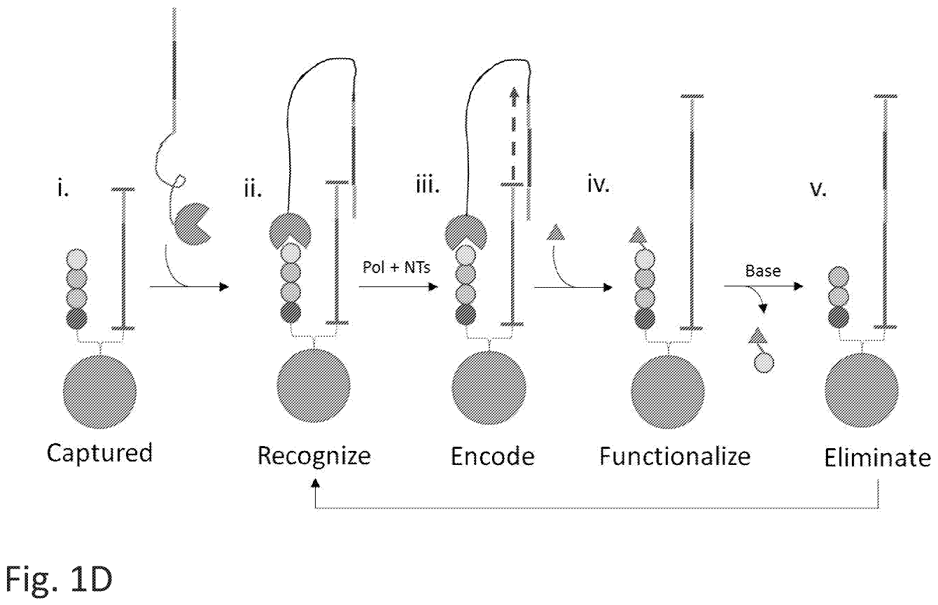

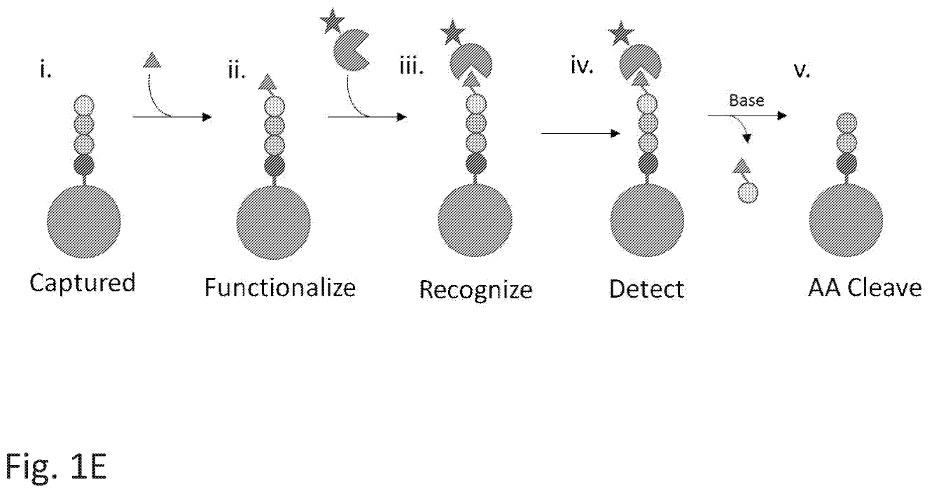

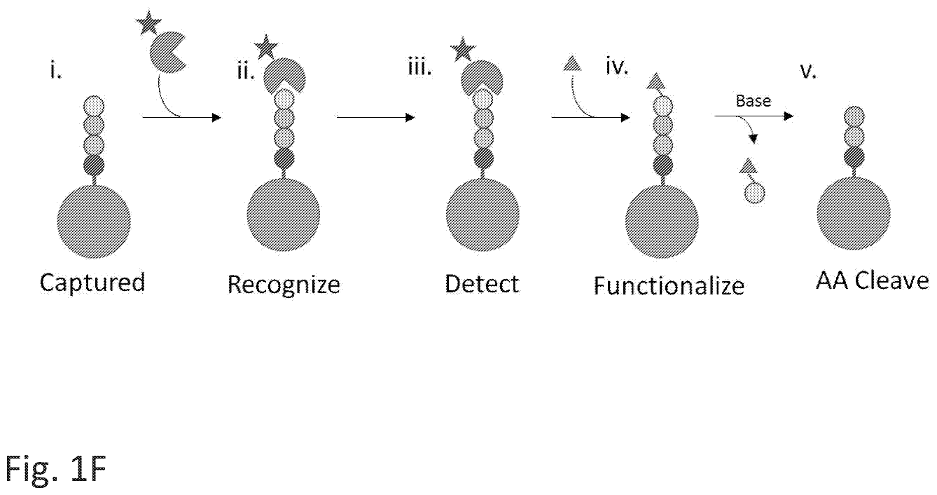

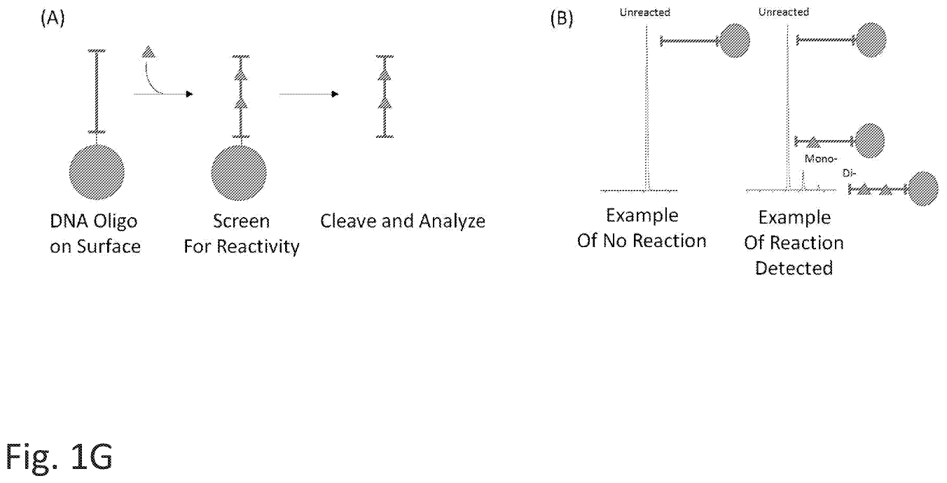

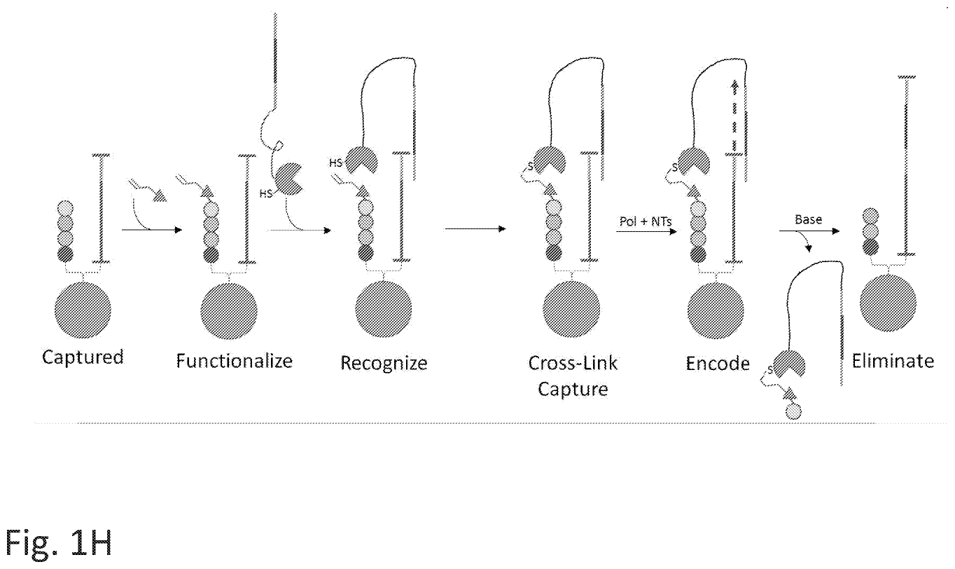

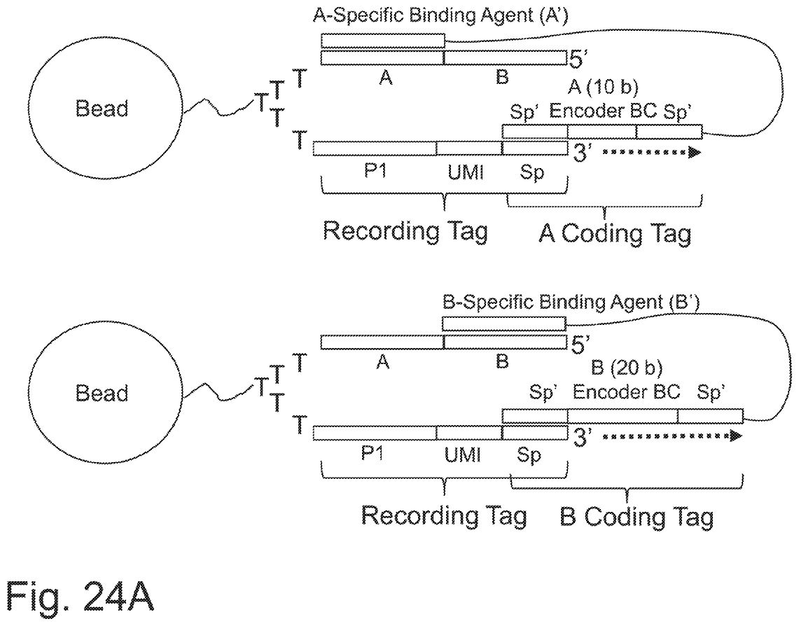

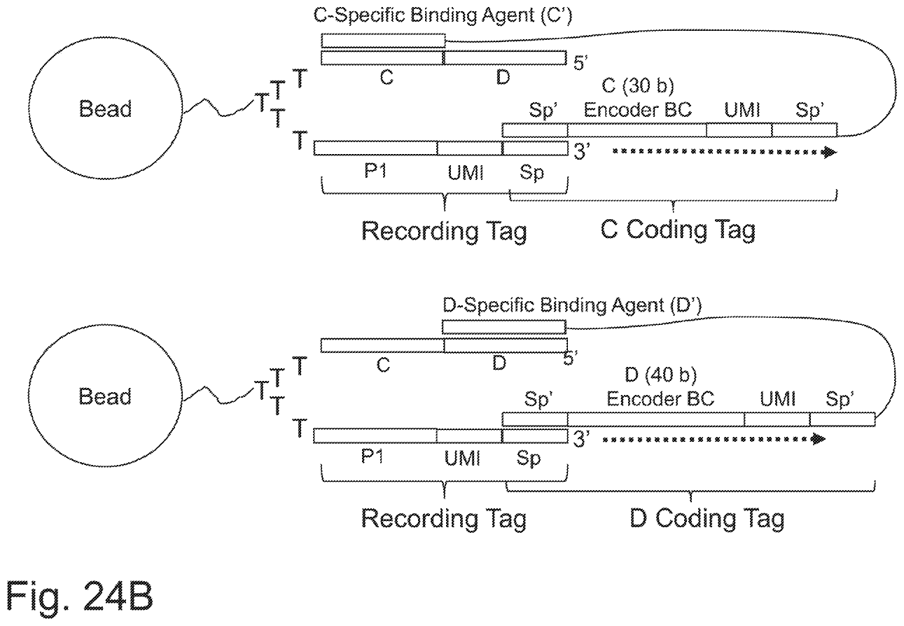

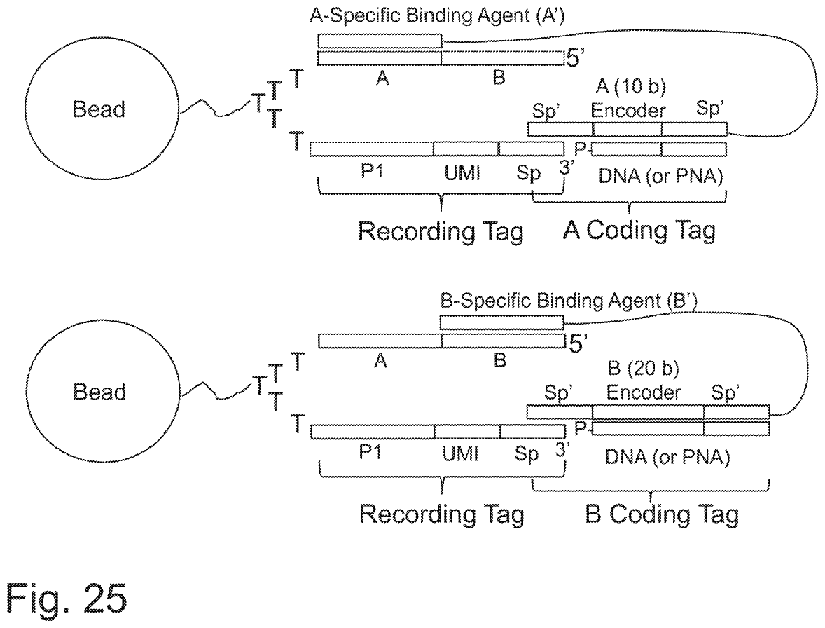

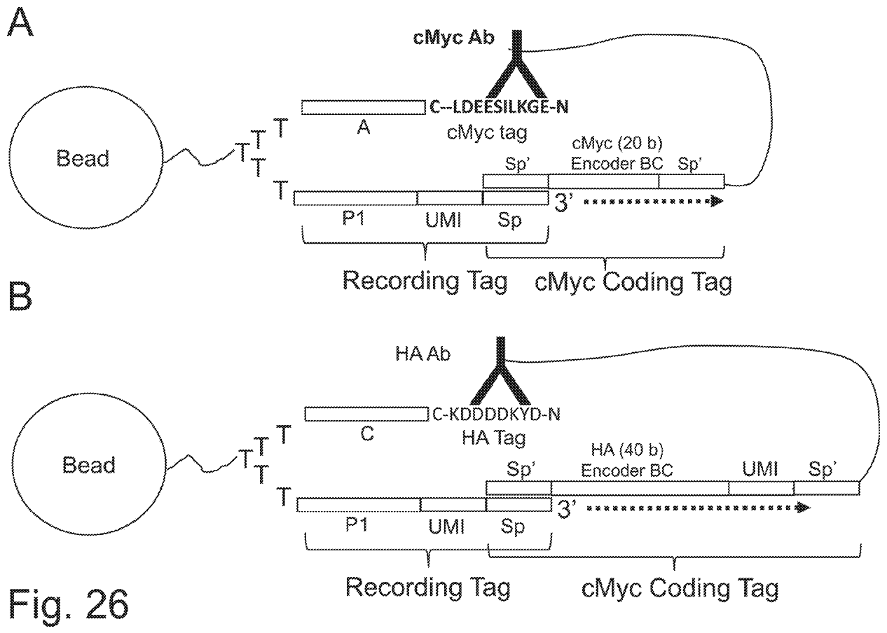

The mild reaction conditions involved make it possible to perform these reactions in the presence of acid-sensitive moieties, such as nucleic acids. Data provided herein, see the Examples and FIGS. 53-54, shows that nucleic acids are stable toward the conditions used for activation (e.g., functionalization) of an NTAA according to the methods of the invention, and to the conditions used to eliminate the functionalized NTAA. As a result, the methods can be combined with technology that utilizes nucleic acid tags to record information about each NTAA that is functionalized and removed, as the reactions are occurring. The nucleic acids are stable to the conditions used for functionalization and cleavage of the NTAA of a polypeptide as shown by data herein. Thus the invention also provides a method to use the NTAA cleavage chemistry disclosed herein in combination with nucleic acids that can be used to record sequence information about the polypeptide as the functionalization and cleavage reactions occur. This provides a method to create a polynucleotide that encodes information about the polypeptide structure, thus permitting the user to utilize the rapid and robust sequencing methods known in the art to read the sequence of the original polynucleotide. These methods are illustrated in FIGS. 1-55 herein.

The following enumerated embodiments represent certain aspects of the invention.
1. A method to cleave an N-terminal amino acid residue from a peptidic compound of Formula (I)

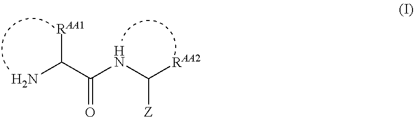

wherein the method comprises:
(1) converting the peptidic compound to a guanidinyl derivative of Formula (II), or a tautomer thereof:

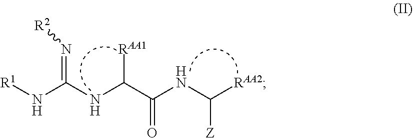

and
(2) contacting the guanidinyl derivative with a suitable medium to produce a compound of Formula (III)

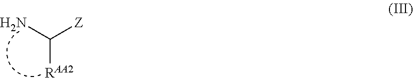

wherein:
R¹ is R³, NHR³, —NHC(O)—R³, or —NH—SO₂—R³
R² is H, R⁴, OH, OR⁴, NH₂, or —NHR⁴;
R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
and wherein two R′ or two R″ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
R^{AA1} and R^{AA2} are each independently selected amino acid side chains;
   and the dashed semi-circle connecting R^{AA1} and/or R^{AA2} to the nearest N atom indicates that R^{AA1} and/or R^{AA2} can optionally cyclize onto the designated N atom; and
Z is —COOH, CONH₂, or an amino acid or a polypeptide that is optionally attached to a carrier or solid support.

In many embodiments of this method, R¹ and R² are not both H in the compound of Formula (II). In a preferred example of this embodiment, R² is H or R⁴. R^{AA1} and R^{AA2} each represent an amino acid side chain, which may be that of a natural amino acid or an unnatural amino acid. The amino acid side chains may have post-translational modifications. In particular examples of this embodiment, R^{AA1} and R^{AA2} are independently selected from the common or proteinogenic amino acids, and may optionally be modified to include one or more PTMs commonly occurring on natural proteins in vivo. The 5-membered heteroaryl in these embodiments is typically a 5-membered ring comprising one to three heteroatoms selected from N, O and S as ring members. The 6-membered heteroaryl in these embodiments is typically a 6-membered ring comprising one to three nitrogen atoms as ring members.
2. The method of embodiment 1, wherein Z is a polypeptide.
3. The method of embodiment 1 or 2, wherein Z is a polypeptide attached to a solid support.
4. The method of embodiment 3, wherein the polypeptide is attached directly or indirectly to the solid support.

In this embodiment, the polypeptide Z can be directly attached to a solid support by conventional methods, typically utilizing a C-terminal carboxyl group to form an amide or ester with an amine or hydroxyl on the solid support. Alternatively, the polypeptide may be connected by any suitable linking group to the solid support; thus in some embodiments, the polypeptide may be attached to a nucleic acid that is in turn attached to the solid support, either covalently or by non-covalent means such as binding to a complementary sequence on the solid support.
5. The method of embodiment 4, wherein the polypeptide is covalently attached to the solid support.
6. The method of any one of embodiments 1-5, wherein the polypeptide is attached to a nucleic acid that is optionally covalently joined to a solid support.

In some of these embodiments, the polypeptide is attached to a nucleic acid that is free in solution, thus serving as a carrier. In some of these embodiments, the polypeptide is attached to a nucleic acid, usually by covalent attachment. In some of these embodiments, the nucleic acid is immobilized to a solid support by non-covalent forces such as by binding to a complementary nucleic acid affixed to the solid support. In other of these embodiments, the nucleic acid is covalently attached to a solid support.
7. The method of any one of embodiments 1-6, wherein the solid support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELBA plate, a spinning interferometry disc, a nitrocellulose membrane, a nitrocellulose-based polymer surface, a nanoparticle, or a microsphere.
8. The method of embodiment 7, wherein the support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.
9. The method of any one of embodiments 1-8, wherein the polypeptide is attached directly or indirectly to a carrier. Suitable carriers include nucleic acids, oligosaccharides, labels such as fluorophores that can be used to track or identify the polypeptide, and binding groups such as avidin or streptavidin that can be used to localize the polypeptide.
10. The method of any one of embodiments 1-9, wherein at least one of the amino acid side chains in the compound of Formula (I) comprises a post-translational modification. The PTM may be on R^{AA1} or R^{AA2}, or an an amino acid side chain in group Z.
11. The method of any one of embodiments 1-10, wherein the suitable medium for step (2) has pH above 5, preferably between about 5 and 14, and optionally includes a hydroxide, carbonate, phosphate, sulfate, or amine. In some embodiments, the pH is between 5 and 13, or between 7 and 10. In some embodiments, the pH is between 5 and 9. In some embodiments, the suitable medium is a basic medium that comprises some water and has a pH between about 8 and 14, and optionally comprises ammonium hydroxide or hydrazine. In some embodiments, the suitable medium comprises a buffering agent to help keep pH between 7 and 14, or between 8 and 13.
12. The method of embodiment 11, wherein the suitable medium comprises ammonia or an amino compound.

In any of embodiments 1-12, the suitable medium may comprise ammonia or ammonium hydroxide, optionally in combination with a water-miscible solvent such as acetonitrile, THF, or DMSO. When R² is H and R¹ is an optionally substituted phenyl, 5-membered heteroaryl, 6-membered heteroaryl, or C₁₋₆ alkyl in the compound of Formula (II) as described in Embodiment 1, the medium may comprise ammonium hydroxide, typically between 5 and 20% ammonium hydroxide for step 2. The conditions for the second step may also include heating the mixture to a temperature above ambient temperature, e.g. to a temperature between 40° C. and 100° C., typically between 45° C. and 75° C.
13. The method of embodiment 11, wherein the medium comprises a diheteronucleophile.

In these embodiments, the diheteronucleophile is often a hydrazine or hydroxylamine compound, such as a compound selected from these compounds:

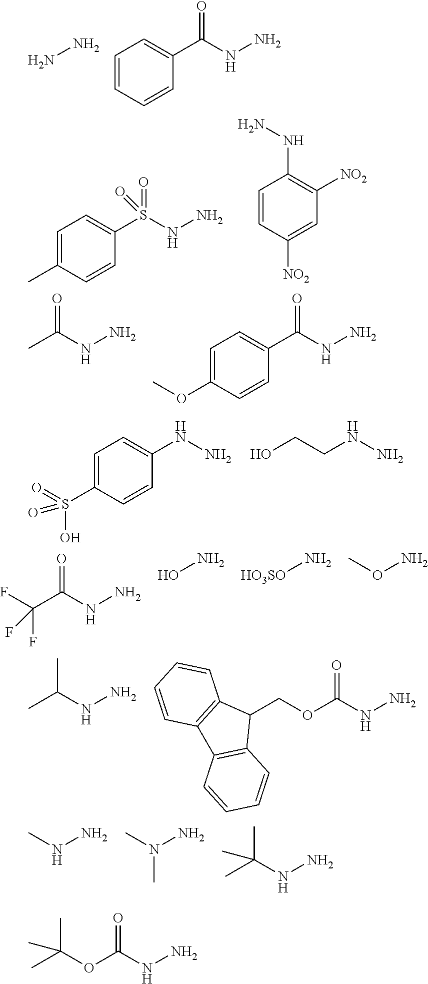

This method is especially suitable for use when R² in Formula (II) is H, and 10 in Formula (II) is NH₂ or NHR⁴. In these embodiments, hydrazine or a substituted hydrazine of the formula R⁴—NH—NH₂ can be used to both form the compound of Formula (II), for example via the reaction in Embodiment 18 below, and to promote elimination of the functionalized NTAA to provide the compound of Formula (III).
14. The method of any one of embodiments 1-13, wherein R² is H, and optionally 10 is not H.
15. The method of any one of embodiments 1-14, wherein R¹ is NH₂.
16. The method of any one of embodiments 1-14, wherein R¹ is phenyl optionally substituted with halo, C₁₋₃ alkyl, C₁₋₃ alkoxy, C₁₋₃haloalkyl, NO₂, CN, COOR′, or CON(R′)₂, where each R′ is independently H or C₁₋₃ alkyl,
   and wherein two R′ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂.
17. The method of embodiment 1, wherein the compound of Formula (I) is of the formula (IA):

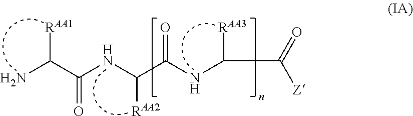

and the compound of Formula (III) is a compound of the formula (IIIA):

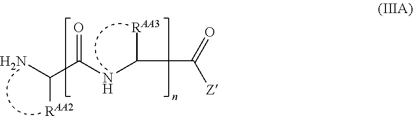

where n is an integer from 1 to 1000;
R^{AA1} and R^{AA2} are as defined in embodiment 1;
the dashed semi-circle connecting R^{AA1} and R^{AA2} and R^{AA3} to the adjacent N atom indicates that R^{AA1} and/or R^{AA2} and/or R^{AA3} can optionally cyclize onto the designated adjacent N atom; and
each R^{AA3} is independently selected from amino acid side chains, including natural and non-natural amino acids;
and Z′ is OH or NH₂, or Z′ is O or N that is attached to a carrier or solid support.

In these embodiments, n is typically between 1 and 500, or between 1 and 100.
18. The method of any one of embodiments 1-14, wherein the guanidinyl derivative of Formula (II) is produced by converting the peptidic compound of Formula (I) to a compound of the formula (IV):

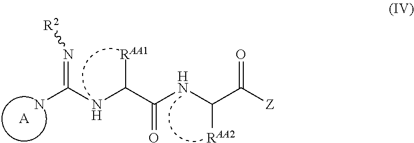

wherein ring A is a 5-6 membered heteroaryl ring containing up to three N atoms as ring members, optionally fused to an additional 5-6 membered heteroaryl or phenyl ring, and wherein the 5-6 membered heteroaryl ring and optional additional 5-6 membered heteroaryl or phenyl ring are each optionally substituted with up to four groups selected from C₁₋₄ alkyl, C₁₋₄ alkoxy, —OH, halo, C₁₋₄ haloalkyl, NO₂, COOR, CONR₂, —SO₂R*, and —NR₂;
wherein each R is independently selected from H and C₁₋₃ alkyl, optionally substituted with OH, OR*, —NH₂, and —NR*₂; and
each R* is C₁₋₃ alkyl, optionally substituted with OH, C₁₋₂ alkoxy, —NH₂, or CN; or a salt thereof;
wherein two R or two R* on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
   the dashed semi-circle connecting R^{AA1} and R^{AA2} to the nearest N atom indicates that R^{AA1} and/or R^{AA2} optionally cyclize onto the designated N atom;
   then contacting this compound with a diheteronucleophile, optionally in the presence of a buffer, to produce the compound of Formula (II).

In these embodiments, R², R^{AA1}, R^{AA2}, and Z are as defined in embodiment 1, or they can be as defined in any of the preceding embodiments. In preferred examples of these embodiments, A is a 5-membered heteroaryl ring containing up to three N atoms as ring members, and the 5-6 membered heteroaryl group when present is typically a 5-membered ring comprising one to three heteroatoms selected from N, O and S as ring members, or a 6-membered ring comprising one to three nitrogen atoms as ring members. The step of contacting the compound with a diheteronucleophile can comprise contacting the compound of Formula (IV) with hydrazine or a C₁-C₆ alkylhydrazine, optionally in the presence of a phosphate or carbonate buffer that provides a pH between 8 and 13.
19. The method of embodiment 18, wherein the peptidic compound of Formula (I) is converted to a compound of Formula (IV) by contacting the compound of Formula (I) with a compound of the formula:

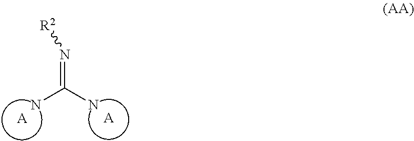

wherein:
   R² is H, R⁴, OH, OR⁴, NH₂, or —NHR⁴;
   R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂, where each R″ is independently H or C₁₋₃ alkyl;
ring A a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
   wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, and CN;
to form the compound of Formula (IV).

In a preferred example of this embodiment, R² is H or R⁴. In many embodiments of this method, R¹ and R² are not both H in the compound of Formula (II). The 5-6 membered heteroaryl group when present is typically a 5-membered heteroaryl ring comprising one to three heteroatoms selected from N, O and S as ring members, or a 6-membered heteroaryl ring comprising one to three nitrogen atoms as ring members.
20. The method of embodiment 18 or 19, wherein ring A is selected from:

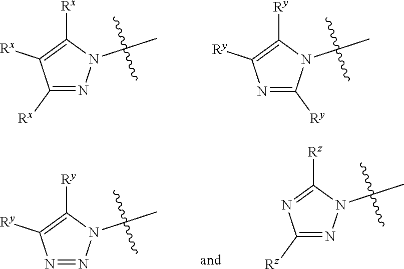

wherein:
each R^{x}, R^{y} and R^{z} is independently selected from H, halo, C₁₋₂ alkyl, C₁₋₂ haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂haloalkyl, NO₂, SO₂(C₁₋₂ alkyl), COOR^{#}, and C(O)N(R^{#})₂,
and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂haloalkyl, NO₂, SO₂(C₁₋₂ alkyl), COOR^{#}, and C(O)N(R^{#})₂;
wherein each R^{#} is independently H or C₁₋₂ alkyl; and wherein two R# on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
or a salt thereof.

In these embodiments, the 5-membered heteroaryl group, when present, can be a 5-membered ring comprising one to three heteroatoms selected from N, O and S as ring members, and the 6-membered heteroaryl group when present can be a 6-membered ring comprising one to three nitrogen atoms as ring members.
21. The method of embodiment 20, wherein Ring A is selected from:

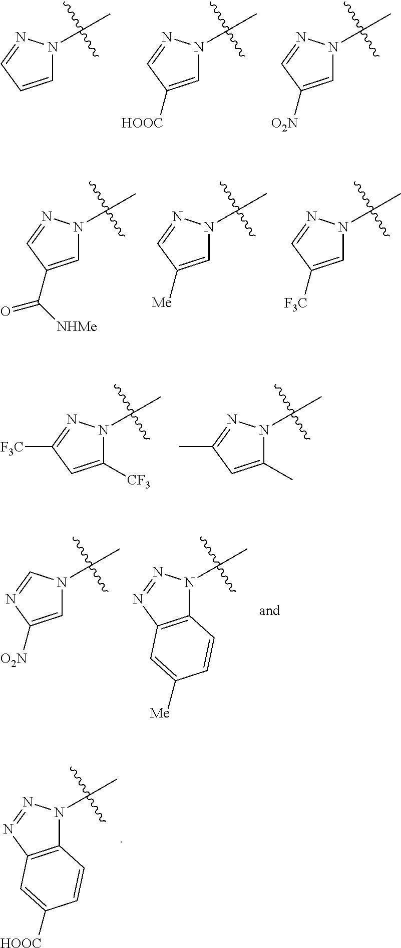

22. The method of embodiment 1, wherein the compound of Formula (II) is produced by contacting a compound of Formula (I) with an isothiocyanate of Formula R³—NCS to form a thiourea compound of the formula

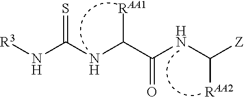

or a salt thereof; wherein
   R³ is H or an optionally substituted group selected from phenyl, 5-membered heteroaryl, 6-membered heteroaryl, C₁₋₃ haloalkyl, and C₁₋₆ alkyl,
      wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
      where each R′ is independently H or C₁₋₃ alkyl;
the dashed semi-circle connecting R^{AA1} and R^{AA2} to the nearest N atom indicates that R^{AA1} and/or R^{AA2} can optionally cyclize onto the designated N atom;
then contacting the thiourea compound with an amine compound of the formula R²—NH₂;
to produce the compound of Formula (II).
23. The method of embodiment 22, wherein R³ is phenyl optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂,
   where each R′ is independently H or C₁₋₃ alkyl, and wherein two R′ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂.
24. The method of any of embodiments 18-23, wherein the suitable medium in step (2) comprises NH₃ or an amine of the formula (C₁₋₆)alkyl-NH₂.
25. The method of embodiment 24, wherein step (2) comprises heating the compound of Formula (II) in a mixture comprising ammonium hydroxide.
26. The method of any of embodiments 18-23, wherein the suitable medium in step (2) comprises a diheteronucleophile.

In these embodiments, the diheteronucleophile is often a hydrazine or hydroxylamine compound. This method is especially suitable for use when R² in Formula (II) is H, and R¹ in Formula (II) is NH₂ or NHR⁴. In these embodiments, hydrazine or a substituted hydrazine of the formula R⁴—NH—NH₂ can be used to both form the compound of Formula (II), for example via the reaction in Embodiment 18 below, and to promote elimination of the functionalized NTAA to provide the compound of Formula (III).
27. The method of embodiment 26, wherein the diheteronucleophile is selected from:

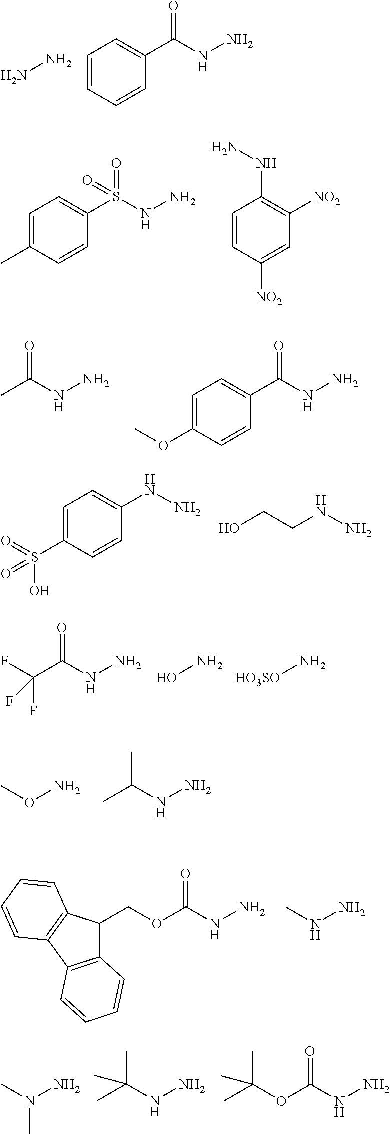

28. The method of any one of embodiments 1-27, wherein R^{AA1} and R^{AA2} are each independently selected from H and C₁₋₆ alkyl optionally substituted with one or two groups independently selected from —OR⁵, —N(R⁵)₂, —SR⁵, —COOR⁵, CON(R⁵)₂, —NR⁵—C(═NR⁵)—N(R⁵)₂, phenyl, imidazolyl, and indolyl, where phenyl, imidazolyl and indolyl are each optionally substituted with halo, C₁₋₃ alkyl, C₁₋₃ haloalkyl, —OH, C₁₋₃ alkoxy, CN, COOR⁵, or CON(R⁵)₂;
   each R⁵ is independently selected from H and C₁₋₂ alkyl, and wherein two R⁵ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂.
29. The method of any one of embodiments 1-28, wherein each R^{AA1} and R^{AA2} is independently selected from the side chains of the proteinogenic amino acids, optionally including one or more post-translational modifications.
30. A compound of the Formula:

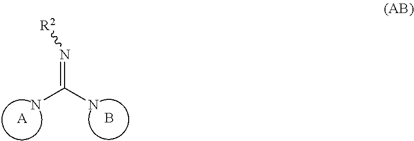

wherein:
   R² is H, R⁴, OH, OR⁴, NH₂, or —NHR⁴;
   R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein each phenyl, 5-membered heteroaryl, and 6-membered heteroaryl is optionally substituted with one or two members selected from halo, —OH, C₁₋₃ alkyl, C₁₋₃ alkoxy, C₁₋₃ haloalkyl, NO₂, CN, COOR″, and CON(R″)₂,
      where each R″ is independently H or C₁₋₃ alkyl;
ring A and ring B are each independently a 5-membered heteroaryl ring containing up to three N atoms as ring members and each is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
   wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
with the proviso that Ring A and Ring B are not both unsubstituted imidazole, and that Ring A and Ring B are not both unsubstituted benzotriazole;
or a salt thereof.

In a preferred example of this embodiment, R² is H or R⁴. In these embodiments, In these embodiments, the 5-membered heteroaryl group, when present, can be a 5-membered ring comprising one to three heteroatoms selected from N, O and S as ring members, and the 6-membered heteroaryl group when present can be a 6-membered ring comprising one to three nitrogen atoms as ring members. In some of these embodiments, neither ring A nor ring B is unsubstituted imidazole or unsubstituted benzotriazole.
31. The compound of embodiment 30, wherein R² is H.
32. The compound of embodiment 30 or 31, wherein Ring A and Ring B are the same.

Specific compounds of this embodiment include:

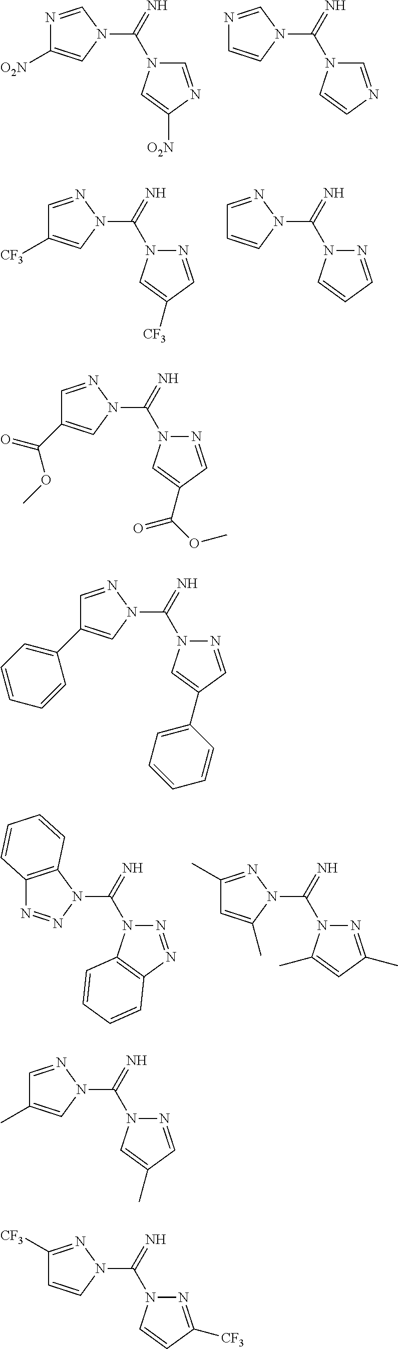

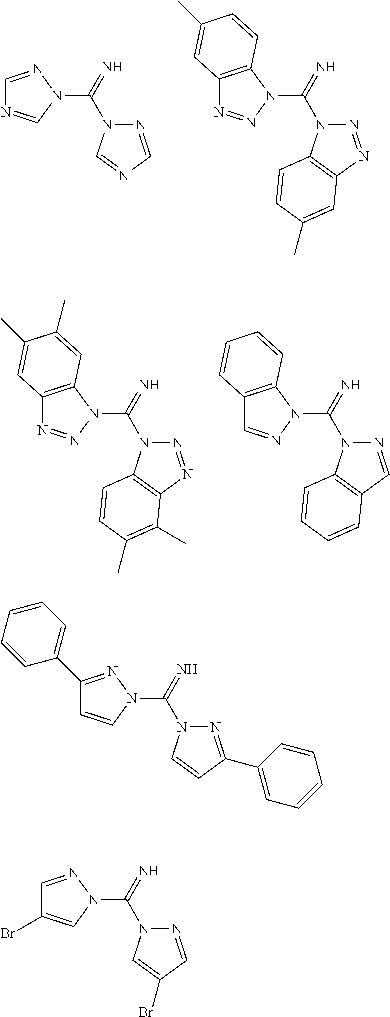 [SMILES: N=C(n1cncn1)n1cncn1 ; Cc1ccc2c(c1)nnn2C(=N)n1nnc2cc(C)ccc21 ; Cc1cc2nnn(C(=N)n3nnc4c(C)c(C)ccc43)c2cc1C ; N=C(n1ncc2ccccc21)n1ncc2ccccc21 ; N=C(n1ccc(-c2ccccc2)n1)n1ccc(-c2ccccc2)n1 ; N=C(n1cc(Br)cn1)n1cc(Br)cn1]

33. The compound of any one of embodiments 30-32, wherein each 5-6 membered heteroaryl ring is independently selected and contains 1 or 2 heteroatoms selected from N, O and S as ring members. In these embodiments, each 5-membered heteroaryl group present can be a 5-membered ring comprising one or two heteroatoms selected from N, O and S as ring members, and the 6-membered heteroaryl group can be a 6-membered ring comprising one to two nitrogen atoms as ring members.
34. The compound of any one of embodiments 30-33, wherein Ring A and Ring B are selected from:

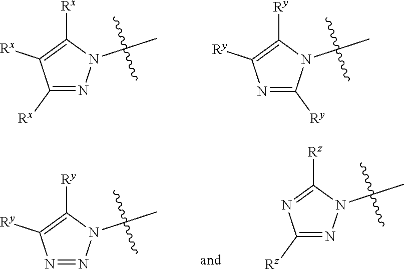

wherein:
each R^{x}, R^{y} and R^{z} is independently selected from H, halo, C₁₋₂ alkyl, C₁₋₂haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂haloalkyl, NO₂, SO₂(C₁₋₂ alkyl), COOR^{#}, and C(O)N(R^{#})₂,
   and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;
   wherein each R^{#} is independently H or C₁₋₂ alkyl; and wherein two R^{#} on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
   or a salt thereof.

In these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
35. The compound of embodiment 34, wherein Ring A and Ring B are the same and are selected from:

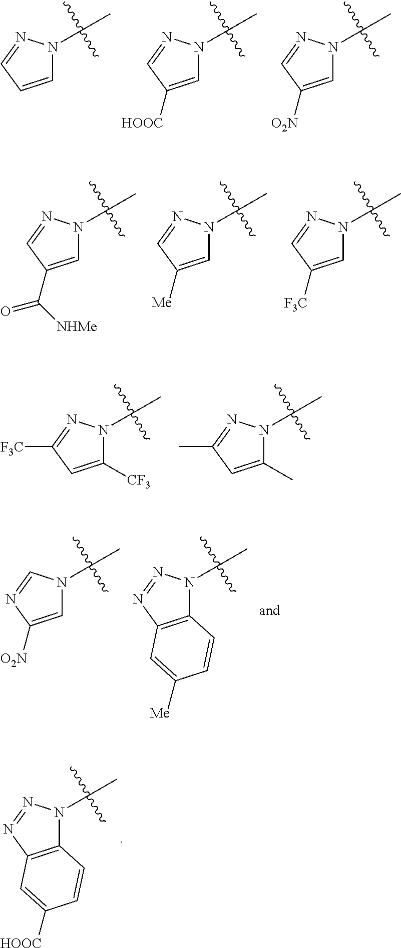

36. The compound of embodiment 30, which is selected from the following:

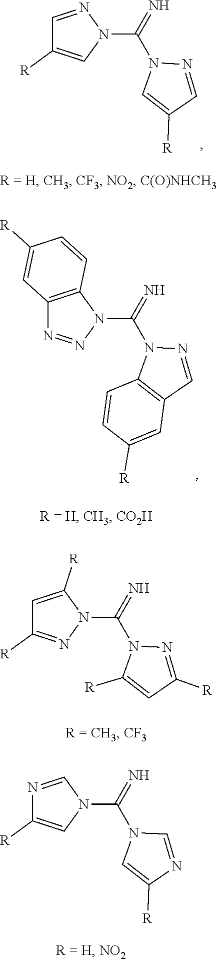

37. A compound of Formula (II):

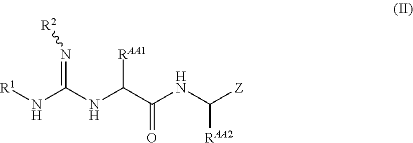

or a tautomer thereof,
   wherein:
R¹ is R³, NHR³, —NHC(O)—R³, or —NH—SO₂—R³;
R² is H, R⁴, OH, OR⁴, NH₂, or —NHR⁴;
R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
wherein two R′ or two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C1-2 alkoxy, or CN;
R^{AA1} and R^{AA2} are each independently selected from H and C₁₋₆ alkyl optionally substituted with one or two groups independently selected from —OR⁵, —N(R⁵)₂, —SR⁵, —COOR⁵, CON(R⁵)₂, —NR⁵—C(═NR⁵)—N(R⁵)₂, phenyl, imidazolyl, and indolyl, where phenyl, imidazolyl and indolyl are each optionally substituted with halo, C₁₋₃ alkyl, C₁₋₃ haloalkyl, —OH, C₁₋₃ alkoxy, CN, COOR⁵, or CON(R⁵)₂;
   each R⁵ is independently selected from H and C₁₋₂ alkyl;
and Z is —COOH, CONH₂, or an amino acid or polypeptide that is optionally attached to a carrier or surface; or a salt thereof.

In a preferred example of this embodiment, R² is H or R⁴. In some examples, R¹ and R² are not both H. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
38. The compound of embodiment 30, wherein R¹ is NH₂.
39. The compound of embodiment 30, wherein R¹ is R³, and R³ is optionally not H.
40. The compound of any one of embodiments 30-32, wherein R² is H.
41. The compound of any one of embodiments 37-40, wherein Z is a polypeptide attached to a solid support.
42. The compound of embodiment 41, wherein the polypeptide is attached directly or indirectly to the solid support.
43. The compound of any one of embodiments 37-42, wherein the polypeptide is attached to a nucleic acid that is optionally covalently attached to a solid support.
44. The compound of embodiment 42 or 43, wherein the solid support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.
45. The compound of embodiment 44, wherein the support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.
46. The compound of any one of embodiments 37-45, which is isolated at a pH of 8 or below 8.
47. A compound of Formula (IV):

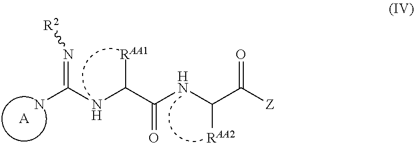

R² is H, R⁴, OH, OR⁴, NH₂, or —NHR⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
wherein two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
   wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
   R^{AA1} and R^{AA2} are each independently selected amino acid side chains;
      and the dashed semi-circle connecting R^{AA1} and/or R^{AA2} to the nearest N atom indicates that R^{AA1} and/or R^{AA2} can optionally cyclize onto the designated N atom; and
   Z is —COOH, CONH₂, or an amino acid or a polypeptide that is optionally attached to a carrier or solid support;
      or a salt thereof.

In a preferred example of this embodiment, R² is H or R⁴. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
48. The compound of embodiment 47, wherein R² is H.
49. The compound of embodiment 47 or 48, wherein Ring A is selected from:

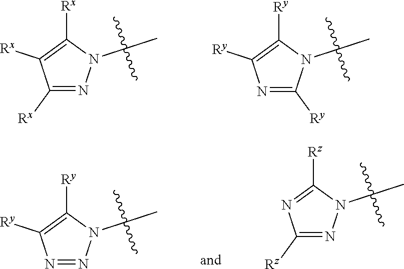

wherein:
each R^{x}, R^{y} and R^{z} is independently selected from H, halo, C₁₋₂ alkyl, C₁₋₂haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂haloalkyl, NO₂, SO₂(C₁₋₂ alkyl), COOR^{#}, and C(O)N(R⁴)₂,
   and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;
   wherein each R^{#} is independently H or C₁₋₂ alkyl; and wherein two R# on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
or a salt thereof
50. The compound of any one of embodiments 47-49, wherein Ring A is selected from:

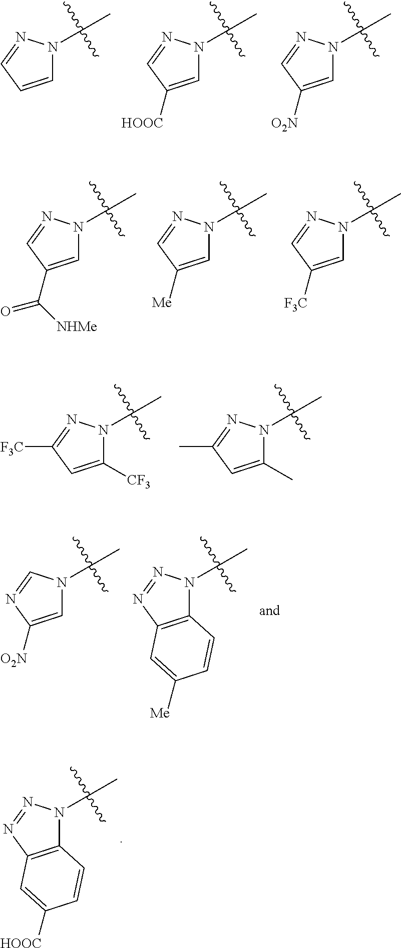

51. The compound of any of embodiments 47-50, wherein Z is an amino acid or polypeptide that is attached to a solid support.
52. The compound of embodiment 51, wherein Z is a polypeptide is attached directly or indirectly to a solid support.
53. The compound of embodiment 52 wherein the polypeptide is covalently attached to the solid support.
54. The compound of any one of embodiments 47-53, wherein Z is an amino acid or polypeptide that is attached to a nucleic acid that is optionally covalently attached to a solid support.
55. The compound of any one of embodiments 47-54, wherein the solid support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.
56. The compound of embodiment 55, wherein the solid support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.
57. The compound of any one of embodiments 47-50, wherein the compound of Formula (IV) is a compound of the formula:

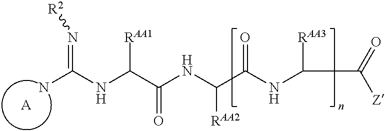

where n is an integer from 1 to 1000;
R^{AA1}, R^{AA2}, and each R^{AA3} is independently selected from the side chains of natural proteinogenic amino acids, optionally comprising post-translational modifications; and Z′ is OH or NH₂ or an amino acid connected directly or indirectly to a carrier or a solid support.

In a preferred example of this embodiment, R² is H or R⁴. In examples of this embodiment, n is 1-500, or n is 1-100. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
58. The compound of any one of embodiments 47-57, which comprises at least one amino acid side chain having a chemical or biological modification.
59. A method to identify the N-terminal amino acid residue of a peptidic compound of the Formula (I):

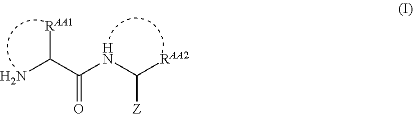

wherein the method comprises:
(1) converting the compound of Formula (I) to a guanidinyl derivative of Formula (II) or a tautomer thereof:

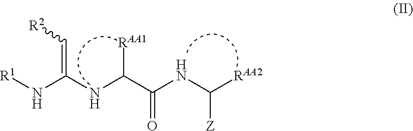

wherein:
R¹ is R³, NHR³, —NHC(O)—R³, or —NH—SO₂—R³
R² is H, R⁴, OH, OR⁴, NH₂, or —NHR⁴;
R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
wherein two R′ or two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C1-2 alkoxy, or CN;
R^{AA1} and R^{AA2} are each independently selected amino acid side chains, optionally including a post-translational modification;
   and the dashed semi-circle connecting R^{AA1} and/or R^{AA2} to the nearest N atom indicates that R^{AA1} and/or R^{AA2} can optionally cyclize onto the designated N atom; and
and Z is —COOH, CONH₂, or an amino acid or polypeptide that is optionally attached to a carrier or solid surface;
(2) contacting the guanidinyl derivative with a suitable medium to induce elimination of the modified N-terminal amino acid and produce at least one cleavage product selected from:

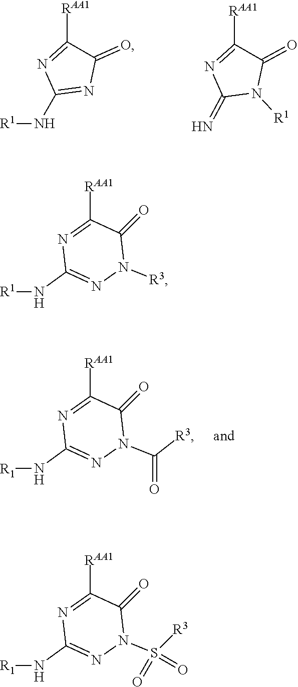

(when R¹ is NHR³, —NHC(O)—R³, or —NH—SO₂—R³, respectively) or a tautomer thereof; and
(3) determining the structure or identity of the at least one cleavage product to identify the N-terminal amino acid of the compound of Formula (I).

In a preferred example of this embodiment, R² is H or R⁴. In certain examples of this embodiment, R¹ and R² are not both H. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
60. The method of embodiment 59, wherein R^{AA1} and R^{AA2} are each independently selected from H and C₁₋₆ alkyl optionally substituted with one or two groups independently selected from —OW, —N(R⁵)₂, —SR⁵, —SeR⁵, —COOR⁵, CON(R⁵)₂, —NR⁵—C(═NR⁵)—N(R⁵)₂, phenyl, imidazolyl, and indolyl, where phenyl, imidazolyl and indolyl are each optionally substituted with halo, C₁₋₃ alkyl, C₁₋₃ haloalkyl, —OH, C₁₋₃ alkoxy, CN, COOR⁵, or CON(R⁵)₂; and
   each R⁵ is independently selected from H and C₁₋₂ alkyl.
61. The method of embodiment 59 or 60, wherein R^{AA1} is the side chain of one of the proteinogenic amino acids.
62. The method of any one of embodiments 59-61, wherein R^{AA2} is the side chain of one of the proteinogenic amino acids.
63. The method of any one of embodiments 59-62, wherein R¹ is phenyl optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂,
   where each R′ is independently H or C₁₋₃ alkyl.
64. The method of any one of embodiments 59-62, wherein R¹ is NH₂.
65. The method of any one of embodiments 59-64, wherein R² is H.
66. The method of any of embodiments 59-65, wherein Z is an amino acid or polypeptide that is attached to a solid support.
67. The method of any one of embodiments 59-66, wherein the solid support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.
68. The method of any one of embodiments 59-67, wherein the step of converting the compound of Formula (I) to a compound of Formula (II) comprises contacting the compound of Formula (I) with a compound of Formula (AA):

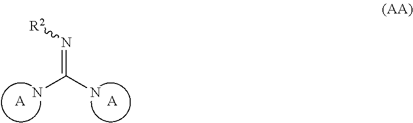

wherein:
   R² is H, R⁴, OH, OR⁴, NH₂, or —NHR⁴;
   R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
      where each R″ is independently H or C₁₋₃ alkyl;
ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
   wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
to form a compound of Formula (IV)

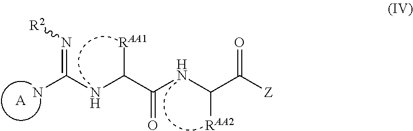

then contacting the compound of Formula (IV) with a diheteronucleophile to form the compound of Formula (II) and at least one of the cleavage products of embodiment 59.

In a preferred example of this embodiment, R² is H or R⁴. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
69. The method of embodiment 68, Therein the diheteronucleophile is selected from

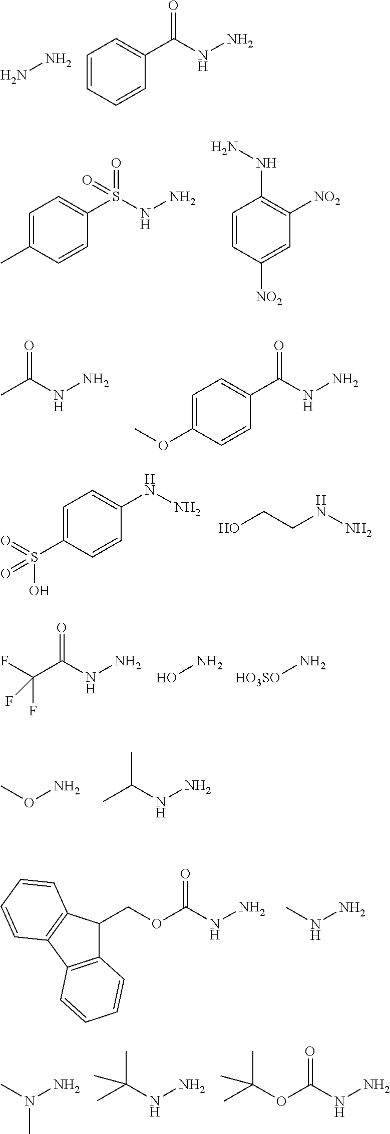

70. The method of any one of embodiments 59-69, wherein the step of converting the compound of Formula (I) to a compound of Formula (II) comprises contacting the compound of Formula (I) with a compound of Formula R³—NCS to form a thiourea of Formula

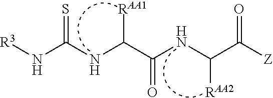

or a salt thereof, wherein:
   R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
      where each R′ is independently H or C₁₋₃ alkyl;
   R^{AA1}, R^{AA2}, R², and Z are as defined in embodiment 59, and the dashed semi-circle connecting R^{AA1} and R^{AA2} to the nearest N atoms indicates that R^{AA1} and/or R^{AA2} can optionally cyclize onto the designated N atom;
   then contacting the thiourea compound with an amine of the formula R²—NH₂ to produce the compound of Formula (II).

In some embodiments of this method, R³ is an optionally substituted phenyl.
71. The method of any one of embodiments 59-70, wherein R² is H.
72. A method for analyzing a polypeptide, comprising the steps of:
   (a) providing the polypeptide optionally associated directly or indirectly with a recording tag;
   (b) functionalizing the N-terminal amino acid (NTAA) of the polypeptide with a chemical reagent, wherein the chemical reagent is either:
      (b1) a compound of Formula (AA):

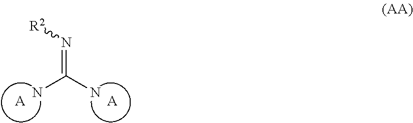

wherein:
   R² is H, R⁴, OH, OR⁴, NH₂, or —NHR⁴;
   R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
   wherein two R or two R″ or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
or
(b2) a compound of the formula R³—NCS;
wherein R³ is H or an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
to provide an initial NTAA functionalized polypeptide;
optionally treating the initial NTAA functionalized polypeptide with an amine of Formula R²—NH₂ or with a diheteronucleophile to form a secondary NTAA functionalized polypeptide;
and optionally treating the initial NTAA functionalized polypeptide or the secondary NTAA functionalized polypeptide with a suitable medium to eliminate the NTAA and form an N-terminally truncated polypeptide;
(c) contacting the polypeptide with a first binding agent comprising a first binding portion capable of binding to the polypeptide, or to the initial NTAA functionalized polypeptide, or to the secondary NTAA functionalized polypeptide, or to the N-terminally truncated polypeptide; and either
   (c1) a first coding tag with identifying information regarding the first binding agent, or
   (c2) a first detectable label;
(d) (d1) transferring the information of the first coding tag, if present, to the recording tag to generate an extended recording tag and analyzing the extended recording tag, or
   (d2) detecting the first detectable label, if present.

In a preferred example of this embodiment, R² is H or R⁴. In some examples of this embodiment, 10 and R² are not both H. In some examples, R³ is optionally substituted phenyl. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
73. The method of embodiment 72, further comprising repeating steps (b) through (d) to determine the sequence of at least a part of the polypeptide.
74. The method of embodiment 72 or embodiment 73, wherein the binding portion is capable of binding to:
   a non-functionalized NTAA of the polypeptide;
   the initial NTAA functionalized polypeptide; or
   the secondary NTAA functionalized polypeptide; or
   the N-terminally truncated polypeptide.
75. The method any one of embodiments 74, wherein the binding portion is capable of binding to:
   a product from step (b1) after contacting the polypeptide with the compound of Formula (AA);
   a product from step (b2) after contacting the polypeptide with the compound of the formula R³—NCS; or
   a product from step (b1) contacted with the amine of Formula R²—NH₂ or with the diheteronucleophile; or
   a product from step (b2) contacted with the amine of Formula R²—NH₂ or with the diheteronucleophile.
76. The method of any one of embodiments 72-75, wherein step (a) further comprises contacting the polypeptide with one or more enzymes under conditions suitable to cleave an N-terminal amino acid of the polypeptide, (e.g., a proline aminopeptidase, a proline iminopeptidase (PIP), a pyroglutamate aminopeptidase (pGAP), an asparagine amidohydrolase, a peptidoglutaminase asparaginase, a protein glutaminase, or a homolog thereof).
77. The method of any one of embodiments 72-75, wherein:
   step (a) comprises providing the polypeptide and an associated recording tag joined to a support (e.g., a solid support);
   step (a) comprises providing the polypeptide joined to an associated recording tag in a solution;
   step (a) comprises providing the polypeptide associated indirectly with a recording tag; or
   the polypeptide is not associated with a recording tag in step (a).
78. The method of embodiment 72 or 77, wherein:
   step (b) is conducted before step (c);
   step (b) is conducted before step (d);
   step (b) is conducted after step (c) and before step (d);
   step (b) is conducted after both step (c) and step (d);
   step (c) is conducted before step (b);
   step (c) is conducted after step (b); and/or
   step (c) is conducted before step (d).
79. The method of embodiment 72 or 77, wherein:
   steps (a), (b), (c1), and (d1) occur in sequential order;
   steps (a), (c1), (b), and (d1) occur in sequential order;
   steps (a), (c1), (d1), and (b) occur in sequential order;
   steps (a), (b1), (c1), and (d1) occur in sequential order;
   steps (a), (b2), (c1), and (d1) occur in sequential order;
   steps (a), (c1), (b1), and (d1) occur in sequential order;
   steps (a), (c1), (b2), and (d1) occur in sequential order;
   steps (a), (c1), (d1), and (b1) occur in sequential order;
   steps (a), (c1), (d1), and (b2) occur in sequential order;
   steps (a), (b), (c2), and (d2) occur in sequential order;
   steps (a), (c2), (b), and (d2) occur in sequential order; or
   steps (a), (c2), (d2), and (b) occur in sequential order.
80. The method of any one of embodiments 72-79, wherein step (c) further comprises contacting the polypeptide with a second (or higher order) binding agent comprising a second (or higher order) binding portion capable of binding to a functionalized NTAA other than the functionalized NTAA of step (b) and a coding tag with identifying information regarding the second (or higher order) binding agent.
81. The method of embodiment 80, wherein:
   contacting the polypeptide with the second (or higher order) binding agent occurs in sequential order following the polypeptide being contacted with the first binding agent; or
   contacting the polypeptide with the second (or higher order) binding agent occurs simultaneously with the polypeptide being contacted with the first binding agent.
82. The method of any one of embodiments 72-81, wherein the polypeptide is a protein or a fragment of a protein from a biological sample.
83. The method of any one of embodiments 72-82, wherein the recording tag comprises a nucleic acid, an oligonucleotide, a modified oligonucleotide, a DNA molecule, a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof.
84. The method of embodiment 83, wherein:
   the DNA molecule is backbone modified, sugar modified, or nucleobase modified; or
   the DNA molecule has nucleobase protecting groups such as Alloc, electrophilic protecting groups such as thiaranes, acetyl protecting groups, nitrobenzyl protecting groups, sulfonate protecting groups, or traditional base-labile protecting groups including Ultramild reagents.
85. The method of any one of embodiments 72-84, wherein the recording tag comprises a universal priming site.
86. The method of embodiment 85, wherein the universal priming site comprises a priming site for amplification, sequencing, or both.
87. The method of embodiments 72-86, where the recording tag comprises a unique molecule identifier (UMI).
88. The method of any one of embodiments 72-87, wherein the recording tag comprises a barcode.
89. The method of any one of embodiments 72-88, wherein the recording tag comprises a spacer at its 3′-terminus.
90. The method of any one of embodiments 72-89, wherein the polypeptide and the associated recording tag are covalently joined to the support.
91. The method of any one of embodiments 72-90, wherein the support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.
92. The method of embodiment 91, wherein:
   the support comprises gold, silver, a semiconductor or quantum dots;
   the nanoparticle comprises gold, silver, or quantum dots; or
   the support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.
93. The method of any one of embodiments 72-92, wherein a plurality of polypeptides and associated recording tags are joined to a support.
94. The method of embodiment 93, wherein the plurality of polypeptides are spaced apart on the support, wherein the average distance between the polypeptides is about ≥20 nm.
95. The method of any one of embodiments 72-94, wherein the binding portion of the binding agent comprises a peptide or protein.
96. The method of any one of embodiments 72-95, wherein the binding portion of the binding agent comprises an aminopeptidase or variant, mutant, or modified protein thereof; an aminoacyl tRNA synthetase or variant, mutant, or modified protein thereof; an anticalin or variant, mutant, or modified protein thereof; a ClpS (such as ClpS2) or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.
97. The method of any one of embodiments 72-96, wherein:
   the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the polypeptide; or
   the binding agent binds to a NTAA-functionalized single amino acid residue, a NTAA-functionalized dipeptide, a NTAA-functionalized tripeptide, or a NTAA-functionalized polypeptide.
98. The method of any one of embodiments 72-97, wherein the binding portion of the binding agent is capable of selectively binding to the polypeptide.
99. The method of any one of embodiments 72-98, wherein the coding tag is DNA molecule, an RNA molecule, a BNA molecule, an XNA molecule, a LNA molecule, a PNA molecule, a γPNA molecule, or a combination thereof
100. The method of any one of embodiments 72-99, wherein the coding tag comprises an encoder or barcode sequence.
101. The method of any one of embodiments 72-100, wherein the coding tag further comprises a spacer, a binding cycle specific sequence, a unique molecular identifier, a universal priming site, or any combination thereof.
102. The method of any one of embodiments 72-101, wherein the binding portion and the coding tag are joined by a linker.
103. The method of any one of embodiments 72-102, wherein the binding portion and the coding tag are joined by a SpyTag/SpyCatcher peptide-protein pair, a SnoopTag/SnoopCatcher peptide-protein pair, or a HaloTag/HaloTag ligand pair.
104. The method of any one of embodiments 72-103, wherein:
   transferring the information of the coding tag to the recording tag is mediated by a DNA ligase or an RNA ligase;
   transferring the information of the coding tag to the recording tag is mediated by a DNA polymerase, an RNA polymerase, or a reverse transcriptase; or
   transferring the information of the coding tag to the recording tag is mediated by chemical ligation.
105. The method of embodiment 104, wherein the chemical ligation is performed using single-stranded DNA.
106. The method of embodiment 105, wherein the chemical ligation is performed using double-stranded DNA.
107. The method of any one of embodiments 72-106, wherein analyzing the extended recording tag comprises a nucleic acid sequencing method.
108. The method of embodiment 107, wherein:
   the nucleic acid sequencing method is sequencing by synthesis, sequencing by ligation, sequencing by hybridization, polony sequencing, ion semiconductor sequencing, or pyrosequencing; or
   the nucleic acid sequencing method is single molecule real-time sequencing, nanopore-based sequencing, or direct imaging of DNA using advanced microscopy.
109. The method of any one of embodiments 72-108, wherein the extended recording tag is amplified prior to analysis
110. The method of any one of embodiments 72-109, further comprising the step of adding a cycle label.
111. The method of embodiment 110, wherein the cycle label provides information regarding the order of binding by the binding agents to the polypeptide.
112. The method of embodiment 110 or embodiment 111, wherein:
   the cycle label is added to the coding tag;
   the cycle label is added to the recording tag;
   the cycle label is added to the binding agent; or
   the cycle label is added independent of the coding tag, recording tag, and binding agent.
113. The method of any one of embodiments 72-112, wherein the order of coding tag information contained on the extended recording tag provides information regarding the order of binding by the binding agents to the polypeptide.
114. The method of any one of embodiments 72-113, wherein frequency of the coding tag information contained on the extended recording tag provides information regarding the frequency of binding by the binding agents to the polypeptide.
115. The method of any one of embodiments 72-114, wherein a plurality of extended recording tags representing a plurality of polypeptides is analyzed in parallel.
116. The method of embodiment 115, wherein the plurality of extended recording tags representing a plurality of polypeptides is analyzed in a multiplexed assay.
117. The method of embodiment 115 or 116, wherein the plurality of extended recording tags undergoes a target enrichment assay prior to analysis.
118. The method of any one of embodiments 115-117, wherein the plurality of extended recording tags undergoes a subtraction assay prior to analysis.
119. The method of any one of embodiments 115-118, wherein the plurality of extended recording tags undergoes a normalization assay to reduce highly abundant species prior to analysis.
120. The method of any one of embodiments 72-119, which comprises treating the NTAA functionalized polypeptide with a non-acid medium to eliminate the NTAA.
121. The method of embodiment 120, wherein the suitable medium has a pH between 5 and 14. In some embodiments, the pH is between 8 and 14, or between 8 and 13.
122. The method of embodiment 120 or embodiment 121, wherein the suitable medium in step (2) comprises NH₃ or a primary amine.
123. The method of any one of embodiments 120-122, wherein eliminating the NTAA is performed step (a), step (b), step (c), and/or step (d).
124. The method of any one of embodiments 72-123, wherein the NTAA is eliminated by chemical cleavage under suitable conditions.
125. The method of embodiment 124, wherein the NTAA is eliminated by chemical cleavage induced by ammonia, a primary amine or a diheteronucleophile.
126. The method of embodiment 124, wherein the chemical cleavage is induced by ammonia.
127. The method of embodiment 126, wherein chemical cleavage is induced by a primary amine of the formula R²—NH₂, wherein R² is C₁₋₆ alkyl, which is optionally substituted with one or two members selected from halo, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl.
128. The method of embodiment 126, wherein chemical cleavage is induced by a diheteronucleophile selected from

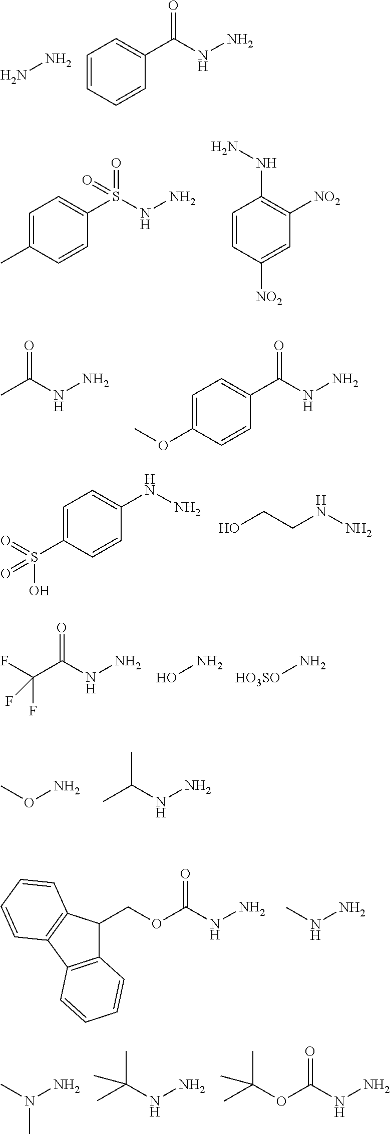

129. The method of any one of embodiments 72-128, wherein at least one binding agent binds to a terminal amino acid residue, terminal di-amino-acid residues, or terminal tri-amino-acid residues.
130. The method of any one of embodiments 72-129, wherein at least one binding agent binds to a post-translationally modified amino acid.
131. The method of any one of embodiments 72-130, wherein the chemical reagent comprises a compound of Formula (AA):

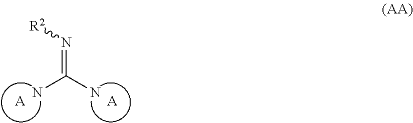

wherein Ring A is selected from:

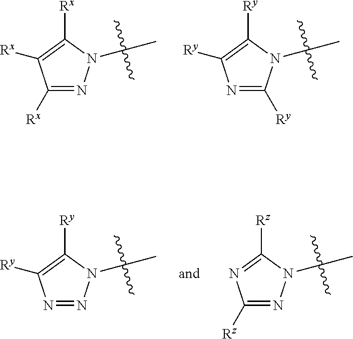

wherein:
each R^{x}, R^{y} and R^{z} is independently selected from H, halo, C₁₋₂ alkyl, C₁₋₂ haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂ haloalkyl, NO₂, SO₂(C₁₋₂ alkyl), COOR^{#}, and C(O)N(R⁴)₂,
   and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂haloalkyl, NO₂, SO₂(C₁₋₂ alkyl), COOR^{#}, and C(O)N(R^{#})₂;
   wherein each R^{#} is independently H or C₁₋₂ alkyl; and wherein two R^{#} on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂.

In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members. Specific examples of compounds of Formula (AA) for use in the methods and kits herein include:

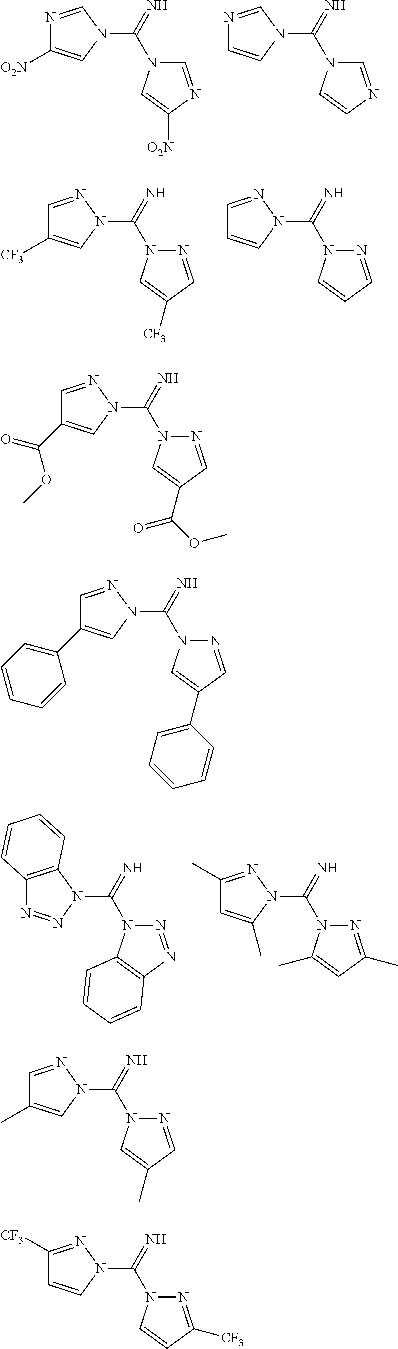

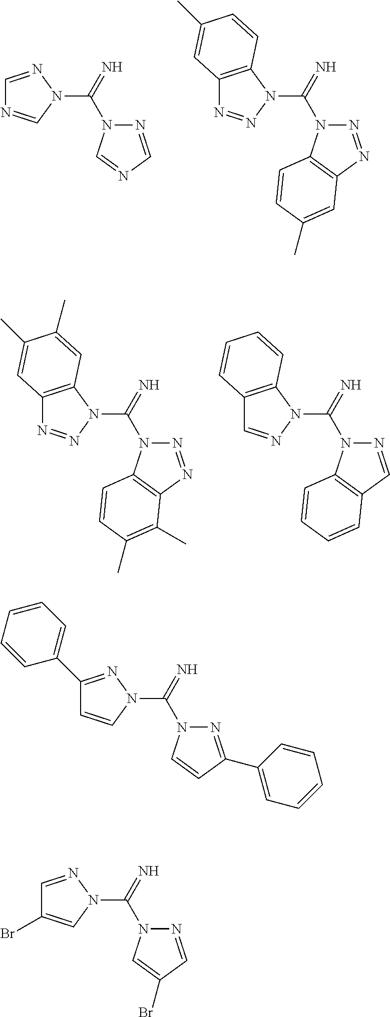 [SMILES: N=C(n1cncn1)n1cncn1 ; Cc1ccc2c(c1)nnn2C(=N)n1nnc2cc(C)ccc21 ; Cc1cc2nnn(C(=N)n3nnc4c(C)c(C)ccc43)c2cc1C ; N=C(n1ncc2ccccc21)n1ncc2ccccc21 ; N=C(n1ccc(-c2ccccc2)n1)n1ccc(-c2ccccc2)n1 ; N=C(n1cc(Br)cn1)n1cc(Br)cn1]

132. The method of embodiment 131, wherein ring A is selected from:

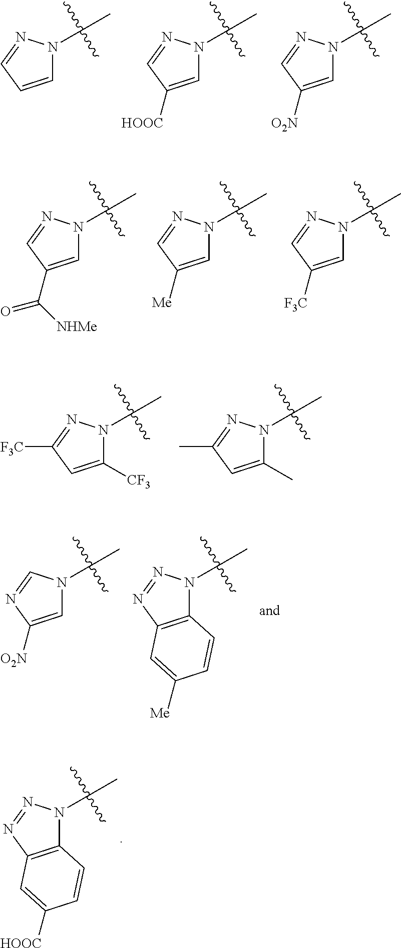

133. The method of any one of embodiments 72-132, wherein the chemical reagent is a compound of the formula R³—NCS, wherein R³ is phenyl, optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂,
   where each R′ is independently H or C₁₋₃ alkyl,
and wherein two R′ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂.
134. The method of any one of embodiments 72-133, wherein R² is H.
135. A kit for analyzing a polypeptide, comprising:
   (a) a reagent for functionalizing the N-terminal amino acid (NTAA) of the polypeptide, wherein the reagent comprises a compound of the formula (AA):

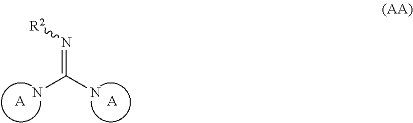

wherein each Ring A is selected from:

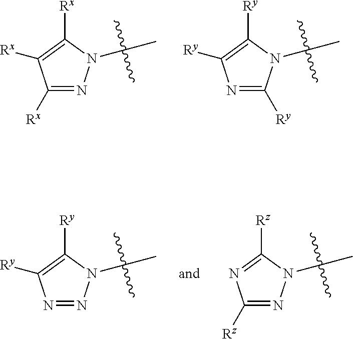

R² is H, R⁴, OH, OR⁴, NH₂, or —NHR⁴;
   R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
   where each R″ is independently H or C₁₋₃ alkyl;
   each R^{x}, R^{y} and R^{z} is independently selected from H, halo, C₁₋₂ alkyl, C₁₋₂ haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂ haloalkyl, NO₂, SO₂(C₁₋₂ alkyl), COOR^{#}, and C(O)N(R^{#})₂,
   and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;
   wherein each R^{#} is independently H or C₁₋₂ alkyl;
   and wherein two R# on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂;
(b) a plurality of binding agents, each comprising a binding portion capable of binding to the NTAA of a polypeptide either before or after the NTAA is functionalized by reaction with the compound of Formula (AA);
   (b1) a coding tag with identifying information regarding the binding agent, or
   (b2) a detectable label; and
(c) a reagent for transferring the information of the first coding tag to the recording tag to generate an extended recording tag; and optionally
(d) a reagent for analyzing the extended recording tag or a reagent for detecting the first detectable label.

In a preferred embodiment, R² is H. In certain of these embodiments, each 5-membered heteroaryl group present can be a 5-membered ring comprising one to three heteroatoms selected from N, O and S as ring members, and the 6-membered heteroaryl group can be a 6-membered ring comprising one to three nitrogen atoms as ring members.
136. The kit of embodiment 135, wherein the binding portion is capable of binding to:
   a non-functionalized NTAA or a NTAA that has been functionalized by the reagent in (a).
137. The kit of embodiment 135 or 136, further comprising a reagent for providing the polypeptide optionally associated directly or indirectly with a recording tag.
138. The kit of any one of embodiments 135-137, wherein:
   the reagent for providing the polypeptide is configured to provide the polypeptide and an associated recording tag joined to a support (e.g., a solid support);
   the reagent for providing the polypeptide is configured to provide the polypeptide associated directly with a recording tag in a solution;
   the reagent for providing the polypeptide is configured to provide the polypeptide associated indirectly with a recording tag; or
   the reagent for providing the polypeptide is configured to provide the polypeptide which is not associated with a recording tag.
139. The kit of any one of embodiments 135-138, wherein the kit further comprises a diheteronucleophile.
140. The kit of embodiment 139, wherein the diheteronucleophile is selected from:

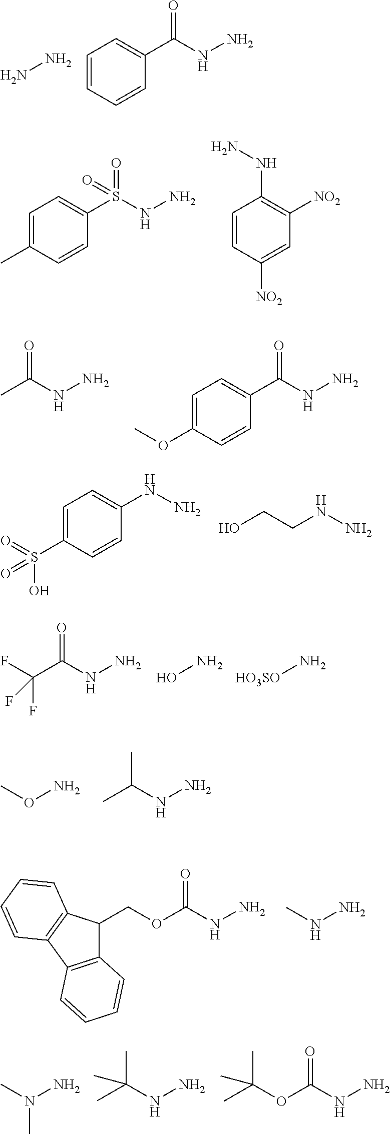

141. The kit of any one of embodiments 135-140, wherein the kit comprises two or more different binding agents.
142. The kit of any one of embodiments 135-141, further comprising a reagent for eliminating the functionalized NTAA to expose a new NTAA.
143. The kit of embodiment 141 or embodiment 142, wherein:
   the reagent for eliminating the functionalized NTAA comprises ammonia, a primary amine, or a diheteronucleophile.
144. The kit of any one of embodiments 142-143, wherein the reagent for eliminating the functionalized NTAA comprises a buffering agent with a pH between 7 and 14. In some embodiments, the pH is between 8 and 14, and in some embodiments the pH is between 8 and 13.
145. The kit of any one of embodiments 135-144, wherein the recording tag comprises a universal priming site.
146. The kit of embodiment 145, wherein the universal priming site comprises a priming site for amplification, sequencing, or both.
147. The kit of any one of embodiments 135-146, where the recording tag comprises a unique molecule identifier (UMI).
148. The kit of any one of embodiments 135-147, wherein:
   the recording tag comprises a barcode; or
   the recording tag comprises a spacer at its 3′-terminus.
149. The kit of any one of embodiments 135-148, wherein the reagents for providing the polypeptide and an associated recording tag joined to a support provide for covalent linkage of the polypeptide and the associated recording tag on the support.
150. The kit of any one of embodiments 145-149, wherein the support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.
151. The kit of embodiment 150, wherein:
   the support comprises gold, silver, a semiconductor or quantum dots;
   the nanoparticle comprises gold, silver, or quantum dots; or
   the support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.
152. The kit of any one of embodiments 135-151, wherein the reagents for providing the polypeptide and an associated recording tag joined to a support provide for a plurality of polypeptides and associated recording tags that are joined to a support.
153. The kit of embodiment 152, wherein the plurality of polypeptides are spaced apart on the support, wherein the average distance between the polypeptides is about ≥20 nm.
154. The kit of any one of embodiments 135-153, wherein the binding agent is a peptide or protein.
155. The kit of any one of embodiments 135-154, wherein the binding agent comprises an aminopeptidase or variant, mutant, or modified protein thereof; an aminoacyl tRNA synthetase or variant, mutant, or modified protein thereof; an anticalin or variant, mutant, or modified protein thereof; a ClpS or variant, mutant, or modified protein thereof; or a modified small molecule that binds amino acid(s), i.e. vancomycin or a variant, mutant, or modified molecule thereof; or an antibody or binding fragment thereof; or any combination thereof.
156. The kit of any one of embodiments 135-155, wherein the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the analyte or polypeptide.
157. The kit of any one of embodiments 135-156, wherein the binding agent binds to a NTAA-functionalized single amino acid residue, a NTAA-functionalized dipeptide, a NTAA-functionalized tripeptide, or a NTAA-functionalized polypeptide.
158. The kit of any one of embodiments 135-157, wherein the binding agent is capable of selectively binding to the polypeptide.
159. The kit of any one of embodiments 135-158, wherein the coding tag is DNA molecule, an RNA molecule, a BNA molecule, an XNA molecule, a LNA molecule, a PNA molecule, a γPNA molecule, or a combination thereof.
160. The kit of any one of embodiments 135-159, wherein the coding tag comprises an encoder or barcode sequence.
161. The kit of any one of embodiments 135-160, wherein the coding tag further comprises a spacer, a binding cycle specific sequence, a unique molecular identifier, a universal priming site, or any combination thereof.
162. The kit of any one of embodiments 135-161, wherein:
   the binding portion and the coding tag in the binding agent are joined by a linker; or
   the binding portion and the coding tag are joined by a SpyTag/SpyCatcher peptide-protein pair, a SnoopTag/SnoopCatcher peptide-protein pair, or a HaloTag/HaloTag ligand pair.
163. The kit of any one of embodiments 135-162, wherein:
   the reagent for transferring the information of the coding tag to the recording tag comprises a DNA ligase or an RNA ligase;
   the reagent for transferring the information of the coding tag to the recording tag comprises a DNA polymerase, an RNA polymerase, or a reverse transcriptase; or
   the reagent for transferring the information of the coding tag to the recording tag comprises a chemical ligation reagent.
164. The kit of embodiment 163, wherein:
   the chemical ligation reagent is for use with single-stranded DNA; or
   the chemical ligation reagent is for use with double-stranded DNA.
165. The kit of any one of embodiments 135-164;
   further comprising a ligation reagent comprised of two DNA or RNA ligase variants, an adenylated variant and a constitutively non-adenylated variant; or
   further comprising a ligation reagent comprised of a DNA or RNA ligase and a DNA/RNA deadenylase.
   166. The kit of any one of embodiments 135-165, wherein the kit additionally comprises reagents for nucleic acid sequencing methods.
167. The kit of embodiment 166, wherein:
   the nucleic acid sequencing method is sequencing by synthesis, sequencing by ligation, sequencing by hybridization, polony sequencing, ion semiconductor sequencing, or pyrosequencing; or
   the nucleic acid sequencing method is single molecule real-time sequencing, nanopore-based sequencing, or direct imaging of DNA using advanced microscopy.
168. The kit of any one of embodiments 135-167, wherein the kit additionally comprises reagents for amplifying the extended recording tag.
169. The kit of any one of embodiments 135-168, further comprising reagents for adding a cycle label.
170. The kit of embodiment 169, wherein the cycle label provides information regarding the order of binding by the binding agents to the polypeptide.
171. The kit of embodiment 169 or embodiment 170, wherein:
   the cycle label can be added to the coding tag;
   the cycle label can be added to the recording tag;
   the cycle label can be added to the binding agent; or
   the cycle label can be added independent of the coding tag, recording tag, and binding agent.
172. The kit of any one of embodiments 135-171, wherein the order of coding tag information contained on the extended recording tag provides information regarding the order of binding by the binding agents to the polypeptide.
173. The kit of any one of embodiments 135-172, wherein frequency of the coding tag information contained on the extended recording tag provides information regarding the frequency of binding by the binding agents to the polypeptide.
174. The kit of any one of embodiments 135-173, which is configured for analyzing one or more polypeptides from a sample comprising a plurality of protein complexes, proteins, or polypeptides.
175. The kit of embodiment 174, further comprising means for partitioning the plurality of protein complexes, proteins, or polypeptides within the sample into a plurality of compartments, wherein each compartment comprises a plurality of compartment tags optionally joined to a support (e.g., a solid support), wherein the plurality of compartment tags are the same within an individual compartment and are different from the compartment tags of other compartments.
176. The kit of embodiment 174 or 175, further comprising a reagent for fragmenting the plurality of protein complexes, proteins, and/or polypeptides into a plurality of polypeptides.
177. The kit of embodiment 176, wherein:
   the compartment is a microfluidic droplet;
   the compartment is a microwell; or
   the compartment is a separated region on a surface.
178. The kit of any one of embodiments 173-177, wherein each compartment comprises on average a single cell.
179. The kit of any one of embodiments 173-178, further comprising a reagent for labeling the plurality of protein complexes, proteins, or polypeptides with a plurality of universal DNA tags.
180. The kit of any one of embodiments 175-179, wherein the reagent for transferring the compartment tag information to the recording tag associated with a polypeptide comprises a primer extension or ligation reagent.
181. The kit of any one of embodiments 175-180, wherein:
   the support is a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics; a microtitre well, an ELISA plate, a spinning interferometry disc, a nitrocellulose membrane, a nitrocellulose-based polymer surface, a nanoparticle, or a microsphere; or
   the support comprises a bead.
182. The kit of embodiment 181, wherein the bead is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.
183. The kit of any one of embodiments 175-182, wherein the compartment tag comprises a single stranded or double stranded nucleic acid molecule.
184. The kit of any one of embodiments 175-183, wherein the compartment tag comprises a barcode and optionally a UMI.
185. The kit of embodiment 184, wherein:
   the support is a bead and the compartment tag comprises a barcode, further wherein beads comprising the plurality of compartment tags joined thereto are formed by split-and-pool synthesis; or
   the support is a bead and the compartment tag comprises a barcode, further wherein beads comprising a plurality of compartment tags joined thereto are formed by individual synthesis or immobilization.
186. The kit of any one of embodiments 175-185, wherein the compartment tag is a component within a recording tag, wherein the recording tag optionally further comprises a spacer, a barcode sequence, a unique molecular identifier, a universal priming site, or any combination thereof.
187. The kit of any one of embodiments 175-185, wherein the compartment tags further comprise a functional moiety capable of reacting with an internal amino acid, the peptide backbone, or N-terminal amino acid on the plurality of protein complexes, proteins, or polypeptides.
188. The kit of embodiment 187, wherein:
   the functional moiety is an aldehyde, an azide/alkyne, a moiety for a Staudinger reaction, or a maleimide/thiol, or an epoxide/nucleophile, or an inverse electron domain Diels-Alder (iEDDA) group; or the functional moiety is an aldehyde group.
189. The kit of any one of embodiments 175-188, wherein the plurality of compartment tags is formed by: printing, spotting, ink-jetting the compartment tags into the compartment, or a combination thereof.
190. The kit of any one of embodiments 175-189, wherein the compartment tag further comprises a polypeptide.
191. The kit of embodiment 190, wherein the compartment tag polypeptide comprises a protein ligase recognition sequence.
192. The kit of embodiment 191, wherein the protein ligase is butelase I or a homolog thereof.
193. The kit of any one of embodiments 175-192, wherein the reagent for fragmenting the plurality of polypeptides comprises a protease.
194. The kit of embodiment 193, wherein the protease is a metalloprotease.
195. The kit of embodiment 194, further comprising a reagent for modulating the activity of the metalloprotease, e.g., a reagent for photo-activated release of metallic cations of the metalloprotease.
196. The kit of any one of embodiments 175-195, further comprising a reagent for subtracting one or more abundant proteins from the sample prior to partitioning the plurality of polypeptides into the plurality of compartments.
197. The kit of any one of embodiment 175-196 further comprising a reagent for releasing the compartment tags from the support prior to joining of the plurality of polypeptides with the compartment tags.
198. The kit of embodiment 197, further comprising a reagent for joining the compartment tagged polypeptides to a support in association with recording tags.
199. The kit of any one of embodiments 175-198, further comprising one or more enzymes to remove the N-terminal amino acid of the polypeptide, e.g., a proline aminopeptidase, a proline iminopeptidase (PIP), a pyroglutamate aminopeptidase (pGAP), an asparagine amidohydrolase, a peptidoglutaminase asparaginase, a protein glutaminase, or a homolog thereof
200. A binding agent comprising a binding portion capable of binding to the N-terminal portion of a modified polypeptide of Formula (II)

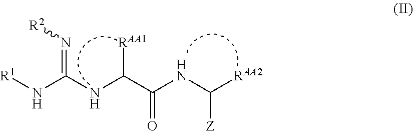

according to embodiment 37,
or Formula (IV)

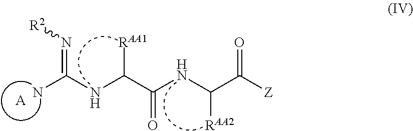

according to embodiment 47,
or a thiourea of formula

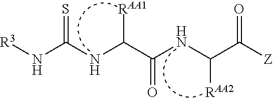

according to embodiment 22,
or of a side reaction product selected from

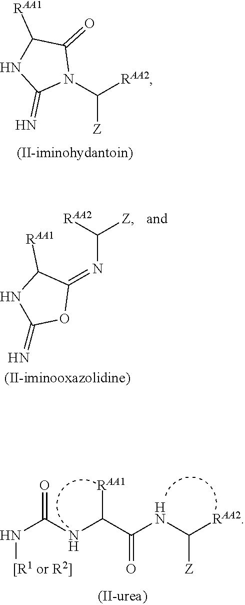

wherein R¹, R², Z, R^{AA1} and R^{AA2} are as defined for Formula (II), e.g. in Embodiment 37;
   or a side product of formula:

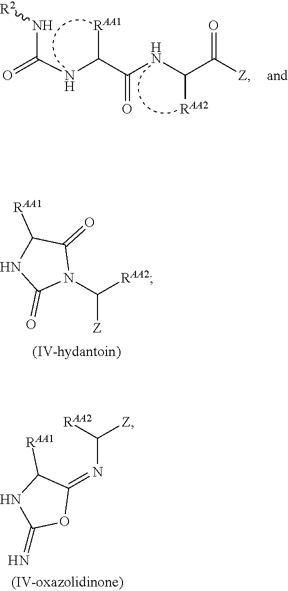

wherein R¹, R², ring A, Z, R^{AA1} and R^{AA2} are as defined for Formula (IV), e.g. in Embodiment 47.
201. The binding agent of embodiment 200, wherein the binding agent binds to the N-terminal portion of a modified polypeptide comprising an N-terminal amino acid residue, an N-terminal dipeptide, or an N-terminal tripeptide of the polypeptide.
202. The binding agent of embodiment 200 or 201, which comprises an aminopeptidase or variant, mutant, or modified protein thereof; an aminoacyl tRNA synthetase or variant, mutant, or modified protein thereof; an anticalin or variant, mutant, or modified protein thereof; a ClpS or variant, mutant, or modified protein thereof; or a modified small molecule that binds amino acid(s), i.e. vancomycin or a variant, mutant, or modified molecule thereof; or an antibody or binding fragment thereof; or any combination thereof
203. The binding agent of any one of embodiments 200-202, which is capable of selectively binding to the polypeptide.
204. The binding agent of any one of embodiments 200-203, further comprising a coding tag comprising identifying information regarding the binding moiety.
205. The binding agent of embodiment 204, wherein the binding agent and the coding tag are joined by a linker or a binding pair.
206. The binding agent of embodiment 204 or embodiment 205, wherein the coding tag is DNA molecule, an RNA molecule, a BNA molecule, an XNA molecule, a LNA molecule, a PNA molecule, a γPNA molecule, or a combination thereof.
207. The binding agent of any one of embodiments 204-206, wherein the coding tag further comprises a spacer, a binding cycle specific sequence, a unique molecular identifier, a universal priming site, or any combination thereof
208. A kit comprising a plurality of binding agents of any one of embodiments 200-207.

### Methods of Analyzing Polypeptides

In some embodiments, the provided methods and reagents for cleaving an amino acid from a polypeptide is applicable for use in methods of analyzing the polypeptides. In some embodiments, the polypeptide is cleaved in a cyclic process using any of the methods and reagents described herein for cleaving an N-terminal amino acid (NTAA). In some embodiments, the cyclic process includes functionalization of the NTAA followed by elimination or removal of the NTAA. In some embodiments, the removed NTAA is analyzed by protein analysis methods. In some embodiments, the polypeptide analysis methods include cycles of NTAA functionalization, NTAA elimination, NTAA binding by a binding agent, and transfer of information from the binding agent (e.g., a coding tag associated with the binding agent) to a recording tag associated with the polypeptide.

In some embodiments of the methods for analyzing a polypeptide, step (a) comprises providing the polypeptide joined to a support (e.g., a solid support). In some embodiments of the methods for analyzing a polypeptide, step (a) comprises providing the polypeptide and an associated recording tag joined to a support (e.g., a solid support). In some embodiments, step (a) comprises providing the polypeptide joined to an associated recording tag in a solution. In some embodiments, step (a) comprises providing the polypeptide associated indirectly with a recording tag. In some embodiments, the polypeptide is not associated with a recording tag in step (a). In one embodiment, the recording tag and/or the polypeptide are configured to be immobilized directly or indirectly to a support. In a further embodiment, the recording tag is configured to be immobilized to the support, thereby immobilizing the polypeptide associated with the recording tag. In another embodiment, the polypeptide is configured to be immobilized to the support, thereby immobilizing the recording tag associated with the polypeptide. In yet another embodiment, each of the recording tag and the polypeptide is configured to be immobilized to the support. In still another embodiment, the recording tag and the polypeptide are configured to co-localize when both are immobilized to the support. In some embodiments, the distance between (i) a polypeptide and (ii) a recording tag for information transfer between the recording tag and the coding tag of a binding agent bound to the polypeptide, is less than about 10⁻⁶ nm, about 10⁻⁶ nm, about 10⁻⁵ nm, about 10⁻⁴ nm, about 0.001 nm, about 0.01 nm, about 0.1 nm, about 0.5 nm, about 1 nm, about 2 nm, about 5 nm, or more than about 5 nm, or of any value in between the above ranges.

In some embodiments, the order of some of the steps in the process for a degradation-based peptide or polypeptide analysis assay can be reversed or be performed in various orders. For example, in some embodiments, the NTAA functionalization can be conducted before and/or after the polypeptide is bound to the binding agent. In some embodiments of any of the methods described herein, the N-terminal amino acid (NTAA) of the polypeptide is functionalized (step (b)) before the polypeptide is contacted with a first binding agent (step (c)). In some embodiments, the N-terminal amino acid (NTAA) of the polypeptide is functionalized (step (b)) after the polypeptide is contacted with a first binding agent (step (c)), but before the transferring of the information (step (d1)) or detecting the first detectable label (step (d2)). In some embodiments, the N-terminal amino acid (NTAA) of the polypeptide is functionalized (step (b)) after the polypeptide is contacted with a first binding agent (step (c)) and after the transferring of the information (step (d1)) or detecting the first detectable label (step (d2)). In some embodiments, the N-terminal amino acid (NTAA) of the polypeptide is functionalized (step (b)) after the polypeptide is contacted with a first binding agent (step (c)), and after the transferring of the information (step (d1)) or detecting the first detectable label (step (d2)). In some embodiments, the polypeptide is contacted with a binding agent (step (c)) before the N-terminal amino acid (NTAA) of the polypeptide is functionalized (step (b)). In some embodiments, the polypeptide is contacted with a binding agent (step (c)) after the N-terminal amino acid (NTAA) of the polypeptide is functionalized (step (b)). In some embodiments, the polypeptide is contacted with a binding agent (step (c)) before the transferring of the information (step (d)). In some embodiments, the one or more binding agents is removed or released from the polypeptides. For example, removal of the binding agent from the polypeptide can be performed prior to or after the functionalization of the NTAA. In some cases, the binding agent is removed or released from the polypeptide after the transferring of information or detecting of a detectable label.

Provided in some aspects are methods for analyzing a polypeptide, comprising the steps of: (a) providing the polypeptide optionally associated directly or indirectly with a recording tag; (b) functionalizing the N-terminal amino acid (NTAA) of the polypeptide with a chemical reagent to yield a functionalized NTAA; (c) contacting the polypeptide with a first binding agent comprising a first binding portion capable of binding to the functionalized NTAA and (c1) a first coding tag with identifying information regarding the first binding agent, or (c2) a first detectable label; (d) (d1) transferring the information of the first coding tag to the recording tag to generate a first extended recording tag and analyzing the extended recording tag, or (d2) detecting the first detectable label, and (e) eliminating the functionalized NTAA to expose a new NTAA. In some embodiments, step (a) comprises providing the polypeptide and an associated recording tag joined to a support (e.g., a solid support). In some embodiments, step (a) comprises providing the polypeptide joined to an associated recording tag in a solution. In some embodiments, step (a) comprises providing the polypeptide associated indirectly with a recording tag. In some embodiments, the polypeptide is not associated with a recording tag in step (a). In some embodiments of any of the methods described herein, the chemical reagent of step (b) for functionalizing the N-terminal amino acid (NTAA) of the polypeptide comprises a compound selected from a compound any one of Formula (AA) or Formula (AB), or a salt or conjugate thereof, as described herein. In some embodiments of any of the methods described herein, the chemical reagent of step (b) for functionalizing the N-terminal amino acid (NTAA) of the polypeptide comprises a compound of the formula R³—NCS or a salt or conjugate thereof, as described herein. In some embodiments, the polypeptide is further treated with an amine of Formula R²—NH₂ or with a diheteronucleophile to form a secondary functionalized NTAA.

In some embodiments, the methods further include (f) functionalizing the new NTAA of the polypeptide with a chemical reagent to yield a newly functionalized NTAA; (g) contacting the polypeptide with a second (or higher order) binding agent comprising a second (or higher order) binding portion capable of binding to the newly functionalized NTAA and (g1) a second coding tag with identifying information regarding the second (or higher order) binding agent, or (g2) a second detectable label; (h) (h1) transferring the information of the second coding tag to the first extended recording tag to generate a second extended recording tag and analyzing the second extended recording tag, or (h2) detecting the second detectable label, and (i) eliminating the functionalized NTAA to expose a new NTAA. In some embodiments of any of the methods described herein, the chemical reagent of step (f) for functionalizing the N-terminal amino acid (NTAA) of the polypeptide comprises a compound selected from a compound any one of Formula (AA) or a salt or conjugate thereof, as described herein. In some embodiments of any of the methods described herein, the chemical reagent of step (f) for functionalizing the N-terminal amino acid (NTAA) of the polypeptide comprises a compound selected from a compound of Formula (AA), Formula (AB), a compound of the formula R³—NCS, an amine of Formula R²—NH₂ or with a diheteronucleophile, or a salt or conjugate thereof, as described herein, or any combinations thereof. Suitable compounds of Formula (AA) for use in the methods and kits herein include:

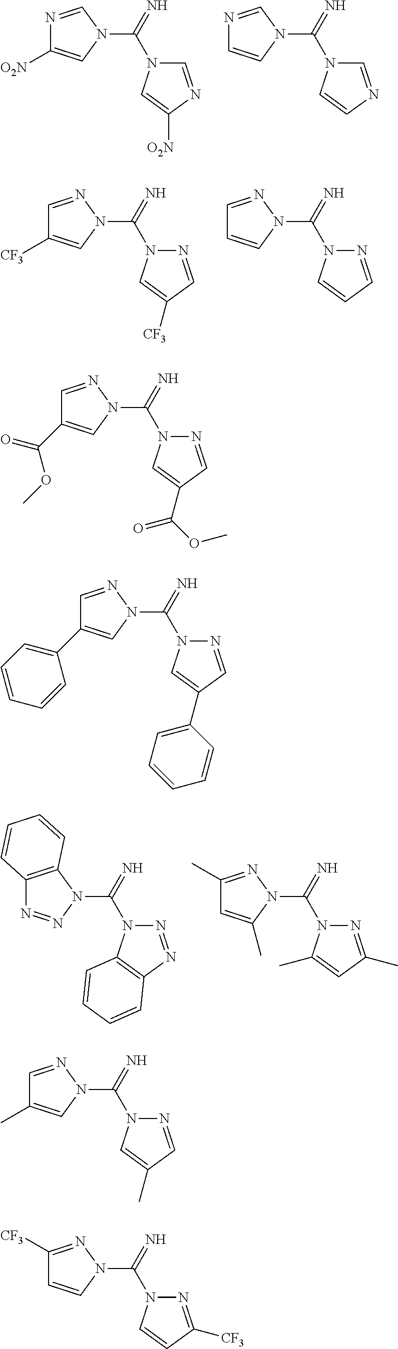

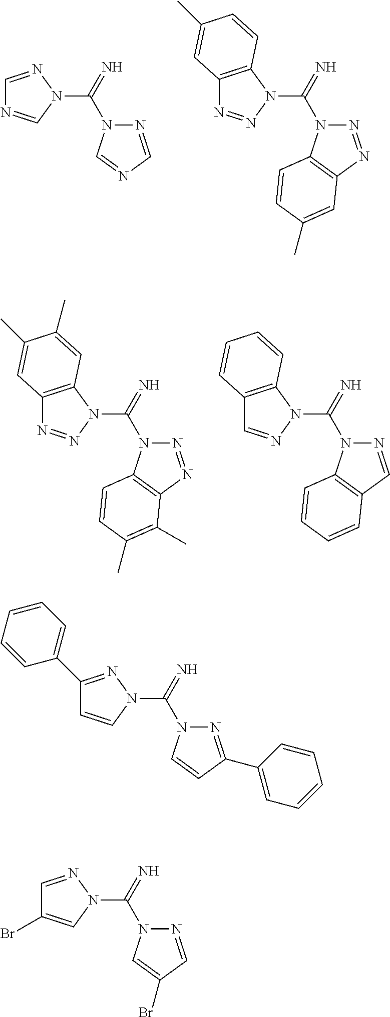 [SMILES: N=C(n1cncn1)n1cncn1 ; Cc1ccc2c(c1)nnn2C(=N)n1nnc2cc(C)ccc21 ; Cc1cc2nnn(C(=N)n3nnc4c(C)c(C)ccc43)c2cc1C ; N=C(n1ncc2ccccc21)n1ncc2ccccc21 ; N=C(n1ccc(-c2ccccc2)n1)n1ccc(-c2ccccc2)n1 ; N=C(n1cc(Br)cn1)n1cc(Br)cn1]

In some of any such embodiments, the binding agents (e.g., first order, second order, or any higher order binding agents) is capable of binding or configured to bind a non-functionalized NTAA or a functionalized NTAA. In some embodiments, the functionalized NTAA is an initial functionalized NTAA or a secondary functionalized NTAA. In some embodiments, the functionalized NTAA is an NTAA treated with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof. In some examples, the functionalized NTAA is a product from step (b1) after contacting the polypeptide with the compound of Formula AA. In some examples, the functionalized NTAA is a product from step (b2) after contacting the polypeptide with the compound of the formula R³—NCS. In some examples, the functionalized NTAA is a product from step (b1) further contacted with the amine of Formula R²—NH₂ or with the diheteronucleophile. In some examples, the functionalized NTAA is a product from step (b2) further contacted with the amine of Formula R²—NH₂ or with the diheteronucleophile.

In some embodiments, the binding agent (e.g., first order, second order, or any higher order binding agent) is capable of binding or configured to bind a side product from treating the polypeptide with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof. Side products that can occur in Step 1 are generated through certain conditions that occur during increased pH (e.g., pH >8) and/or increased temperature of the system. General side products formed for all NTAA are described as 1) iminohydantoin; where the adjacent amide intramolecularly reacts with the imino carbon of the functionalized N-terminal amino acid to produce the hydantoin-like ring, and 2) urea; where the functionalized N-terminal amino acid undergoes base-promoted hydrolysis stemming from the solvent. Side products that can arise from a compound of Formula (II) as described herein include:

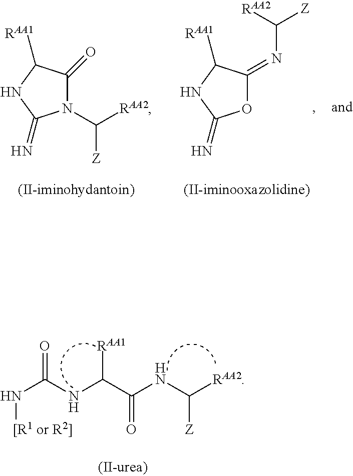

wherein R¹, R², Z, R^{AA1} and R^{AA2} are as defined for Formula (II), e.g., in Embodiment 37. Side products that can arise from a compound of Formula (IV) as described herein include:

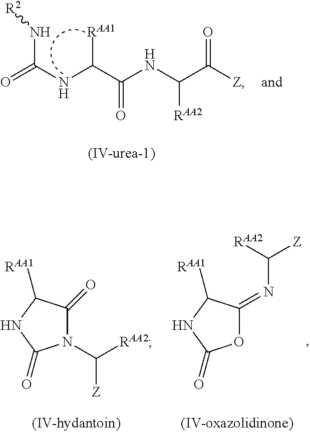

wherein R¹, R², ring A, Z, R^{AA1} and R^{AA2} are as defined for Formula (IV), e.g., in Embodiment 47.

In some cases, these side products are considered to be irreversible and subsequent elimination or removal of the NTAA is not possible. In some embodiments of the methods of the invention, binding agents specific for one or more of these side products can be used to detect the occurrence of these species and to determine the identity of the NTAA even though the NTAA was not cleaved.

In some cases, caveats exist depending on the functionality of the NTAA side chain. In some instances, where the N-terminal amino acid is proline, after functionalization of the N-terminus, the neighboring amide reacts with the functionalized N-terminus to cyclize and forms a [5,5] bicyclic ring. Where the N-terminal residue is asparagine, the terminal amide of side chain can also react with the functionalized N-terminus to form a pyrimidinone. Where the N-terminus is Serine or Threonine, the primary or secondary hydroxyl oxygen can react with the functionalized N-terminal imine and cyclize to form an iminooxazoline. Similarly if the N-terminal residue is cysteine, the thiol will form a cyclized product with the functionalized N-terminal amine resulting in an iminothiazoline. All of these side products can undergo reaction with a diheteronucleophile to form an aminoguanidine intermediate, which can then undergo elimination.

In some embodiments of any of the methods provided herein, the polypeptide is associated directly with a recording tag. In some embodiments, the polypeptide is associated directly with a recording tag on a support (e.g., a solid support). In some embodiments, the polypeptide is associated directly with a recording tag in a solution. In some embodiments, the polypeptide is associated indirectly with a recording tag. In some embodiments, the polypeptide is associated indirectly with a recording tag on a support (e.g., a solid support). In some embodiments, the polypeptide is associated indirectly with a recording tag in a solution.

In some embodiments of any of the methods provided herein, the polypeptide is not associated with an oligonucleotide, such as a recording tag. In some embodiments, the methods for analyzing a polypeptide comprises the steps of: (a) providing the polypeptide; (b) functionalizing the N-terminal amino acid (NTAA) of the polypeptide with a chemical reagent; (c) contacting the polypeptide with a first binding agent comprising a first binding portion capable of binding to the functionalized NTAA and (c2) a first detectable label; and (d2) detecting the first detectable label. In some embodiments, the method further comprises (e) eliminating the functionalized NTAA to expose a new NTAA.

In some embodiments, step (b) is conducted before step (c), after step (c) and before step (d2), or after step (d2). In some embodiments, steps (a), (b), (c), and (d2) occur in sequential order. In some embodiments, steps (a), (c), (b), and (d2) occur in sequential order. In some embodiments, steps (a), (c), (d2) and (b) occur in sequential order. In some embodiments of any of the methods described herein, the chemical reagent of step (b) for functionalizing the N-terminal amino acid (NTAA) of the polypeptide comprises a compound selected from a compound of any one of a compound any one of Formula (AA), Formula (AB), a compound of the formula R³—NCS, an amine of Formula R²—NH₂ or with a diheteronucleophile, or a salt or conjugate thereof, as described herein, or any combinations thereof.

In some embodiments, steps (a), (b), (c1), and (d1) occur in sequential order. In some embodiments, steps (a), (c1), (b), and (d1) occur in sequential order. In some embodiments, steps (a), (c1), (d1), and (b) occur in sequential order. In some embodiments, steps (a), (b2), (c1), and (d1) occur in sequential order. In some embodiments, steps (a), (b1), (c1), and (d1) occur in sequential order. In some embodiments, steps (a), (c1), (b1), and (d1) occur in sequential order. In some embodiments, steps (a), (c1), (b2), and (d1) occur in sequential order. In some embodiments, steps (a), (c1), (d1), and (b1) occur in sequential order. In some embodiments, steps (a), (c1), (d1), and (b2) occur in sequential order. In some embodiments, steps (a), (b), (c2), and (d2) occur in sequential order. In some embodiments, steps (a), (c2), (b), and (d2) occur in sequential order. In some embodiments, steps (a), (c2), (d2), and (b) occur in sequential order.

In some embodiments, the methods further include (f) functionalizing the new NTAA of the polypeptide with a chemical reagent to yield a newly functionalized NTAA; (g) contacting the polypeptide with a second (or higher order) binding agent comprising a second (or higher order) binding portion capable of binding to the newly functionalized NTAA and (g2) a second detectable label; (h2) detecting the second detectable label, and (i) eliminating the functionalized NTAA to expose a new NTAA. In some embodiments, step (f) is conducted before step (g), after step (g) and before step (h2), or after step (h2). In some embodiments, steps (f), (g), and (h2) occur in sequential order. In some embodiments, steps (g), (f), and (h2) occur in sequential order. In some embodiments, steps (g), (h2) and (f) occur in sequential order. In some embodiments of any of the methods described herein, the chemical reagent of step (f) for functionalizing the N-terminal amino acid (NTAA) of the polypeptide comprises a compound selected from a compound any one of a compound any one of Formula (AA), Formula (AB), a compound of the formula R³—NCS, an amine of Formula R²—NH₂ or with a diheteronucleophile, or a salt or conjugate thereof, as described herein, or any combinations thereof.

In some embodiments of any of the methods described herein, the N-terminal amino acid (NTAA) of the polypeptide is functionalized (step (b) or step (f)) before the polypeptide is contacted with a binding agent (step (c) or step (g)). In some embodiments, the N-terminal amino acid (NTAA) of the polypeptide is functionalized (step (f)) after the polypeptide is contacted with a binding agent (step (c) or step (g)), but before the transferring of the information (step (d1) or step (h1)) or detecting the detectable label (step (d2) or step (h2)). In some embodiments, the N-terminal amino acid (NTAA) of the polypeptide is functionalized (step (b) or step (f)) after the polypeptide is contacted with a binding agent (step (c) or step (g)) and after the transferring of the information (step (d1) or step (h1)) or detecting the first detectable label (step (d2) or step (h2)).

In some embodiments of any of the methods described herein, steps (f), (g), (h), and (i) are repeated for multiple amino acids in the polypeptide. In some embodiments, steps (f), (g), (h), and (i) are repeated for two or more amino acids in the polypeptide. In some embodiments, steps (f), (g), (h), and (i) are repeated for up to about 10 amino acids, up to about 20 amino acids, up to about 30 amino acids, up to about 40 amino acids, up to about 50 amino acids, up to about 60 amino acids, up to about 70 amino acids, up to about 80 amino acids, up to about 90 amino acids, or up to about 100 amino acids. In some embodiments, steps (f), (g), (h), and (i) are repeated for up to about 100 amino acids. In some embodiments, steps (f), (g), (h), and (i) are repeated for at least about 100 amino acids, at least about 200 amino acids, or at least about 500 amino acids.

In some embodiments, step (c) further comprises contacting the polypeptide with a second (or higher order) binding agent comprising a second (or higher order) binding portion capable of binding to a functionalized NTAA other than the functionalized NTAA of step (b) and a coding tag with identifying information regarding the second (or higher order) binding agent. In some embodiments, contacting the polypeptide with the second (or higher order) binding agent occurs in sequential order following the polypeptide being contacted with the first binding agent. In some embodiments, contacting the polypeptide with the second (or higher order) binding agent occurs simultaneously with the polypeptide being contacted with the first binding agent. In some embodiments, contacting the polypeptide with the second (or higher order) binding agent occurs in sequential order following the polypeptide being contacted with the first binding agent. In some embodiments, contacting the polypeptide with the second (or higher order) binding agent occurs simultaneously with the polypeptide being contacted with the first binding agent.

In some embodiments, the second (or higher order) binding agent may be contacted with the polypeptide in a separate binding cycle reaction from the first binding agent. In some embodiments, the higher order binding agent is a third (or higher order binding agent). The third (or higher order) binding agent may be contacted with the polypeptide in a separate binding cycle reaction from the first binding agent and the second binding agent. In one embodiment, a n^{th} binding agent is contacted with the polypeptide at the n^{th} binding cycle, and information is transferred from the n^{th} coding tag (of the n^{th} binding agent) to the extended recording tag formed in the (n−1)^{th} binding cycle in order to form a further extended recording tag (the n^{th} extended recording tag), wherein n is an integer of 2, 3, 4, 5, 6, 7, 8, 9, 10, 11, 12, 13, 14, 15, 16, 17, 18, 19, 20, 21, 22, 23, 24, 25, 26, 27, 28, 29, 30, or about 50, about 100, about 150, about 200, or more. Similarly, a (n+1)^{th} binding agent is contacted with the polypeptide at the (n+1)^{th} binding cycle, and so on.

Alternatively, the third (or higher order) binding agent may be contacted with the polypeptide in a single binding cycle reaction with the first binding agent, and the second binding agent. In this case, binding cycle specific sequences such as binding cycle specific coding tags may be used. For example, the coding tags may comprise binding cycle specific spacer sequences, such that only after information is transferred from the n^{th} coding tag to the (n−1)^{th} extended recording tag to form the n^{th} extended recording tag, will then the (n+1)^{th} binding agent (which may or may not already be bound to the analyte) be able to transfer information of the (n+1)^{th} binding tag to the n^{th} extended recording tag.

In some embodiments, the polypeptide is obtained by fragmenting a protein from a biological sample. Examples of biological samples include, but are not limited to cells (both primary cells and cultured cell lines), cell lysates or extracts, cell organelles or vesicles, including exosomes, tissues and tissue extracts; biopsy; fecal matter; bodily fluids (such as blood, whole blood, serum, plasma, urine, lymph, bile, cerebrospinal fluid, interstitial fluid, aqueous or vitreous humor, colostrum, sputum, amniotic fluid, saliva, anal and vaginal secretions, perspiration and semen, a transudate, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; research samples including extracellular fluids, extracellular supernatants from cell cultures, inclusion bodies in bacteria, cellular compartments including mitochondrial compartments, and cellular periplasm.

In some embodiments, the recording tag comprises a nucleic acid, an oligonucleotide, a modified oligonucleotide, a DNA molecule, a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the DNA molecule is backbone modified, sugar modified, or nucleobase modified. In some embodiments, the DNA molecule has nucleobase protecting groups such as Alloc, electrophilic protecting groups such as thiranes, acetyl protecting groups, nitrobenzyl protecting groups, sulfonate protecting groups, or traditional base-labile protecting groups including Ultramild reagents.

In some embodiments, the recording tag comprises a universal priming site. In some embodiments, the universal priming site comprises a priming site for amplification, sequencing, or both. In some embodiments, the recording tag comprises a unique molecule identifier (UMI). In some embodiments, the recording tag comprises a barcode. In some embodiments, the recording tag comprises a spacer at its 3′-terminus. In some embodiments, the recording tag comprises a spacer at its 5′-terminus. In some embodiments, the polypeptide and the associated recording tag are covalently joined to the support.

In some embodiments, the support is a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere. In some embodiments, the support comprises gold, silver, a semiconductor or quantum dots. In some embodiments, the nanoparticle comprises gold, silver, or quantum dots. In some embodiments, the support is a polystyrene bead, a polymer bead, an agarose bead, an acrylamide bead, a solid core bead, a porous bead, a paramagnetic bead, glass bead, or a controlled pore bead.

In some embodiments, a plurality of polypeptides and associated recording tags are joined to a support. In some embodiments, the plurality of polypeptides are spaced apart on the support, wherein the average distance between the polypeptides is about ≥20 nm. In some embodiments, the average distance between the polypeptides is about ≥30 nm, about ≥40 nm, about ≥50 nm, about ≥60 nm, about ≥70 nm, about ≥80 nm, about ≥100 nm, or about ≥500 nm. In other embodiments, the average distance between polypeptides is about ≤500 nm, about ≤100 nm, about ≤80 nm, about ≤70 nm, about ≤60 nm, about ≤50 nm, about ≤40 nm, about ≤30 nm, or about ≤20 nm.

In some embodiments, the binding portion of the binding agent comprises a peptide or protein. In some embodiments, the binding portion of the binding agent comprises an aminopeptidase or variant, mutant, or modified protein thereof; an aminoacyl tRNA synthetase or variant, mutant, or modified protein thereof; an anticalin or variant, mutant, or modified protein thereof; a ClpS (such as ClpS2) or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

In some embodiments, the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the polypeptide. In some embodiments, the binding agent binds to a NTAA-functionalized single amino acid residue, a NTAA-functionalized dipeptide, a NTAA-functionalized tripeptide, or a NTAA-functionalized polypeptide.

In some embodiments, the binding portion of the binding agent is capable of selectively binding to the polypeptide. In some embodiments, the binding agent selectively binds to a functionalized NTAA. For example, the binding agent may selectively bind to the NTAA after the NTAA is treated or functionalized with a chemical reagent, wherein the chemical reagent comprises at least one compound selected from any of the compounds presented herein, such as compounds of Formula (AA), Formula (AB), a compound of the formula R³—NCS, an amine of Formula R²—NH₂ or with a diheteronucleophile, or a salt or conjugate thereof, as described herein. In some embodiments, the binding agent is a non-cognate binding agent. In some aspects, the binding agent is configured to bind or recognize a portion of the polypeptide that comprises an NTAA that is treated or functionalized with a chemical reagent as described herein. In some instances, the binding agent may bind the chemically modified NTAA and one or more additional amino acid residues.

In some embodiments, at least one binding agent binds to a terminal amino acid residue, terminal di-amino-acid residues, or terminal tri-amino-acid residues. In some embodiments, at least one binding agent binds to a post-translationally modified amino acid. In some cases, the binding agents bind to a non-functionalized or non-chemically modified NTAA. In some cases, the binding agents bind to a functionalized NTAA or chemically modified NTAA. In some embodiments, the functionalized NTAA is an NTAA treated with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof. In some embodiments, the binding agents (e.g., first order, second order, or any higher order binding agents) is capable of binding or configured to bind to a side product from treating the polypeptide with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof.

In some embodiments, the coding tag is DNA molecule, an RNA molecule, a BNA molecule, an XNA molecule, a LNA molecule, a PNA molecule, a γPNA molecule, or a combination thereof. In some embodiments, the coding tag comprises an encoder or barcode sequence. In some embodiments, the coding tag further comprises a spacer, a binding cycle specific sequence, a unique molecular identifier, a universal priming site, or any combination thereof. In some embodiments, the coding tag comprises a nucleic acid, an oligonucleotide, a modified oligonucleotide, a DNA molecule, a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the DNA molecule is backbone modified, sugar modified, or nucleobase modified. In some embodiments, the DNA molecule has nucleobase protecting groups such as Alloc, electrophilic protecting groups such as thiranes, acetyl protecting groups, nitrobenzyl protecting groups, sulfonate protecting groups, or traditional base-labile protecting groups including Ultramild reagents.

In some embodiments, the binding portion and the coding tag are joined by a linker. In some embodiments, the binding portion and the coding tag are joined by a SpyTag/SpyCatcher peptide-protein pair, a SnoopTag/SnoopCatcher peptide-protein pair, or a HaloTag/HaloTag ligand pair.

In some embodiments, transferring the information of the coding tag to the recording tag is mediated by a DNA ligase or an RNA ligase. In some embodiments, transferring the information of the coding tag to the recording tag is mediated by a DNA polymerase, an RNA polymerase, or a reverse transcriptase. In some embodiments, transferring the information of the coding tag to the recording tag is mediated by chemical ligation. In some embodiments, the chemical ligation is performed using single-stranded DNA. In some embodiments, the chemical ligation is performed using double-stranded DNA.

In some embodiments, analyzing the extended recording tag comprises a nucleic acid sequencing method. In some embodiments, the nucleic acid sequencing method is sequencing by synthesis, sequencing by ligation, sequencing by hybridization, polony sequencing, ion semiconductor sequencing, or pyrosequencing. In some embodiments, the nucleic acid sequencing method is single molecule real-time sequencing, nanopore-based sequencing, or direct imaging of DNA using advanced microscopy.

In some embodiments, the extended recording tag is amplified prior to analysis. The extended recording tag can be amplified using any method known in the art, for example, using PCR or linear amplification methods.

In some embodiments, the method further includes the step of adding a cycle label. In some embodiments, the cycle label provides information regarding the order of binding by the binding agents to the polypeptide. In some embodiments, the cycle label is added to the coding tag. In some embodiments, the cycle label is added to the recording tag. In some embodiments, the cycle label is added to the binding agent. In some embodiments, the cycle label is added independent of the coding tag, recording tag, and binding agent.

In some embodiments, the order of coding tag information contained on the extended recording tag provides information regarding the order of binding by the binding agents to the polypeptide. In some embodiments, the frequency of the coding tag information contained on the extended recording tag provides information regarding the frequency of binding by the binding agents to the polypeptide.

In some embodiments, a plurality of extended recording tags representing a plurality of polypeptides is analyzed in parallel. In some embodiments, the plurality of extended recording tags representing a plurality of polypeptides is analyzed in a multiplexed assay. In some embodiments, the plurality of extended recording tags undergoes a target enrichment assay prior to analysis. In some embodiments, the plurality of extended recording tags undergoes a subtraction assay prior to analysis. In some embodiments, the plurality of extended recording tags undergoes a normalization assay to reduce highly abundant species prior to analysis. In any of the embodiments disclosed herein, multiple polypeptide samples, wherein a population of polypeptides within each sample are labeled with recording tags comprising a sample specific barcode, can be pooled. Such a pool of polypeptide samples may be subjected to binding cycles within a single-reaction tube.

In some embodiments, the NTAA is eliminated by chemical elimination or enzymatic elimination from the polypeptide. In some embodiments, the NTAA is eliminated by treatment with a base, an amine, or a diheteronucleophile, or any combination thereof. The functionalization and elimination of terminal amino acid moieties are discussed in more detail in the sections that follow.

Provided in some aspects are methods of sequencing a polypeptide comprising: (a) affixing the polypeptide to a support or substrate, or providing the polypeptide in a solution; (b) functionalizing the N-terminal amino acid (NTAA) of the polypeptide with a chemical reagent, wherein the chemical reagent comprises a compound of Formula (AB) or Formula (AA) as described herein; (c) contacting the polypeptide with a plurality of binding agents each comprising a binding portion capable of binding to the functionalized NTAA and a detectable label; (d) detecting the detectable label of the binding agent bound to the polypeptide, thereby identifying the N-terminal amino acid of the polypeptide; (e) eliminating the functionalized NTAA to expose a new NTAA; and (f) repeating steps (b) to (d) or steps (b) to (e) to determine the sequence of at least a portion of the polypeptide.

In some embodiments, step (b) is conducted before step (c). In some embodiments, step (b) is conducted after step (c) and before step (d). In some embodiments, step (b) is conducted after both step (c) and step (d). In some embodiments, steps (a), (b), (c), (d), and (e) occur in sequential order. In some embodiments, steps (a), (c), (b), (d), and (e) occur in sequential order. In some embodiments, steps (a), (c), (d), (b), and (e) occur in sequential order.

In some embodiments of any of the methods described herein, the polypeptide is obtained by fragmenting a protein from a biological sample. In some embodiments, the support or substrate is a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.

In some embodiments of any of the methods described herein, the NTAA is eliminated by chemical cleavage or enzymatic cleavage from the polypeptide. In some embodiments, the NTAA is eliminated by treatment with an amine, a base, a diheteronucleophile, or any combination thereof.

In some embodiments of any of the methods described herein, the polypeptide is covalently affixed to the support or substrate. In some embodiments, the support or substrate is optically transparent. In some embodiments, the support or substrate comprises a plurality of spatially resolved attachment points and step a) comprises affixing the polypeptide to a spatially resolved attachment point.

In some embodiments of any of the methods described herein, the binding portion of the binding agent comprises a peptide or protein. In some embodiments, the binding portion of the binding agent comprises an aminopeptidase or variant, mutant, or modified protein thereof; an aminoacyl tRNA synthetase or variant, mutant, or modified protein thereof; an anticalin or variant, mutant, or modified protein thereof; a ClpS (such as ClpS2) or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

In some embodiments, the chemical reagent comprises a conjugate of the formula:

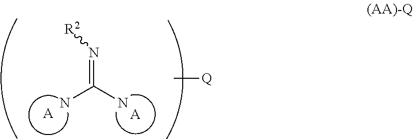

wherein R² and ring A are as defined for Formula (AA) in any one of the embodiments above, and Q is a ligand;

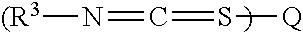

wherein R³ is as defined for Formula (III) in any one of the embodiments above, and Q is a ligand.

In some embodiments, the chemical reagent used to functionalize the terminal amino acid of a polypeptide comprises a conjugate of Formula (AA)-Q, are as defined above, and Q is a ligand.

In some embodiments, the ligand Q is a pendant group or binding site (e.g., the site to which the binding agent binds). In some embodiments, the polypeptide binds covalently to a binding agent. In some embodiments, the polypeptide comprises a functionalized NTAA which includes a ligand group that is capable of covalent binding to a binding agent. In certain embodiments, the polypeptide comprises a functionalized NTAA with a compound of Formula (AA)-Q, wherein the Q binds covalently to a binding agent. In some embodiments, a coupling reaction is carried out to create a covalent linkage between the polypeptide and the binding agent (e.g., a covalent linkage between the ligand Q and a functional group on the binding agent).

In some embodiments, the chemical reagent used to functionalize the terminal amino acid of a polypeptide comprises a conjugate of Formula (I)-Q

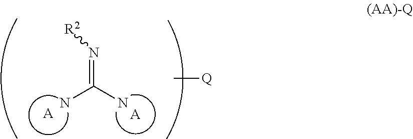

In some embodiments, Q is selected from the group consisting of —C₁₋₆ alkyl, —C₂₋₆alkenyl, —C₂₋₆alkynyl, aryl, heteroaryl, heterocyclyl, —N═C═S, —CN, —C(O)Rⁿ, —C(O)OR^{o}, —SR^{p} or —S(O)₂R^{q}; wherein the —C₁₋₆alkyl, —C₂₋₆alkenyl, —C₂₋₆alkynyl, aryl, heteroaryl, and heterocyclyl are each unsubstituted or substituted, and Rⁿ, R^{o}, R^{p}, and R^{q} are each independently selected from the group consisting of —C₁₋₆ alkyl, —C₁₋₆haloalkyl, —C₂₋₆ alkenyl, —C₂₋₆ alkynyl, aryl, heteroaryl, and heterocyclyl. In some embodiments, Q is selected from the group consisting of

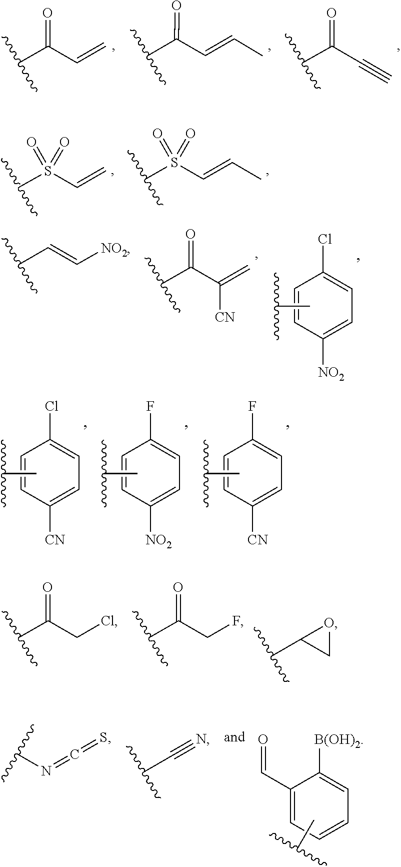

In some embodiments, Q is a fluorophore. In some embodiments, Q is selected from a lanthanide, europium, terbium, XL665, d2, quantum dots, green fluorescent protein, red fluorescent protein, yellow fluorescent protein, fluorescein, rhodamine, eosin, Texas red, cyanine, indocarbocyanine, ocacarbocyanine, thiacarbocyanine, merocyanine, pyridyloxadole, benzoxadiazole, cascade blue, nile red, oxazine 170, acridine orange, proflavin, auramine, malachite green crystal violet, porphine phtalocyanine, and bilirubin.

Provided in some embodiments are methods of sequencing a plurality of polypeptide molecules in a sample comprising: (a) affixing the polypeptide molecules in the sample to a plurality of spatially resolved attachment points on a support or substrate;

(b) functionalizing the N-terminal amino acid (NTAA) of the polypeptide molecules with a chemical reagent, wherein the chemical reagent comprises a compound selected from the group consisting of
(i) a compound of Formula (AA), and
(ii) a compound of the Formula R³—NCS;

(c) contacting the polypeptides with a plurality of binding agents each comprising a binding portion capable of binding to the functionalized NTAA and a detectable label;

(d) for a plurality of polypeptides molecule that are spatially resolved and affixed to the support or substrate, optically detecting the fluorescent label of the probe bound to each polypeptide;

(e) eliminating the functionalized NTAA of each of the polypeptides; and

(f) repeating steps b) to d) to determine the sequence of at least a portion of one or more of the plurality of polypeptide molecules that are spatially resolved and affixed to the support or substrate. In some embodiments, the polypeptide is further contacted with an amine of Formula R²—NH₂ or with a diheteronucleophile in step (b).

In some embodiments, step (b) is conducted before step (c). In some embodiments, step (b) is conducted after step (c) and before step (d). In some embodiments, step (b) is conducted after both step (c) and step (d). In some embodiments, steps (a), (b), (c), (d), and (e) occur in sequential order. In some embodiments, steps (a), (c), (b), (d), and (e) occur in sequential order. In some embodiments, steps (a), (c), (d), (b), and (e) occur in sequential order. In some embodiments, an additional step of contacting the polypeptide(s) with one or more enzymes to eliminate the NTAA (e.g., a proline aminopeptidase), typically either before or after steps (a)-(e) is included. In some embodiments, a functionalized NTAA is eliminated via chemical and/or biological (e.g., enzymatic) means to expose a new NTAA.

Provided in some embodiments are methods of sequencing a plurality of polypeptide molecules in a sample comprising functionalizing the N-terminal amino acid (NTAA) of the polypeptide with a chemical reagent and contacting the polypeptide with a binding agent capable of binding to the functionalized NTAA. In some aspects, the binding agent comprises a coding tag containing identifying information regarding the binding agent. In some aspects, the binding agent further comprises one or more detectable labels such as fluorescent labels, in addition to the binding moiety. In some embodiments of any of the methods presented herein, the fluorescent label is a fluorescent moiety, color-coded nanoparticle or quantum dot.

In some embodiments of any of the methods presented herein, the sample comprises a biological fluid, cell extract or tissue extract. In some embodiments, the method further comprises comparing the sequence of at least one polypeptide molecule determined in step e) to a reference protein sequence database. In some embodiments, the method further comprises comparing the sequences of each polypeptide determined in step e), grouping similar polypeptide sequences and counting the number of instances of each similar polypeptide sequence.

In some embodiments, functionalization of the NTAA using a chemical reagent comprising a compound of Formula (AA) and the subsequent elimination are as depicted in the following scheme:

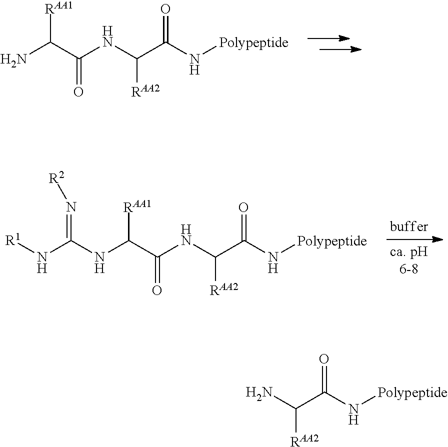

wherein R¹ and R² are as defined above and R^{AA1} is the side chain of the NTAA of a polypeptide.

In some embodiments, the product of the elimination step is determined by the amino acid side chain of the functionalized NTAA that has been eliminated from the polypeptide. In some embodiments, the product of the functionalized NTAA that has been eliminated from the polypeptide is in linear form. In some embodiments, the product of the elimination step is comprised of the two terminal amino acids. In some embodiments, the functionalized NTAA that has been eliminated from the polypeptide comprises a ring. In some embodiments, the elimination product of a NTAA functionalized with a compound of Formula (AA) comprises a compound selected from

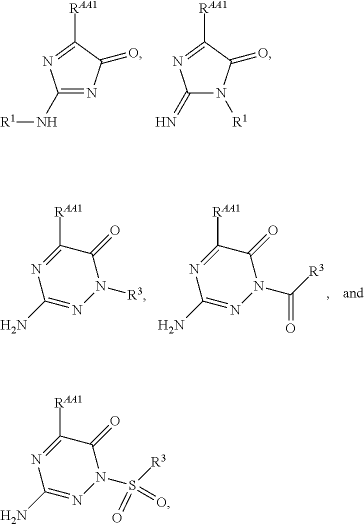

and the tautomers of these. Each of these products includes the side chain of the NTAA that has been removed, thus identification of the cyclic cleavage product provides the identity of the NTAA that was removed.

In certain embodiments, the NTAA have been blocked prior to the NTAA functionalization step (particularly the original N-terminus of the protein). If so, there are a number of approaches to unblock the N-terminus, such as removing N-acetyl blocks with acyl peptide hydrolase (APH) (Farries, Harris et al. 1991). A number of other methods of unblocking the N-terminus of a peptide are known in the art (see, e.g., Krishna et al., 1991, Anal. Biochem. 199:45-50; Leone et al., 2011, Curr. Protoc. Protein Sci., Chapter 11: Unit 11.7; Fowler et al., 2001, Curr. Protoc. Protein Sci., Chapter 11: Unit 11.7, each of which is hereby incorporated by reference in its entirety).

In some embodiments, the polypeptide is obtained by fragmenting a protein from a biological sample. Examples of biological samples include, but are not limited to cells (both primary cells and cultured cell lines), cell lysates or extracts, cell organelles or vesicles, including exosomes, tissues and tissue extracts; biopsy; fecal matter; bodily fluids (such as blood, whole blood, serum, plasma, urine, lymph, bile, cerebrospinal fluid, interstitial fluid, aqueous or vitreous humor, colostrum, sputum, amniotic fluid, saliva, anal and vaginal secretions, perspiration and semen, a transudate, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; research samples including extracellular fluids, extracellular supernatants from cell cultures, inclusion bodies in bacteria, cellular compartments including mitochondrial compartments, and cellular periplasm. A peptide, polypeptide, protein, or protein complex may comprise a standard, naturally occurring amino acid, a modified amino acid (e.g., post-translational modification), an amino acid analog, an amino acid mimetic, or any combination thereof.

In some embodiments of any of the methods described herein, the polypeptide is covalently affixed to a support or substrate. In some embodiments, the support or substrate can be any support surface including, but not limited to, a bead, a microbead, an array, a glass surface, a silicon surface, a plastic surface, a filter, a membrane, a PTFE membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, silica, polyanhydrides, polyglycolic acid, polyvinylchloride, polylactic acid, polyorthoesters, functionalized silane, polypropylfumerate, collagen, glycosaminoglycans, polyamino acids, or any combination thereof. In certain embodiments, a solid support is a bead, for example, a polystyrene bead, a polymer bead, a polyacrylate bead, an agarose bead, a cellulose bead, a dextran bead, an acrylamide bead, a solid core bead, a porous bead, a paramagnetic bead, a glass bead, a silica-based bead, or a controlled pore bead, or any combinations thereof.

Provided in some aspects are methods of sequencing a polypeptide comprising: (a) affixing the polypeptide to a support or substrate, or providing the polypeptide in a solution; (b) functionalizing the N-terminal amino acid (NTAA) of the polypeptide with a chemical reagent, wherein the chemical reagent comprises a compound selected from the group consisting of

(i) a compound of Formula (AA):

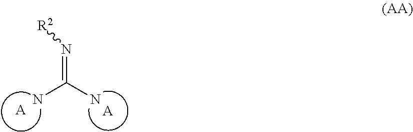

or a salt or conjugate thereof,

wherein:
R² is H or R⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
   wherein two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN; ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;

wherein each R is independently selected from H and C₁₋₃ alkyl optionally substituted with OH, OR*, —NH₂, —NHR*, or —NR*₂; and

each R* is C₁₋₃ alkyl, optionally substituted with OH, oxo, C₁₋₂ alkoxy, or CN;
wherein two R or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

or
a compound of the formula

R³—N═C═S
wherein R³ is an optionally substituted group selected from phenyl, 5-membered heteroaryl, 6-membered heteroaryl, C₁₋₃ haloalkyl, and C₁₋₆ alkyl,
   wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
   where each R′ is independently H or C₁₋₃ alkyl;
wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN.

### Terminal Amino Acid (TAA) Functionalization and Elimination Methods

In certain embodiments, a terminal amino acid (e.g., NTAA or CTAA) of a polypeptide is functionalized. In some embodiments, the terminal amino acid is functionalized prior to contacting the polypeptide with a binding agent in the methods described herein. In some embodiments, the terminal amino acid is functionalized after contacting the polypeptide with a binding agent in the methods described herein.

In some embodiments, the terminal amino acid is functionalized by contacting the polypeptide with a chemical reagent. In some embodiments, the terminal amino acid to be functionalized is the N-terminal amino acid, which can be functionalized with a reagent of Formula (AA) as described above, or with a reagent of formula R³—NCS as described above. In each case, the initially formed functionalized NTAA can then be converted under mild conditions to a compound of Formula (II)

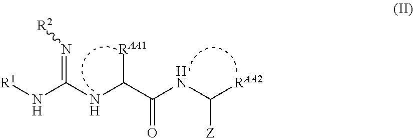

or a tautomer thereof as described herein.

The compounds of Formula (II) undergo cleavage to remove the functionalized NTAA, leaving a truncated polypeptide corresponding to the starting polypeptide with the NTAA removed. Elimination of the functionalized NTAA provides a cleavage by-product.

In some embodiments, the product of the elimination step comprises the functionalized NTAA that has been eliminated from the polypeptide. In some embodiments, the product the functionalized NTAA that has been eliminated from the polypeptide is in linear form. In some embodiments, the functionalized NTAA that has been eliminated from the polypeptide comprises a ring. In some embodiments, the functionalized NTAA that has been eliminated from the polypeptide comprises a ring. In some embodiments, the elimination product of a NTAA functionalized with a compound of Formula (AA) comprises a compound selected from

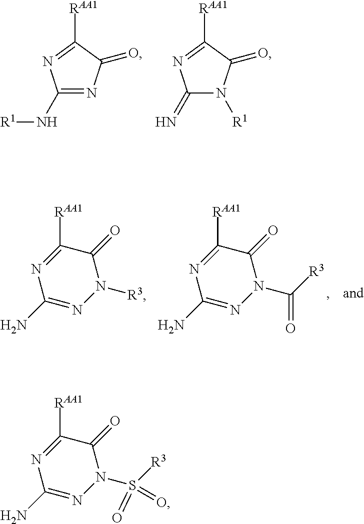

and the tautomers of these. Each of these products includes the side chain of the NTAA that has been removed, thus identification of the cyclic cleavage product provides the identity of the NTAA that was removed.

In any of the embodiments provided herein, the functionalized NTAA is removed by a suitable reagent. Typically the formulation for NTAA removal is 1-100 mM of suitable reagent for NTAA removal in a non-nucleophilic medium at a pH of about 5-10. The medium typically comprises a buffering agent such as sodium/potassium phosphate, PBS, acetate, carbonate, bicarbonate, tertiary amine salts (e.g., N-ethylmorpholinium acetate, triethylammonium acetate, HEPES, MOPS, MES, POPSO, CAPSO, other Good's buffers, etc.), chloride, or TRIS. The medium is typically aqueous and optionally comprises 0-80% of a water-miscible organic solvent, such as dimethylsulfoxide, N,N-dimethylformamide, N,N-dimethylacetamide, methanol, N-methylpyrrolidone, ethanol, or acetonitrile or a combination of two or more of these. The mixture is typically maintained at 25° C.-100° C. for 10-60 minutes in the medium to effect removal of the NTAA. An example of a suitable medium is water with phosphate, sodium chloride, tween 20 (surfactant) at pH 5-10, and is heated at 25° C.-60° C. for 1 to 60 minutes containing a suitable reagent such as a diheteronucleophile. In some embodiments, the elimination is performed using an aqueous formulation that includes 0.1M to 2.0M sodium, potassium, cesium, or ammonium phosphate buffer or sodium, potassium, or ammonium carbonate buffer at a pH 5.5-9.5 at 50-100° C. for 5-60 minutes. In some embodiments, the suitable reagent for NTAA elimination comprises a hydroxide, ammonia, or a diheteronucleophile, typically at a concentration of 0.15M-4.5M In some embodiments, the functionalized NTAA is eliminated using ammonia or ammonium hydroxide. In some embodiments, elimination of the functionalized NTAA is induced by treatment with a diheteronucleophile such as hydrazine or one of the hydrazine derivatives described herein. In some embodiments, the functionalized NTAA can be eliminated using a buffered solution without an amine, typically a mildly acidic or mildly basic (pH 5-9) medium, and in other embodiments ammonia, or a diheteronucleophilic amine such as one selected from this group A is present in the medium.

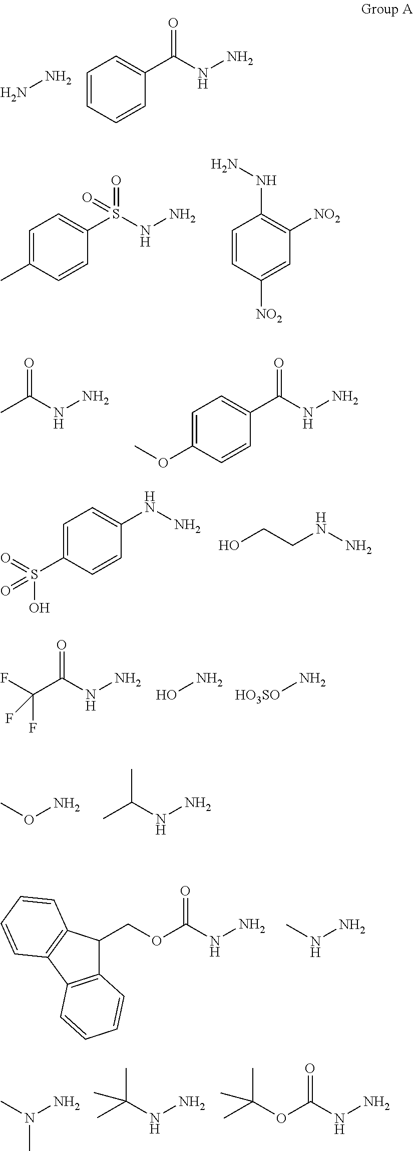

is present in the medium to promote elimination of the functionalized NTAA. In a preferred embodiment (NTH), the diheteronucleophilic reagent is hydrazine.

In some embodiments, the polypeptide may be treated with one or more enzymes to eliminate the NTAA. In some examples, the polypeptide may be treated with an enzyme to eliminate the functionalized NTAA. In some cases, the polypeptide is treated with one or more enzymes before, during, or after the process of modifying the NTAA. The methods of the invention may include an optional step of treating a polypeptide with an enzyme to remove one or more NTAAs before, during, or after treatment with any of the provided chemical reagents; and kits for practicing methods of the invention may optionally include an enzyme to remove one or more NTAAs for use in this fashion. In some of any such embodiments, the polypeptide may be treated with a combination of enzymes to remove one or more NTAAs. In some embodiments, functionalized NTAAs of various polypeptides in a sample is eliminated via chemical and/or biological (e.g., enzymatic) means to expose a new NTAA.

In some embodiments, the enzyme eliminates an NTAA from the polypeptide that is an asparagine. In some embodiments, the enzyme eliminates an NTAA from the polypeptide that is a proline. In some embodiments, the enzyme eliminates an NTAA from the polypeptide that is a serine. In some embodiments, the enzyme eliminates an NTAA from the polypeptide that is a threonine. In some embodiments, the enzyme eliminates an NTAA from the polypeptide that is a glutamine. In some examples, asparagine may be treated with an enzyme to transform the residue into asparatate. In some examples, glutamine may be treated with an enzyme to transform the residue into glutamate. See e.g., Ito et al., 2012, Appl Environ Microbiol. 78(15): 5182-5188; Yamaguchi et al., 2001, Eur J Biochem. 268(5):1410-21; Stewart et al., 1994, J Biol Chem. 269(38):23509-17; Stewart et al., 1995, J Biol Chem. 270(1):25-8.

In some cases, pyroglutamate occurs at the N-terminus of peptides and proteins in nature. It is a natural amino acid ubiquitously existing in plant, bacterial, and mammalian cells, and carries out important biological functions in the form of signaling peptides and immunoglobulin (Eduardo et al., (2010) Front Neuroendocrinol., 134-156; Bochtler et al., (2018) Front. Microbiol., 9:230; Pohl et al., (1991) Proceedings of the National Academy of Sciences, 88 (22) 10059-10063; Wu et al., (2017) mBio 8 (1) e02231-16). It arises when the amino group of the N-terminal glutamine or glutamate cyclizes with its side chain spontaneously or assisted with glutaminyl cyclase (Schilling et al., (2008) Biological Chemistry, 389(8), 983-991). N-terminal pyroglutamate peptides can also be readily converted from its N-terminal glutamine peptide counterpart in laboratory when treated with mild acid or at elevated temperature. In one example, conjugating N-terminal glutamine peptides to a surface using strained-promoted alkyne-azide cycloaddition (SPAAC) reaction may result in pyroglutamate formation. During the conjugation reaction, azido peptides are treated with DBCO beads in 100 mM HEPES pH 7.5 at 60° C. overnight and N-terminal glutamine cyclizes to furnish a pyroglutamate.

In another example, a peptide may form a pyroglutamate when treated with a chemical reagent (e.g., diheterocyclic methanimine). For example, under conditions where the N-terminal amino acid is glutamine (Gln; Q) a cyclization stemming from the N-terminal amine readily occurs on the primary amide of the glutamine side chain resulting in pyroglutamate formation. During this step, the P1 amino acid is eliminated and newly formed N-terminal glutamine may cyclize to form pyroglutamate. For example, pyroglutamate may form under the elimination reaction condition with 1 M ammonium phosphate pH 6.0 at 95° C. for 30 min. Once pyroglutamate is formed, the once N-terminal amine can no longer undergo functionalization, it may be desirable to remove pyroglutamate from the N-terminus using an enzymatic approach before applying the chemical NTAA elimination methods described above. In another example, under conditions where the N-terminal amino acid is serine (Ser, S), a cyclization stemming from the serine side-chain on to the modified N-terminal amine results in iminooxazolidine formation. Once iminooxazolidine formation occurs, it may be desirable to remove iminooxazolidine from the N-terminus using an enzymatic approach before applying the chemical NTAA elimination methods described above.

In some specific examples, the polypeptide is treated with a proline aminopeptidase, a proline iminopeptidase (PIP), a pyroglutamate aminopeptidase (pGAP), an asparagine amidohydrolase, a peptidoglutaminase asparaginase, and/or a protein glutaminase, or a homolog thereof. This may be done before applying a chemical NTAA elimination step as described herein. In some embodiments, an enzyme treatment is compatible with the treatment with the provided chemical reagents and/or with steps performed in the polypeptide analysis assay. See e.g., Ito et al., 2012, Appl Environ Microbiol. 78(15): 5182-5188; Yamaguchi et al., 2001, Eur J Biochem. 268(5):1410-21; Stewart et al., 1994, J Biol Chem. 269(38):23509-17; Stewart et al., 1995, J Biol Chem. 270(1):25-8.

In some embodiments, the method includes functionalizing the N-terminal amino acid (NTAA) of the polypeptide with a chemical reagent, contacting the polypeptide with a binding agent capable of binding to the functionalized NTAA, treating the polypeptide with an enzyme (e.g., to transform or remove an NTAA), and eliminating the functionalized NTAA to expose a new NTAA (e.g., using a chemical reagent). In some aspects, the treatment of the polypeptide with the enzyme (e.g., to transform or remove an NTAA) can be performed in various orders with respect to treatment of the polypeptide with other reagents. In some examples, treating the polypeptide with an enzyme (e.g., to transform or remove an NTAA) is performed after contacting the polypeptide with a binding agent capable of binding to the functionalized NTAA. In some particular cases, treating the polypeptide with an enzyme (e.g., to transform or remove an NTAA) is performed after functionalizing the N-terminal amino acid (NTAA) of the polypeptide with a chemical reagent. In some instances, the polypeptides may be treated with more than one enzyme (e.g., one at a time or as a mixture) to transform and/or remove various NTAAs.

### Polypeptides

In some aspects, the present disclosure relates to the analysis and modification of polypeptides. A polypeptide may comprise L-amino acids, D-amino acids, or both. A polypeptide may comprise a standard, naturally occurring amino acid, a modified amino acid (e.g., post-translational modification), an amino acid analog, an amino acid mimetic, or any combination thereof. In some embodiments, the polypeptide is naturally occurring, synthetically produced, or recombinantly expressed. In any of the aforementioned embodiments, the polypeptide may further comprise a post-translational modification.

Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, β-amino acids, Homo-amino acids, Proline and Pyruvic acid derivatives, 3-substituted Alanine derivatives, Glycine derivatives, Ring-substituted Phenylalanine and Tyrosine Derivatives, Linear core amino acids, and N-methyl amino acids.

A polypeptide analyzed according the methods disclosed herein may be obtained from a suitable source or sample, including but not limited to: biological samples, such as cells (both primary cells and cultured cell lines), cell lysates or extracts, cell organelles or vesicles, including exosomes, tissues and tissue extracts; biopsy; fecal matter; bodily fluids (such as blood, whole blood, serum, plasma, urine, lymph, bile, cerebrospinal fluid, interstitial fluid, aqueous or vitreous humor, colostrum, sputum, amniotic fluid, saliva, anal and vaginal secretions, perspiration and semen, a transudate, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; research samples including extracellular fluids, extracellular supernatants from cell cultures, inclusion bodies in bacteria, cellular compartments including mitochondrial compartments, and cellular periplasm.

In certain embodiments, the polypeptide is a protein or a protein complex. Amino acid sequence information and post-translational modifications of the polypeptide are transduced into a nucleic acid encoded library that can be analyzed via next generation sequencing methods.

A polypeptide may comprise L-amino acids, D-amino acids, or both. A polypeptide may comprise a standard, naturally occurring amino acid, a modified amino acid (e.g., post-translational modification), an amino acid analog, an amino acid mimetic, or any combination thereof. In some embodiments, the polypeptide is naturally occurring, synthetically produced, or recombinantly expressed. In any of the aforementioned embodiments, the polypeptide may further comprise a post-translational modification.

Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, β-amino acids, Homo-amino acids, Proline and Pyruvic acid derivatives, 3-substituted Alanine derivatives, Glycine derivatives, Ring-substituted Phenylalanine and Tyrosine Derivatives, Linear core amino acids, and N-methyl amino acids.

A post-translational modification (PTM) of a polypeptide or amino acid may be a chemical modification or an enzymatic modification of one or more amino acid side chains, and may occur on one or more amino acid side chains in a polypeptide. In some embodiments of the compounds and methods herein, at least one side chain of a proteinogenic amino acid or of one of the common natural amino acids comprises a PTM. Examples of post-translation modifications include, but are not limited to, acylation, acetylation, alkylation (including methylation), azidation, biotinylation, butyrylation, carbamylation, carbonylation, citrullination, deamidation, deiminiation, diphthamide formation, disulfide bridge formation, eliminylation, flavin attachment, formylation, gamma-carboxylation, glutamylation, glycylation, glycosylation (e.g., S-linked, N-linked, O-linked, C-linked, phosphoglycosylation), glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propargylation, propionylation, retinylidene Schiff base formation, S-glutathionylation, S-nitrosylation, S-sulfenylation, selenation, succinylation, sulfation, sulfoglycosylation, sulfination, sumoylation, ubiquitination, and C-terminal amidation. A post-translational modification includes modifications of the amino terminus and/or the carboxyl terminus of a peptide, polypeptide, or protein. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini of a peptide, polypeptide, or protein. Post-translational modification can regulate a protein's “biology” within a cell, e.g., its activity, structure, stability, or localization. Phosphorylation is the most common post-translational modification and plays an important role in regulation of protein, particularly in cell signaling (Prabakaran et al., 2012, Wiley Interdiscip Rev Syst Biol Med 4: 565-583). The addition of sugars to proteins, such as glycosylation, has been shown to promote protein folding, improve stability, and modify regulatory function. The attachment of lipids to proteins enables targeting to the cell membrane.

In certain embodiments, the polypeptide used in the methods herein can be fragmented from a larger protein or protein complex. For example, the fragmented polypeptide can be obtained by fragmenting a polypeptide, protein or protein complex from a sample, such as a biological sample. The polypeptide, protein or protein complex can be fragmented by any means known in the art, including fragmentation by a protease or endopeptidase. In some embodiments, fragmentation of a polypeptide, protein or protein complex is targeted by use of a specific protease or endopeptidase. A specific protease or endopeptidase binds and cleaves at a specific consensus sequence (e.g., TEV protease which is specific for ENLYFQ\S consensus sequence, SEQ ID NO: 141). In other embodiments, fragmentation of a peptide, polypeptide, or protein is non-targeted or random by use of a non-specific protease or endopeptidase. A non-specific protease may bind and cleave at a specific amino acid residue rather than a consensus sequence (e.g., proteinase K is a non-specific serine protease). Proteinases and endopeptidases are well known in the art, and examples of such that can be used to cleave a protein or polypeptide into smaller peptide fragments include proteinase K, trypsin, chymotrypsin, pepsin, thermolysin, thrombin, Factor Xa, furin, endopeptidase, papain, pepsin, subtilisin, elastase, enterokinase, Genenase™ I, Endoproteinase LysC, Endoproteinase AspN, Endoproteinase GluC, etc. (Granvogl et al., 2007, Anal Bioanal Chem 389: 991-1002). In certain embodiments, a peptide, polypeptide, or protein is fragmented by proteinase K, or optionally, a thermolabile version of proteinase K to enable rapid inactivation. Proteinase K is quite stable in denaturing reagents, such as urea and SDS, enabling digestion of completely denatured proteins. Protein and polypeptide fragmentation into peptides can be performed before or after attachment of a DNA tag or DNA recording tag.

In some embodiments, the polypeptide to be analyzed is first treated with one or more enzymes to transform or remove particular amino acids. For example, the polypeptide is treated with a proline aminopeptidase, a proline iminopeptidase (PIP), a pyroglutamate aminopeptidase (pGAP), an N-terminal asparagine amidohydrolase (e.g. NTAN1/PNAD or NH₂-terminal asparagine deamidase or NH2-terminal asparagine amidohydrolase), a peptidoglutaminase asparaginase, and/or a protein glutaminase, or a homolog thereof. In some embodiments, the polypeptide to be analyzed is first contacted with a proline aminopeptidase under conditions suitable to remove an N-terminal proline, if present.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, .NTCB+Ni (2-nitro-5-thiocyanobenzoic acid), etc.

In certain embodiments, following enzymatic or chemical elimination, the resulting polypeptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. An elimination reaction may be monitored, preferably in real time, by spiking the protein or polypeptide sample with a short test FRET (fluorescence resonance energy transfer) polypeptide comprising a peptide sequence containing a proteinase or endopeptidase elimination site. In the intact FRET peptide, a fluorescent group and a quencher group are attached to either end of the peptide sequence containing the elimination site, and fluorescence resonance energy transfer between the quencher and the fluorophore leads to low fluorescence. Upon elimination of the test peptide by a protease or endopeptidase, the quencher and fluorophore are separated giving a large increase in fluorescence. A elimination reaction can be stopped when a certain fluorescence intensity is achieved, allowing a reproducible elimination end point to be achieved.

A sample of polypeptides can undergo protein fractionation methods prior to attachment to a solid support, where proteins or peptides are separated by one or more properties such as cellular location, molecular weight, hydrophobicity, or isoelectric point, or protein enrichment methods. Alternatively, or additionally, protein enrichment methods may be used to select for a specific protein or peptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. In the case of immunoglobulin molecules, analysis of the sequence and abundance or frequency of hypervariable sequences involved in affinity binding are of particular interest, particularly as they vary in response to disease progression or correlate with healthy, immune, and/or or disease phenotypes. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, such as PROTIA and PROT20 (Sigma-Aldrich).

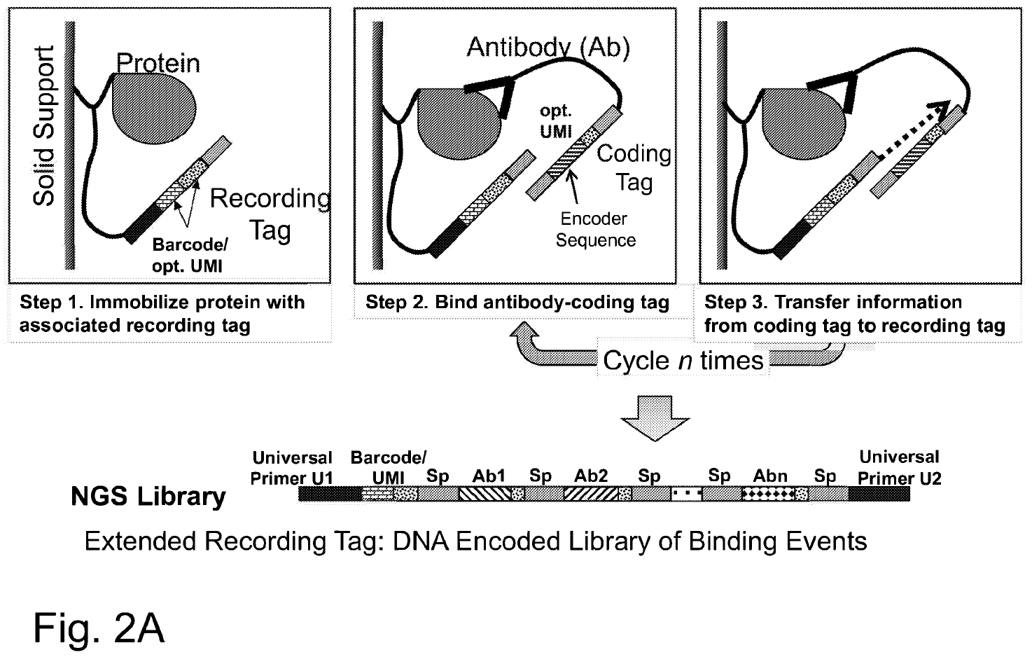

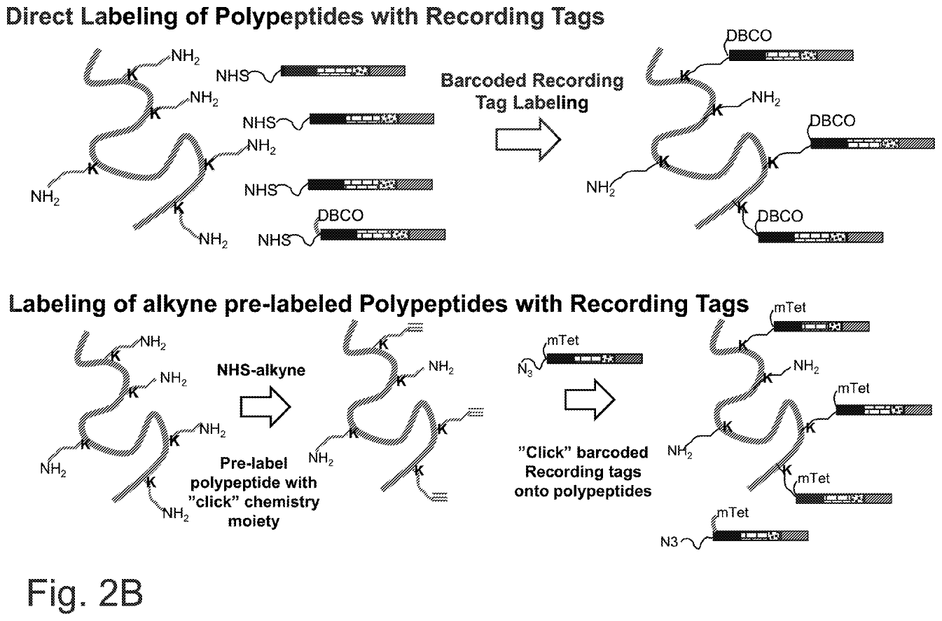

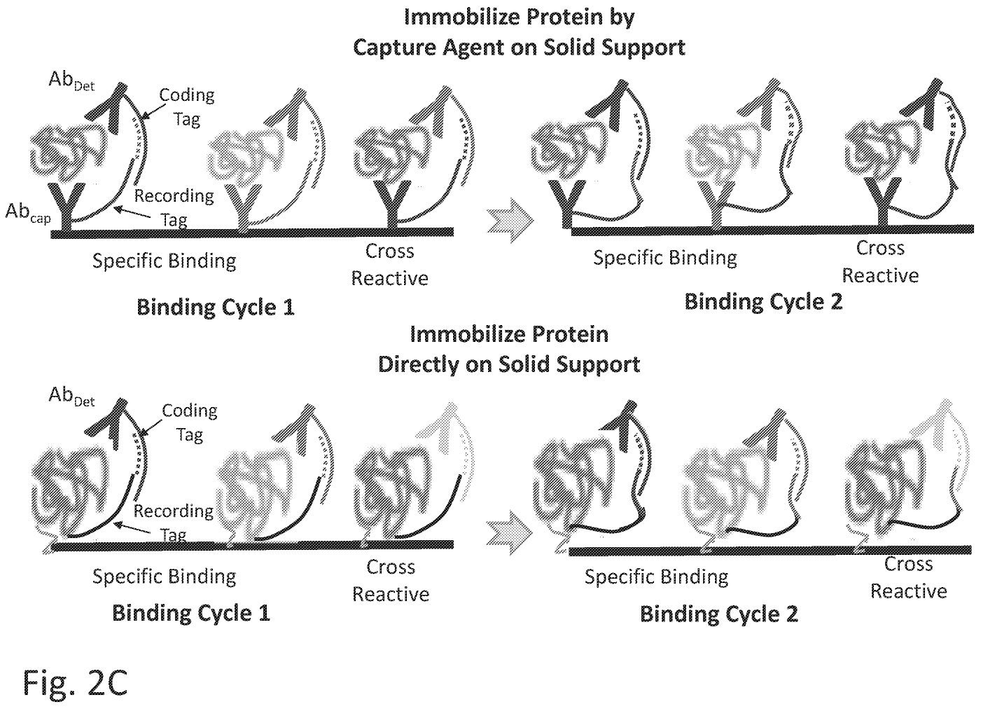

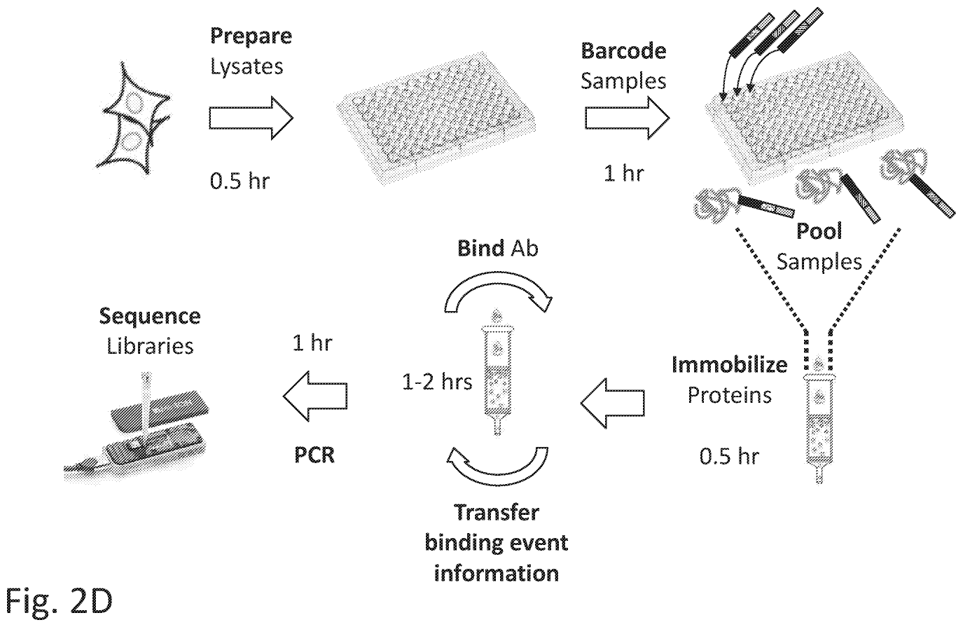

In certain embodiments, the polypeptide is labeled with DNA recording tags through standard amine coupling chemistries (see, e.g., FIGS. 2B, 2C, 28, 29, 31, 40). The ε-amino group (e.g., of lysine residues) and the N-terminal amino group are particularly susceptible to labeling with amine-reactive coupling agents, depending on the pH of the reaction (Mendoza and Vachet 2009). In a particular embodiment (see, e.g., FIG. 2B and FIG. 29), the recording tag is comprised of a reactive moiety (e.g., for conjugation to a solid surface, a multifunctional linker, or a polypeptide), a linker, a universal priming sequence, a barcode (e.g., compartment tag, partition barcode, sample barcode, fraction barcode, or any combination thereof), an optional UMI, and a spacer (Sp) sequence for facilitating information transfer to/from a coding tag. In another embodiment, the protein can be first labeled with a universal DNA tag, and the barcode-Sp sequence (representing a sample, a compartment, a physical location on a slide, etc.) are attached to the protein later through and enzymatic or chemical coupling step. (see, e.g., FIGS. 20, 30, 31, 40). A universal DNA tag comprises a short sequence of nucleotides that are used to label a polypeptide and can be used as point of attachment for a barcode (e.g., compartment tag, recording tag, etc.). For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein. In a particular embodiment, the protein is labeled with a universal DNA tag prior to proteinase digestion into peptides. The universal DNA tags on the labeled peptides from the digest can then be converted into an informative and effective recording tag.

In certain embodiments, a polypeptide can be immobilized to a solid support by known methods such as an affinity capture reagent (and optionally covalently crosslinked), wherein the recording tag is associated with the affinity capture reagent directly, or alternatively, the protein can be directly immobilized to the solid support with a recording tag (see, e.g., FIG. 2C).

### Providing the Polypeptide Joined to a Support or in Solution

In some embodiments, polypeptides of the present disclosure are joined to a surface of a solid support (also referred to as “substrate surface”). The solid support can be any porous or non-porous support surface including, but not limited to, a bead, a microbead, an array, a glass surface, a silicon surface, a plastic surface, a filter, a membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, or any combination thereof. Solid supports further include thin film, membrane, bottles, dishes, fibers, woven fibers, shaped polymers such as tubes, particles, beads, microparticles, or any combination thereof. For example, when solid surface is a bead, the bead can include, but is not limited to, a polystyrene bead, a polymer bead, an agarose bead, an acrylamide bead, a solid core bead, a porous bead, a paramagnetic bead, glass bead, or a controlled pore bead.

In certain embodiments, a solid support is a flow cell. Flow cell configurations may vary among different next generation sequencing platforms. For example, the Illumina flow cell is a planar optically transparent surface similar to a microscope slide, which contains a lawn of oligonucleotide anchors bound to its surface. Template DNA, comprise adapters ligated to the ends that are complimentary to oligonucleotides on the flow cell surface. Adapted single-stranded DNAs are bound to the flow cell and amplified by solid-phase “bridge” PCR prior to sequencing. The 454 flow cell (454 Life Sciences) supports a “picotiter” plate, a fiber optic slide with ˜1.6 million 75-picoliter wells. Each individual molecule of sheared template DNA is captured on a separate bead, and each bead is compartmentalized in a private droplet of aqueous PCR reaction mixture within an oil emulsion. Template is clonally amplified on the bead surface by PCR, and the template-loaded beads are then distributed into the wells of the picotiter plate for the sequencing reaction, ideally with one or fewer beads per well. SOLiD (Supported Oligonucleotide Ligation and Detection) instrument from Applied Biosystems, like the 454 system, amplifies template molecules by emulsion PCR. After a step to cull beads that do not contain amplified template, bead-bound template is deposited on the flow cell. A flow cell may also be a simple filter frit, such as a TWIST™ DNA synthesis column (Glen Research).

In certain embodiments, a solid support is a bead, which may refer to an individual bead or a plurality of beads. In some embodiments, the bead is compatible with a selected next generation sequencing platform that will be used for downstream analysis (e.g., SOLiD or 454). In some embodiments, a solid support is an agarose bead, a paramagnetic bead, a polystyrene bead, a polymer bead, an acrylamide bead, a solid core bead, a porous bead, a glass bead, or a controlled pore bead. In further embodiments, a bead may be coated with a binding functionality (e.g., amine group, affinity ligand such as streptavidin for binding to biotin labeled polypeptide, antibody) to facilitate binding to a polypeptide.

Proteins, polypeptides, or peptides can be joined to the solid support, directly or indirectly, by any means known in the art, including covalent and non-covalent interactions, or any combination thereof (see, e.g., Chan et al., 2007, PLoS One 2:e1164; Cazalis et al., Bioconj. Chem. 15:1005-1009; Soellner et al., 2003, J. Am. Chem. Soc. 125:11790-11791; Sun et al., 2006, Bioconjug. Chem. 17-52-57; Decreau et al., 2007, J. Org. Chem. 72:2794-2802; Camarero et al., 2004, J. Am. Chem. Soc. 126:14730-14731; Girish et al., 2005, Bioorg. Med. Chem. Lett. 15:2447-2451; Kalia et al., 2007, Bioconjug. Chem. 18:1064-1069; Watzke et al., 2006, Angew Chem. Int. Ed. Engl. 45:1408-1412; Parthasarathy et al., 2007, Bioconjugate Chem. 18:469-476; and Bioconjugate Techniques, G. T. Hermanson, Academic Press (2013), and are each hereby incorporated by reference in their entirety). For example, the peptide may be joined to the solid support by a ligation reaction. Alternatively, the solid support can include an agent or coating to facilitate joining, either direct or indirectly, the peptide to the solid support. Any suitable molecule or materials may be employed for this purpose, including proteins, nucleic acids, carbohydrates and small molecules. For example, in one embodiment the agent is an affinity molecule. In another example, the agent is an azide group, which group can react with an alkynyl group in another molecule to facilitate association or binding between the solid support and the other molecule.

Proteins, polypeptides, or peptides can be joined to the solid support using methods referred to as “click chemistry.” For this purpose, any reaction which is rapid and substantially irreversible can be used to attach proteins, polypeptides, or peptides to the solid support. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyltetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothiocyanate, an aldehyde, an epoxide, or the like.

In some embodiments the polypeptide and solid support are joined by a functional group capable of formation by reaction of two complementary reactive groups, for example a functional group which is the product of one of the foregoing “click” reactions. In various embodiments, functional group can be formed by reaction of an aldehyde, oxime, hydrazone, hydrazide, alkyne, amine, azide, acylazide, acylhalide, nitrile, nitrone, sulfhydryl, disulfide, sulfonyl halide, isothiocyanate, imidoester, activated ester (e.g., N-hydroxysuccinimide ester, pentynoic acid STP ester), ketone, α,β-unsaturated carbonyl, alkene, maleimide, α-haloimide, epoxide, aziridine, tetrazine, tetrazole, phosphine, biotin or thiirane functional group with a complementary reactive group. An exemplary reaction is a reaction of an amine (e.g., primary amine) with an N-hydroxysuccinimide ester or isothiocyanate.

In yet other embodiments, the functional group comprises an alkene, ester, amide, thioester, disulfide, carbocyclic, heterocyclic or heteroaryl group. In further embodiments, the functional group comprises an alkene, ester, amide, thioester, thiourea, disulfide, carbocyclic, heterocyclic or heteroaryl group. In other embodiments, the functional group comprises an amide or thiourea. In some more specific embodiments, functional group is a triazolyl functional group, an amide, or thiourea functional group.

In some embodiments, iEDDA click chemistry is used for immobilizing polypeptides to a solid support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction.

In some embodiments, the substrate surface is functionalized with TCO, and the recording tag-labeled protein, polypeptide, peptide is immobilized to the TCO coated substrate surface via an attached m-tetrazine moiety (FIG. 34).

In some embodiments, polypeptides are immobilized to a surface of a solid support by its C-terminus, N-terminus, or an internal amino acid, for example, via an amine, carboxyl, or sulfydryl group. Standard activated supports used in coupling to amine groups include CNBr-activated, NETS-activated, aldehyde-activated, azlactone-activated, and CDI-activated supports. Standard activated supports used in carboxyl coupling include carbodiimide-activated carboxyl moieties coupling to amine supports. Cysteine coupling can employ maleimide, idoacetyl, and pyridyl disulfide activated supports. An alternative mode of peptide carboxy terminal immobilization uses anhydrotrypsin, a catalytically inert derivative of trypsin that binds peptides containing lysine or arginine residues at their C-termini without cleaving them.

In certain embodiments, a polypeptide is immobilized to a solid support via covalent attachment of a solid surface bound linker to a lysine group of the protein, polypeptide, or peptide.

Recording tags can be attached to the protein, polypeptide, or peptides pre- or post-immobilization to the solid support. For example, proteins, polypeptides, or peptides can be first labeled with recording tags and then immobilized to a solid surface via a recording tag comprising at two functional moieties for coupling (see, FIG. 28). One functional moiety of the recording tag couples to the protein, and the other functional moiety immobilizes the recording tag-labeled protein to a solid support.

In other embodiments, polypeptides are immobilized to a solid support prior to labeling of the proteins, polypeptides or peptides with recording tags. For example, proteins can first be derivatized with reactive groups such as click chemistry moieties. The activated protein molecules can then be attached to a suitable solid support and then labeled with recording tags using the complementary click chemistry moiety. As an example, proteins derivatized with alkyne and mTet moieties may be immobilized to beads derivatized with azide and TCO and attached to recording tags labeled with azide and TCO.

It is understood that the methods provided herein for attaching polypeptides to the solid support may also be used to attach recording tags to the solid support or attach recording tags to polypeptides.

In certain embodiments, the surface of a solid support is passivated (blocked) to minimize non-specific absorption to binding agents. A “passivated” surface refers to a surface that has been treated with outer layer of material to minimize non-specific binding of a binding agent. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), and diamond-like carbon (DLC), DLC+PEG (Stavis et al., 2011, Proc. Natl. Acad. Sci. USA 108:983-988) and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol, (PVA), and proteins like BSA and casein. Alternatively, density of proteins, polypeptide, or peptides can be titrated on the surface or within the volume of a solid substrate by spiking a competitor or “dummy” reactive molecule when immobilizing the proteins, polypeptides or peptides to the solid substrate (see, FIG. 36A).

A suitable spacing frequency can be determined empirically using a functional assay and can be accomplished by dilution and/or by spiking a “dummy” spacer molecule that competes for attachments sites on the substrate surface. For example, PEG-5000 (MW 5000) is used to block the interstitial space between peptides on the substrate surface (e.g., bead surface). In addition, the peptide is coupled to a functional moiety that is also attached to a PEG-5000 molecule. In a preferred embodiment, this is accomplished by coupling a mixture of NHS-PEG-5000-TCO+NHS-PEG-5000-Methyl to amine-derivatized beads. The stoichiometric ratio between the two PEGs (TCO vs. methyl) is titrated to generate an appropriate density of functional coupling moieties (TCO groups) on the substrate surface; the methyl-PEG is inert to coupling. The effective spacing between TCO groups can be calculated by measuring the density of TCO groups on the surface. In certain embodiments, the mean spacing between coupling moieties (e.g., TCO) on the solid surface is at least 50 nm, at least 100 nm, at least 250 nm, or at least 500 nm. After PEG5000-TCO/methyl derivitization of the beads, the excess NH₂ groups on the surface are quenched with a reactive anhydride (e.g. acetic or succinic anhydride).

In some embodiments, the spacing is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some preferred embodiments, the substrate surface (e.g., bead surface) comprises NHS moieties. In some embodiments, a mixture of mPEGₙ-NH₂ and NH₂-PEGₙ-mTet is added to the activated beads (wherein n is any number, such as 1-100). The ratio between the mPEG₃-NH₂ (not available for coupling) and NH₂-PEG24-mTet (available for coupling) is titrated to generate an appropriate density of functional moieties available to attach the analyte on the substrate surface. In certain embodiments, the mean spacing between coupling moieties (e.g., NH₂-PEG4-mTet) on the solid surface is at least 50 nm, at least 100 nm, at least 250 nm, or at least 500 nm. In some specific embodiments, the ratio of NH₂-PEGₙ-mTet to mPEG₃-NH₂ is about or greater than 1:1000, about or greater than 1:10,000, about or greater than 1:100,000, or about or greater than 1:1,000,000. In some further embodiments, the capture nucleic acid attaches to the NH₂-PEGₙ-mTet.

In certain embodiments where multiple polypeptides are immobilized on the same solid support, the polypeptides can be spaced appropriately to reduce the occurrence of or prevent a cross-binding or inter-molecular event, e.g., where a binding agent binds to a first polypeptides and its coding tag information is transferred to a recording tag associated with a neighboring polypeptides rather than the recording tag associated with the first polypeptide. To control polypeptide spacing on the solid support, the density of functional coupling groups (e.g., TCO) may be titrated on the substrate surface (see, FIG. 34). In some embodiments, multiple polypeptides are spaced apart on the surface or within the volume (e.g., porous supports) of a solid support at a distance of about 50 nm to about 500 nm, or about 50 nm to about 400 nm, or about 50 nm to about 300 nm, or about 50 nm to about 200 nm, or about 50 nm to about 100 nm. In some embodiments, multiple polypeptides are spaced apart on the surface of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple polypeptides are spaced apart on the surface of a solid support with an average distance of at least 50 nm. In some embodiments, polypeptides are spaced apart on the surface or within the volume of a solid support such that, empirically, the relative frequency of inter- to intra-molecular events is <1:10; <1:100; <1:1,000; or <1:10,000. A suitable spacing frequency can be determined empirically using a functional assay (see, Example 31), and can be accomplished by dilution and/or by spiking a “dummy” spacer molecule that competes for attachments sites on the substrate surface.

For example, as shown in FIG. 34, PEG-5000 (MW˜5000) is used to block the interstitial space between peptides on the substrate surface (e.g., bead surface). In addition, the peptide is coupled to a functional moiety that is also attached to a PEG-5000 molecule. In some embodiments, this is accomplished by coupling a mixture of NHS-PEG-5000-TCO+NHS-PEG-5000-Methyl to amine-derivatized beads (see FIG. 34). The stoichiometric ratio between the two PEGs (TCO vs. methyl) is titrated to generate an appropriate density of functional coupling moieties (TCO groups) on the substrate surface; the methyl-PEG is inert to coupling. The effective spacing between TCO groups can be calculated by measuring the density of TCO groups on the surface. In certain embodiments, the mean spacing between coupling moieties (e.g., TCO) on the solid surface is at least 50 nm, at least 100 nm, at least 250 nm, or at least 500 nm. After PEG5000-TCO/methyl derivatization of the beads, the excess NH₂ groups on the surface are quenched with a reactive anhydride (e.g. acetic or succinic anhydride).

In particular embodiments, the polypeptide(s) and/or the recording tag(s) are immobilized on a substrate or support at a density such that the interaction between (i) a coding agent bound to a first polypeptide (particularly, the coding tag in that bound coding agent), and (ii) a second polypeptide and/or its recording tag, is reduced, minimized, or completely eliminated. Therefore, false positive assay signals resulting from “intermolecular” engagement can be reduced, minimized, or eliminated.

In certain embodiments, the density of the polypeptides and/or the recording tags on a substrate is determined for each type of polypeptide. For example, the longer a denatured polypeptide chain is, the lower the density should be in order to reduce, minimize, or prevent “intermolecular” interactions. In certain aspects, increasing the spacing between the polypeptide molecules and/or the recording tags (i.e., lowering the density) increases the signal to background ratio of the presently disclosed assays.

In some embodiments, the polypeptide molecules and/or the recording tags are deposited or immobilized on a substrate at an average density of about 0.0001 molecule/μm², 0.001 molecule/μm², 0.01 molecule/μm², 0.1 molecule/μm², 1 molecule/μm², about 2 molecules/μm², about 3 molecules/μm², about 4 molecules/μm², about 5 molecules/μm², about 6 molecules/μm², about 7 molecules/μm², about 8 molecules/μm², about 9 molecules/μm², or about 10 molecules/μm². In other embodiments, the polypeptide(s) and/or the recording tag(s) are deposited or immobilized at an average density of about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, or about 200 molecules/μm² on a substrate. In other embodiments, the polypeptide(s) and/or the recording tag(s) are deposited or immobilized at an average density of about 1 molecule/mm², about 10 molecules/mm², about 50 molecules/mm², about 100 molecules/mm², about 150 molecules/mm², about 200 molecules/mm², about 250 molecules/mm², about 300 molecules/mm², about 350 molecules/mm², 400 molecules/mm², about 450 molecules/mm², about 500 molecules/mm², about 550 molecules/mm², about 600 molecules/mm², about 650 molecules/mm², about 700 molecules/mm², about 750 molecules/mm², about 800 molecules/mm², about 850 molecules/mm², about 900 molecules/mm², about 950 molecules/mm², or about 1000 molecules/mm². In still other embodiments, the polypeptide(s) and/or the recording tag(s) are deposited or immobilized on a substrate at an average density between about 1×10³ and about 0.5×10⁴ molecules/mm², between about 0.5×10⁴ and about 1×10⁴ molecules/mm², between about 1×10⁴ and about 0.5×10⁵ molecules/mm², between about 0.5×10⁵ and about 1×10⁵ molecules/mm², between about 1×10⁵ and about 0.5×10⁶ molecules/mm², or between about 0.5×10⁶ and about 1×10⁶ molecules/mm². In other embodiments, the average density of the polypeptide(s) and/or the recording tag(s) deposited or immobilized on a substrate can be, for example, between about 1 molecule/cm² and about 5 molecules/cm², between about 5 and about 10 molecules/cm², between about 10 and about 50 molecules/cm², between about 50 and about 100 molecules/cm², between about 100 and about 0.5×10³ molecules/cm², between about 0.5×10³ and about 1×10³ molecules/cm², 1×10³ and about 0.5×10⁴ molecules/cm², between about 0.5×10⁴ and about 1×10⁴ molecules/cm², between about 1×10⁴ and about 0.5×10⁵ molecules/cm², between about 0.5×10⁵ and about 1×10⁵ molecules/cm², between about 1×10⁵ and about 0.5×10⁶ molecules/cm², or between about 0.5×10⁶ and about 1×10⁶ molecules/cm².

In certain embodiments, the concentration of the binding agents in a solution is controlled to reduce background and/or false positive results of the assay.

In some embodiments, the concentration of a binding agent is about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1000 nM.

In some embodiments, the ratio between the soluble binding agent molecules and the immobilized polypeptides and/or the recording tags is about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10⁴:1, about 10⁵:1, about 10⁶:1, or higher, or any ratio in between the above listed ratios. Higher ratios between the soluble binding agent molecules and the immobilized polypeptide(s) and/or the recording tag(s) can be used to drive the binding and/or the coding tag/recoding tag information transfer to completion. This may be particularly useful for detecting and/or analyzing low abundance polypeptides in a sample.

### Recording Tags

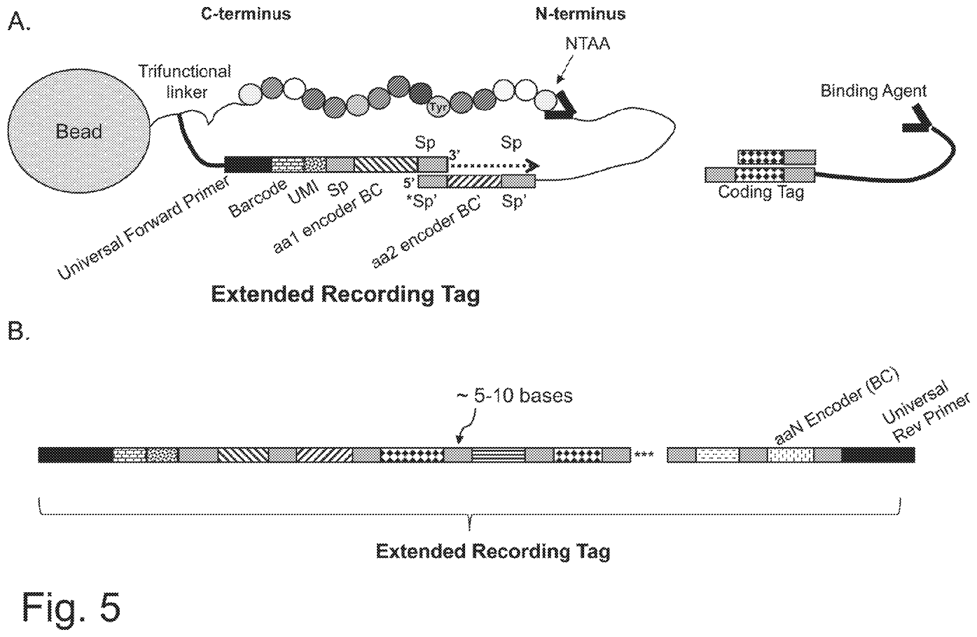

At least one recording tag is associated or co-localized directly or indirectly with the polypeptide and joined to the solid support (see, e.g., FIG. 5). A recording tag may comprise DNA, RNA, or polynucleotide analogs including PNA, γPNA, GNA, BNA, XNA, TNA, polynucleotide analogs, or a combination thereof. A recording tag may be single stranded, or partially or completely double stranded. A recording tag may have a blunt end or overhanging end. In certain embodiments, upon binding of a binding agent to a polypeptide, identifying information of the binding agent's coding tag is transferred to the recording tag to generate an extended recording tag. Further extensions to the extended recording tag can be made in subsequent binding cycles.

A recording tag can be joined to the solid support, directly or indirectly (e.g., via a linker), by any means known in the art, including covalent and non-covalent interactions, or any combination thereof. For example, the recording tag may be joined to the solid support by a ligation reaction. Alternatively, the solid support can include an agent or coating to facilitate joining, either direct or indirectly, of the recording tag, to the solid support. Strategies for immobilizing nucleic acid molecules to solid supports (e.g., beads) have been described in U.S. Pat. No. 5,900,481; Steinberg et al. (2004, Biopolymers 73:597-605); Lund et al., 1988 (Nucleic Acids Res. 16: 10861-10880); and Steinberg et al. (2004, Biopolymers 73:597-605), each of which is incorporated herein by reference in its entirety.

In certain embodiments, the co-localization of a polypeptide and associated recording tag is achieved by conjugating polypeptide and recording tag to a bifunctional linker attached directly to the solid support surface Steinberg et al. (2004, Biopolymers 73:597-605). In further embodiments, a trifunctional moiety is used to derivitize the solid support (e.g., beads), and the resulting bifunctional moiety is coupled to both the polypeptide and recording tag.

Methods and reagents (e.g., click chemistry reagents and photoaffinity labelling reagents) such as those described for attachment of polypeptides and solid supports, may also be used for attachment of recording tags.

In a particular embodiment, a single recording tag is attached to a polypeptide, preferably via the attachment to a de-blocked N- or C-terminal amino acid. In another embodiment, multiple recording tags are attached to the polypeptide, preferably to the lysine residues or peptide backbone. In some embodiments, a polypeptide labeled with multiple recording tags is fragmented or digested into smaller peptides, with each peptide labeled on average with one recording tag.

In certain embodiments, a recording tag comprises an optional, unique molecular identifier (UMI), which provides a unique identifier tag for each polypeptide to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual polypeptides. In some embodiments, within a library of polypeptides, each polypeptide is associated with a single recording tag, with each recording tag comprising a unique UMI. In other embodiments, multiple copies of a recording tag are associated with a single polypeptide, with each copy of the recording tag comprising the same UMI. In some embodiments, a UMI has a different base sequence than the spacer or encoder sequences within the binding agents' coding tags to facilitate distinguishing these components during sequence analysis.

In certain embodiments, a recording tag comprises a barcode, e.g., other than the UMI if present. A barcode is a nucleic acid molecule of about 3 to about 30 bases, about 3 to about 25 bases, about 3 to about 20 bases, about 3 to about 10 bases, about 3 to about 10 bases, about 3 to about 8 bases in length. In some embodiments, a barcode is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. In one embodiment, a barcode allows for multiplex sequencing of a plurality of samples or libraries. A barcode may be used to identify a partition, a fraction, a compartment, a sample, a spatial location, or library from which the polypeptide derived. Barcodes can be used to de-convolute multiplexed sequence data and identify sequence reads from an individual sample or library. For example, a barcoded bead is useful for methods involving emulsions and partitioning of samples, e.g., for purposes of partitioning the proteome.

A barcode can represent a compartment tag in which a compartment, such as a droplet, microwell, physical region on a solid support, etc. is assigned a unique barcode. The association of a compartment with a specific barcode can be achieved in any number of ways such as by encapsulating a single barcoded bead in a compartment, e.g., by direct merging or adding a barcoded droplet to a compartment, by directly printing or injecting a barcode reagent to a compartment, etc. The barcode reagents within a compartment are used to add compartment-specific barcodes to the polypeptide or fragments thereof within the compartment. Applied to protein partitioning into compartments, the barcodes can be used to map analysed peptides back to their originating protein molecules in the compartment. This can greatly facilitate protein identification. Compartment barcodes can also be used to identify protein complexes.

In other embodiments, multiple compartments that represent a subset of a population of compartments may be assigned a unique barcode representing the subset.

Alternatively, a barcode may be a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). Polypeptides from many different samples can be labeled with recording tags with sample-specific barcodes, and then all the samples pooled together prior to immobilization to a solid support, cyclic binding, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes. For example, the sample can be split and barcoded, and one portion processed using binding agents to low abundance analytes, and the other portion processed using binding agents to higher abundance analytes. In a particular embodiment, this approach helps to adjust the dynamic range of a particular protein analyte assay to lie within the “sweet spot” of standard expression levels of the protein analyte.

In certain embodiments polypeptides from multiple different samples are labeled with recording tags containing sample-specific barcodes. The multi-sample barcoded polypeptides can be mixed together prior to a cyclic binding reaction. In this way, a highly-multiplexed alternative to a digital reverse phase protein array (RPPA) is effectively created (Guo, Liu et al. 2012, Assadi, Lamerz et al. 2013, Akbani, Becker et al. 2014, Creighton and Huang 2015). The creation of a digital RPPA-like assay has numerous applications in translational research, biomarker validation, drug discovery, clinical, and precision medicine.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO:133) or an Illumina P7 primer (5′-CAAGCAGAAGACGGCATACGAGAT-3′-SEQ ID NO:134).

In certain embodiments, a recording tag comprises a spacer at its terminus, e.g., 3′ end. As used herein reference to a spacer sequence in the context of a recording tag includes a spacer sequence that is identical to the spacer sequence associated with its cognate binding agent, or a spacer sequence that is complementary to the spacer sequence associated with its cognate binding agent. The terminal, e.g., 3′, spacer on the recording tag permits transfer of identifying information of a cognate binding agent from its coding tag to the recording tag during the first binding cycle (e.g., via annealing of complementary spacer sequences for primer extension or sticky end ligation).

In one embodiment, the spacer sequence is about 1-20 bases in length, about 2-12 bases in length, or 5-10 bases in length. The length of the spacer may depend on factors such as the temperature and reaction conditions of the primer extension reaction for transferring coding tag information to the recording tag.

In a preferred embodiment, the spacer sequence in the recording is designed to have minimal complementarity to other regions in the recording tag; likewise, the spacer sequence in the coding tag should have minimal complementarity to other regions in the coding tag. In other words, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

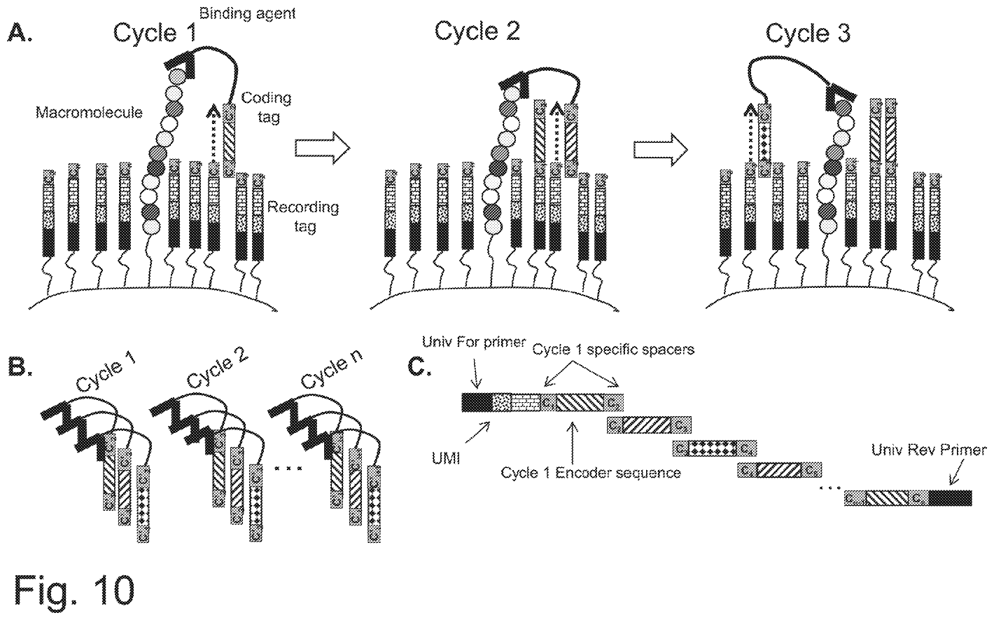

As described for the binding agent spacers, in some embodiments, the recording tags associated with a library of polypeptides share a common spacer sequence. In other embodiments, the recording tags associated with a library of polypeptides have binding cycle specific spacer sequences that are complementary to the binding cycle specific spacer sequences of their cognate binding agents, which can be useful when using non-concatenated extended recording tags (see FIG. 10).

The collection of extended recording tags can be concatenated after the fact (see, e.g., FIG. 10). After the binding cycles are complete, the bead solid supports, each bead comprising on average one or fewer than one polypeptide per bead, each polypeptide having a collection of extended recording tags that are co-localized at the site of the polypeptide, are placed in an emulsion. The emulsion is formed such that each droplet, on average, is occupied by at most 1 bead. An optional assembly PCR reaction is performed in-emulsion to amplify the extended recording tags co-localized with the polypeptide on the bead and assemble them in co-linear order by priming between the different cycle specific sequences on the separate extended recording tags (Xiong, Peng et al. 2008). Afterwards the emulsion is broken and the assembled extended recording tags are sequenced.

In another embodiment, the DNA recording tag is comprised of a universal priming sequence (U1), one or more barcode sequences (BCs), and a spacer sequence (Sp1) specific to the first binding cycle. In the first binding cycle, binding agents employ DNA coding tags comprised of an Sp1 complementary spacer, an encoder barcode, and optional cycle barcode, and a second spacer element (Sp2). The utility of using at least two different spacer elements is that the first binding cycle selects one of potentially several DNA recording tags and a single DNA recording tag is extended resulting in a new Sp2 spacer element at the end of the extended DNA recording tag. In the second and subsequent binding cycles, binding agents contain just the Sp2′ spacer rather than Sp1′. In this way, only the single extended recording tag from the first cycle is extended in subsequent cycles. In another embodiment, the second and subsequent cycles can employ binding agent specific spacers.

In some embodiments, a recording tag comprises from 5′ to 3′ direction: a universal forward (or 5′) priming sequence, a UMI, and a spacer sequence. In some embodiments, a recording tag comprises from 5′ to 3′ direction: a universal forward (or 5′) priming sequence, an optional UMI, a barcode (e.g., sample barcode, partition barcode, compartment barcode, spatial barcode, or any combination thereof), and a spacer sequence. In some other embodiments, a recording tag comprises from 5′ to 3′ direction: a universal forward (or 5′) priming sequence, a barcode (e.g., sample barcode, partition barcode, compartment barcode, spatial barcode, or any combination thereof), an optional UMI, and a spacer sequence.

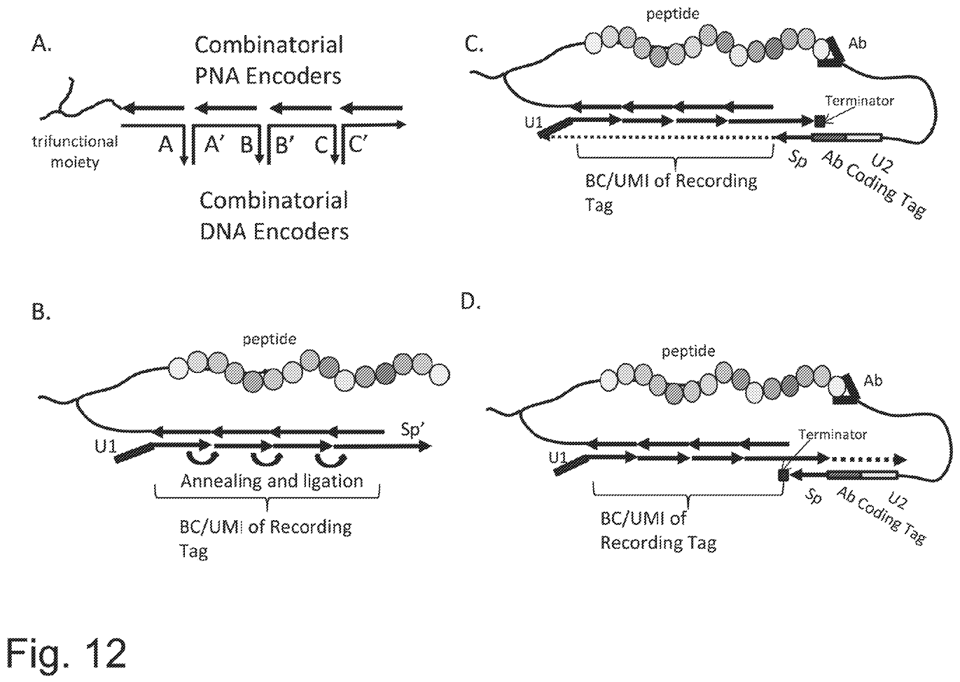

Combinatorial approaches may be used to generate UMIs from modified DNA and PNAs. In one example, a UMI may be constructed by “chemical ligating” together sets of short word sequences (4-15mers), which have been designed to be orthogonal to each other (Spiropulos and Heemstra 2012). A DNA template is used to direct the chemical ligation of the “word” polymers. The DNA template is constructed with hybridizing arms that enable assembly of a combinatorial template structure simply by mixing the sub-components together in solution (see, FIG. 12C). In certain embodiments, there are no “spacer” sequences in this design. The size of the word space can vary from 10's of words to 10,000's or more words. In certain embodiments, the words are chosen such that they differ from one another to not cross hybridize, yet possess relatively uniform hybridization conditions. In one embodiment, the length of the word will be on the order of 10 bases, with about 1000's words in the subset (this is only 0.1% of the total 10-mer word space ˜4¹⁰=1 million words). Sets of these words (1000 in subset) can be concatenated together to generate a final combinatorial UMI with complexity=1000ⁿ power. For 4 words concatenated together, this creates a UMI diversity of 10¹² different elements. These UMI sequences will be appended to the polypeptide at the single molecule level. In one embodiment, the diversity of UMIs exceeds the number of molecules of polypeptides to which the UMIs are attached. In this way, the UMI uniquely identifies the polypeptide of interest. The use of combinatorial word UMI's facilitates readout on high error rate sequencers, (e.g., nanopore sequencers, nanogap tunneling sequencing, etc.) since single base resolution is not required to read words of multiple bases in length. Combinatorial word approaches can also be used to generate other identity-informative components of recording tags or coding tags, such as compartment tags, partition barcodes, spatial barcodes, sample barcodes, encoder sequences, cycle specific sequences, and barcodes. Methods relating to nanopore sequencing and DNA encoding information with error-tolerant words (codes) are known in the art (see, e.g., Kiah et al., 2015, Codes for DNA sequence profiles. IEEE International Symposium on Information Theory (ISIT); Gabrys et al., 2015, Asymmetric Lee distance codes for DNA-based storage. IEEE Symposium on Information Theory (ISIT); Laure et al., 2016, Coding in 2D: Using Intentional Dispersity to Enhance the Information Capacity of Sequence-Coded Polymer Barcodes. Angew. Chem. Int. Ed. doi:10.1002/anie.201605279; Yazdi et al., 2015, IEEE Transactions on Molecular, Biological and Multi-Scale Communications 1:230-248; and Yazdi et al., 2015, Sci Rep 5:14138, each of which is incorporated by reference in its entirety). Thus, in certain embodiments, an extended recording tag, an extended coding tag, or a di-tag construct in any of the embodiments described herein is comprised of identifying components (e.g., UMI, encoder sequence, barcode, compartment tag, cycle specific sequence, etc.) that are error correcting codes. In some embodiments, the error correcting code is selected from: Hamming code, Lee distance code, asymmetric Lee distance code, Reed-Solomon code, and Levenshtein-Tenengolts code. For nanopore sequencing, the current or ionic flux profiles and asymmetric base calling errors are intrinsic to the type of nanopore and biochemistry employed, and this information can be used to design more robust DNA codes using the aforementioned error correcting approaches. An alternative to employing robust DNA nanopore sequencing barcodes, one can directly use the current or ionic flux signatures of barcode sequences (U.S. Pat. No. 7,060,507, incorporated by reference in its entirety), avoiding DNA base calling entirely, and immediately identify the barcode sequence by mapping back to the predicted current/flux signature as described by Laszlo et al. (2014, Nat. Biotechnol. 32:829-833, incorporated by reference in its entirety). In this paper, Laszlo et al. describe the current signatures generated by the biological nanopore, MspA, when passing different word strings through the nanopore, and the ability to map and identify DNA strands by mapping resultant current signatures back to an in silico prediction of possible current signatures from a universe of sequences (2014, Nat. Biotechnol. 32:829-833). Similar concepts can be applied to DNA codes and the electrical signal generated by nanogap tunneling current-based DNA sequencing (Ohshiro et al., 2012, Sci Rep 2: 501).

Thus, in certain embodiments, the identifying components of a coding tag, recording tag, or both are capable of generating a unique current or ionic flux or optical signature, wherein the analysis step of any of the methods provided herein comprises detection of the unique current or ionic flux or optical signature in order to identify the identifying components. In some embodiments, the identifying components are selected from an encoder sequence, barcode, UMI, compartment tag, cycle specific sequence, or any combination thereof.

In certain embodiments, all or substantially amount of the polypeptides (e.g., at least 50%, 55%, 60%, 65%, 70%, 75%, 80%, 85%, 90%, 95%, 96%, 97%, 98%, 99%, or 100%) within a sample are labeled with a recording tag. Labeling of the polypeptides may occur before or after immobilization of the polypeptides to a solid support.

In other embodiments, a subset of polypeptides within a sample are labeled with recording tags. In a particular embodiment, a subset of polypeptides from a sample undergo targeted (analyte specific) labeling with recording tags. Targeted recording tag labeling of proteins may be achieved using target protein-specific binding agents (e.g., antibodies, aptamers, etc.) that are linked a short target-specific DNA capture probe, e.g., analyte-specific barcode, which anneal to complementary target-specific bait sequence, e.g., analyte-specific barcode, in recording tags (see, FIG. 28A). The recording tags comprise a reactive moiety for a cognate reactive moiety present on the target protein (e.g., click chemistry labeling, photoaffinity labeling). For example, recording tags may comprise an azide moiety for interacting with alkyne-derivatized proteins, or recording tags may comprise a benzophenone for interacting with native proteins, etc. (see FIGS. 28A-B). Upon binding of the target protein by the target protein specific binding agent, the recording tag and target protein are coupled via their corresponding reactive moieties (see, FIG. 28B-C). After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein.

In one example, antibodies specific for a set of target proteins can be labeled with a DNA capture probe (e.g., analyte barcode BC_{A} in FIG. 28) that hybridizes with recording tags designed with complementary bait sequence (e.g., analyte barcode BC_{A}′ in FIG. 28). Sample-specific labeling of proteins can be achieved by employing DNA-capture probe labeled antibodies hybridizing with complementary bait sequence on recording tags comprising of sample-specific barcodes.

In another example, target protein-specific aptamers are used for targeted recording tag labeling of a subset of proteins within a sample. A target specific-aptamer is linked to a DNA capture probe that anneals with complementary bait sequence in a recording tag. The recording tag comprises a reactive chemical or photo-reactive chemical probes (e.g. benzophenone (BP)) for coupling to the target protein having a corresponding reactive moiety. The aptamer binds to its target protein molecule, bringing the recording tag into close proximity to the target protein, resulting in the coupling of the recording tag to the target protein.

Photoaffinity (PA) protein labeling using photo-reactive chemical probes attached to small molecule protein affinity ligands has been previously described (Park, Koh et al. 2016). Typical photo-reactive chemical probes include probes based on benzophenone (reactive diradical, 365 nm), phenyldiazirine (reactive carbon, 365 nm), and phenylazide (reactive nitrene free radical, 260 nm), activated under irradiation wavelengths as previously described (Smith and Collins 2015). In a preferred embodiment, target proteins within a protein sample are labeled with recording tags comprising sample barcodes using the method disclosed by Li et al., in which a bait sequence in a benzophenone labeled recording tag is hybridized to a DNA capture probe attached to a cognate binding agent (e.g., nucleic acid aptamer (see FIG. 28) (Li, Liu et al. 2013). For photoaffinity labeled protein targets, the use of DNA/RNA aptamers as target protein-specific binding agents are preferred over antibodies since the photoaffinity moiety can self-label the antibody rather than the target protein. In contrast, photoaffinity labeling is less efficient for nucleic acids than proteins, making aptamers a better vehicle for DNA-directed chemical or photo-labeling. Similar to photo-affinity labeling, one can also employ DNA-directed chemical labeling of reactive lysine's (or other moieties) in the proximity of the aptamer binding site in a manner similar to that described by Rosen et al. (Rosen, Kodal et al. 2014, Kodal, Rosen et al. 2016).

In the aforementioned embodiments, other types of linkages besides hybridization can be used to link the target specific binding agent and the recording tag (see, FIG. 28A). For example, the two moieties can be covalently linked, using a linker that is designed to be cleaved and release the binding agent once the captured target protein (or other polypeptide) is covalently linked to the recording tag as shown in FIG. 28B. A suitable linker can be attached to various positions of the recording tag, such as the 3′ end, or within the linker attached to the 5′ end of the recording tag.

### Binding Agents and Coding Tags

The methods described herein use a binding agent capable of binding to the polypeptide. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule). In some embodiments, the binding agent binds to a non-functionalized NTAA or a functionalized NTAA. In some embodiments, the functionalized NTAA can include an NTAA treated with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof. In some embodiments, the binding agents (e.g., first order, second order, or any higher order binding agents) are capable of binding to or configured to bind to a side product from treating the polypeptide with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof. Also provided herein are kits comprising a plurality of binding agents.

In certain embodiments, a binding agent may be designed to bind covalently. Covalent binding can be designed to be conditional or favored upon binding to the correct moiety. For example, an NTAA and its cognate NTAA-specific binding agent may each be modified with a reactive group such that once the NTAA-specific binding agent is bound to the cognate NTAA, a coupling reaction is carried out to create a covalent linkage between the two. Non-specific binding of the binding agent to other locations that lack the cognate reactive group would not result in covalent attachment. In some embodiments, the polypeptide comprises a ligand that is capable of forming a covalent bond to a binding agent. In some embodiments, the polypeptide comprises a functionalized NTAA which includes a ligand group that is capable of covalent binding to a binding agent. Covalent binding between a binding agent and its target allows for more stringent washing to be used to remove binding agents that are non-specifically bound, thus increasing the specificity of the assay.

In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and/or Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In an example of non-selective binding, a binding agent may bind to two or more of the twenty standard amino acids.

In the practice of the methods disclosed herein, the ability of a binding agent to selectively bind a feature or component of a polypeptide need only be sufficient to allow transfer of its coding tag information to the recording tag associated with the polypeptide, transfer of the recording tag information to the coding tag, or transferring of the coding tag information and recording tag information to a di-tag molecule. Thus, selectively need only be relative to the other binding agents to which the polypeptide is exposed. It should also be understood that selectivity of a binding agent need not be absolute to a specific amino acid, but could be selective to a class of amino acids, such as amino acids with nonpolar or nonpolar side chains, or with electrically (positively or negatively) charged side chains, or with aromatic side chains, or some specific class or size of side chains, and the like.

In a particular embodiment, the binding agent has a high affinity and high selectivity for the polypeptide of interest. In particular, a high binding affinity with a low off-rate is efficacious for information transfer between the coding tag and recording tag. In certain embodiments, a binding agent has a Kd of <500 nM, <100 nM, <50 nM, <10 nM, <5 nM, <1 nM, <0.5 nM, or <0.1 nM. In a particular embodiment, the binding agent is added to the polypeptide at a concentration >10×, >100×, or >1000× its Kd to drive binding to completion. A detailed discussion of binding kinetics of an antibody to a single protein molecule is described in Chang et al. (Chang, Rissin et al. 2012).

To increase the affinity of a binding agent to small N-terminal amino acids (NTAAs) of peptides, the NTAA may be modified with an “immunogenic” hapten, such as dinitrophenol (DNP). This can be implemented in a cyclic sequencing approach using Sanger's reagent, dinitrofluorobenzene (DNFB), which attaches a DNP group to the amine group of the NTAA. Commercial anti-DNP antibodies have affinities in the low nM range (˜8 nM, LO-DNP-2) (Bilgicer, Thomas et al. 2009); as such it stands to reason that it should be possible to engineer high-affinity NTAA binding agents to a number of NTAAs modified with DNP (via DNFB) and simultaneously achieve good binding selectivity for a particular NTAA. In another example, an NTAA may be modified with sulfonyl nitrophenol (SNP) using 4-sulfonyl-2-nitrofluorobenzene (SNFB). Similar affinity enhancements may also be achieved with alternative NTAA modifiers, such as an acetyl group or an amidinyl (guanidinyl) group.

In certain embodiments, a binding agent may bind to an NTAA, a CTAA, an intervening amino acid, dipeptide (sequence of two amino acids), tripeptide (sequence of three amino acids), or higher order peptide of a peptide molecule. In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native amino acid. In some examples, the binding agent binds to an unmodified or native dipeptide (sequence of two amino acids), tripeptide (sequence of three amino acids), or higher order peptide of a peptide molecule. In some examples, a binding agent may bind to an N-terminal or C-terminal diamino acid moiety. A binding agent may be engineered for high affinity for a native or unmodified NTAA, high specificity for a native or unmodified NTAA, or both. In some embodiments, binding agents can be developed through directed evolution of promising affinity scaffolds using phage display.

In some embodiments, the binding agent is partially specific or selective. In some aspects, the binding agent preferentially binds one or more amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a specific terminal amino acid and one or more penultimate amino acid. In some cases, the binding agent preferentially binds to one or more specific terminal amino acid(s) and one penultimate amino acid. For example, a binding agent may preferentially bind AA, AC, and AG or a binding agent may preferentially bind AA, CA, and GA. In some specific examples, binding agents with different specificities can share the same coding tag. In some specific cases, the binding agent is at least partially selective for the chemical modification of the N-terminal amino acid. For example, a binding agent may preferentially bind chemically modified-AA, chemically modified-AC, and chemically modified-AG.

In certain embodiments, a binding agent may bind to a post-translational modification of an amino acid. In some embodiments, a peptide comprises one or more post-translational modifications, which may be the same of different. The NTAA, CTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. A list of lectins recognizing various glycosylation states (e.g., core-fucose, sialic acids, N-acetyl-D-lactosamine, mannose, N-acetyl-glucosamine) include: A, AAA, AAL, ABA, ACA, ACG, ACL, AOL, ASA, BanLec, BC2L-A, BC2LCN, BPA, BPL, Calsepa, CGL2, CNL, Con, ConA, DBA, Discoidin, DSA, ECA, EEL, F17AG, Gal1, Gal1-S, Gal2, Gal3, Gal3C-S, Gal7-S, Gal9, GNA, GRFT, GS-I, GS-II, GSL-I, GSL-II, HHL, HIHA, HPA, I, II, Jacalin, LBA, LCA, LEA, LEL, Lentil, Lotus, LSL-N, LTL, MAA, MAH, MAL I, Malectin, MOA, MPA, MPL, NPA, Orysata, PA-IIL, PA-IL, PALa, PHA-E, PHA-L, PHA-P, PHAE, PHAL, PNA, PPL, PSA, PSL1a, PTL, PTL-I, PWM, RCA120, RS-Fuc, SAMB, SBA, SJA, SNA, SNA-I, SNA-II, SSA, STL, TJA-I, TJA-II, TxLCI, UDA, UEA-I, UEA-II, VFA, VVA, WFA, WGA (see, Zhang et al., 2016, MABS 8:524-535).

In certain embodiments, a binding agent may bind to a modified or labeled NTAA (e.g., an NTAA that has been functionalized by a reagent comprising a compound of any one of Formula (AA), Formula (AB), a compound of the formula R³—NCS, an amine of Formula R²—NH₂ or with a diheteronucleophile, or a salt or conjugate thereof, as described herein, or any combinations thereof). A modified or labeled NTAA can be one that is functionalized with PITC, 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), an acetylating reagent, a guanidinylation reagent, a thioacylation reagent, a thioacetylation reagent, or a thiobenzylation reagent, or a reagent comprising a compound of Formula (AA), Formula (AB), a compound of the formula R³—NCS, an amine of Formula R²—NH₂ or with a diheteronucleophile, or a salt or conjugate thereof, as described herein, or any combinations thereof.

In certain embodiments, a binding agent can be an aptamer (e.g., peptide aptamer, DNA aptamer, or RNA aptamer), an antibody, an anticalin, an ATP-dependent Clp protease adaptor protein (ClpS), an antibody binding fragment, an antibody mimetic, a peptide, a peptidomimetic, a protein, or a polynucleotide (e.g., DNA, RNA, peptide nucleic acid (PNA), a γPNA, bridged nucleic acid (BNA), xeno nucleic acid (XNA), glycerol nucleic acid (GNA), or threose nucleic acid (TNA), or a variant thereof).

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactivity component(s) of an antibody molecule that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab)₂ fragments, single chain antibody fragments (scFv), miniantibodies, diabodies, crosslinked antibody fragments, Affibody™, nanobodies, single domain antibodies, DVD-Ig molecules, alphabodies, affimers, affitins, cyclotides, molecules, and the like. Immunoreactive products derived using antibody engineering or protein engineering techniques are also expressly within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize a peptide can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres. (see, Jayasena, 1999, Clin Chem 45:1628-50; Kusser 2000, J. Biotechnol. 74: 27-39; Colas, 2000, Curr Opin Chem Biol 4:54-9). Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC, 1-fluoro-2,4-dinitrobenzene (using Sanger's reagent, DNFB), dansyl chloride (using DNS-Cl, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). A binding agent can also be designed or modified, and utilized, to specifically bind a modified NTAA or modified by a compound of Formula (AA), Formula (AB), a compound of the formula R³—NCS, an amine of Formula R²—NH₂ or with a diheteronucleophile, or a salt or conjugate thereof, as described herein, or any combinations thereof. Strategies for directed evolution of proteins are known in the art (e.g., reviewed by Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

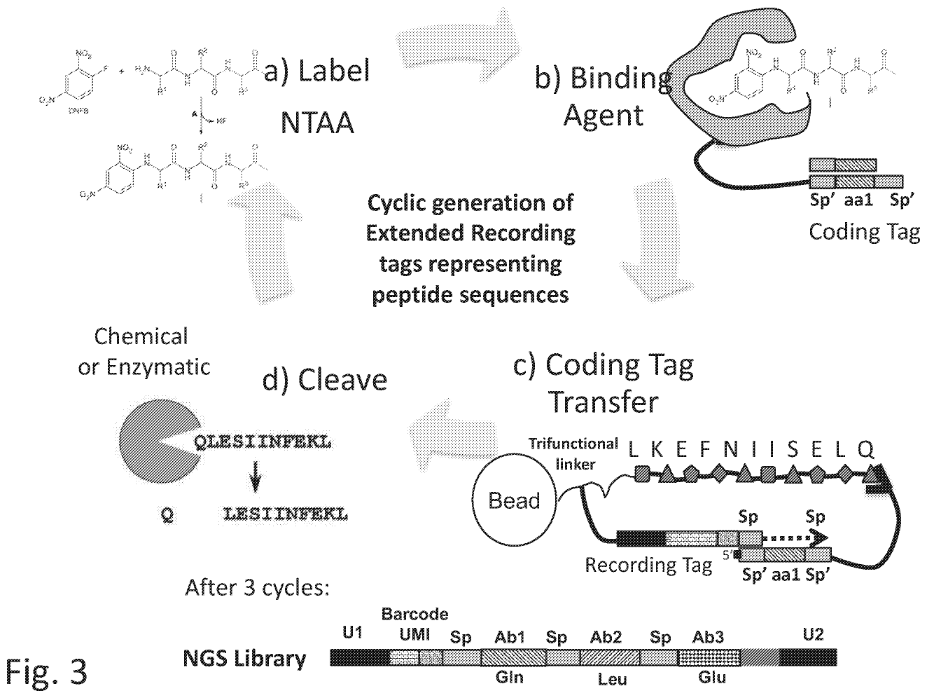

In some embodiments, a binding agent that selectively binds to a functionalized NTAA can be utilized. For example, the NTAA may be reacted with phenylisothiocyanate (PITC) to form a phenylthiocarbamoyl-NTAA derivative. In this manner, the binding agent may be fashioned to selectively bind both the phenyl group of the phenylthiocarbamoyl moiety as well as the alpha-carbon R group of the NTAA. Use of PITC in this manner allows for subsequent elimination of the NTAA by Edman degradation as discussed below. In another embodiment, the NTAA may be reacted with Sanger's reagent (DNFB), to generate a DNP-labeled NTAA (see FIG. 3). Optionally, DNFB is used with an ionic liquid such as 1-ethyl-3-methylimidazolium bis[(trifluoromethyl)sulfonyl]imide ([emim][Tf2N]), in which DNFB is highly soluble. In this manner, the binding agent may be engineered to selectively bind the combination of the DNP and the R group on the NTAA. The addition of the DNP moiety provides a larger “handle” for the interaction of the binding agent with the NTAA, and should lead to a higher affinity interaction. In yet another embodiment, a binding agent may be an aminopeptidase that has been engineered to recognize the DNP-labeled NTAA providing cyclic control of aminopeptidase degradation of the peptide. Once the DNP-labeled NTAA is eliminated, another cycle of DNFB derivitization is performed in order to bind and eliminate the newly exposed NTAA. In preferred particular embodiment, the aminopeptidase is a monomeric metallo-protease, such an aminopeptidase activated by zinc (Calcagno and Klein 2016). In another example, a binding agent may selectively bind to an NTAA that is modified with sulfonyl nitrophenol (SNP), e.g., by using 4-sulfonyl-2-nitrofluorobenzene (SNFB). In yet another embodiment, a binding agent may selectively bind to an NTAA that is acetylated or amidinated. In some embodiments, a binding agent may bind to an NTAA that is modified with a compound of Formula (AA), Formula (AB), a compound of the formula R³—NCS, an amine of Formula R²—NH₂ or with a diheteronucleophile, or a salt or conjugate thereof, as described herein, or any combinations thereof.

Other reagents that may be used to functionalize the NTAA include trifluoroethyl isothiocyanate, allyl isothiocyanate, and dimethylaminoazobenzene isothiocyanate.

Isothiocyates, in the presence of ionic liquids, have been shown to have enhanced reactivity to primary amines. Ionic liquids are excellent solvents (and serve as a catalyst) in organic chemical reactions and can enhance the reaction of isothiocyanates with amines to form thioureas. An example is the use of the ionic liquid 1-butyl-3-methyl-imidazolium tetraflouoraborate [Bmim][BF4] for rapid and efficient functionalization of aromatic and aliphatic amines by phenyl isothiocyanate (PITC) (Le, Chen et al. 2005). Edman degradation involves the reaction of isothiocyanates, such at PITC, with the amino N-terminus of peptides. As such, in one embodiment ionic liquids are used to improve the efficiency of the Edman elimination process by providing milder functionalization and elimination conditions. For instance, the use of 5% (vol./vol.) PITC in ionic liquid [Bmim][BF4] at 25° C. for 10 min. is more efficient than functionalization under standard Edman PITC derivatization conditions which employ 5% (vol./vol.) PITC in a solution containing pyridine, ethanol, and ddH2O (1:1:1 vol./vol./vol.) at 55° C. for 60 min (Wang, Fang et al. 2009). In a preferred embodiment, internal lysine, tyrosine, histidine, and cysteine amino acids are blocked within the polypeptide prior to fragmentation into peptides. In this way, only the peptide α-amine group of the NTAA is accessible for modification during the peptide sequencing reaction. This is particularly relevant when using DNFB (Sanger' reagent) and dansyl chloride.

A binding agent may be engineered for high affinity for a modified NTAA, high specificity for a modified NTAA, or both. In some embodiments, binding agents can be developed through directed evolution of promising affinity scaffolds using phage display.

Engineered aminopeptidase mutants that bind to and cleave individual or small groups of labelled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., 2015, Anal. Biochem. 488:6-8). However, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10). Aminopeptidases may be engineered to specifically bind to 20 different NTAAs representing the standard amino acids that are labeled with a specific moiety (e.g., PTC, DNP, SNP, modified with a diheterocyclic methanimine etc.). Control of the stepwise degradation of the N-terminus of the peptide is achieved by using engineered aminopeptidases that are only active (e.g., binding activity or catalytic activity) in the presence of the label. In another example, Havranak et al. (U.S. Patent Publication 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In another example, highly-selective engineered ClpSs have also been described in the literature. Emili et al. describe the directed evolution of an E. coli ClpS protein via phage display, resulting in four different variants with the ability to selectively bind NTAAs for aspartic acid, arginine, tryptophan, and leucine residues (U.S. Pat. No. 9,566,335, incorporated by reference in its entirety). In one embodiment, the binding moiety of the binding agent comprises a member of the evolutionarily conserved ClpS family of adaptor proteins involved in natural N-terminal protein recognition and binding or a variant thereof. The ClpS family of adaptor proteins in bacteria are described in Schuenemann et al., (2009), “Structural basis of N-end rule substrate recognition in Escherichia coli by the ClpAP adaptor protein ClpS,” EMBO Reports 10(5), and Roman-Hernandez et al., (2009), “Molecular basis of substrate selection by the N-end rule adaptor protein ClpS,” PNAS 106(22):8888-93. See also Guo et al., (2002), JBC 277(48): 46753-62, and Wang et al., (2008), “The molecular basis of N-end rule recognition,” Molecular Cell 32: 406-414. In some embodiments, the amino acid residues corresponding to the ClpS hydrophobic binding pocket identified in Schuenemann et al. are modified in order to generate a binding moiety with the desired selectivity.

In one embodiment, the binding moiety comprises a member of the UBR box recognition sequence family, or a variant of the UBR box recognition sequence family. UBR recognition boxes are described in Tasaki et al., (2009), JBC 284(3): 1884-95. For example, the binding moiety may comprise UBR1, UBR2, or a mutant, variant, or homologue thereof.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the binding agent comprises a polypeptide, such as a modified member of the ClpS family of adaptor proteins, such as a variant of a E. Coli ClpS binding polypeptide, and a detectable label. In one embodiment, the detectable label is optically detectable. In some embodiments, the detectable label comprises a fluorescent moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g. DNP, SNP, acetylated, modified with a diheterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer and Skerra 2012). Engineering of alternative scaffolds for new binding functions has also been reviewed by Banta et al. (2013, Annu. Rev. Biomed. Eng. 15:93-113).

The functional affinity (avidity) of a given monovalent binding agent may be increased by at least an order of magnitude by using a bivalent or higher order multimer of the monovalent binding agent (Vauquelin and Charlton 2013). Avidity refers to the accumulated strength of multiple, simultaneous, non-covalent binding interactions. An individual binding interaction may be easily dissociated. However, when multiple binding interactions are present at the same time, transient dissociation of a single binding interaction does not allow the binding protein to diffuse away and the binding interaction is likely to be restored. An alternative method for increasing avidity of a binding agent is to include complementary sequences in the coding tag attached to the binding agent and the recording tag associated with the polypeptide.

In some embodiments, a binding agent can be utilized that selectively binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

Other potential scaffolds that can be engineered to generate binders for use in the methods described herein include: an anticalin, an amino acid tRNA synthetase (aaRS), ClpS, an Affilin®, an Adnectin™, a T cell receptor, a zinc finger protein, a thioredoxin, GST A1-1, DARPin, an affimer, an affitin, an alphabody, an avimer, a Kunitz domain peptide, a monobody, a single domain antibody, EETI-II, HPSTI, intrabody, lipocalin, PHD-finger, V(NAR) LDTI, evibody, Ig(NAR), knottin, maxibody, neocarzinostatin, pVIII, tendamistat, VLR, protein A scaffold, MTI-II, ecotin, GCN4, Im9, kunitz domain, microbody, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2).

A binding agent may be engineered to withstand higher temperatures and mild-denaturing conditions (e.g., presence of urea, guanidinium thiocyanate, ionic solutions, etc.). The use of denaturants helps reduce secondary structures in the surface bound peptides, such as α-helical structures, β-hairpins, β-strands, and other such structures, which may interfere with binding of binding agents to linear peptide epitopes. In one embodiment, an ionic liquid such as 1-ethyl-3-methylimidazolium acetate ([EMIM]+[ACE] is used to reduce peptide secondary structure during binding cycles (Lesch, Heuer et al. 2015).

In some aspects, the binding agent comprises a coding tag containing identifying information regarding the binding agent. For example, the coding tag information associated with a specific binding agent may be in any format capable and suitable for transfer to a recording tag using a variety of methods. In some aspects, the binding agent further comprises one or more detectable labels such as fluorescent labels, in addition to the binding moiety. A binding agent described may comprise a coding tag containing identifying information regarding the binding agent. A coding tag is a nucleic acid molecule of about 3 bases to about 100 bases that provides unique identifying information for its associated binding agent. A coding tag may comprise about 3 to about 90 bases, about 3 to about 80 bases, about 3 to about 70 bases, about 3 to about 60 bases, about 3 bases to about 50 bases, about 3 bases to about 40 bases, about 3 bases to about 30 bases, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be composed of DNA, RNA, polynucleotide analogs, or a combination thereof. Polynucleotide analogs include PNA, γPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

A coding tag comprises an encoder sequence that provides identifying information regarding the associated binding agent. An encoder sequence is about 3 bases to about 30 bases, about 3 bases to about 20 bases, about 3 bases to about 10 bases, or about 3 bases to about 8 bases. In some embodiments, an encoder sequence is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. The length of the encoder sequence determines the number of unique encoder sequences that can be generated. Shorter encoding sequences generate a smaller number of unique encoding sequences, which may be useful when using a small number of binding agents. Longer encoder sequences may be desirable when analyzing a population of polypeptides. For example, an encoder sequence of 5 bases would have a formula of 5′-NNNNN-3′ (SEQ ID NO:135), wherein N may be any naturally occurring nucleotide, or analog. Using the four naturally occurring nucleotides A, T, C, and G, the total number of unique encoder sequences having a length of 5 bases is 1,024. In some embodiments, the total number of unique encoder sequences may be reduced by excluding, for example, encoder sequences in which all the bases are identical, at least three contiguous bases are identical, or both. In a specific embodiment, a set of ≥50 unique encoder sequences are used for a binding agent library.

In some embodiments, identifying components of a coding tag or recording tag, e.g., the encoder sequence, barcode, UMI, compartment tag, partition barcode, sample barcode, spatial region barcode, cycle specific sequence or any combination thereof, is subject to Hamming distance, Lee distance, asymmetric Lee distance, Reed-Solomon, Levenshtein-Tenengolts, or similar methods for error-correction. Hamming distance refers to the number of positions that are different between two strings of equal length. It measures the minimum number of substitutions required to change one string into the other. Hamming distance may be used to correct errors by selecting encoder sequences that are reasonable distance apart. Thus, in the example where the encoder sequence is 5 base, the number of useable encoder sequences is reduced to 256 unique encoder sequences (Hamming distance of 1→4⁴ encoder sequences=256 encoder sequences). In another embodiment, the encoder sequence, barcode, UMI, compartment tag, cycle specific sequence, or any combination thereof is designed to be easily read out by a cyclic decoding process (Gunderson, 2004, Genome Res. 14:870-7). In another embodiment, the encoder sequence, barcode, UMI, compartment tag, partition barcode, spatial barcode, sample barcode, cycle specific sequence, or any combination thereof is designed to be read out by low accuracy nanopore sequencing, since rather than requiring single base resolution, words of multiple bases (˜5-20 bases in length) need to be read. A subset of 15-mer, error-correcting Hamming barcodes that may be used in the methods of the present disclosure are set forth in SEQ ID NOS:1-65 and their corresponding reverse complementary sequences as set forth in SEQ ID NO:66-130.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-translationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. A spacer sequence is about 1 base to about 20 bases, about 1 base to about 10 bases, about 5 bases to about 9 bases, or about 4 bases to about 8 bases. In some embodiments, a spacer is about 1 base, 2 bases, 3 bases, 4 bases, 5 bases, 6 bases, 7 bases, 8 bases, 9 bases, 10 bases, 11 bases, 12 bases, 13 bases, 14 bases, 15 bases or 20 bases in length. In some embodiments, a spacer within a coding tag is shorter than the encoder sequence, e.g., at least 1 base, 2, bases, 3 bases, 4 bases, 5 bases, 6, bases, 7 bases, 8 bases, 9 bases, 10 bases, 11 bases, 12 bases, 13 bases, 14 bases, 15 bases, 20 bases, or 25 bases shorter than the encoder sequence. In other embodiments, a spacer within a coding tag is the same length as the encoder sequence. In certain embodiments, the spacer is binding agent specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. A 5′ spacer on a coding tag (see FIG. 5A, “*Sp′”) may optionally contain pseudo complementary bases to a 3′ spacer on the recording tag to increase Tₘ (Lehoud et al., 2008, Nucleic Acids Res. 36:3409-3419).

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. This embodiment is useful for subsequent PCR assembly of non-concatenated extended recording tags after the binding cycles are completed (see FIG. 10). In some embodiments, a spacer sequence comprises a sufficient number of bases to anneal to a complementary spacer sequence in a recording tag or extended recording tag to initiate a primer extension reaction or sticky end ligation reaction.

A cycle specific spacer sequence can also be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle dependent spacer sequences. More specifically, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. Coding tags of binding agents from the first binding cycle are capable of annealing to recording tags via complementary cycle 1 specific spacer sequences. Upon transfer of the coding tag information to the recording tag, the cycle 2 specific spacer sequence is positioned at the 3′ terminus of the extended recording tag at the end of binding cycle 1. Coding tags of binding agents from the second binding cycle are capable of annealing to the extended recording tags via complementary cycle 2 specific spacer sequences. Upon transfer of the coding tag information to the extended recording tag, the cycle 3 specific spacer sequence is positioned at the 3′ terminus of the extended recording tag at the end of binding cycle 2, and so on through “n” binding cycles. This embodiment provides that transfer of binding information in a particular binding cycle among multiple binding cycles will only occur on (extended) recording tags that have experienced the previous binding cycles. However, sometimes a binding agent will fail to bind to a cognate polypeptide. Oligonucleotides comprising binding cycle specific spacers after each binding cycle as a “chase” step can be used to keep the binding cycles synchronized even if the event of a binding cycle failure. For example, if a cognate binding agent fails to bind to a polypeptide during binding cycle 1, adding a chase step following binding cycle 1 using oligonucleotides comprising both a cycle 1 specific spacer, a cycle 2 specific spacer, and a “null” encoder sequence. The “null” encoder sequence can be the absence of an encoder sequence or, preferably, a specific barcode that positively identifies a “null” binding cycle. The “null” oligonucleotide is capable of annealing to the recording tag via the cycle 1 specific spacer, and the cycle 2 specific spacer is transferred to the recording tag. Thus, binding agents from binding cycle 2 are capable of annealing to the extended recording tag via the cycle 2 specific spacer despite the failed binding cycle 1 event. The “null” oligonucleotide marks binding cycle 1 as a failed binding event within the extended recording tag.

In some preferred embodiments, binding cycle-specific encoder sequences are used in coding tags. Binding cycle-specific encoder sequences may be accomplished either via the use of completely unique analyte (e.g., NTAA)-binding cycle encoder barcodes or through a combinatoric use of an analyte (e.g., NTAA) encoder sequence joined to a cycle-specific barcode (see FIG. 35). The advantage of using a combinatoric approach is that fewer total barcodes need to be designed. For a set of 20 analyte binding agents used across 10 cycles, only 20 analyte encoder sequence barcodes and 10 binding cycle specific barcodes need to be designed. In contrast, if the binding cycle is embedded directly in the binding agent encoder sequence, then a total of 200 independent encoder barcodes may need to be designed. An advantage of embedding binding cycle information directly in the encoder sequence is that the total length of the coding tag can be minimized when employing error-correcting barcodes. In some embodiments, error-correcting barcodes are useful on a nanopore readout. The use of error-tolerant barcodes allows highly accurate barcode identification using sequencing platforms and approaches that are more error-prone, but have other advantages such as rapid speed of analysis, lower cost, and/or more portable instrumentation. One such example is a nanopore-based sequencing readout. In some embodiments, coding tags associated with binding agents used to bind in an alternating cycles comprises different binding cycle specific spacer sequences. For example, a coding tag for binding agents used in the first binding cycle comprise a “cycle 1” specific spacer sequence, a coding tag for binding agents used in the second binding cycle comprise a “cycle 2” specific spacer sequence, a coding tag for binding agents used in the third binding cycle also comprises the “cycle 1” specific spacer sequence, a coding tag for binding agents used in the fourth binding cycle comprises the “cycle 2” specific spacer sequence. In this manner, cycle specific spacers are not needed for every cycle.

In some embodiments, a coding tag comprises a cleavable or nickable DNA strand within the second (3′) spacer sequence proximal to the binding agent (see, FIG. 32). For example, the 3′ spacer may have one or more uracil bases that can be nicked by uracil-specific excision reagent (USER). USER generates a single nucleotide gap at the location of the uracil. In another example, the 3′ spacer may comprise a recognition sequence for a nicking endonuclease that hydrolyzes only one strand of a duplex. Preferably, the enzyme used for cleaving or nicking the 3′ spacer sequence acts only on one DNA strand (the 3′ spacer of the coding tag), such that the other strand within the duplex belonging to the (extended) recording tag is left intact. These embodiments is particularly useful in assays analysing proteins in their native conformation, as it allows the non-denaturing removal of the binding agent from the (extended) recording tag after primer extension has occurred and leaves a single stranded DNA spacer sequence on the extended recording tag available for subsequent binding cycles.

The coding tags may also be designed to contain palindromic sequences. Inclusion of a palindromic sequence into a coding tag allows a nascent, growing, extended recording tag to fold upon itself as coding tag information is transferred. The extended recording tag is folded into a more compact structure, effectively decreasing undesired inter-molecular binding and primer extension events.

In some embodiments, a coding tag comprises analyte-specific spacer that is capable of priming extension only on recording tags previously extended with binding agents recognizing the same analyte. An extended recording tag can be built up from a series of binding events using coding tags comprising analyte-specific spacers and encoder sequences. In one embodiment, a first binding event employs a binding agent with a coding tag comprised of a generic 3′ spacer primer sequence and an analyte-specific spacer sequence at the 5′ terminus for use in the next binding cycle; subsequent binding cycles then use binding agents with encoded analyte-specific 3′ spacer sequences. This design results in amplifiable library elements being created only from a correct series of cognate binding events. Off-target and cross-reactive binding interactions will lead to a non-amplifiable extended recording tag. In one example, a pair of cognate binding agents to a particular polypeptide analyte is used in two binding cycles to identify the analyte. The first cognate binding agent contains a coding tag comprised of a generic spacer 3′ sequence for priming extension on the generic spacer sequence of the recording tag, and an encoded analyte-specific spacer at the 5′ end, which will be used in the next binding cycle. For matched cognate binding agent pairs, the 3′ analyte-specific spacer of the second binding agent is matched to the 5′ analyte-specific spacer of the first binding agent. In this way, only correct binding of the cognate pair of binding agents will result in an amplifiable extended recording tag. Cross-reactive binding agents will not be able to prime extension on the recording tag, and no amplifiable extended recording tag product generated. This approach greatly enhances the specificity of the methods disclosed herein. The same principle can be applied to triplet binding agent sets, in which 3 cycles of binding are employed. In a first binding cycle, a generic 3′ Sp sequence on the recording tag interacts with a generic spacer on a binding agent coding tag. Primer extension transfers coding tag information, including an analyte specific 5′ spacer, to the recording tag. Subsequent binding cycles employ analyte specific spacers on the binding agents' coding tags.

In certain embodiments, a coding tag may further comprise a unique molecular identifier for the binding agent to which the coding tag is linked. A UMI for the binding agent may be useful in embodiments utilizing extended coding tags or di-tag molecules for sequencing readouts, which in combination with the encoder sequence provides information regarding the identity of the binding agent and number of unique binding events for a polypeptide.

In another embodiment, a coding tag includes a randomized sequence (a set of N's, where N=a random selection from A, C, G, T, or a random selection from a set of words). After a series of “n” binding cycles and transfer of coding tag information to the (extended) recording tag, the final extended recording tag product will be composed of a series of these randomized sequences, which collectively form a “composite” unique molecule identifier (UMI) for the final extended recording tag. If for instance each coding tag contains an (NN) sequence (4*4=16 possible sequences), after 10 sequencing cycles, a combinatoric set of 10 distributed 2-mers is formed creating a total diversity of 16¹⁰˜10¹² possible composite UMI sequences for the extended recording tag products. Given that a peptide sequencing experiment uses ˜10⁹ molecules, this diversity is more than sufficient to create an effective set of UMIs for a sequencing experiment. Increased diversity can be achieved by simply using a longer randomized region (NNN, NNNN, NNNNN, etc.; SEQ ID NO: 135 and 136) within the coding tag.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag may comprise a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

A coding tag is joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin).

In some embodiments, a binding agent is joined to a coding tag via SpyCatcher-SpyTag interaction (see, FIG. 43B). The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Bioconjugate Techniques, G. T. Hermanson, Academic Press (2013)).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In certain embodiments, a polypeptide is also contacted with a non-cognate binding agent. As used herein, a non-cognate binding agent is referring to a binding agent that is selective for a different polypeptide feature or component than the particular polypeptide being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the n^{th} NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n−1 amino acid of the peptide to the n−1 NTAA (e.g., tyrosine), and the peptide was then contacted with the same three binding agents, the binding agent selective for tyrosine would be second binding agent capable of selectively binding to the n−1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

### Cyclic Transfer of Coding Tag Information to Recording Tags

In the methods described herein, upon binding of a binding agent to a polypeptide, identifying information of its linked coding tag is transferred to a recording tag associated with the polypeptide, thereby generating an “extended recording tag.” An extended recording tag may comprise information from a binding agent's coding tag representing each binding cycle performed. However, an extended recording tag may also experience a “missed” binding cycle, e.g., because a binding agent fails to bind to the polypeptide, because the coding tag was missing, damaged, or defective, because the primer extension reaction failed. Even if a binding event occurs, transfer of information from the coding tag to the recording tag may be incomplete or less than 100% accurate, e.g., because a coding tag was damaged or defective, because errors were introduced in the primer extension reaction). Thus, an extended recording tag may represent 100%, or up to 95%, 90%, 85%, 80%, 75%, 70%, 65%, 60%, 65%, 55%, 50%, 45%, 40%, 35%, 30% of binding events that have occurred on its associated polypeptide. Moreover, the coding tag information present in the extended recording tag may have at least 30%, 35%, 40%, 45%, 50%, 55%, 60%, 65%, 70%, 75%, 80%, 85%, 90%, 95%, or 100% identity the corresponding coding tags.

In certain embodiments, an extended recording tag may comprise information from multiple coding tags representing multiple, successive binding events. In these embodiments, a single, concatenated extended recording tag can be representative of a single polypeptide (see, FIG. 2A). As referred to herein, transfer of coding tag information to a recording tag also includes transfer to an extended recording tag as would occur in methods involving multiple, successive binding events.

In certain embodiments, the binding event information is transferred from a coding tag to a recording tag in a cyclic fashion (see FIGS. 2A and 2C). Cross-reactive binding events can be informatically filtered out after sequencing by requiring that at least two different coding tags, identifying two or more independent binding events, map to the same class of binding agents (cognate to a particular protein). An optional sample or compartment barcode can be included in the recording tag, as well an optional UMI sequence. The coding tag can also contain an optional UMI sequence along with the encoder and spacer sequences. Universal priming sequences (U1 and U2) may also be included in extended recording tags for amplification and NGS sequencing (see FIG. 2A).

Coding tag information associated with a specific binding agent may be transferred to a recording tag using a variety of methods. In certain embodiments, information of a coding tag is transferred to a recording tag via primer extension (Chan, McGregor et al. 2015). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template (see, FIGS. 5-7). In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites. Alternatively, the nascent recording tag can also be coated with RecA (or related homologues such as uvsX) to facilitate invasion of the 3′ terminus into a completely double stranded coding tag (Bell et al., 2012, Nature 491:274-278). This configuration prevents the double stranded coding tag from interacting with internal recording tag elements, yet is susceptible to strand invasion by the RecA coated 3′ tail of the extended recording tag (Bell, et al., 2015, Elife 4: e08646). The presence of a single-stranded binding protein can facilitate the strand displacement reaction.

In some embodiments, a DNA polymerase that is used for primer extension possesses strand-displacement activity and has limited or is devoid of 3′-5 exonuclease activity. Several of many examples of such polymerases include Klenow exo- (Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9° N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage T7 gene 2.5 protein, phage Pf3 SSB, replication protein A RPA32 and RPA14 subunits (Wold, 1997); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage T7 helicase/primase, phage T4 gene 41 helicase, E. coli Rep helicase, E. coli recBCD helicase, recA, E. coli and eukaryotic topoisomerases (Champoux, 2001).

Mis-priming or self-priming events, such as when the terminal spacer sequence of the recoding tag primes extension self-extension may be minimized by inclusion of single stranded binding proteins (T4 gene 32, E. coli SSB, etc.), DMSO (1-10%), formamide (1-10%), BSA (10-100 ug/ml), TMACl (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, T7 DNA polymerase exo- (Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In embodiments using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. (FIG. 43A). In this manner, the extended recording tag (with or without a non-templated adenosine base) can anneal to the coding tag and undergo primer extension.

Alternatively, addition of non-templated base can be reduced by employing a mutant polymerase (mesophilic or thermophilic) in which non-templated terminal transferase activity has been greatly reduced by one or more point mutations, especially in the 0-helix region (see U.S. Pat. No. 7,501,237) (Yang, Astatke et al. 2002). Pfu exo-, which is 3′ exonuclease deficient and has strand-displacing ability, also does not have non-templated terminal transferase activity.

In another embodiment, polymerase extension buffers are comprised of 40-120 mM buffering agent such as Tris-Acetate, Tris-HCl, HEPES, etc. at a pH of 6-9.

Self-priming/mis-priming events initiated by self-annealing of the terminal spacer sequence of the extended recording tag with internal regions of the extended recording tag may be minimized by including pseudo-complementary bases in the recording/extended recording tag (Lahoud, Timoshchuk et al. 2008), (Hoshika, Chen et al. 2010). Pseudo-complementary bases show significantly reduced hybridization affinities for the formation of duplexes with each other due the presence of chemical modification. However, many pseudo-complementary modified bases can form strong base pairs with natural DNA or RNA sequences. In certain embodiments, the coding tag spacer sequence is comprised of multiple A and T bases, and commercially available pseudo-complementary bases 2-aminoadenine and 2-thiothymine are incorporated in the recording tag using phosphoramidite oligonucleotide synthesis. Additional pseudocomplementary bases can be incorporated into the extended recording tag during primer extension by adding pseudo-complementary nucleotides to the reaction (Gamper, Arar et al. 2006).

To minimize non-specific interaction of the coding tag labeled binding agents in solution with the recording tags of immobilized proteins, competitor (also referred to as blocking) oligonucleotides complementary to recording tag spacer sequences are added to binding reactions to minimize non-specific interaction s (FIG. 32A-D). Blocking oligonucleotides are relatively short. Excess competitor oligonucleotides are washed from the binding reaction prior to primer extension, which effectively dissociates the annealed competitor oligonucleotides from the recording tags, especially when exposed to slightly elevated temperatures (e.g., 30-50° C.). Blocking oligonucleotides may comprise a terminator nucleotide at its 3′ end to prevent primer extension.

In certain embodiments, the annealing of the spacer sequence on the recording tag to the complementary spacer sequence on the coding tag is metastable under the primer extension reaction conditions (i.e., the annealing Tm is similar to the reaction temperature). This allows the spacer sequence of the coding tag to displace any blocking oligonucleotide annealed to the spacer sequence of the recording tag.

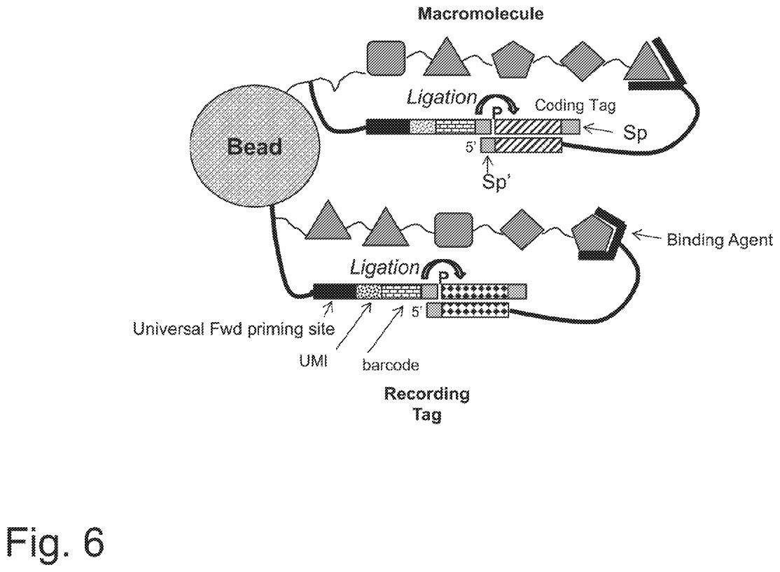

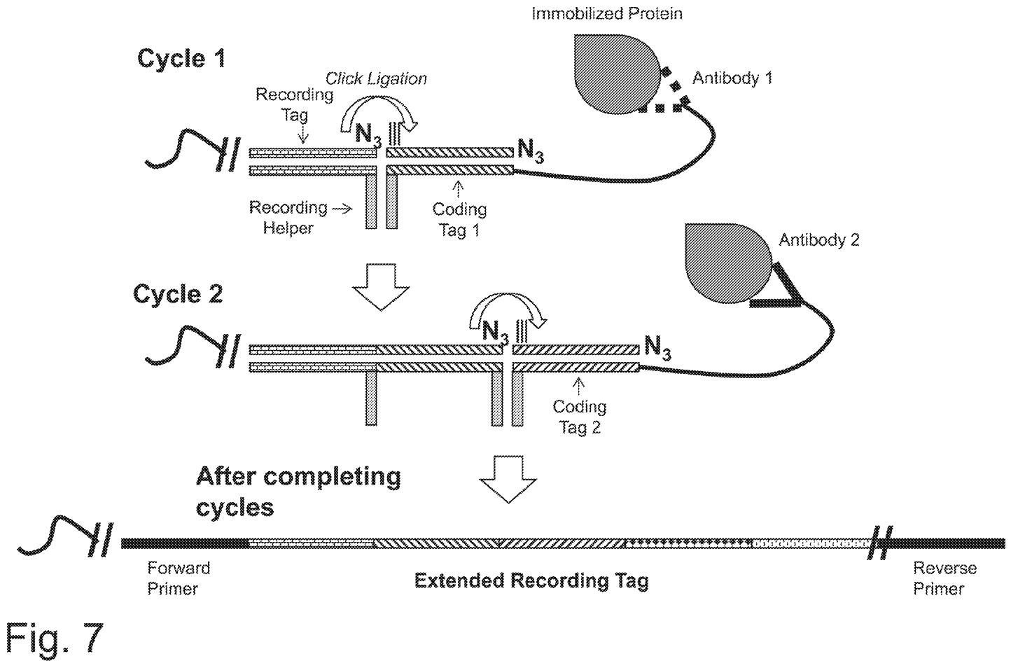

Coding tag information associated with a specific binding agent may also be transferred to a recording tag via ligation (see, e.g., FIGS. 6 and 7). Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase, 9° N DNA ligase, Electroligase®. Alternatively, a ligation may be a chemical ligation reaction (see FIG. 7). In the illustration, a spacer-less ligation is accomplished by using hybridization of a “recording helper” sequence with an arm on the coding tag. The annealed complement sequences are chemically ligated using standard chemical ligation or “click chemistry” (Gunderson, Huang et al. 1998, Peng, Li et al. 2010, El-Sagheer, Cheong et al. 2011, El-Sagheer, Sanzone et al. 2011, Sharma, Kent et al. 2012, Roloff and Seitz 2013, Litovchick, Clark et al. 2014, Roloff, Ficht et al. 2014).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivitizing the 5′ N-terminus with a cysteinyl moiety and the 3′ C-terminus with a thioester moiety. Such modified PNAs easily couple using standard native chemical ligation conditions (Roloff et al., 2013, Bioorgan. Med. Chem. 21:3458-3464).

In some embodiments, coding tag information can be transferred using topoisomerase. Topoisomerase can be used be used to ligate a topo-charged 3′ phosphate on the recording tag to the 5′ end of the coding tag, or complement thereof (Shuman et al., 1994, J. Biol. Chem. 269:32678-32684).

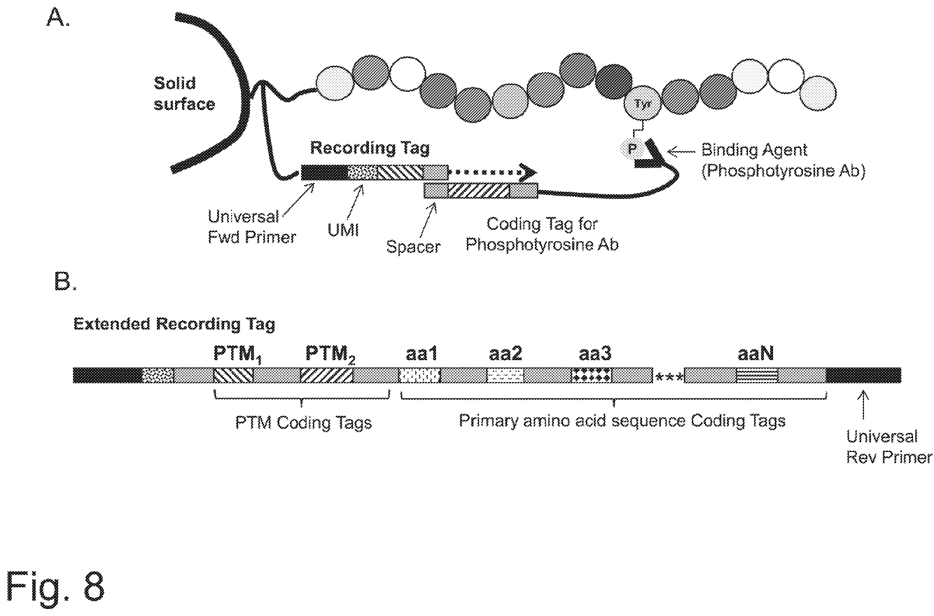

As described herein, a binding agent may bind to a post-translationally modified amino acid. Thus, in certain embodiments, an extended recording tag comprises coding tag information relating to amino acid sequence and post-translational modifications of the polypeptide. In some embodiments, detection of internal post-translationally modified amino acids (e.g., phosphorylation, glycosylation, succinylation, ubiquitination, S-Nitrosylation, methylation, N-acetylation, lipidation, etc.) is be accomplished prior to detection and elimination of terminal amino acids (e.g., NTAA). In one example, a peptide is contacted with binding agents for PTM modifications, and associated coding tag information are transferred to the recording tag as described above (see FIG. 8A). Once the detection and transfer of coding tag information relating to amino acid modifications is complete, the PTM modifying groups can be removed before detection and transfer of coding tag information for the primary amino acid sequence using N-terminal or C-terminal degradation methods. Thus, resulting extended recording tags indicate the presence of post-translational modifications in a peptide sequence, though not the sequential order, along with primary amino acid sequence information (see FIG. 8B).

In some embodiments, detection of internal post-translationally modified amino acids may occur concurrently with detection of primary amino acid sequence. In one example, an NTAA (or CTAA) is contacted with a binding agent specific for a post-translationally modified amino acid, either alone or as part of a library of binding agents (e.g., library composed of binding agents for the 20 standard amino acids and selected post-translational modified amino acids). Successive cycles of terminal amino acid elimination and contact with a binding agent (or library of binding agents) follow. Thus, resulting extended recording tags indicate the presence and order of post-translational modifications in the context of a primary amino acid sequence.

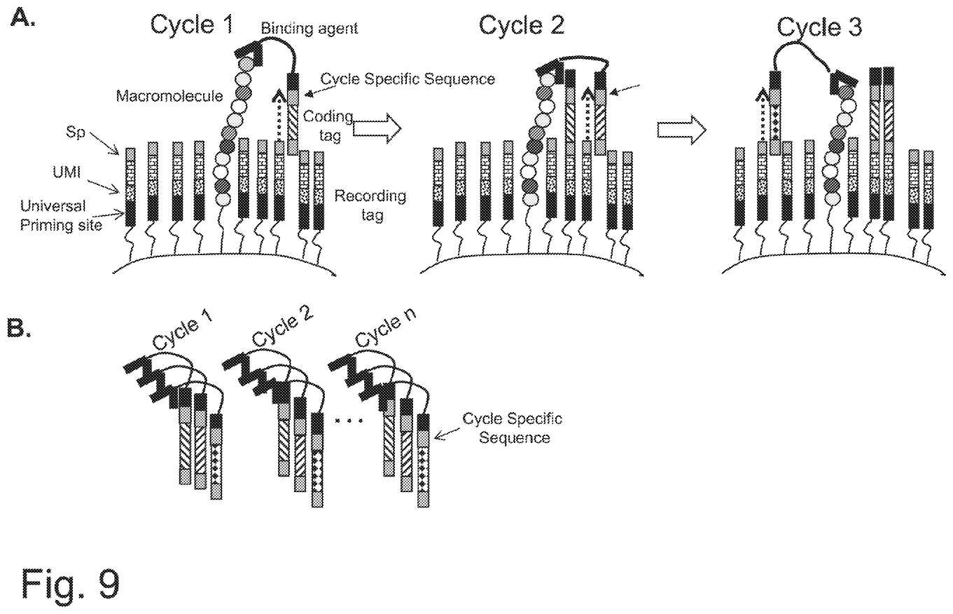

In certain embodiments, an ensemble of recording tags may be employed per polypeptide to improve the overall robustness and efficiency of coding tag information transfer (see, e.g., FIG. 9). The use of an ensemble of recording tags associated with a given polypeptide rather than a single recording tag improves the efficiency of library construction due to potentially higher coupling yields of coding tags to recording tags, and higher overall yield of libraries. The yield of a single concatenated extended recording tag is directly dependent on the stepwise yield of concatenation, whereas the use of multiple recording tags capable of accepting coding tag information does not suffer the exponential loss of concatenation.

An example of such an embodiment is shown in FIGS. 9 and 10. In FIGS. 9A and 10A, multiple recording tags are associated with a single polypeptide (by spatial co-localization or confinement of a single polypeptide to a single bead) on a solid support. Binding agents are exposed to the solid support in cyclical fashion and their corresponding coding tag transfers information to one of the co-localized multiple recording tags in each cycle. In the example shown in FIG. 9A, the binding cycle information is encoded into the spacer present on the coding tag. For each binding cycle, the set of binding agents is marked with a designated cycle-specific spacer sequence (FIGS. 9A and 9B). For example, in the case of NTAA binding agents, the binding agents to the same amino acid residue are be labelled with different coding tags or comprise cycle-specific information in the spacer sequence to denote both the binding agent identity and cycle number.

As illustrated in FIG. 9A, in a first cycle of binding (Cycle 1), a plurality of NTAA binding agents is contacted with the polypeptide. The binding agents used in Cycle 1 possess a common spacer sequence that is complementary to the spacer sequence of the recording tag. The binding agents used in Cycle 1 also possess a 3′-spacer sequence comprising Cycle 1 specific sequence. During binding Cycle 1, a first NTAA binding agent binds to the free terminus of the polypeptide, the complementary sequences of the common spacer sequence in the first coding tag and recording tag anneal, and the information of a first coding tag is transferred to a cognate recording tag via primer extension from the common spacer sequence. Following removal of the NTAA to expose a new NTAA, binding Cycle 2 contacts a plurality of NTAA binding agents that possess a common spacer sequence that is complementary to the spacer sequence of a recording tag. The binding agents used in Cycle 2 also possess a 3′-spacer sequence comprising Cycle 2 specific sequence. A second NTAA binding agent binds to the NTAA of the polypeptide, and the information of a second coding tag is transferred to a recording tag via primer extension. These cycles are repeated up to “n” binding cycles, generating a plurality of extended recording tags co-localized with the single polypeptide, wherein each extended recording tag possesses coding tag information from one binding cycle. Because each set of binding agents used in each successive binding cycle possess cycle specific spacer sequences in the coding tags, binding cycle information can be associated with binding agent information in the resulting extended recording tags

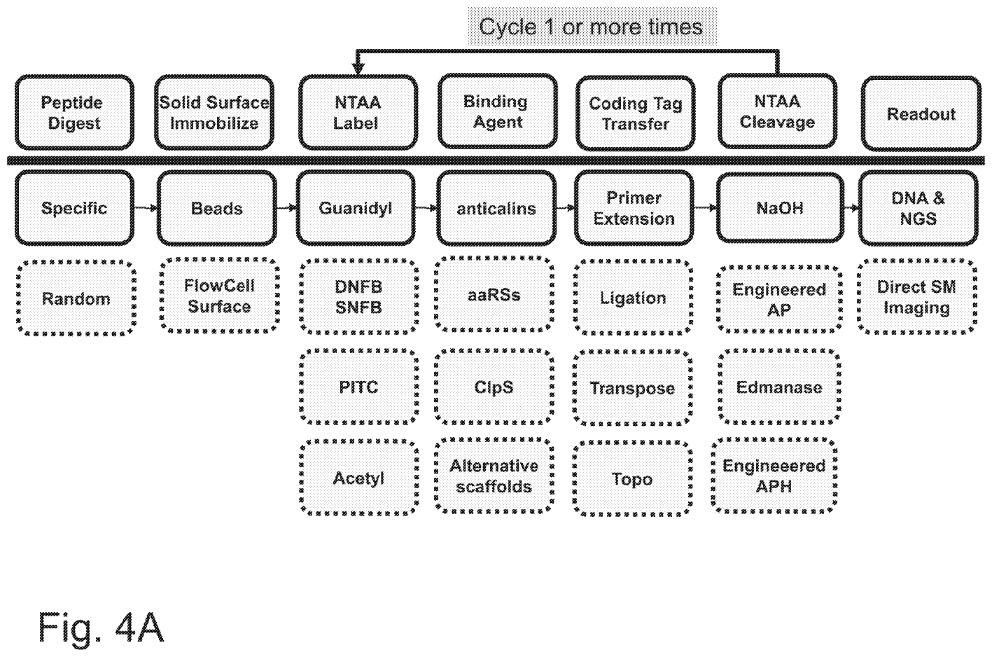

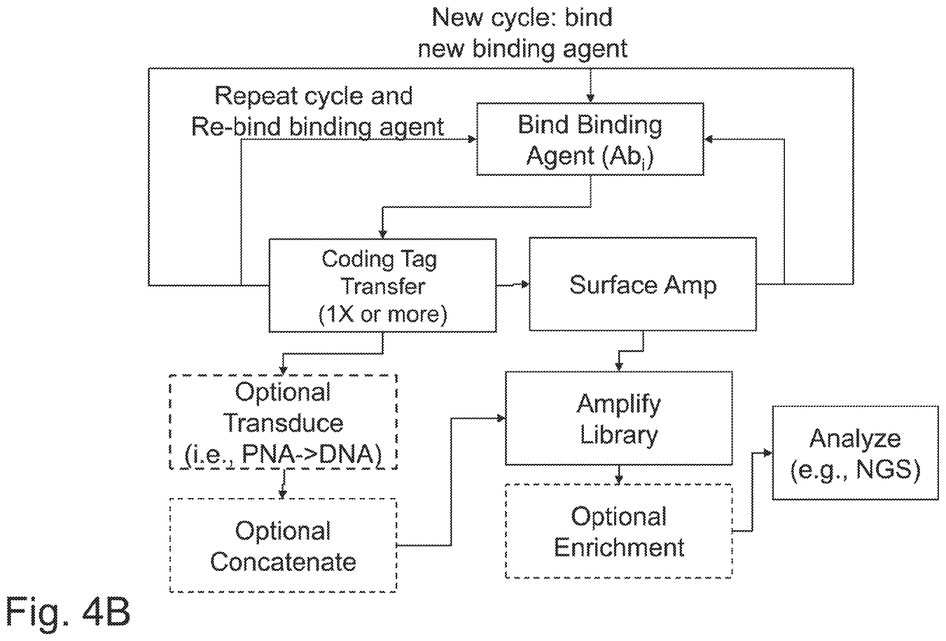

In an alternative embodiment, multiple recording tags are associated with a single polypeptide on a solid support (e.g., bead) as in FIG. 9A, but in this case binding agents used in a particular binding cycle have coding tags flanked by a cycle-specific spacer for the current binding cycle and a cycle specific spacer for the next binding cycle (FIGS. 10A and 10B). The reason for this design is to support a final assembly PCR step (FIG. 10C) to convert the population of extended recording tags into a single co-linear, extended recording tag. A library of single, co-linear extended recording tag can be subjected to enrichment, subtraction and/or normalization methods prior to sequencing. In the first binding cycle (Cycle 1), upon binding of a first binding agent, the information of a coding tag comprising a Cycle 1 specific spacer (C′1) is transferred to a recording tag comprising a complementary Cycle 1 specific spacer (C1) at its terminus. In the second binding cycle (Cycle 2), upon binding of a second binding agent, the information of a coding tag comprising a Cycle 2 specific spacer (C′2) is transferred to a different recording tag comprising a complementary Cycle 2 specific spacer (C2) at its terminus. This process continues until the n^{th} binding cycle. In some embodiments, the n^{th} coding tag in the extended recording tag is capped with a universal reverse priming sequence, e.g., the universal reverse priming sequence can be incorporated as part of the n^{th} coding tag design or the universal reverse priming sequence can be added in a subsequent reaction after the n^{th} binding cycle, such as an amplification reaction using a tailed primer. In some embodiments, at each binding cycle a polypeptide is exposed to a collection of binding agents joined to coding tags comprising identifying information regarding their corresponding binding agents and binding cycle information (FIG. 9 and FIG. 10). In a particular embodiment, following completion of the n^{th} binding cycle, the bead substrates coated with extended recording tags are placed in an oil emulsion such that on average there is fewer than or approximately equal to 1 bead/droplet. Assembly PCR is then used to amplify the extended recording tags from the beads, and the multitude of separate recording tags are assembled collinear order by priming via the cycle specific spacer sequences within the separate extended recording tags (FIG. 10C) (Xiong et al., 2008, FEMS Microbiol. Rev. 32:522-540). Alternatively, instead of using cycle-specific spacer with the binding agents' coding tags, a cycle specific spacer can be added separately to the extended recording tag during or after each binding cycle. One advantage of using a population of extended recording tags, which collectively represent a single polypeptide vs. a single concatenated extended recording tag representing a single polypeptide is that a higher concentration of recording tags can increase efficiency of transfer of the coding tag information. Moreover, a binding cycle can be repeated several times to ensure completion of cognate binding events. Furthermore, surface amplification of extended recording tags may be able to provide redundancy of information transfer (see FIG. 4B). If coding tag information is not always transferred, it should in most cases still be possible to use the incomplete collection of coding tag information to identify polypeptides that have very high information content, such as proteins. Even a short peptide can embody a very large number of possible protein sequences. For example, a 10-mer peptide has 20¹⁰ possible sequences. Therefore, partial or incomplete sequence that may contain deletions and/or ambiguities can often still be mapped uniquely.

In some embodiments, in which proteins in their native conformation are being queried, the cyclic binding assays are performed with binding agents harbouring coding tags comprised of a cleavable or nickable DNA strand within the spacer element proximal to the binding agent (FIG. 32). For example, the spacer proximal to the binding agent may have one or more uracil bases that can be nicked by uracil-specific excision reagent (USER). In another example, the spacer proximal to the binding agent may comprise a recognition sequence for a nicking endonuclease that hydrolyzes only one strand of a duplex. This design allows the non-denaturing removal of the binding agent from the extended recording tag and creates a free single stranded DNA spacer element for subsequent immunoassay cycles. In some embodiment, a uracil base is incorporated into the coding tag to permit enzymatic USER removal of the binding agent after the primer extension step (FIGS. 32E-F). After USER excision of uracils, the binding agent and truncated coding tag can be removed under a variety of mild conditions including high salt (4M NaCl, 25% formamide) and mild heat to disrupt the protein-binding agent interaction. The other truncated coding tag DNA stub remaining annealed on the recording tag (FIG. 32F) readily dissociates at slightly elevated temperatures.

Coding tags comprised of a cleavable or nickable DNA strand within the spacer element proximal to the binding agent also allows for a single homogeneous assay for transferring of coding tag information from multiple bound binding agents (see FIG. 33). In some embodiments, the coding tag proximal to the binding agent comprises a nicking endonuclease sequence motif, which is recognized and nicked by a nicking endonuclease at a defined sequence motif in the context of dsDNA. After binding of multiple binding agents, a combined polymerase extension (devoid of strand-displacement activity)+nicking endonuclease reagent mix is used to generate repeated transfers of coding tags to the proximal recording tag or extended recording tag. After each transfer step, the resulting extended recording tag-coding tag duplex is nicked by the nicking endonuclease releasing the truncated spacer attached to the binding agent and exposing the extended recording tag 3′ spacer sequence, which is capable of annealing to the coding tags of additional proximal bound binding agents (FIGS. 33B-D). The placement of the nicking motif in the coding tag spacer sequence is designed to create a metastable hybrid, which can easily be exchanged with a non-cleaved coding tag spacer sequence. In this way, if two or more binding agents simultaneously bind the same protein molecule, binding information via concatenation of coding tag information from multiply bound binding agents onto the recording tag occurs in a single reaction mix without any cyclic reagent exchanges (FIGS. 33C-D). This embodiment is particularly useful for the next generation protein assay (NGPA), especially with polyclonal antibodies (or mixed population of monoclonal antibody) to multivalent epitopes on a protein.

For embodiments involving analysis of denatured proteins, polypeptides, and peptides, the bound binding agent and annealed coding tag can be removed following primer extension by using highly denaturing conditions (e.g., 0.1-0.2 N NaOH, 6M Urea, 2.4 M guanidinium isothiocyanate, 95% formamide, etc.).

### Cyclic Transfer of Recording Tag Information to Coding Tags or Di-Tag Constructs

In another aspect, rather than writing information from the coding tag to the recording tag following binding of a binding agent to a polypeptide, information may be transferred from the recording tag comprising an optional UMI sequence (e.g. identifying a particular peptide or protein molecule) and at least one barcode (e.g., a compartment tag, partition barcode, sample barcode, spatial location barcode, etc.), to the coding tag, thereby generating an extended coding tag (see FIG. 11A). In certain embodiments, the binding agents and associated extended coding tags are collected following each binding cycle and, optionally, prior to Edman degradation chemistry steps. In certain embodiments, the coding tags comprise a binding cycle specific tag. After completion of all the binding cycles, such as detection of NTAAs in cyclic Edman degradation, the complete collection of extended coding tags can be amplified and sequenced, and information on the peptide determined from the association between UMI (peptide identity), encoder sequence (NTAA binding agent), compartment tag (single cell or subset of proteome), binding cycle specific sequence (cycle number), or any combination thereof. Library elements with the same compartment tag/UMI sequence map back to the same cell, subset of proteome, molecule, etc. and the peptide sequence can be reconstructed. This embodiment may be useful in cases where the recording tag sustains too much damage during the Edman degradation process.

Provided herein are methods for analyzing a plurality of polypeptides, comprising: (a) providing a plurality of polypeptides and associated recording tags joined to a solid support; (b) contacting the plurality of polypeptides with a plurality of binding agents capable of binding to the plurality of polypeptides, wherein each binding agent comprises a coding tag with identifying information regarding the binding agent; (c) (i) transferring the information of the polypeptide associated recording tags to the coding tags of the binding agents that are bound to the polypeptides to generate extended coding tags (see FIG. 11A); or (ii) transferring the information of polypeptide associated recording tags and coding tags of the binding agents that are bound to the polypeptides to a di-tag construct (see FIG. 11B); (d) collecting the extended coding tags or di-tag constructs; (e) optionally repeating steps (b)-(d) for one or more binding cycles; (f) analyzing the collection of extended coding tags or di-tag constructs.

In certain embodiments, the information transfer from the recording tag to the coding tag can be accomplished using a primer extension step where the 3′ terminus of recording tag is optionally blocked to prevent primer extension of the recording tag (see, e.g., FIG. 11A). The resulting extended coding tag and associated binding agent can be collected after each binding event and completion of information transfer. In an example illustrated in FIG. 11B, the recording tag is comprised of a universal priming site (U2′), a barcode (e.g., compartment tag “CT”), an optional UMI sequence, and a common spacer sequence (Sp1). In certain embodiments, the barcode is a compartment tag representing an individual compartment, and the UMI can be used to map sequence reads back to a particular protein or peptide molecule being queried. As illustrated in the example in FIG. 11B, the coding tag is comprised of a common spacer sequence (Sp2′), a binding agent encoder sequence, and universal priming site (U3). Prior to the introduction of the coding tag-labeled binding agent, an oligonucleotide (U2) that is complementary to the U2′ universal priming site of the recording tag and comprises a universal priming sequence U1 and a cycle specific tag, is annealed to the recording tag U2′. Additionally, an adapter sequence, Sp1′-Sp2, is annealed to the recording tag Sp1. This adapter sequence also capable of interacting with the Sp2′ sequence on the coding tag, bringing the recording tag and coding tag in proximity to each other. A gap-fill extension ligation assay is performed either prior to or after the binding event. If the gap fill is performed before the binding cycle, a post-binding cycle primer extension step is used to complete di-tag formation. After collection of di-tags across a number of binding cycles, the collection of di-tags is sequenced, and mapped back to the originating peptide molecule via the UMI sequence. It is understood that to maximize efficacy, the diversity of the UMI sequences must exceed the diversity of the number of single molecules tagged by the UMI.

In certain embodiments, the polypeptide may be obtained by fragmenting a protein from a biological sample.

The recording tag may be a DNA molecule, RNA molecule, PNA molecule, BNA molecule, XNA molecule, LNA molecule a γPNA molecule, or a combination thereof. The recording tag comprises a UMI identifying the polypeptide to which it is associated. In certain embodiments, the recording tag further comprises a compartment tag. The recording tag may also comprise a universal priming site, which may be used for downstream amplification. In certain embodiments, the recording tag comprises a spacer at its 3′ terminus. A spacer may be complementary to a spacer in the coding tag. The 3′-terminus of the recording tag may be blocked (e.g., photo-labile 3′ blocking group) to prevent extension of the recording tag by a polymerase, facilitating transfer of information of the polypeptide associated recording tag to the coding tag or transfer of information of the polypeptide associated recording tag and coding tag to a di-tag construct.

The coding tag comprises an encoder sequence identifying the binding agent to which the coding agent is linked. In certain embodiments, the coding tag further comprises a unique molecular identifier (UMI) for each binding agent to which the coding tag is linked. The coding tag may comprise a universal priming site, which may be used for downstream amplification. The coding tag may comprise a spacer at its 3′-terminus. The spacer may be complementary to the spacer in the recording tag and can be used to initiate a primer extension reaction to transfer recording tag information to the coding tag. The coding tag may also comprise a binding cycle specific sequence, for identifying the binding cycle from which an extended coding tag or di-tag originated.

Transfer of information of the recording tag to the coding tag may be effected by primer extension or ligation. Transfer of information of the recording tag and coding tag to a di-tag construct may be generated using a gap fill reaction, primer extension reaction, or both.

A di-tag molecule comprises functional components similar to that of an extended recording tag. A di-tag molecule may comprise a universal priming site derived from the recording tag, a barcode (e.g., compartment tag) derived from the recording tag, an optional unique molecular identifier (UMI) derived from the recording tag, an optional spacer derived from the recording tag, an encoder sequence derived from the coding tag, an optional unique molecular identifier derived from the coding tag, a binding cycle specific sequence, an optional spacer derived from the coding tag, and a universal priming site derived from the coding tag.

In certain embodiments, the recording tag can be generated using combinatorial concatenation of barcode encoding words. The use of combinatorial encoding words provides a method by which annealing and chemical ligation can be used to transfer information from a PNA recording tag to a coding tag or di-tag construct (see, e.g., FIGS. 12A-D). In certain embodiments where the methods of analyzing a peptide disclosed herein involve elimination of a terminal amino acid via an Edman degradation, it may be desirable employ recording tags resistant to the harsh conditions of Edman degradation, such as PNA. One harsh step in the Edman degradation protocol is anhydrous TFA treatment to eliminate the N-terminal amino acid. This step will typically destroy DNA. PNA, in contrast to DNA, is highly-resistant to acid hydrolysis. The challenge with PNA is that enzymatic methods of information transfer become more difficult, i.e., information transfer via chemical ligation is a preferred mode. In FIG. 11B, recording tag and coding tag information are written using an enzymatic gap-fill extension ligation step, but this is not currently feasibly with PNA template, unless a polymerase is developed that uses PNA. The writing of the barcode and UMI from the PNA recording tag to a coding tag is problematic due to the requirement of chemical ligation, products which are not easily amplified. Methods of chemical ligation have been extensively described in the literature (Gunderson et al. 1998, Genome Res. 8:1142-1153; Peng et al., 2010, Eur. J. Org. Chem. 4194-4197; El-Sagheer et al., 2011, Org. Biomol. Chem. 9:232-235; El-Sagheer et al., 2011, Proc. Natl. Acad. Sci. USA 108:11338-11343; Litovchick et al., 2014, Artif. DNA PNA XNA 5: e27896; Roloff et al., 2014, Methods Mol. Biol. 1050:131-141).

To create combinatorial PNA barcodes and UMI sequences, a set of PNA words from an n-mer library can be combinatorially ligated. If each PNA word derives from a space of 1,000 words, then four combined sequences generate a coding space of 1,000⁴=10¹² codes. In this way, from a starting set of 4,000 different DNA template sequences, over 10¹² PNA codes can be generated (FIG. 12A). A smaller or larger coding space can be generated by adjusting the number of concatenated words, or adjusting the number of elementary words. As such, the information transfer using DNA sequences hybridized to the PNA recording tag can be completed using DNA word assembly hybridization and chemical ligation (see FIG. 12B). After assembly of the DNA words on the PNA template and chemical ligation of the DNA words, the resulting intermediate can be used to transfer information to/from the coding tag (see FIG. 12C and FIG. 12D).

In certain embodiments, the polypeptide and associated recording tag are covalently joined to the solid support. The solid support may be a bead, a porous bead, a porous matrix, an array, a glass surface, a silicon surface, a plastic surface, a filter, a membrane, a PTFE membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere. The solid support may be a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof. In some embodiments, the support comprises gold, silver, a semiconductor or quantum dots. In some embodiments, the support is a nanoparticle and the nanoparticle comprises gold, silver, or quantum dots. In some embodiments, the support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.

In certain embodiments, the binding agent is a protein or a polypeptide. In some embodiments, the binding agent is a modified or variant aminopeptidase, a modified or variant amino acyl tRNA synthetase, a modified or variant anticalin, a modified or variant ClpS, or a modified or variant antibody or binding fragment thereof. In certain embodiments, the binding agent binds to a single amino acid residue, a di-peptide, a tri-peptide, or a post-translational modification of the peptide. In some embodiments, the binding agent binds to an N-terminal amino acid residue, a C-terminal amino acid residue, or an internal amino acid residue. In some embodiments, the binding agent binds to an N-terminal peptide, a C-terminal peptide, or an internal peptide. In some embodiments, the binding agent is a site-specific covalent label of an amino acid of post-translational modification of a peptide.

In certain embodiments, following contacting the plurality of polypeptides with a plurality of binding agents in step (b), complexes comprising the polypeptide and associated binding agents are dissociated from the solid support and partitioned into an emulsion of droplets or microfluidic droplets. In some embodiments, each microfluidic droplet comprises at most one complex comprising the polypeptide and the binding agents.

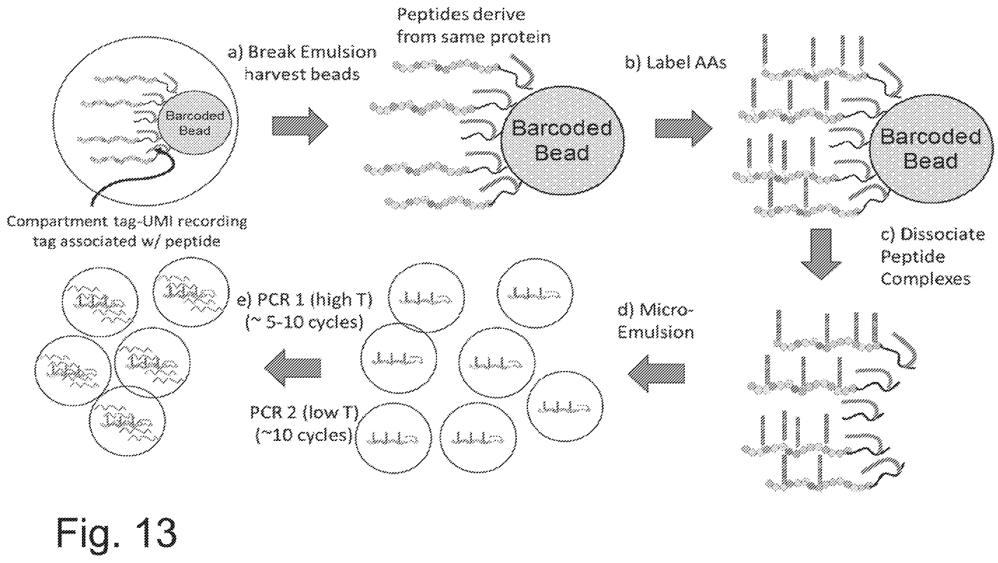

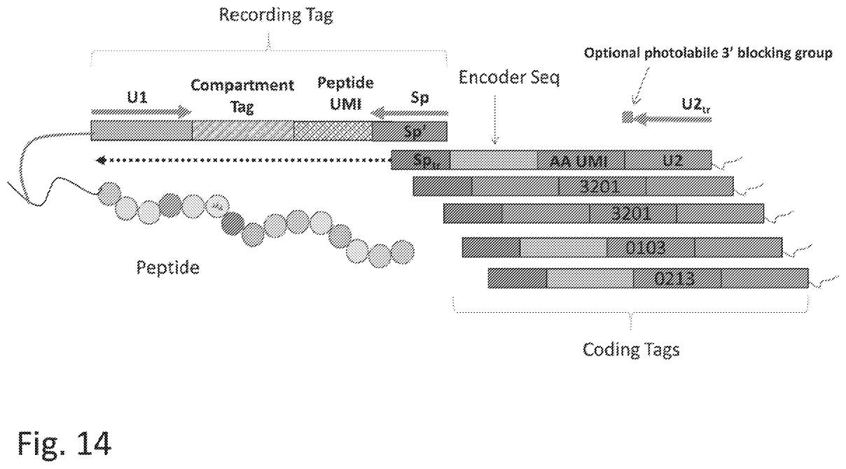

In certain embodiments, the recording tag is amplified prior to generating an extended coding tag or di-tag construct. In embodiments where complexes comprising the polypeptide and associated binding agents are partitioned into droplets or microfluidic droplets such that there is at most one complex per droplet, amplification of recording tags provides additional recording tags as templates for transferring information to coding tags or di-tag constructs (see FIG. 13 and FIG. 14). Emulsion fusion PCR may be used to transfer the recording tag information to the coding tag or to create a population of di-tag constructs.

The collection of extended coding tags or di-tag constructs that are generated may be amplified prior to analysis. Analysis of the collection of extended coding tags or di-tag constructs may comprise a nucleic acid sequencing method. The sequencing by synthesis, sequencing by ligation, sequencing by hybridization, polony sequencing, ion semiconductor sequencing, or pyrosequencing. The nucleic acid sequencing method may be single molecule real-time sequencing, nanopore-based sequencing, or direct imaging of DNA using advanced microscopy.

Edman degradation and methods that chemically label N-terminal amines such as PITC, Sanger's agent (DNFB), SNFB, acetylation reagents, amidination (guanidinylation) reagents, etc. can also functionalize internal amino acids and the exocyclic amines on standard nucleic acid or PNA bases such as adenine, guanine, and cytosine. In certain embodiments, the peptide's ε-amines of lysine residues are blocked with an acid anhydride, a guandination agent, or similar blocking reagent, prior to sequencing. Although exocyclic amines of DNA bases are much less reactive the primary N-terminal amine of peptides, controlling the reactivity of amine reactive agents toward N-terminal amines reducing non-target activity toward internal amino acids and exocyclic amines on DNA bases is important to the sequencing assay. The selectivity of the modification reaction can be modulated by adjusting reaction conditions such as pH, solvent (aqueous vs. organic, aprotic, non-polar, polar aprotic, ionic liquids, etc.), bases and catalysts, co-solvents, temperature, and time. In addition, reactivity of exocyclic amines on DNA bases is modulated by whether the DNA is in ssDNA or dsDNA form. To minimize modification, prior to NTAA chemical modification, the recording tag can be hybridized with complementary DNA probes: P1′, {Sample BCs}′, {Sp-BC}′, etc. In another embodiment, the use of nucleic acids having protected exocyclic amines can also be used (Ohkubo, Kasuya et al. 2008). In yet another embodiment, “less reactive” amine labeling compounds, such as SNFB, mitigates off-target labeling of internal amino acids and exocylic amines on DNA (Carty and Hirs 1968). SNFB is less reactive than DNFB due to the fact that the para sulfonyl group is more electron withdrawing the para nitro group, leading to less active fluorine substitution with SNFB than DNFB.

Titration of coupling conditions and coupling reagents to optimize NTAA ε-amine modification and minimize off-target amino acid modification or DNA modification is possible through careful selection of chemistry and reaction conditions (concentrations, temperature, time, pH, solvent type, etc.). For instance, DNFB is known to react with secondary amines more readily in aprotic solvents such as acetonitrile versus in water. Mild modification of the exocyclic amines may still allow a complementary probe to hybridize the sequence but would likely disrupt polymerase-based primer extension. It is also possible to protect the exocylic amine while still allowing hydrogen bonding. This was described in a recent publication in which protected bases are still capable of hybridizing to targets of interest (Ohkubo, Kasuya et al. 2008). In one embodiment, an engineered polymerase is used to incorporate nucleotides with protected bases during extension of the recording tag on a DNA coding tag template. In another embodiment, an engineered polymerase is used to incorporate nucleotides on a recording tag PNA template (w/ or w/o protected bases) during extension of the coding tag on the PNA recording tag template. In another embodiment, the information can be transferred from the recording tag to the coding tag by annealing an exogenous oligonucleotide to the PNA recording tag. Specificity of hybridization can be facilitated by choosing UMIs which are distinct in sequence space, such as designs based on assembly of n-mer words (Gerry, Witowski et al. 1999). While Edman-like N-terminal peptide degradation sequencing can be used to determine the linear amino acid sequence of the peptide, an alternative embodiment can be used to perform partial compositional analysis of the peptide with methods utilizing extended recording tags, extended coding tags, and di-tags. Binding agents or chemical labels can be used to identify both N-terminal and internal amino acids or amino acid modifications on a peptide. Chemical agents can covalently modify amino acids (e.g., label) in a site-specific manner (Sletten and Bertozzi 2009, Basle, Joubert et al. 2010) (Spicer and Davis 2014). A coding tag can be attached to a chemical labeling agent that targets a single amino acid, to facilitate encoding and subsequent identification of site-specific labeled amino acids (see, FIG. 13).

Peptide compositional analysis does not require cyclic degradation of the peptide, and thus circumvents issues of exposing DNA containing tags to harsh Edman chemistry. In a cyclic binding mode, one can also employ extended coding tags or di-tags to provide compositional information (amino acids or dipeptide/tripeptide information), PTM information, and primary amino acid sequence. In one embodiment, this composition information can be read out using an extended coding tag or di-tag approach described herein. If combined with UMI and compartment tag information, the collection of extended coding tags or di-tags provides compositional information on the peptides and their originating compartmental protein or proteins. The collection of extended coding tags or di-tags mapping back to the same compartment tag (and ostensibly originating protein molecule) is a powerful tool to map peptides with partial composition information. Rather than mapping back to the entire proteome, the collection of compartment tagged peptides is mapped back to a limited subset of protein molecules, greatly increasing the uniqueness of mapping.

Binding agents used herein may recognize a single amino acid, dipeptide, tripeptide, or even longer peptide sequence motifs. Tessler (2011, Digital Protein Analysis: Technologies for Protein Diagnostics and Proteomics through Single Molecule Detection. Ph.D., Washington University in St. Louis) demonstrated that relatively selective dipeptide antibodies can be generated for a subset of charged dipeptide epitopes (Tessler 2011). The application of directed evolution to alternate protein scaffolds (e.g., aaRSs, anticalins, ClpSs, etc.) and aptamers may be used to expand the set of dipeptide/tripeptide binding agents. The information from dipeptide/tripeptide compositional analysis coupled with mapping back to a single protein molecule may be sufficient to uniquely identify and quantitate each protein molecule. At a maximum, there are a total of 400 possible dipeptide combinations. However, a subset of the most frequent and most antigenic (charged, hydrophilic, hydrophobic) dipeptide should suffice to which to generate binding agents. This number may constitute a set of 40-100 different binding agents. For a set of 40 different binding agents, the average 10-mer peptide has about an 80% chance of being bound by at least one binding agent. Combining this information with all the peptides deriving from the same protein molecule may allow identification of the protein molecule. All this information about a peptide and its originating protein can be combined to give more accurate and precise protein sequence characterization.

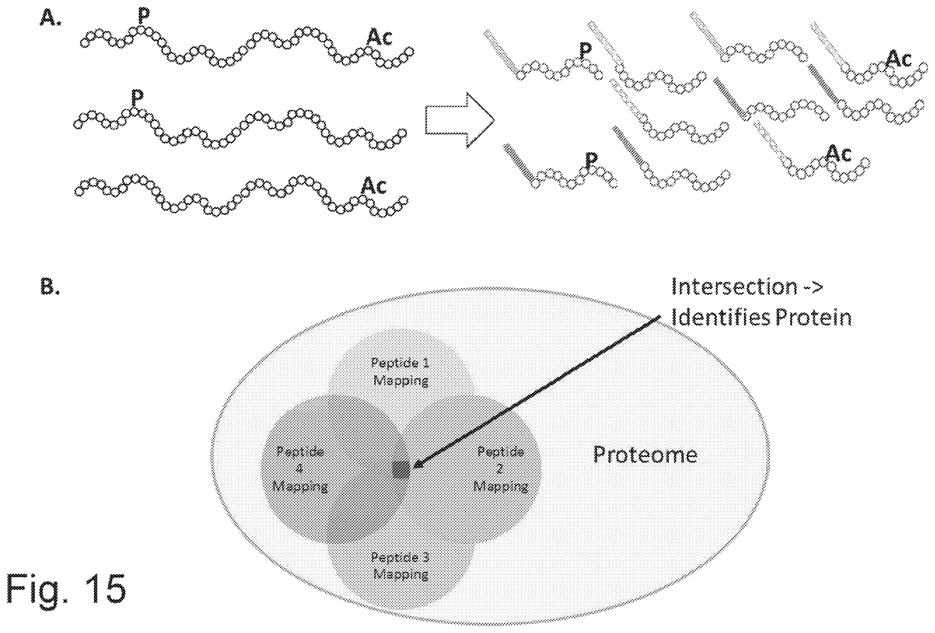

A recent digital protein characterization assay has been proposed that uses partial peptide sequence information (Swaminathan et al., 2015, PLoS Comput. Biol. 11:e1004080) (Yao, Docter et al. 2015). Namely, the approach employs fluorescent labeling of amino acids which are easily labeled using standard chemistry such as cysteine, lysine, arginine, tyrosine, aspartate/glutamate (Basle, Joubert et al. 2010). The challenge with partial peptide sequence information is that the mapping back to the proteome is a one-to-many association, with no unique protein identified. This one-to-many mapping problem can be solved by reducing the entire proteome space to limited subset of protein molecules to which the peptide is mapped back. In essence, a single partial peptide sequence may map back to 100's or 1000's of different protein sequences, however if it is known that a set of several peptides (for example, 10 peptides originating from a digest of a single protein molecule) all map back to a single protein molecule contained in the subset of protein molecules within a compartment, then it is easier to deduce the identity of the protein molecule. For instance, an intersection of the peptide proteome maps for all peptides originating from the same molecule greatly restricts the set of possible protein identities (see FIG. 15).

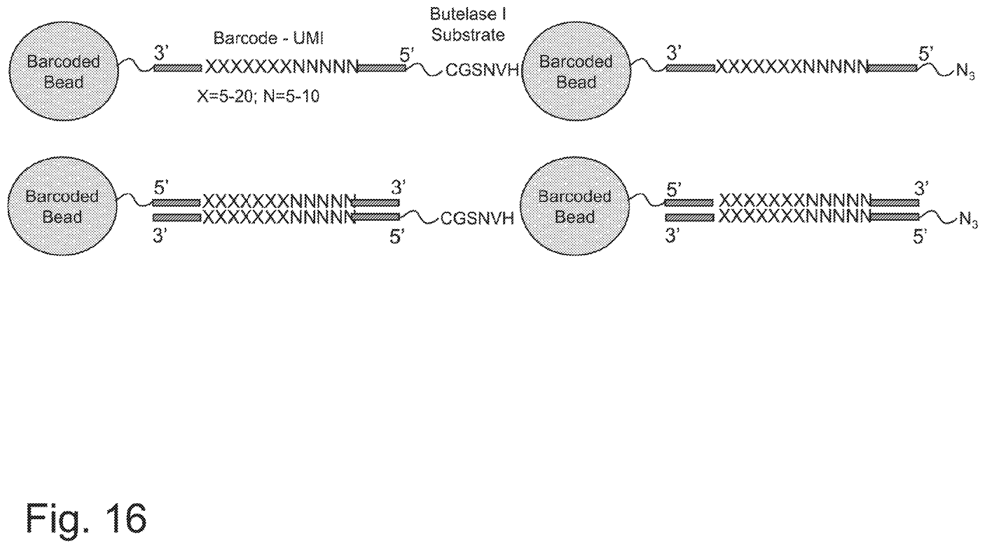

In particular, mappability of a partial peptide sequence or composition is significantly enhanced by making innovative use of compartmental tags and UMIs. Namely, the proteome is initially partitioned into barcoded compartments, wherein the compartmental barcode is also attached to a UMI sequence. The compartment barcode is a sequence unique to the compartment, and the UMI is a sequence unique to each barcoded molecule within the compartment (see FIG. 16). In one embodiment, this partitioning is accomplished using methods similar to those disclosed in PCT Publication WO2016/061517, which is incorporated by reference in its entirety, by direct interaction of a DNA tag labeled polypeptide with the surface of a bead via hybridization to DNA compartment barcodes attached to the bead (see FIG. 31). A primer extension step transfers information from the bead-linked compartment barcode to the DNA tag on the polypeptide (FIG. 20). In another embodiment, this partitioning is accomplished by co-encapsulating UMI containing, barcoded beads and protein molecules into droplets of an emulsion. In addition, the droplet optionally contains a protease that digests the protein into peptides. A number of proteases can be used to digest the reporter tagged polypeptides (Switzar, Giera et al. 2013). Co-encapsulation of enzymatic ligases, such as butelase I, with proteases may will call for modification to the enzyme, such as pegylation, to make it resistant to protease digestion (Frokjaer and Otzen 2005, Kang, Wang et al. 2010). After digestion, the peptides are ligated to the barcode-UMI tags. In some embodiments, the barcode-UMI tags are retained on the bead to facilitate downstream biochemical manipulations (see FIG. 13).

After barcode-UMI ligation to the peptides, the emulsion is broken and the beads harvested. The barcoded peptides can be characterized by their primary amino acid sequence, or their amino acid composition. Both types of information about the peptide can be used to map it back to a subset of the proteome. In general, sequence information maps back to a much smaller subset of the proteome than compositional information. Nonetheless, by combining information from multiple peptides (sequence or composition) with the same compartment barcode, it is possible to uniquely identify the protein or proteins from which the peptides originate. In this way, the entire proteome can be characterized and quantitated. Primary sequence information on the peptides can be derived by performing a peptide sequencing reaction with extended recording tag creation of a DNA Encoded Library (DEL) representing the peptide sequence. In some embodiments, the recording tag is comprised of a compartmental barcode and UMI sequence. This information is used along with the primary or PTM amino acid information transferred from the coding tags to generate the final mapped peptide information.

An alternative to peptide sequence information is to generate peptide amino acid or dipeptide/tripeptide compositional information linked to compartmental barcodes and UMIs. This is accomplished by subjecting the beads with UMI-barcoded peptides to an amino acid labeling step, in which select amino acids (internal) on each peptide are site-specifically labeled with a DNA tag comprising amino acid code information and another amino acid UMI (AA UMI) (see, FIG. 13). The amino acids (AAs) most tractable to chemical labeling are lysines, arginines, cysteines, tyrosines, tryptophans, and aspartates/glutamates, but it may also be feasible to develop labeling schemes for the other AAs as well (Mendoza and Vachet, 2009). A given peptide may contain several AAs of the same type. The presence of multiple amino acids of the same type can be distinguished by virtue of the attached AA UMI label. Each labeling molecule has a different UMI within the DNA tag enabling counting of amino acids. An alternative to chemical labeling is to “label” the AAs with binding agents. For instance, a tyrosine-specific antibody labeled with a coding tag comprising AA code information and an AA UMI could be used mark all the tyrosines of the peptides. The caveat with this approach is the steric hindrance encountered with large bulky antibodies, ideally smaller scFvs, anticalins, or ClpS variants would be used for this purpose.

In one embodiment, after tagging the AAs, information is transferred between the recording tag and multiple coding tags associated with bound or covalently coupled binding agents on the peptide by compartmentalizing the peptide complexes such that a single peptide is contained per droplet and performing an emulsion fusion PCR to construct a set of extended coding tags or di-tags characterizing the amino acid composition of the compartmentalized peptide. After sequencing the di-tags, information on peptides with the same barcodes can be mapped back to a single protein molecule.

In a particular embodiment, the tagged peptide complexes are disassociated from the bead (see FIG. 13), partitioned into small mini-compartments (e.g., micro-emulsion) such that on average only a single labeled/bound binding agent peptide complex resides in a given compartment. In a particular embodiment, this compartmentalization is accomplished through generation of micro-emulsion droplets (Shim, Ranasinghe et al. 2013, Shembekar, Chaipan et al. 2016). In addition to the peptide complex, PCR reagents are also co-encapsulated in the droplets along with three primers (U1, Sp, and U2ₜᵣ). After droplet formation, a few cycles of emulsion PCR are performed (˜5-10 cycles) at higher annealing temperature such than only U1 and Sp anneal and amplify the recording tag product (see FIG. 13). After this initial 5-10 cycles of PCR, the annealing temperature is reduced such that U2ₜᵣ and the Spₜᵣ on the amino acid code tags participate in the amplification, and another ˜10 rounds are performed. The three-primer emulsion PCR effectively combines the peptide UMI-barcode with all the AA code tags generating a di-tag library representation of the peptide and its amino acid composition. Other modalities of performing the three primer PCR and concatenation of the tags can also be employed. Another embodiment is the use of a 3′ blocked U2 primer activated by photo-deblocking, or addition of an oil soluble reductant to initiate 3′ deblocking of a labile blocked 3′ nucleotide. Post-emulsion PCR, another round of PCR can be performed with common primers to format the library elements for NGS sequencing.

In this way, the different sequence components of the library elements are used for counting and classification purposes. For a given peptide (identified by the compartment barcode-UMI combination), there are many library elements, each with an identifying AA code tag and AA UMI (see FIG. 13). The AA code and associated UMI is used to count the occurrences of a given amino acid type in a given peptide. Thus the peptide (perhaps a GluC, LysC, or Endo AsnN digest) is characterized by its amino acid composition (e.g., 2 Cys, 1 Lys, 1 Arg, 2 Tyr, etc.) without regard to spatial ordering. This nonetheless provides a sufficient signature to map the peptide to a subset of the proteome, and when used in combination with the other peptides derived from the same protein molecule, to uniquely identify and quantitate the protein.

### Processing and Analysis of Extended Recording Tags, Extended Coding Tags, or Di-Tags

Extended recording tag, extended coding tag, and di-tag libraries representing the polypeptide(s) of interest can be processed and analysed using a variety of nucleic acid sequencing methods. Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

A library of extended recording tags, extended coding tags, or di-tags may be amplified in a variety of ways. A library of extended recording tags, extended coding tags, or di-tags may undergo exponential amplification, e.g., via PCR or emulsion PCR. Emulsion PCR is known to produce more uniform amplification (Hori, Fukano et al. 2007). Alternatively, a library of extended recording tags, extended coding tags, or di-tags may undergo linear amplification, e.g., via in vitro transcription of template DNA using T7 RNA polymerase. The library of extended recording tags, extended coding tags, or di-tags can be amplified using primers compatible with the universal forward priming site and universal reverse priming site contained therein. A library of extended recording tags, extended coding tags, or di-tags can also be amplified using tailed primers to add sequence to either the 5′-end, 3′-end or both ends of the extended recording tags, extended coding tags, or di-tags. Sequences that can be added to the termini of the extended recording tags, extended coding tags, or di-tags include library specific index sequences to allow multiplexing of multiple libraries in a single sequencing run, adaptor sequences, read primer sequences, or any other sequences for making the library of extended recording tags, extended coding tags, or di-tags compatible for a sequencing platform. An example of a library amplification in preparation for next generation sequencing is as follows: a 20 μl PCR reaction volume is set up using an extended recording tag library eluted from ˜1 mg of beads (˜10 ng), 200 uM dNTP, 1 μM of each forward and reverse amplification primers, 0.5 μl (1U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of extended recording tags, extended coding tags, or di-tags can undergo target enrichment. Target enrichment can be used to selectively capture or amplify extended recording tags representing polypeptides of interest from a library of extended recording tags, extended coding tags, or di-tags before sequencing. Target enrichment for protein sequence is challenging because of the high cost and difficulty in producing highly-specific binding agents for target proteins. Antibodies are notoriously non-specific and difficult to scale production across thousands of proteins. The methods of the present disclosure circumvent this problem by converting the protein code into a nucleic acid code which can then make use of a wide range of targeted DNA enrichment strategies available for DNA libraries. Peptides of interest can be enriched in a sample by enriching their corresponding extended recording tags. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., 2010, Nature Methods 7: 111-118; Bodi et al., J. Biomol. Tech. 2013, 24:73-86; Ballester et al., 2016, Expert Review of Molecular Diagnostics 357-372; Mertes et al., 2011, Brief Funct. Genomics 10:374-386; Nilsson et al., 1994, Science 265:2085-8; each of which are incorporated herein by reference in their entirety).

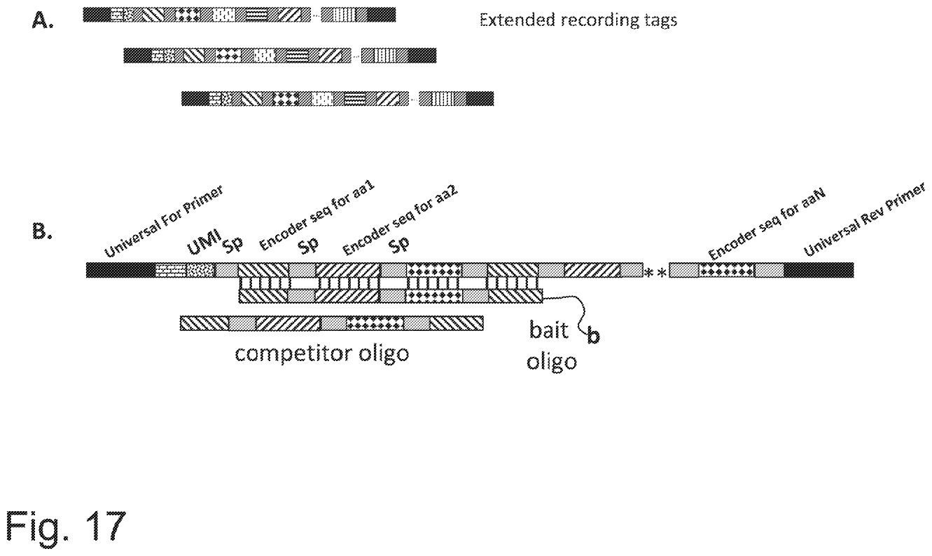

In one embodiment, a library of extended recording tags, extended coding tags, or di-tags is enriched via a hybrid capture-based assay (see, e.g., FIG. 17A and FIG. 17B). In a hybrid-capture based assay, the library of extended recording tags, extended coding tags, or di-tags is hybridized to target-specific oligonucleotides or “bait oligonucleotide” that are labelled with an affinity tag (e.g., biotin). Extended recording tags, extended coding tags, or di-tags hybridized to the target-specific oligonucleotides are “pulled down” via their affinity tags using an affinity ligand (e.g., streptavidin coated beads), and background (non-specific) extended recording tags are washed away (see, e.g., FIG. 17). The enriched extended recording tags, extended coding tags, or di-tags are then obtained for positive enrichment (e.g., eluted from the beads).

For bait oligonucleotides synthesized by array-based “in situ” oligonucleotide synthesis and subsequent amplification of oligonucleotide pools, competing baits can be engineered into the pool by employing several sets of universal primers within a given oligonucleotide array. For each type of universal primer, the ratio of biotinylated primer to non-biotinylated primer controls the enrichment ratio. The use of several primer types enables several enrichment ratios to be designed into the final oligonucleotide bait pool.

A bait oligonucleotide can be designed to be complementary to an extended recording tag, extended coding tag, or di-tag representing a polypeptide of interest. The degree of complementarity of a bait oligonucleotide to the spacer sequence in the extended recording tag, extended coding tag, or di-tag can be from 0% to 100%, and any integer in between. This parameter can be easily optimized by a few enrichment experiments. In some embodiments, the length of the spacer relative to the encoder sequence is minimized in the coding tag design or the spacers are designed such that they unavailable for hybridization to the bait sequences. One approach is to use spacers that form a secondary structure in the presence of a cofactor. An example of such a secondary structure is a G-quadruplex, which is a structure formed by two or more guanine quartets stacked on top of each other (Bochman, Paeschke et al. 2012). A guanine quartet is a square planar structure formed by four guanine bases that associate through Hoogsteen hydrogen bonding. The G-quadruplex structure is stabilized in the presence of a cation, e.g., K+ ions vs. Li+ ions.

To minimize the number of bait oligonucleotides employed, a set of relatively unique peptides from each protein can be bioinformatically identified, and only those bait oligonucleotides complementary to the corresponding extended recording tag library representations of the peptides of interest are used in the hybrid capture assay. Sequential rounds or enrichment can also be carried out, with the same or different bait sets.

To enrich the entire length of a polypeptide in a library of extended recording tags, extended coding tags, or di-tags representing fragments thereof (e.g., peptides), “tiled” bait oligonucleotides can be designed across the entire nucleic acid representation of the protein.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

Targeted enrichment methods can also be used in a negative selection mode to selectively remove extended recording tags, extended coding tags, or di-tags from a library before sequencing. Thus, in the example described above using biotinylated bait oligonucleotides and streptavidin coated beads, the supernatant is retained for sequencing while the bait-oligonucleotide:extended recording tag, extended coding tag, or di-tag hybrids bound to the beads are not analysed. Examples of undesirable extended recording tags, extended coding tags, or di-tags that can be removed are those representing over abundant polypeptide species, e.g., for proteins, albumin, immunoglobulins, etc.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment (FIG. 17). The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended recording tag, extended coding tag, or di-tag library. This approach works best for defined length libraries originating from peptides generated by site-specific protease digestion such as trypsin, LysC, GluC, etc. In one example, normalization can be accomplished by denaturing a double-stranded library and allowing the library elements to re-anneal. The abundant library elements re-anneal more quickly than less abundant elements due to the second-order rate constant of bimolecular hybridization kinetics (Bochman, Paeschke et al. 2012). The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., 2002, Genome Res. 12:1935-42) which destroys the dsDNA library elements.

Any combination of fractionation, enrichment, and subtraction methods, of the polypeptides before attachment to the solid support and/or of the resulting extended recording tag library can economize sequencing reads and improve measurement of low abundance species.

In some embodiments, a library of extended recording tags, extended coding tags, or di-tags is concatenated by ligation or end-complementary PCR to create a long DNA molecule comprising multiple different extended recorder tags, extended coding tags, or di-tags, respectively (Du et al., 2003, BioTechniques 35:66-72; Muecke et al., 2008, Structure 16:837-841; U.S. Pat. No. 5,834,252, each of which is incorporated by reference in its entirety). This embodiment is preferable for nanopore sequencing in which long strands of DNA are analyzed by the nanopore sequencing device.

In some embodiments, direct single molecule analysis is performed on an extended recording tag, extended coding tag, or di-tag (see, e.g., Harris et al., 2008, Science 320:106-109). The extended recording tags, extended coding tags, or di-tags can be analysed directly on the solid support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labelled of decoding oligonucleotides (Gunderson et al., 2004, Genome Res. 14:970-7) can be used to ascertain both the identity and order of the coding tags within the extended recording tag. In some embodiments, the binding agents may be labelled with cycle-specific coding tags as described above (see also, Gunderson et al., 2004, Genome Res. 14:970-7). Cycle-specific coding tags will work for both a single, concatenated extended recording tag representing a single polypeptide, or for a collection of extended recording tags representing a single polypeptide.

Following sequencing of the extended reporter tag, extended coding tag, or di-tag libraries, the resulting sequences can be collapsed by their UMIs and then associated to their corresponding polypeptides and aligned to the totality of the proteome. Resulting sequences can also be collapsed by their compartment tags and associated to their corresponding compartmental proteome, which in a particular embodiment contains only a single or a very limited number of protein molecules. Both protein identification and quantification can easily be derived from this digital peptide information.

In some embodiments, the coding tag sequence can be optimized for the particular sequencing analysis platform. In a particular embodiment, the sequencing platform is nanopore sequencing. In some embodiments, the sequencing platform has a per base error rate of >5%, >10%, >15%, >20%, >25%, or >30%. For example, if the extended recording tag is to be analyzed using a nanopore sequencing instrument, the barcode sequences (e.g., encoder sequences) can be designed to be optimally electrically distinguishable in transit through a nanopore. Peptide sequencing according to the methods described herein may be well-suited for nanopore sequencing, given that the single base accuracy for nanopore sequencing is still rather low (75%-85%), but determination of the “encoder sequence” should be much more accurate (>99%). Moreover, a technique called duplex interrupted nanopore sequencing (DI) can be employed with nanopore strand sequencing without the need for a molecular motor, greatly simplifying the system design (Derrington, Butler et al. 2010). Readout of the extended recording tag via DI nanopore sequencing requires that the spacer elements in the concatenated extended recording tag library be annealed with complementary oligonucleotides. The oligonucleotides used herein may comprise LNAs, or other modified nucleic acids or analogs to increase the effective Tm of the resultant duplexes. As the single-stranded extended recording tag decorated with these duplex spacer regions is passed through the pore, the double strand region will become transiently stalled at the constriction zone enabling a current readout of about three bases adjacent to the duplex region. In a particular embodiment for DI nanopore sequencing, the encoder sequence is designed in such a way that the three bases adjacent to the spacer element create maximally electrically distinguishable nanopore signals (Derrington et al., 2010, Proc. Natl. Acad. Sci. USA 107:16060-5). As an alternative to motor-free DI sequencing, the spacer element can be designed to adopt a secondary structure such as a G-quartet, which will transiently stall the extended recording tag, extended coding tag, or di-tag as it passes through the nanopore enabling readout of the adjacent encoder sequence (Shim, Tan et al. 2009, Zhang, Zhang et al. 2016). After proceeding past the stall, the next spacer will again create a transient stall, enabling readout of the next encoder sequence, and so forth.

The methods disclosed herein can be used for analysis, including detection, quantitation and/or sequencing, of a plurality of polypeptides simultaneously (multiplexing). Multiplexing as used herein refers to analysis of a plurality of polypeptides in the same assay. The plurality of polypeptides can be derived from the same sample or different samples. The plurality of polypeptides can be derived from the same subject or different subjects. The plurality of polypeptides that are analyzed can be different polypeptides, or the same polypeptide derived from different samples. A plurality of polypeptides includes 2 or more polypeptides, 5 or more polypeptides, 10 or more polypeptides, 50 or more polypeptides, 100 or more polypeptides, 500 or more polypeptides, 1000 or more polypeptides, 5,000 or more polypeptides, 10,000 or more polypeptides, 50,000 or more polypeptides, 100,000 or more polypeptides, 500,000 or more polypeptides, or 1,000,000 or more polypeptides.

Sample multiplexing can be achieved by upfront barcoding of recording tag labeled polypeptide samples. Each barcode represents a different sample, and samples can be pooled prior to cyclic binding assays or sequence analysis. In this way, many barcode-labeled samples can be simultaneously processed in a single tube. This approach is a significant improvement on immunoassays conducted on reverse phase protein arrays (RPPA) (Akbani, Becker et al. 2014, Creighton and Huang 2015, Nishizuka and Mills 2016). In this way, the present disclosure essentially provides a highly digital sample and analyte multiplexed alternative to the RPPA assay with a simple workflow.

### Characterization of Polypeptides Via Cyclic Rounds of NTAA Recognition, Recording Tag Extension, and NTAA Elimination

In certain embodiments, the methods for analyzing a polypeptide provided in the present disclosure comprise multiple binding cycles, where the polypeptide is contacted with a plurality of binding agents, and successive binding of binding agents transfers historical binding information in the form of a nucleic acid based coding tag to at least one recording tag associated with the polypeptide. In this way, a historical record containing information about multiple binding events is generated in a nucleic acid format.

In embodiments relating to methods of analyzing peptide polypeptides using an N-terminal degradation based approach (see, FIG. 3, FIG. 4, FIG. 41, and FIG. 42), following contacting and binding of a first binding agent to an n NTAA of a peptide of n amino acids and transfer of the first binding agent's coding tag information to a recording tag associated with the peptide, thereby generating a first order extended recording tag, the n NTAA is eliminated as described herein. Elimination of the n NTAA converts the n−1 amino acid of the peptide to an N-terminal amino acid, which is referred to herein as an n−1 NTAA. As described herein, the n NTAA may optionally be functionalized with a moiety (e.g., PTC, DNP, SNP, acetyl, amidinyl, modified with a modified with a diheterocyclic methanimine, etc.), which is particularly useful in conjunction with cleavage enzymes that are engineered to bind to a functionalized form of NTAA. In some embodiments, the functionalized NTAA includes a ligand group that is capable of covalent binding to a binding agent. If then NTAA was functionalized, the n−1 NTAA is then functionalized with the same moiety. A second binding agent is contacted with the peptide and binds to the n−1 NTAA, and the second binding agent's coding tag information is transferred to the first order extended recording tag thereby generating a second order extended recording tag (e.g., for generating a concatenated n^{th} order extended recording tag representing the peptide), or to a different recording tag (e.g., for generating multiple extended recording tags, which collectively represent the peptide). Elimination of the n−1 NTAA converts the n−2 amino acid of the peptide to an N-terminal amino acid, which is referred to herein as n−2 NTAA. Additional binding, transfer, elimination, and optionally NTAA functionalization, can occur as described above up to n amino acids to generate an n^{th} order extended recording tag or n separate extended recording tags, which collectively represent the peptide. As used herein, an n “order” when used in reference to a binding agent, coding tag, or extended recording tag, refers to the n binding cycle, wherein the binding agent and its associated coding tag is used or the n binding cycle where the extended recording tag is created.

In some embodiments, contacting of the first binding agent and second binding agent to the polypeptide, and optionally any further binding agents (e.g., third binding agent, fourth binding agent, fifth binding agent, and so on), are performed at the same time. For example, the first binding agent and second binding agent, and optionally any further order binding agents, can be pooled together, for example to form a library of binding agents. In another example, the first binding agent and second binding agent, and optionally any further order binding agents, rather than being pooled together, are added simultaneously to the polypeptide. In one embodiment, a library of binding agents comprises at least 20 binding agents that selectively bind to the 20 standard, naturally occurring amino acids.

In other embodiments, the first binding agent and second binding agent, and optionally any further order binding agents, are each contacted with the polypeptide in separate binding cycles, added in sequential order. In certain embodiments, multiple binding agents are used at the same time, in parallel. This parallel approach saves time and reduces non-specific binding by non-cognate binding agents to a site that is bound by a cognate binding agent (because the binding agents are in competition).

The length of the final extended recording tags generated by the methods described herein is dependent upon multiple factors, including the length of the coding tag (e.g., encoder sequence and spacer), the length of the recording tag (e.g., unique molecular identifier, spacer, universal priming site, bar code), the number of binding cycles performed, and whether coding tags from each binding cycle are transferred to the same extended recording tag or to multiple extended recording tags. In an example for a concatenated extended recording tag representing a peptide and produced by an Edman degradation like elimination method, if the coding tag has an encoder sequence of 5 bases that is flanked on each side by a spacer of 5 bases, the coding tag information on the final extended recording tag, which represents the peptide's binding agent history, is 10 bases×number of cycles. For a 20-cycle run, the extended recording is at least 200 bases (not including the initial recording tag sequence). This length is compatible with standard next generation sequencing instruments.

After the final binding cycle and transfer of the final binding agent's coding tag information to the extended recording tag, the recorder tag can be capped by addition of a universal reverse priming site via ligation, primer extension or other methods known in the art. In some embodiments, the universal forward priming site in the recording tag is compatible with the universal reverse priming site that is appended to the final extended recording tag. In some embodiments, a universal reverse priming site is an Illumina P7 primer (5′-CAAGCAGAAGACGGCATACGAGAT-3′-SEQ ID NO:134) or an Illumina P5 primer (5′-AATGATACGGCGACCACCGA-3′-SEQ ID NO:133). The sense or antisense P7 may be appended, depending on strand sense of the recording tag. An extended recording tag library can be cleaved or amplified directly from the solid support (e.g., beads) and used in traditional next generation sequencing assays and protocols.

In some embodiments, a primer extension reaction is performed on a library of single stranded extended recording tags to copy complementary strands thereof.

The NGPS peptide sequencing assay, which may be referred to as ProteoCode, comprises several chemical and enzymatic steps in a cyclical progression. The fact that NGPS sequencing is single molecule confers several key advantages to the process. The first key advantage of single molecule assay is the robustness to inefficiencies in the various cyclical chemical/enzymatic steps. This is enabled through the use of cycle-specific barcodes present in the coding tag sequence.

Using cycle-specific coding tags, we track information from each cycle. Since this is a single molecule sequencing approach, even 70% efficiency at each binding/transfer cycle in the sequencing process is more than sufficient to generate mappable sequence information. As an example, a ten-base peptide sequence “CPVQLWVDST” (SEQ ID NO:169) might be read as “CPXQXWXDXT” (SEQ ID NO:170) on our sequence platform (where X=any amino acid; the presence an amino acid is inferred by cycle number tracking). This partial amino acid sequence read is more than sufficient to uniquely map it back to the human p53 protein using BLASTP. As such, none of our processes have to be perfect to be robust. Moreover, when cycle-specific barcodes are combined with our partitioning concepts, absolute identification of the protein can be accomplished with only a few amino acids identified out of 10 positions since we know what set of peptides map to the original protein molecule (via compartment barcodes).

Suitable sequencing methods for use in the invention include, but are not limited to, sequencing by hybridization, sequencing by synthesis technology (e.g., HiSeq™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeq™, Illumina), massively parallel semiconductor sequencing (e.g., Ion Torrent), pyrosequencing technology (e.g., GS FLX and GS Junior Systems, Roche/454), and nanopore sequence (e.g., Oxford Nanopore Technologies).

### Protein Normalization Via Fractionation, Compartmentalization, and Limited Binding Capacity Resins.

One of the key challenges with proteomics analysis is addressing the large dynamic range in protein abundance within a sample. Proteins span greater than 10 orders of dynamic range within plasma (even “Top 20” depleted plasma). In certain embodiments, subtraction of certain protein species (e.g., highly abundant proteins) from the sample is performed prior to analysis. This can be accomplished, for example, using commercially available protein depletion reagents such as Sigma's PROT20 immuno-depletion kit, which deplete the top 20 plasma proteins. Additionally, it would be useful to have an approach that greatly reduced the dynamic range even further to a manageable 3-4 orders. In certain embodiments, a protein sample dynamic range can be modulated by fractionating the protein sample using standard fractionation methods, including electrophoresis and liquid chromatography (Zhou, Ning et al. 2012), or partitioning the fractions into compartments (e.g., droplets) loaded with limited capacity protein binding beads/resin (e.g. hydroxylated silica particles) (McCormick 1989) and eluting bound protein. Excess protein in each compartmentalized fraction is washed away.

Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivitized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

### Partitioning of Proteome of a Single Cell or Molecular Subsampling

In another aspect, the present disclosure provides methods for massively-parallel analysis of proteins in a sample using barcoding and partitioning techniques. Current approaches to protein analysis involve fragmentation of protein polypeptides into shorter peptide molecules suitable for peptide sequencing. Information obtained using such approaches is therefore limited by the fragmentation step and excludes, e.g., long range continuity information of a protein, including post-translational modifications, protein-protein interactions occurring in each sample, the composition of a protein population present in a sample, or the origin of the protein polypeptide, such as from a particular cell or population of cells. Long range information of post-translation modifications within a protein molecule (e.g., proteoform characterization) provides a more complete picture of biology, and long range information on what peptides belong to what protein molecule provides a more robust mapping of peptide sequence to underlying protein sequence (see FIG. 15A). This is especially relevant when the peptide sequencing technology only provides incomplete amino acid sequence information, such as information from only 5 amino acid types. By using the partitioning methods disclosed herein, combined with information from a number of peptides originating from the same protein molecule, the identity of the protein molecule (e.g. proteoform) can be more accurately assessed. Association of compartment tags with proteins and peptides derived from same compartment(s) facilitates reconstruction of molecular and cellular information. In typical proteome analysis, cells are lysed and proteins digested into short peptides, disrupting global information on which proteins derive from which cell or cell type, and which peptides derive from which protein or protein complex. This global information is important to understanding the biology and biochemistry within cells and tissues.

Partitioning refers to the random assignment of a unique barcode to a subpopulation of polypeptides from a population of polypeptides within a sample. Partitioning may be achieved by distributing polypeptides into compartments. A partition may be comprised of the polypeptides within a single compartment or the polypeptides within multiple compartments from a population of compartments.

A subset of polypeptides or a subset of a protein sample that has been separated into or on the same physical compartment or group of compartments from a plurality (e.g., millions to billions) of compartments are identified by a unique compartment tag. Thus, a compartment tag can be used to distinguish constituents derived from one or more compartments having the same compartment tag from those in another compartment (or group of compartments) having a different compartment tag, even after the constituents are pooled together.

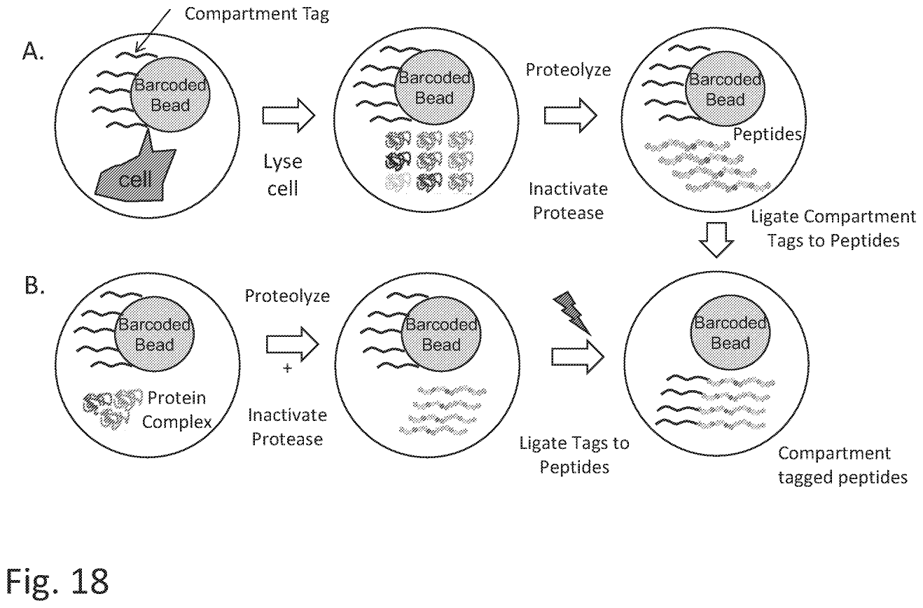

The present disclosure provides methods of enhancing protein analysis by partitioning a complex proteome sample (e.g., a plurality of protein complexes, proteins, or polypeptides) or complex cellular sample into a plurality of compartments, wherein each compartment comprises a plurality of compartment tags that are the same within an individual compartment (save for an optional UMI sequence) and are different from the compartment tags of other compartments (see, FIG. 18-20). The compartments optionally comprise a solid support (e.g., bead) to which the plurality of compartment tags are joined thereto. The plurality of protein complexes, proteins, or polypeptides are fragmented into a plurality of peptides, which are then contacted to the plurality of compartment tags under conditions sufficient to permit annealing or joining of the plurality of peptides with the plurality of compartment tags within the plurality of compartments, thereby generating a plurality of compartment tagged peptides. Alternatively, the plurality of protein complexes, proteins, or polypeptides are joined to a plurality of compartment tags under conditions sufficient to permit annealing or joining of the plurality of protein complexes, proteins or polypeptides with the plurality of compartment tags within a plurality of compartments, thereby generating a plurality of compartment tagged protein complexes, proteins, polypeptides. The compartment tagged protein complexes, proteins, or polypeptides are then collected from the plurality of compartments and optionally fragmented into a plurality of compartment tagged peptides. One or more compartment tagged peptides are analyzed according to any of the methods described herein.

In certain embodiments, compartment tag information is transferred to a recording tag associated with a polypeptide (e.g., peptide) via primer extension (FIG. 5) or ligation (FIG. 6).

In some embodiments, the compartment tags are free in solution within the compartments. In other embodiments, the compartment tags are joined directly to the surface of the compartment (e.g., well bottom of microtiter or picotiter plate) or a bead or bead within a compartment.

A compartment can be an aqueous compartment (e.g., microfluidic droplet) or a solid compartment. A solid compartment includes, for example, a nanoparticle, a microsphere, a microtiter or picotiter well or a separated region on an array, a glass surface, a silicon surface, a plastic surface, a filter, a membrane, nylon, a silicon wafer chip, a flow cell, a flow through chip, a biochip including signal transducing electronics, an ELISA plate, a spinning interferometry disc, a nitrocellulose membrane, or a nitrocellulose-based polymer surface. In certain embodiments, each compartment contains, on average, a single cell.

A solid support can be any support surface including, but not limited to, a bead, a microbead, an array, a glass surface, a silicon surface, a plastic surface, a filter, a membrane, a PTFE membrane, a PTFE membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyactic acid, polyorthoesters, functionalized silane, polypropylfumerate, polyvinylchloride, collagen, glycosaminoglycans, polyamino acids, or any combination thereof. In certain embodiments, a solid support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or any combinations thereof.

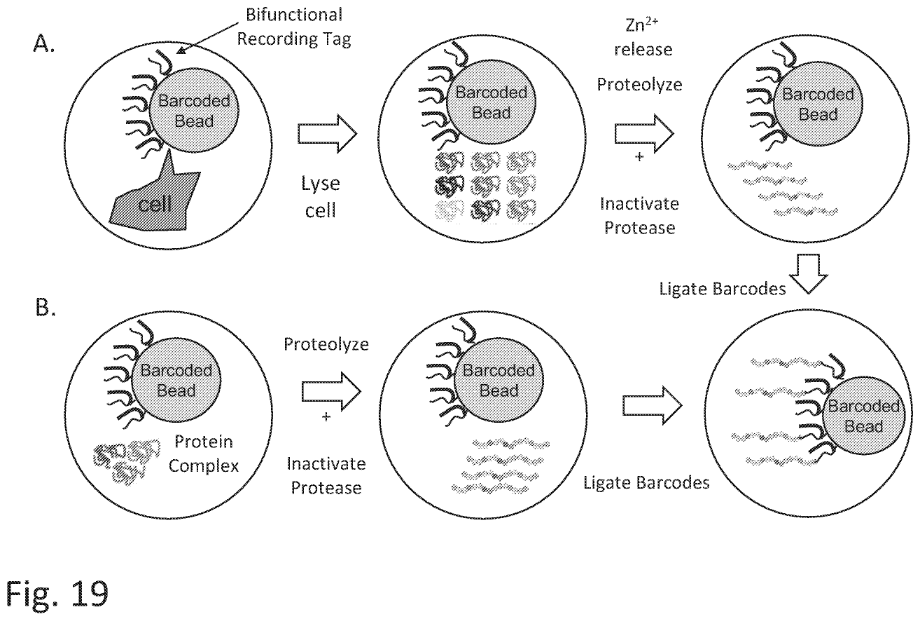

Various methods of partitioning samples into compartments with compartment tagged beads is reviewed in Shembekar et al., (Shembekar, Chaipan et al. 2016). In one example, the proteome is partitioned into droplets via an emulsion to enable global information on protein molecules and protein complexes to be recorded using the methods disclosed herein (see, e.g., FIG. 18 and FIG. 19). In certain embodiments, the proteome is partitioned in compartments (e.g., droplets) along with compartment tagged beads, an activate-able protease (directly or indirectly via heat, light, etc.), and a peptide ligase engineered to be protease-resistant (e.g., modified lysines, pegylation, etc.). In certain embodiments, the proteome can be treated with a denaturant to assess the peptide constituents of a protein or polypeptide. If information regarding the native state of a protein is desired, an interacting protein complex can be partitioned into compartments for subsequent analysis of the peptides derived therefrom.

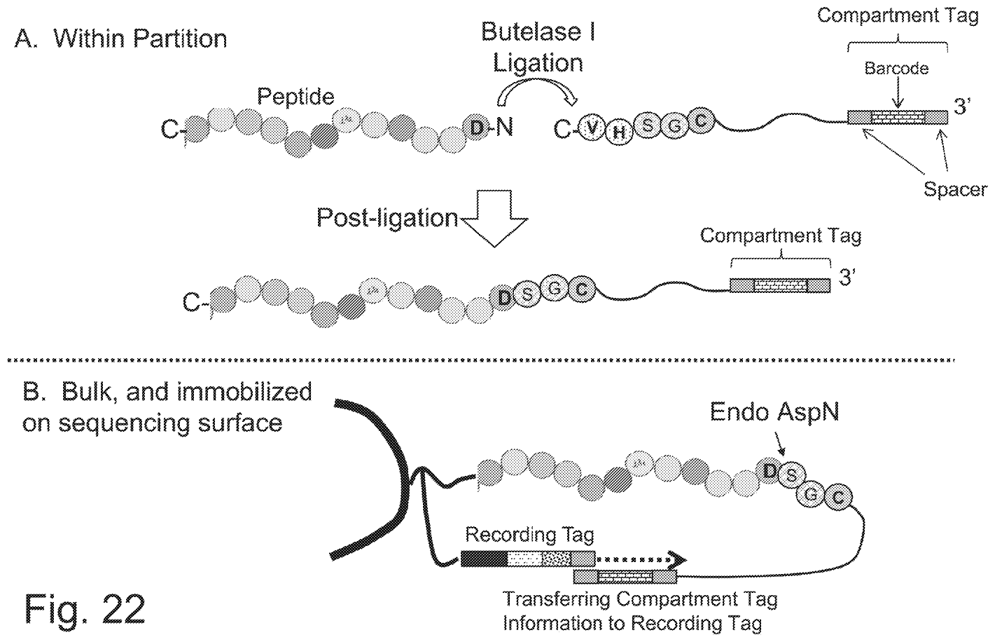

A compartment tag comprises a barcode, which is optionally flanked by a spacer or universal primer sequence on one or both sides. The primer sequence can be complementary to the 3′ sequence of a recording tag, thereby enabling transfer of compartment tag information to the recording tag via a primer extension reaction (see, FIGS. 22A-B). The barcode can be comprised of a single stranded nucleic acid molecule attached to a solid support or compartment or its complementary sequence hybridized to solid support or compartment, or both strands (see, e.g., FIG. 16). A compartment tag can comprise a functional moiety, for example attached to the spacer, for coupling to a peptide. In one example, a functional moiety (e.g., aldehyde) is one that is capable of reacting with the N-terminal amino acid residue on the plurality of peptides. In another example, the functional moiety is capable of reacting with an internal amino acid residue (e.g., lysine or lysine labeled with a “click” reactive moiety) on the plurality of peptides. In another embodiment, the functional moiety may simply be a complementary DNA sequence capable of hybridizing to a DNA tag-labeled protein. Alternatively, a compartment tag can be a chimeric molecule, further comprising a peptide comprising a recognition sequence for a protein ligase (e.g., butelase I or homolog thereof) to allow ligation of the compartment tag to a peptide of interest (see, FIG. 22A). A compartment tag can be a component within a larger nucleic acid molecule, which optionally further comprises a unique molecular identifier for providing identifying information on the peptide that is joined thereto, a spacer sequence, a universal priming site, or any combination thereof. This UMI sequence generally differs among a population of compartment tags within a compartment. In certain embodiments, a compartment tag is a component within a recording tag, such that the same tag that is used for providing individual compartment information is also used to record individual peptide information for the peptide attached thereto.

In certain embodiments, compartment tags can be formed by printing, spotting, ink-jetting the compartment tags into the compartment. In certain embodiments, a plurality of compartment tagged beads is formed, wherein one barcode type is present per bead, via split-and-pool oligonucleotide ligation or synthesis as described by Klein et al., 2015, Cell 161:1187-1201; Macosko et al., 2015, Cell 161:1202-1214; and Fan et al., 2015, Science 347:1258367. Compartment tagged beads can also be formed by individual synthesis or immobilization. In certain embodiments, the compartment tagged beads further comprise bifunctional recording tags, in which one portion comprises the compartment tag comprising a recording tag, and the other portion comprises a functional moiety to which the digested peptides can be coupled (FIG. 19 and FIG. 20).

In certain embodiments, the plurality of proteins or polypeptides within the plurality of compartments is fragmented into a plurality of peptides with a protease. A protease can be a metalloprotease. In certain embodiments, the activity of the metalloprotease is modulated by photo-activated release of metallic cations. Examples of endopeptidases that can be used include: trypsin, chymotrypsin, elastase, thermolysin, pepsin, clostripan, glutamyl endopeptidase (GluC), endopeptidase ArgC, peptidyl-asp metallo-endopeptidase (AspN), endopeptidase LysC and endopeptidase LysN. Their mode of activation varies depending on buffer and divalent cation requirements. Optionally, following sufficient digestion of the proteins or polypeptides into peptide fragments, the protease is inactivated (e.g., heat, fluoro-oil or silicone oil soluble inhibitor, such as a divalent cation chelation agent).

In certain embodiments of peptide barcoding with compartment tags, a protein molecule (optionally, denatured polypeptide) is labeled with DNA tags by conjugation of the DNA tags to ε-amine moieties of the protein's lysine groups or indirectly via click chemistry attachment to a protein/polypeptide pre-labeled with a reactive click moiety such as alkyne (see FIG. 2B and FIG. 20A). The DNA tag-labeled polypeptides are then partitioned into compartments comprising compartment tags (e.g., DNA barcodes bound to beads contained within droplets) (see FIG. 20B), wherein a compartment tag contains a barcode that identifies each compartment. In one embodiment, a single protein/polypeptide molecule is co-encapsulated with a single species of DNA barcodes associated with a bead (see FIG. 20B). In another embodiment, the compartment can constitute the surface of a bead with attached compartment (bead) tags similar to that described in PCT Publication WO2016/061517 (incorporated by reference in its entirety), except as applied to proteins rather than DNA. The compartment tag can comprise a barcode (BC) sequence, a universal priming site (U1′), a UMI sequence, and a spacer sequence (Sp). In one embodiment, concomitant with or after partitioning, the compartment tags are cleaved from the bead and hybridize to the DNA tags attached to the polypeptide, for example via the complementary U1 and U1′ sequences on the DNA tag and compartment tag, respectively. For partitioning on beads, the DNA tag-labeled protein can be directly hybridized to the compartment tags on the bead surface (see, FIG. 20C). After this hybridization step, the polypeptides with hybridized DNA tags are extracted from the compartments (e.g., emulsion “cracked”, or compartment tags cleaved from bead), and a polymerase-based primer extension step is used to write the barcode and UMI information to the DNA tags on the polypeptide to yield a compartment barcoded recording tag (see, FIG. 20D). A LysC protease digestion may be used to cleave the polypeptide into constituent peptides labeled at their C-terminal lysine with a recording tag containing universal priming sequences, a compartment tag, and a UMI (see, FIG. 20E). In one embodiment, the LysC protease is engineered to tolerate DNA-tagged lysine residues. The resultant recording tag labeled peptides are immobilized to a solid substrate (e.g., bead) at an appropriate density to minimize intermolecular interactions between recording tagged peptides (see, FIGS. 20E and 20F).

Attachment of the peptide to the compartment tag (or vice versa) can be directly to an immobilized compartment tag, or to its complementary sequence (if double stranded). Alternatively, the compartment tag can be detached from the solid support or surface of the compartment, and the peptide and solution phase compartment tag joined within the compartment. In one embodiment, the functional moiety on the compartment tag (e.g., on the terminus of oligonucleotide) is an aldehyde which is coupled directly to the amine N-terminus of the peptide through a Schiff base (see FIG. 16). In another embodiment, the compartment tag is constructed as a nucleic acid-peptide chimeric molecule comprising peptide motif (n-X . . . XXCGSHV-c; SEQ ID NO: 139) for a protein ligase. The nucleic acid-peptide compartment tag construct is conjugated to digested peptides using a peptide ligase, such as butelase I or a homolog thereof. Butelase I, and other asparaginyl endopeptidase (AEP) homologues, can be used to ligate the C-terminus of the oligonucleotide-peptide compartment tag construct to the N-terminus of the digested peptides (Nguyen, Wang et al. 2014, Nguyen, Cao et al. 2015). This reaction is fast and highly efficient. The resultant compartment tagged peptides can be subsequently immobilized to a solid support for nucleic-acid peptide analysis as described herein.

In certain embodiments, compartment tags that are joined to a solid support or surface of a compartment are released prior to joining the compartment tags with the plurality of fragmented peptides (see FIG. 18). In some embodiments, following collection of the compartment tagged peptides from the plurality of compartments, the compartment tagged peptides are joined to a solid support in association with recording tags. Compartment tag information can then be transferred from the compartment tag on the compartment tagged peptide to the associated recording tag (e.g., via a primer extension reaction primed from complementary spacer sequences within the recording tag and compartment tag). In some embodiments, the compartment tags are then removed from the compartment tagged peptides prior to peptide analysis according to the methods described herein. In further embodiments, the sequence specific protease (e.g., Endo AspN) that is initially used to digest the plurality of proteins is also used to remove the compartment tag from the N terminus of the peptide after transfer of the compartment tag information to the associated recording tag (see FIG. 22B).

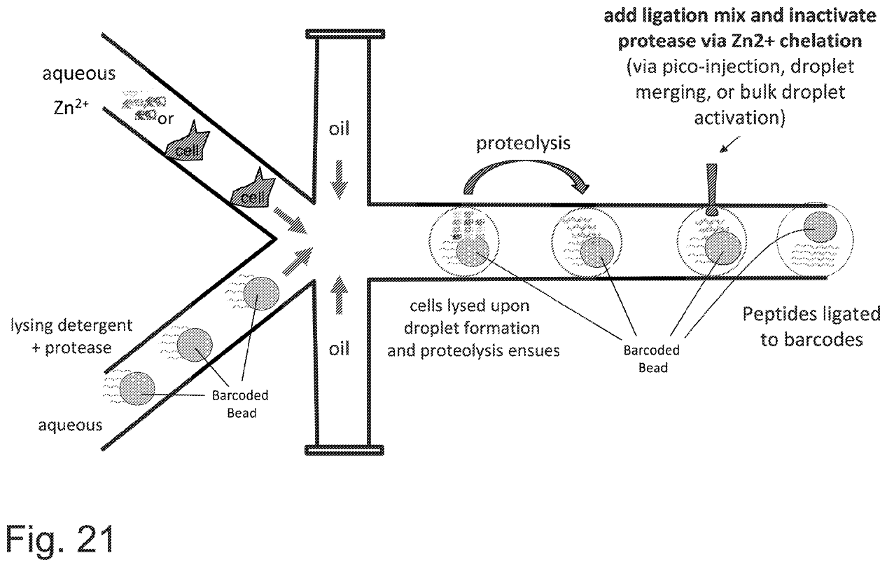

Approaches for compartmental-based partitioning include droplet formation through microfluidic devices using T-junctions and flow focusing, emulsion generation using agitation or extrusion through a membrane with small holes (e.g., track etch membrane), etc. (see, FIG. 21). A challenge with compartmentalization is addressing the interior of the compartment. In certain embodiments, it may be difficult to conduct a series of different biochemical steps within a compartment since exchanging fluid components is challenging. As previously described, one can modify a limited feature of the droplet interior, such as pH, chelating agent, reducing agents, etc. by addition of the reagent to the fluoro-oil of the emulsion. However, the number of compounds that have solubility in both aqueous and organic phases is limited. One approach is to limit the reaction in the compartment to essentially the transfer of the barcode to the molecule of interest.

After labeling of the proteins/peptides with recording tags comprised of compartment tags (barcodes), the protein/peptides are immobilized on a solid-support at a suitable density to favor intramolecular transfer of information from the coding tag of a bound cognate binding agent to the corresponding recording tag/tags attached to the bound peptide or protein molecule. Intermolecular information transfer is minimized by controlling the intermolecular spacing of molecules on the surface of the solid-support.

In certain embodiments, the compartment tags need not be unique for each compartment in a population of compartments. A subset of compartments (two, three, four, or more) in a population of compartments may share the same compartment tag. For instance, each compartment may be comprised of a population of bead surfaces which act to capture a subpopulation of polypeptides from a sample (many molecules are captured per bead). Moreover, the beads comprise compartment barcodes which can be attached to the captured polypeptides. Each bead has only a single compartment barcode sequence, but this compartment barcode may be replicated on other beads within the compartment (many beads mapping to the same barcode). There can be (although not required) a many-to-one mapping between physical compartments and compartment barcodes, moreover, there can be (although not required) a many-to-one mapping between polypeptides within a compartment. A partition barcode is defined as an assignment of a unique barcode to a subsampling of polypeptides from a population of polypeptides within a sample. This partition barcode may be comprised of identical compartment barcodes arising from the partitioning of polypeptides within compartments labeled with the same barcode. The use of physical compartments effectively subsamples the original sample to provide assignment of partition barcodes. For instance, a set of beads labeled with 10,000 different compartment barcodes is provided. Furthermore, suppose in a given assay, that a population of 1 million beads are used in the assay. On average, there are 100 beads per compartment barcode (Poisson distribution). Further suppose that the beads capture an aggregate of 10 million polypeptides. On average, there are 10 polypeptides per bead, with 100 compartments per compartment barcode, there are effectively 1000 polypeptides per partition barcode (comprised of 100 compartment barcodes for 100 distinct physical compartments).

In another embodiment, single molecule partitioning and partition barcoding of polypeptides is accomplished by labeling polypeptides (chemically or enzymatically) with an amplifiable DNA UMI tag (e.g., recording tag) at the N or C terminus, or both (see FIG. 37). DNA tags are attached to the body of the polypeptide (internal amino acids) via non-specific photo-labeling or specific chemical attachment to reactive amino acids such as lysines as illustrated in FIG. 2B. Information from the recording tag attached to the terminus of the peptide is transferred to the DNA tags via an enzymatic emulsion PCR (Williams, Peisajovich et al. 2006, Schutze, Rubelt et al. 2011) or emulsion in vitro transcription/reverse transcription (IVT/RT) step. In the preferred embodiment, a nanoemulsion is employed such that, on average, there is fewer than a single polypeptide per emulsion droplet with size from 50 nm-1000 nm (Nishikawa, Sunami et al. 2012, Gupta, Eral et al. 2016). Additionally, all the components of PCR are included in the aqueous emulsion mix including primers, dNTPs, Mg2+, polymerase, and PCR buffer. If IVT/RT is used, then the recording tag is designed with a T7/SP6 RNA polymerase promoter sequence to generate transcripts that hybridize to the DNA tags attached to the body of the polypeptide (Ryckelynck, Baudrey et al. 2015). A reverse transcriptase (RT) copies the information from the hybridized RNA molecule to the DNA tag. In this way, emulsion PCR or IVT/RT can be used to effectively transfer information from the terminus recording tag to multiple DNA tags attached to the body of the polypeptide.

Encapsulation of cellular contents via gelation in beads is a useful approach to single cell analysis (Tamminen and Virta 2015, Spencer, Tamminen et al. 2016). Barcoding single cell droplets enables all components from a single cell to be labeled with the same identifier (Klein, Mazutis et al. 2015, Gunderson, Steemers et al. 2016, Zilionis, Nainys et al. 2017). Compartment barcoding can be accomplished in a number of ways including direct incorporation of unique barcodes into each droplet by droplet joining (Raindance), by introduction of a barcoded beads into droplets (10× Genomics), or by combinatorial barcoding of components of the droplet post encapsulation and gelation using and split-pool combinatorial barcoding as described by Gunderson et al. (Gunderson, Steemers et al. 2016) and PCT Publication WO2016/130704, incorporated by reference in its entirety. A similar combinatorial labeling scheme can also be applied to nuclei as described by Adey et al. (Vitak, Torkenczy et al. 2017).

The above droplet barcoding approaches have been used for DNA analysis but not for protein analysis. Adapting the above droplet barcoding platforms to work with proteins requires several innovative steps. The first is that barcodes are primarily comprised of DNA sequences, and this DNA sequence information needs to be conferred to the protein analyte. In the case of a DNA analyte, it is relatively straightforward to transfer DNA information onto a DNA analyte. In contrast, transferring DNA information onto proteins is more challenging, particularly when the proteins are denatured and digested into peptides for downstream analysis. This requires that each peptide be labeled with a compartment barcode. The challenge is that once the cell is encapsulated into a droplet, it is difficult to denature the proteins, protease digest the resultant polypeptides, and simultaneously label the peptides with DNA barcodes. Encapsulation of cells in polymer forming droplets and their polymerization (gelation) into porous beads, which can be brought up into an aqueous buffer, provides a vehicle to perform multiple different reaction steps, unlike cells in droplets (Tamminen and Virta 2015, Spencer, Tamminen et al. 2016) (Gunderson, Steemers et al. 2016). Preferably, the encapsulated proteins are crosslinked to the gel matrix to prevent their subsequent diffusion from the gel beads. This gel bead format allows the entrapped proteins within the gel to be denatured chemically or enzymatically, labeled with DNA tags, protease digested, and subjected to a number of other interventions. FIG. 38 depicts exemplary encapsulation and lysis of a single cell in a gel matrix.

### Tissue and Single Cell Spatial Proteomics

Another use of barcodes is the spatial segmentation of a tissue on the surface an array of spatially distributed DNA barcode sequences. If tissue proteins are labelled with DNA recording tags comprising barcodes reflecting the spatial position of the protein within the cellular tissue mounted on the array surface, then the spatial distribution of protein analytes within the tissue slice can later be reconstructed after sequence analysis, much as is done for spatial transcriptomics as described by Stahl et al. (2016, Science 353(6294):78-82) and Crosetto et al. (Corsetto, Bienko et al., 2015). The attachment of spatial barcodes can be accomplished by releasing array-bound barcodes from the array and diffusing them into the tissue section, or alternatively, the proteins in the tissue section can be labeled with DNA recording tags, and then the proteins digested with a protease to release labeled peptides that can diffuse and hybridize to spatial barcodes on the array. The barcode information can then be transferred (enzymatically or chemically) to the recording tags attached to the peptides.

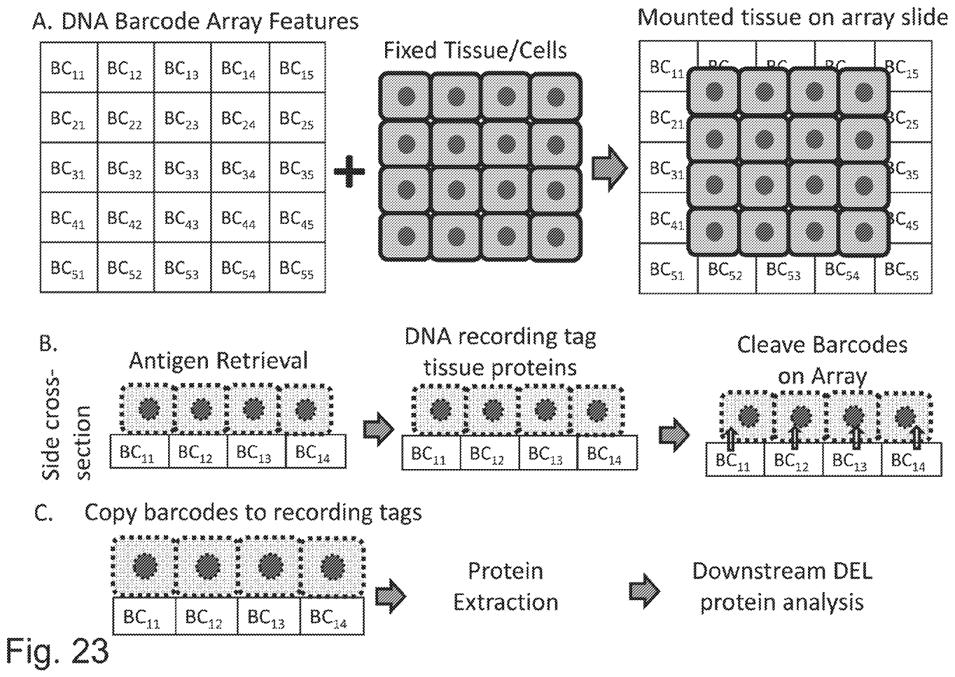

Spatial barcoding of the proteins within a tissue can be accomplished by placing a fixed/permeabilized tissue slice, chemically labelled with DNA recording tags, on a spatially encoded DNA array, wherein each feature on the array has a spatially identifiable barcode (see, FIG. 23). To attach an array barcode to the DNA tag, the tissue slice can be digested with a protease, releasing DNA tag labelled peptides, which can diffuse and hybridize to proximal array features adjacent to the tissue slice. The array barcode information can be transferred to the DNA tag using chemical/enzymatic ligation or polymerase extension. Alternatively, rather than allowing the labelled peptides to diffuse to the array surface, the barcodes sequences on the array can be cleaved and allowed to diffuse into proximal areas on the tissue slice and hybridize to DNA tag-labelled proteins therein. Once again, the barcoding information can be transferred by chemical/enzymatic ligation or polymerase extension. In this second case, protease digestion can be performed following transfer of barcode information. The result of either approach is a collection of recording tag-labelled protein or peptides, wherein the recording tag comprises a barcode harbouring 2-D spatial information of the protein/peptides's location within the originating tissue. Moreover, the spatial distribution of post-translational modifications can be characterized. This approach provides a sensitive and highly-multiplexed in situ digital immunohistochemistry assay, and should form the basis of modern molecular pathology leading to much more accurate diagnosis and prognosis.

In another embodiment, spatial barcoding can be used within a cell to identify the protein constituents/PTMs within the cellular organelles and cellular compartments (Christoforou et al., 2016, Nat. Commun. 7:8992, incorporated by reference in its entirety). A number of approaches can be used to provide intracellular spatial barcodes, which can be attached to proximal proteins. In one embodiment, cells or tissue can be sub-cellular fractionated into constituent organelles, and the different protein organelle fractions barcoded. Other methods of spatial cellular labelling are described in the review by Marx, 2015, Nat Methods 12:815-819, incorporated by reference in its entirety; similar approaches can be used herein.

### Kits

Provided in some aspects are kits for analyzing a polypeptide which contain (a) a reagent for providing the polypeptide optionally associated directly or indirectly with a recording; (b) a reagent for functionalizing the terminal amino acid of the polypeptide, selected from a compound of Formula (AA) as described herein or a compound of Formula R³—NCS as described herein; (c) a binding agent comprising a binding portion capable of binding to the functionalized terminal amino acid and (c1) a coding tag with identifying information regarding the first binding agent, or (c2) a detectable label; and (d) a reagent for transferring the information of the first coding tag to the recording tag to generate an extended recording tag; and optionally (e) a reagent for analyzing the extended recording tag or a reagent for detecting the first detectable label.

In some embodiments of any of the kits provided herein, Q is selected from the group consisting of —C₁₋₆ alkyl, —C₂₋₆ alkenyl, —C₂₋₆ alkynyl, aryl, heteroaryl, heterocyclyl, —N═C═S, —CN, —C(O)Rⁿ, —C(O)OR^{o}, —SR^{p} or —S(O)₂R^{q}; wherein the —C₁₋₆alkyl, —C₂₋₆alkenyl, —C₂₋₆ alkynyl, aryl, heteroaryl, and heterocyclyl are each unsubstituted or substituted, and Rⁿ, R^{o}, R^{p}, and R^{q} are each independently selected from the group consisting of —C₁₋₆alkyl, —C₁₋₆haloalkyl, —C₂₋₆ alkenyl, —C₂₋₆ alkynyl, aryl, heteroaryl, and heterocyclyl. In some embodiments, Q is selected from the group consisting of

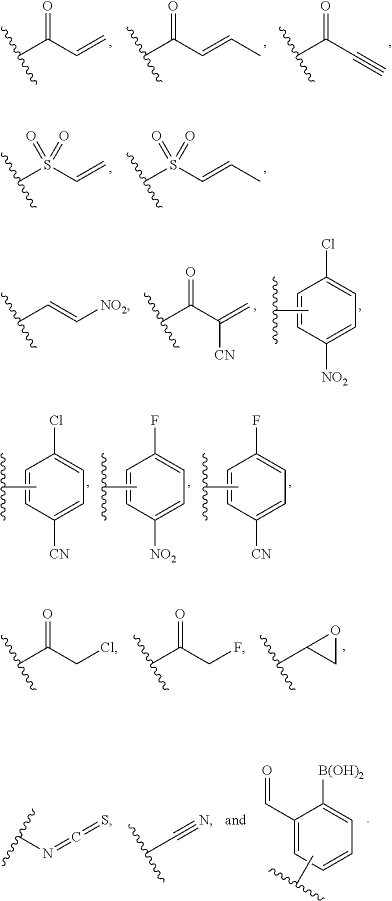

In some embodiments of any of the kits provided herein, Q is a fluorophore.

In some embodiments of any of the kits provided herein, the binding agent binds to a terminal amino acid residue, terminal di-amino-acid residues, or terminal tri-amino-acid residues. In some embodiments, the binding agent binds to a post-translationally modified amino acid.

In some embodiments of any of the kits provided herein, the recording tag comprises a nucleic acid, an oligonucleotide, a modified oligonucleotide, a DNA molecule, a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the DNA molecule is backbone modified, sugar modified, or nucleobase modified. In some embodiments, the DNA molecule has nucleobase protecting groups such as Alloc, electrophilic protecting groups such as thiranes, acetyl protecting groups, nitrobenzyl protecting groups, sulfonate protecting groups, or traditional base-labile protecting groups including Ultramild reagents. In some embodiments, the recording tag comprises a universal priming site. In some embodiments, the universal priming site comprises a priming site for amplification, sequencing, or both. In some embodiments, the recording tag comprises a unique molecule identifier (UMI). In some embodiments, the recording tag comprises a barcode. In some embodiments, the recording tag comprises a spacer at its 3′-terminus.

In some embodiments of any of the kits provided herein, the reagents for providing the polypeptide and an associated recording tag joined to a support provide for covalent linkage of the polypeptide and the associated recording tag on the support. In some embodiments, the support is a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere. In some embodiments, the support comprises gold, silver, a semiconductor or quantum dots. In some embodiments, the support is a nanoparticle and the nanoparticle comprises gold, silver, or quantum dots. In some embodiments, the support is a polystyrene bead, a polymer bead, an agarose bead, an acrylamide bead, a solid core bead, a porous bead, a paramagnetic bead, glass bead, or a controlled pore bead.

In some embodiments of any of the kits provided herein, the reagents for providing the polypeptide and an associated recording tag joined to a support provide for a plurality of polypeptides and associated recording tags that are joined to a support. In some embodiments, the plurality of polypeptides are spaced apart on the support, wherein the average distance between the polypeptides is about ≥20 nm.

Provided in some aspects are kits for analyzing a polypeptide which contain one or more binding agents as provided herein. In some embodiments of any of the kits provided herein, the binding agent is a peptide or protein. In some embodiments, the binding agent comprises an aminopeptidase or variant, mutant, or modified protein thereof; an aminoacyl tRNA synthetase or variant, mutant, or modified protein thereof; an anticalin or variant, mutant, or modified protein thereof; a ClpS or variant, mutant, or modified protein thereof; or a modified small molecule that binds amino acid(s), i.e. vancomycin or a variant, mutant, or modified molecule thereof; or an antibody or binding fragment thereof; or any combination thereof. In some embodiments, the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the polypeptide. In some embodiments, the binding agent is capable of selectively binding to the polypeptide. In some embodiments, the binding agent binds to a NTAA-functionalized single amino acid residue, a NTAA-functionalized dipeptide, a NTAA-functionalized tripeptide, or a NTAA-functionalized polypeptide. For example, the one or more binding agents are capable of binding to a functionalized NTAA is an NTAA treated with a compound selected from a compound any one of Formula (AA), Formula (AB), a compound of the formula R³—NCS, an amine of Formula R²—NH₂ or with a diheteronucleophile, or a salt or conjugate thereof, as described herein, or any combinations thereof. In some embodiments, the binding agent is capable of binding to or configured to bind a side product from treating the polypeptide with any of the provided chemical reagents.

In some embodiments of any of the kits provided herein, the coding tag is DNA molecule, an RNA molecule, a BNA molecule, an XNA molecule, a LNA molecule, a PNA molecule, a γPNA molecule, or a combination thereof. In some embodiments, the coding tag comprises an encoder or barcode sequence. In some embodiments, the coding tag further comprises a spacer, a binding cycle specific sequence, a unique molecular identifier, a universal priming site, or any combination thereof. In some embodiments, the coding tag comprises a nucleic acid, an oligonucleotide, a modified oligonucleotide, a DNA molecule, a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the DNA molecule is backbone modified, sugar modified, or nucleobase modified. In some embodiments, the DNA molecule has nucleobase protecting groups such as Alloc, electrophilic protecting groups such as thiranes, acetyl protecting groups, nitrobenzyl protecting groups, sulfonate protecting groups, or traditional base-labile protecting groups including Ultramild reagents.

In some embodiments of any of the kits provided herein, the binding portion and the coding tag in the binding agent are joined by a linker. In some embodiments, the binding portion and the coding tag are joined by a SpyTag/SpyCatcher peptide-protein pair, a SnoopTag/SnoopCatcher peptide-protein pair, or a HaloTag/HaloTag ligand pair.

In some embodiments of any of the kits provided herein, the reagent for transferring the information of the coding tag to the recording tag comprises a DNA ligase or an RNA ligase. In some embodiments, the reagent for transferring the information of the coding tag to the recording tag comprises a DNA polymerase, an RNA polymerase, or a reverse transcriptase. In some embodiments, the reagent for transferring the information of the coding tag to the recording tag comprises a chemical ligation reagent. In some embodiments, the chemical ligation reagent is for use with single-stranded DNA. In some embodiments, the chemical ligation reagent is for use with double-stranded DNA.

In some embodiments of any of the kits provided herein, further comprising a ligation reagent comprised of two DNA or RNA ligase variants, an adenylated variant and a constitutively non-adenylated variant. In some embodiments, the kit further comprises a ligation reagent comprised of a DNA or RNA ligase and a DNA/RNA deadenylase. In some embodiments, the kit additionally comprises reagents for nucleic acid sequencing methods. In some embodiments, the nucleic acid sequencing method is sequencing by synthesis, sequencing by ligation, sequencing by hybridization, polony sequencing, ion semiconductor sequencing, or pyrosequencing. In some embodiments, the nucleic acid sequencing method is single molecule real-time sequencing, nanopore-based sequencing, or direct imaging of DNA using advanced microscopy.

In some embodiments of any of the kits provided herein, the kit additionally comprises reagents for amplifying the extended recording tag. In some embodiments of any of the kits provided herein, the kit additionally comprises reagents for adding a cycle label. In some embodiments, the cycle label provides information regarding the order of binding by the binding agents to the polypeptide. In some embodiments, the cycle label can be added to the coding tag. In some embodiments, the cycle label can be added to the recording tag. In some embodiments, the cycle label can be added to the binding agent. In some embodiments, the cycle label can be added independent of the coding tag, recording tag, and binding agent. In some embodiments, the order of coding tag information contained on the extended recording tag provides information regarding the order of binding by the binding agents to the polypeptide. In some embodiments, the frequency of the coding tag information contained on the extended recording tag provides information regarding the frequency of binding by the binding agents to the polypeptide.

In some embodiments of any of the kits provided herein, the kit is configured for analyzing one or more polypeptides from a sample comprising a plurality of protein complexes, proteins, or polypeptides.

In some embodiments of any of the kits provided herein, the kit further comprises means for partitioning the plurality of protein complexes, proteins, or polypeptides within the sample into a plurality of compartments, wherein each compartment comprises a plurality of compartment tags optionally joined to a support (e.g., a solid support), wherein the plurality of compartment tags are the same within an individual compartment and are different from the compartment tags of other compartments. In some embodiments, the compartment is a physical compartment, a bead, and/or a region of a surface. In some embodiments, the compartment is the surface of a bead. In some embodiments, the compartment is a physical compartment containing a barcoded bead. In other embodiments, the compartment is the surface of the barcoded bead.

In some embodiments of any of the kits provided herein, the kit further comprises a reagent for fragmenting the plurality of protein complexes, proteins, and/or polypeptides into a plurality of polypeptides. In some embodiments, the compartment is a microfluidic droplet. In some embodiments, the compartment is a microwell. In some embodiments, the compartment is a separated region on a surface. In some embodiments, each compartment comprises on average a single cell.

In some embodiments of any of the kits provided herein, the kit further comprises a reagent for labeling the plurality of protein complexes, proteins, or polypeptides with a plurality of universal DNA tags.

In some embodiments of any of the kits provided herein, the reagent for transferring the compartment tag information to the recording tag associated with a polypeptide comprises a primer extension or ligation reagent. In some embodiments, the compartment tag comprises a single stranded or double stranded nucleic acid molecule. In some embodiments, the compartment tag comprises a barcode and optionally a UMI. In some embodiments, the support is a bead and the compartment tag comprises a barcode, further wherein beads comprising the plurality of compartment tags joined thereto are formed by split-and-pool synthesis. In some embodiments, the support is a bead and the compartment tag comprises a barcode, further wherein beads comprising a plurality of compartment tags joined thereto are formed by individual synthesis or immobilization. In some embodiments, the support is a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere. In some embodiments, the bead is a polystyrene bead, a polymer bead, an agarose bead, an acrylamide bead, a solid core bead, a porous bead, a paramagnetic bead, glass bead, or a controlled pore bead. In some embodiments, the support comprises gold, silver, a semiconductor or quantum dots. In some embodiments, the support is a nanoparticle and the nanoparticle comprises gold, silver, or quantum dots. In some embodiments, the support is a polystyrene bead, a polymer bead, an agarose bead, an acrylamide bead, a solid core bead, a porous bead, a paramagnetic bead, glass bead, or a controlled pore bead.

In some embodiments of any of the kits provided herein, the compartment tag is a component within a recording tag, wherein the recording tag optionally further comprises a spacer, a barcode sequence, a unique molecular identifier, a universal priming site, or any combination thereof. In some embodiments, the compartment tags further comprise a functional moiety capable of reacting with an internal amino acid, the peptide backbone, or N-terminal amino acid on the plurality of protein complexes, proteins, or polypeptides. In some embodiments, the functional moiety is an aldehyde, an azide/alkyne, or a malemide/thiol, or an epoxide/nucleophile, or an inverse electron domain Diels-Alder (iEDDA) group, or a moiety for a Staudinger reaction. In some embodiments, the functional moiety is an aldehyde group. In some embodiments, the plurality of compartment tags is formed by: printing, spotting, ink-jetting the compartment tags into the compartment, or a combination thereof. In some embodiments, the compartment tag further comprises a polypeptide. In some embodiments, the compartment tag polypeptide comprises a protein ligase recognition sequence.

In some embodiments of any of the kits provided herein, the kit comprises a protein ligase, wherein the protein ligase is butelase I or a homolog thereof. In some embodiments of any of the kits provided herein, wherein the reagent for fragmenting the plurality of polypeptides comprises a protease. In some embodiments, the protease is a metalloprotease.

In some embodiments of any of the kits provided herein, the kit further comprises a reagent for modulating the activity of the metalloprotease, e.g., a reagent for photo-activated release of metallic cations of the metalloprotease. In some embodiments, the kit further comprises a reagent for subtracting one or more abundant proteins from the sample prior to partitioning the plurality of polypeptides into the plurality of compartments. In some embodiments, the compartment is a physical compartment, a bead, and/or a region of a surface. In some embodiments, the compartment is the surface of a bead. In some embodiments, the compartment is a physical compartment containing a barcoded bead. In other embodiments, the compartment is the surface of the barcoded bead.

In some embodiments, the kit further comprises a reagent for releasing the compartment tags from the support prior to joining of the plurality of polypeptides with the compartment tags. In some embodiments, the kit further comprises a reagent for joining the compartment tagged polypeptides to a support in association with recording tags.

Provided in other aspects are kits for screening for a polypeptide functionalizing reagent, an amino acid eliminating reagent and/or a reaction condition, comprising: (a) a polynucleotide; (b) a polypeptide functionalizing reagent and/or an amino acid eliminating reagent; and (c) means for assessing the effect of said polypeptide functionalizing reagent, said amino acid eliminating reagent and/or a reaction condition for polypeptide functionalization or elimination on said polynucleotide. In some embodiments, the polypeptide functionalizing reagent comprises a compound of Formula (AA) as described herein, or a salt or conjugate thereof.

Provided in some aspects are kits for sequencing a polypeptide comprising: (a) a reagent for affixing the polypeptide to a support or substrate, or a reagent for providing the polypeptide in a solution; (b) a reagent for functionalizing the N-terminal amino acid (NTAA) of the polypeptide, wherein the reagent comprises a compound of Formula (AA) or R³—NCS as described herein.

In some embodiments, the kit additionally comprises a reagent for eliminating the functionalized NTAA to expose a new NTAA.

In some embodiments, the kit further includes an enzyme to transform or remove particular amino acid residues from the polypeptide, e.g., a proline aminopeptidase, a proline iminopeptidase (PIP), a pyroglutamate aminopeptidase (pGAP), an asparagine amidohydrolase, a peptidoglutaminase asparaginase, and/or a protein glutaminase, or a homolog thereof.

In some embodiments of any of the kits described herein, wherein the polypeptide is obtained by fragmenting a protein from a biological sample. In some embodiments, the support or substrate is a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.

In some embodiments of any of the kits described herein, the reagent for eliminating the functionalized NTAA is an amine of formula R2-NH₂, an amine base, a diheteronucleophile, or a base; or any combination thereof. In some embodiments, the polypeptide is covalently affixed to the support or carrier. In some embodiments, the support or carrier is optically transparent. In some embodiments, the support or carrier comprises a plurality of spatially resolved attachment points and step a) comprises affixing the polypeptide to a spatially resolved attachment point.

In some embodiments, the binding portion of the binding agent comprises a peptide or protein. In some embodiments, the binding portion of the binding agent comprises an aminopeptidase or variant, mutant, or modified protein thereof; an aminoacyl tRNA synthetase or variant, mutant, or modified protein thereof; an anticalin or variant, mutant, or modified protein thereof; a ClpS (such as ClpS2) or variant, mutant, or modified protein thereof; a UBR box protein or variant, mutant, or modified protein thereof; or a modified small molecule that binds amino acid(s), i.e. vancomycin or a variant, mutant, or modified molecule thereof; or an antibody or binding fragment thereof; or any combination thereof.

In some embodiments of any of the kits described herein, the chemical reagent comprises a conjugate selected from the group consisting of

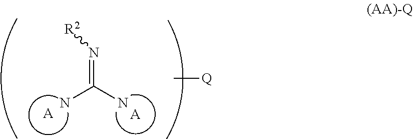

wherein Ring A is selected from:

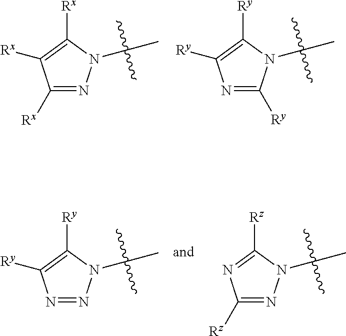

wherein:

each R^{x}, R^{y} and R^{z} is independently selected from H, halo, C₁₋₂ alkyl, C₁₋₂haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂haloalkyl, NO₂, SO₂(C₁₋₂ alkyl), COOR^{#}, and C(O)N(R^{#})₂,

and two R^{x}, R^{y} or R^{z} on adjacent atoms of a ring can optionally be taken together to form a phenyl group fused to the ring, and the fused phenyl can optionally be substituted with one or two groups selected from halo, C₁₋₂ alkyl, C₁₋₂haloalkyl, NO₂, SO₂(C₁₋₂ alkyl), COOR^{#}, and C(O)N(R^{#})₂;

wherein each R^{#} is independently H or C₁₋₂ alkyl, and two R# on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OMe, Me, oxo, NH₂, NHMe and NMe₂; and

Q is a ligand.

In some embodiments, the kit additionally comprises a reagent for eliminating the functionalized NTAA to expose a new NTAA, as described herein. The reagent can be ammonia, ammonium hydroxide, a primary amine, a base such as hydroxide, or a diheteronucleophile such as hydrazine, hydroxylamine, substituted hydrazines, and C₁₋₄ alkoxyamines. In some embodiments of any of the kits described herein, the sample comprises a biological fluid, cell extract or tissue extract. In some embodiments of any of the kits described herein, the fluorescent label is a fluorescent moiety, color-coded nanoparticle or quantum dot.

### EXAMPLES

The following examples are offered to illustrate but not to limit the methods, compositions, and uses of the invention provided herein.

### Example 1: N-Terminal Amino Acid Functionalization and Elimination from Polypeptides

This example describes the assessment of reactions performed with polypeptides including modification (e.g., functionalization) of the N-terminal amino acid (NTAA) of peptides and removal (e.g., elimination) of said modified NTAA.

In general, the tested method included treating a peptide with an isothiocyanate or a derivative thereof (R¹) to functionalize the NTAA by forming a thiourea, and the thiourea is then converted to a guanidine at the NTAA using a second reagent (R²), as shown in Scheme 1. The polypeptides were then treated with a base to eliminate the NTAA. In some cases, the thiourea may be treated with methyl iodide or other oxidization reagents between functionalization and elimination. Furthermore, other bases for promoting cycloelimination after formation of the corresponding guanidine can be used, including but not limited to 0.1 M NaOH, 0.1 M LiOH, 0.1 M Na₃PO₄, and 0.1 M K₂CO₃ buffer, and others.

Functionalization and elimination of the NTAA was tested on the following peptide sequences: GRFSGIY(SEQ ID NO: 142), AALAY (SEQ ID NO: 143), FGAALAWK(N3) (SEQ ID NO: 144), and WTQIFGA (SEQ ID NO: 145). The polypeptides were treated in solution as follows: 1 mM of the test peptide (with the sequence indicated in Table 2A) and 3 mM of phenyl isothiocyanate (PITC) were suspended in acetonitrile/0.5 M triethylamine acetate (TEAA) (1:1). The mixture was heated at 60° C. for 30 minutes. Then, an equal volume of 28% ammonium hydroxide was added. The mixture was heated at 60° C. for 1 hour. For analysis, a portion of the eluted material was injected into an LCMS and monitored by UV. As shown in Table 2A, the observed masses of all four treated peptides indicated that the terminal amino acid was modified and removed by treating with PITC followed by ammonium hydroxide.

TABLE 2A
Assessment of Functionalization and Elimination on Various Peptide Sequences

| | | | Functionalization | | Elimination | | |
|---|---|---|---|---|---|---|---|
| | | | Expected | | Peptide | | |
| | | Peptide | MW | Observed | Sequence | | Observed |
| | | MW | After | MS | After Elim. | Expected | MS |
| R₁ | R₂ | (SEQ ID NO) | Mod. | (M - H) | (SEQ ID NO) | MW | (M + H) |
| PITC | Ammonium | GRFSGIY | 933 | 932 | RFSGIY | 741 | 742 |
| | hydroxide | 798.4 | | | (SEQ ID NO: | | |
| | | (SEQ ID NO: 142) | | | 149) | | |
| PITC | Ammonium | AALAY | 642 | 641 | ALAY | 436 | 437 |
| | hydroxide | 507.3 | | | (SEQ ID NO: | | |
| | | (SEQ ID NO: 143) | | | 148) | | |
| PITC | Ammonium | GFAALAWK(N3) | 1024 | 1024 | GAALAWK | 741 | 741 |
| | hydroxide | 889.0 | | | (N3) | | |
| | | (SEQ ID NO: 144) | | | (SEQ ID NO: | | |
| | | | | | 147) | | |
| PITC | Ammonium | WTQIFGA | 956 | 955 | TQIFGA | 635 | 636 |
| | hydroxide | 821.4 | | | (SEQ ID NO: | | |
| | | (SEQ ID NO: 145) | | | 146) | | |

In addition, various reagents were tested in a reaction substantially as described above except the indicated peptides in Table 2B were treated with various isothiocyanate derivatives in the first step and either ammonium hydroxide, methylamine, isopropylamine, or ethanolamine in the second step. The observed functionalization and elimination using the reagents was confirmed by the observed masses of the treated peptides as shown in Table 2.

TABLE 2B
Assessment of Functionalization and Elimination on
Peptides Treated with Various Reagents

| | | | Functionalization | | Elimination | | |
|---|---|---|---|---|---|---|---|
| | | Peptide | Expected | | Peptide | | |
| | | MW | MW | Observed | Sequence | | Observed |
| | | (SEQ ID | After | MS | After Elim. | Expected | MS |
| R₁ | R₂ | NO) | Mod. | (M − H) | (SEQ ID NO) | MW | (M + H) |
| PITC | Ammonium | WTQIFGA | 956 | 955 | TQIFGA | 635 | 636 |
| 3-Pyridyl | hydroxide | 821.4 | 957 | 957 | (146) | 635 | 636 |
| isothiocyanate | | (145) | | | | | |
| 4-Nitrophenyl | | | 1001 | 1001 | | 635 | 636 |
| isothiocyanate | | | | | | | |
| 2-(4-Morpholino) | | | 993 | 993 | | 635 | 636 |
| ethyl | | | | | | | |
| isothiocyanate | | | | | | | |
| 4-Sulfophenyl | | | 1035 | 1035 | | 635 | 636 |
| isothiocyanate | | | | | | | |
| sodium | | | | | | | |
| Methyl | | | 894 | 894 | | 635 | 636 |
| isothiocyanate | | | | | | | |
| Isopropyl | | | 922 | 922 | | 635 | 636 |
| isothiocyanate | | | | | | | |
| Cyclohexyl | | | 962 | 962 | | 635 | 636 |
| isothiocyanate | | | | | | | |
| 4-Fluorophenyl | | | 974 | 974 | | 635 | 636 |
| isothiocyanate | | | | | | | |
| 4-Methylphenyl | | | 970 | 970 | | 635 | 636 |
| isothiocyanate | | | | | | | |
| PITC | Ammonium | GRFSGIY | 933 | 932 | RFSGIY | 741 | 742 |
| | hydroxide | 798.4 | | | (149) | | |
| | Methylamine | (142) | 933 | 932 | | 741 | 742 |
| | Isopropylamine | | 933 | 932 | | 741 | 742 |
| | Ethanolamine | | 933 | 932 | | 741 | 742 |
| | Ammonium | WTQIFGA | 956 | 955 | TQIFGA | 635 | 636 |
| | hydroxide | 821.4 | | | (146) | | |
| | Methylamine | (145) | 956 | 955 | | 635 | 636 |
| | Isopropylamine | | 956 | 955 | | 635 | 636 |
| | Ethanolamine | | 956 | 955 | | 635 | 636 |

Similar to the functionalization and elimination reactions tested above, various peptides were also tested with hydrazine and hydroxylamine to replace the ammonium hydroxide. The polypeptides were treated in solution as follows: 1 mM of the test peptide (with the sequence indicated in Table 3) and 10 mM of phenyl isothiocyanate (PITC) were suspended in acetonitrile/0.5 M triethylamine acetate (TEAA) (1:1). The mixture was heated at 60° C. for 30 minutes. After modification, the mixture was treated with an equal volume of hydrazine (50˜60%). The elimination reaction was performed at 60° C. 3 hours or 80° C. for 1 hour. Using similar methods as described above, the observed masses of all treated peptides indicated that the NTAA was modified and removed. It was observed that ˜60% of peptides showed NTAA elimination with the reaction performed at 60° C. for 1 hour, and >95% of peptides showed NTAA elimination when the reaction was performed 60° C. 3 hours or 80° C. at 1 hour. In the reaction performed with hydrazine, the elimination reaction had a pH of about 12 and did not require any additional base buffers.

In some cases, the hydrazine was replaced with substituted hydrazine or hydroxylamine HCl (20%).

TABLE 3
Assessment of Functionalization and Elimination on Peptides Treated with
Hydrazine or Hydroxylamine

| | | | Functionalization | | | | |
|---|---|---|---|---|---|---|---|
| | | | Expected | | Elimination | | |
| | | Peptide | MW | Observed | Peptide | | Observed |
| | | MW | After | MS | Sequence | Expected | MS |
| R₁ | R₂ | (SEQ ID NO) | Mod. | (M - H) | After Elim. | MW | (M + H) |
| PITC | Hydrazine | FGAALAWK(N3) | 1024 | 1024 | GAALAW | 742 | 741 |
| | | 889.0 | | | K(N3) | | |
| | | (SEQ ID NO: 144) | | | (SEQ ID | | |
| | | | | | NO: 147) | | |
| | Hydrazine | AALAY | 642 | 641 | ALAY | 436 | 437 |
| | | 507.3 | | | (SEQ ID | | |
| | | (SEQ ID NO: 143) | | | NO: 148) | | |
| | Hydrazine | WTQIFGA | 956 | 955 | TQIFGA | 635 | 636 |
| | | 821.4 | | | (SEQ ID | | |
| | | (SEQ ID NO: 145) | | | NO: 146) | | |
| | Hydrazine | FHAALAWK(N3) | 1104 | 1104 | HAALAW | 822 | 822 |
| | | 969.1 | | | K(N3) | | |
| | | (SEQ ID NO: 150) | | | (SEQ ID | | |
| | | | | | NO: 151) | | |
| | Hydroxylamine | FHAALAWK(N3) | 1104 | 1104 | HAALAW | 822 | 822 |
| | | 969.1 | | | K(N3) | | |
| | | (SEQ ID NO: 150) | | | (SEQ ID | | |
| | | | | | NO: 151) | | |

### Example 2: Synthesis of Diheterocyclic Methanimines

This example describes the synthesis procedures used to prepare diheterocyclic methanimine reagents.

### General Procedure A:

To a glass vial equipped with a magnetic stir bar, 100 mg of cyanogen bromide (0.95 mmol) was added in and dissolved in 1-2 mL of acetone and cooled on an ice bath until later use. In a separate vial, 1.97 mmol of heterocycle was dissolved in 5-6 mL of ethanol and solution was mixed in with the chilled acetone solution. The solution was allowed to stir at 0° C. for 5 minutes before the addition of 800 μL of 2M NaOH (aq.). The vigorously stirred solution was allowed to come to room temperature over the course of 1 hour. A precipitate formed, the solids filtered, and washed with cold ethanol. The resulting solids were obtained without further purification (>95% pure, 20-60% yield).

### General Procedure B:

To a glass vial equipped with a magnetic stir bar, 100 mg of cyanogen bromide (0.95 mmol) was added in and dissolved in 1-2 mL of dichloromethane and stored at 4° C. until further use. In a separate vial, 1.97 mmol of heterocycle was dissolved in 5 mL of dichloromethane. To this, 3 mmol of triethylamine (or diisopropylethylamine) was added and stirred for 10 minutes or until all solids dissolved. This solution was then added dropwise to the cyanogen bromide containing solution. The reaction was allowed to stir at 25° C. for 1-18 hours. Upon completion, monitored by thin layer chromatography (TLC), the reaction was condensed in vacuo and loaded onto a normal phase silica plug. The product was obtained by normal phase flash chromatography (0-60% ethyl acetate in n-heptane). The fractions containing the desired product were pooled and condensed to afford the isolated product (>95% pure, 40-85% yield).

Exemplary diheterocyclic methanimine reagents prepared using the procedures provided include: bis-(4-trifluoromethylpyrazole)methanimine, bis(benzotriazole)methanimine, bis-pyrazole methanimine, bis-(3-trifluoromethylpyrazole)methanimine, bis-(4-methylpyrazole)methanimine, bis-(4-nitroimidazole)methanimine, and bis-(3,5-dimethylpyrazole)methanimine.

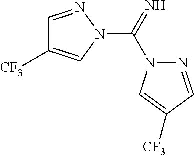 [SMILES: N=C(n1cc(C(F)(F)F)cn1)n1cc(C(F)(F)F)cn1]

bis-(4-trifluoromethylpyrazole)methanimine. Prepared according to general procedure B.

¹H NMR (400 MHz, DMSO-d6): δ 10.758 (1H, s), 9.171 (1H, s), 8.883 (1H, s), 8.412 (1H, s), 8.343 (1H, s)

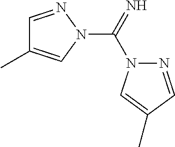

bis-(4-methylpyrazole)methanimine. Prepared according to general procedure B. ¹H NMR (400 MHz, DMSO-d6): δ 9.273 (1H, s), 8.212 (1H, s), 7.986 (1H, s), 7.759 (1H, s), 7.718 (1H, s), 2.109 (3H, s), 2.058 (3H, s)

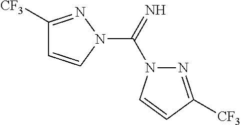 [SMILES: N=C(n1ccc(C(F)(F)F)n1)n1ccc(C(F)(F)F)n1]

bis-(3-trifluoromethylpyrazole)methanimine. Prepared according to general procedure A. ¹H NMR (400 MHz, DMSO-d6): δ 10.915 (1H, s), 8.705 (1H, d, J=2 Hz), 8.427 (1H, d, J=2 Hz), 7.147 (1H, d, J=2 Hz), 7.102, d, J=2 Hz)

### Example 3: Assessment of N-Terminal Amino Acid Functionalization and Elimination

This example demonstrates modification (e.g., functionalization) of the N-terminal amino acid (NTAA) of peptides treated with diheterocyclic methanimine and removal (e.g. elimination) of the NTAA (see Scheme 1). Various diheterocyclic methanimines were isolated using the general procedures A and B as described in Example 2. Functionalization and elimination were assessed in peptides treated with the following reagents: bis-(4-trifluoromethylpyrazole)methanimine, bis-(benzotriazole)methanimine, bis-(pyrazole)methanimine, bis-(3-trifluoromethylpyrazole)methanimine, and bis-(4-methylpyrazole)methanimine, bis-(3,5-dimethylpyrazole)methanimine, bis-(imidazole)methanimine, and bis-(4-nitroimidazole)methanimine.

### A. Functionalization and Elimination of the NTAA:

An aliquot of 5 μL of 6 pools with 10 peptides in each with various amino acid sequences with length ranging from 5 to 10 amino acids (10 mM) dissolved in dimethylsulfoxide (DMSO) was added to 85 μL of buffer (pH ranging from 6 to 9) and 25 μL of acetonitrile (20%). To this, 10 μL of 150 mM diheterocyclic methanimine in DMSO was added, mixed well, and allowed to react at 40° C. for 1 hour. After the one-hour time point, an aliquot was removed from the reaction, quenched with aqueous acetic acid, and analyzed by LCMS. An aliquot of 50% hydrazine derivative (20 μL; in water or DMSO) was added to bring the effective hydrazine concentration to 11% and allowed to react for 1 hour at 40° C. Upon completion, the reaction was quenched with 1M acetic acid (aq.) and monitored by LCMS. The resulting desired product (peptide with NTAA eliminated) can be obtained at 1-97% yields, as shown in Table 4A.

TABLE 4A
Elimination of NTAA from Peptides

| Reagent | % Elimination |
|---|---|
| Hydrazine | 97 |
| Hydroxylamine | 30 |
| Methoxyamine | 13 |
| Hydroxylamine-O-sulfonic acid | 45 |
| N-methylhydrazine | 29 |
| Tert-butyl carbazate | 1 |
| Benzhydrazide | 18 |
| 4-methoxybenzhydrazide | 3 |
| 2-hydroxyethylhydrazine | 6 |
| N-acetylhydrazide | 10 |
| 4-toluenesulfonyl hydrazide | 50 |
| Phenylhydrazine-4-sulfonic acid | 19 |
| 2,4-dinitrophenylhydrazine | 0 |

In some cases, the N-aminoguanidine intermediate was isolated by using diheteronucleophile salts as the hydrazine derivatives, to displace the heterocyclic methanimine functionalized peptide, without producing the desired product peptide with the NTAA eliminated. Using this method, isolation of the intermediate may provide additional control over the reaction (e.g., reduced side product formation of hydrolysis or hydantoin). Further reaction conditions tested included increasing the system's pH to 9 (using trisodium phosphate, sodium hydroxide, lithium hydroxide, potassium hydroxide, or other pH ≥9 buffers) to then convert the N-heteroguanidine to the desired product (peptide with NTAA eliminated), as shown in Table 4B.

TABLE 4B
N-Heteroguanidinyl Functionalization & Base-promoted Elimination

| Reagent | % Functionalization | % Elimination |
|---|---|---|
| Hydrazine HCl | 84 | 100 |
| Hydroxylamine HCl | 90 | 75 |
| Methoxyamine HCl | 76 | 14 |
| Acetylhydrazine | 90 | 5 |

### B. Hydrazine Buffer Combinations

Removal of the N-terminal amino acid (NTAA) of peptides treated with 4-(trifluoromethyl)pyrazole carboxamidine was assessed in the presence of hydrazine and various buffers. 4-(trifluoromethyl)pyrazole carboxamidine functionalized peptide was purified by preparative HPLC. The purified peptide was dissolved in DMSO to a concentration of 5 mM. 5 μL of the peptide solution was added to 35 μL of different buffers (Table 5) and 10 μL of 55% hydrazine hydrate was added to the solution. The reaction was placed in a thermomixer and allowed to react for 1 hour at 40° C. Upon completion, the reaction was quenched with 1M acetic acid and monitored by LCMS. Analysis showed the use of various buffers resulted in varying amounts of desired N-terminal amino acid hydrolysis, aminoguanidine intermediate, and undesired hydantoin product (Table 5). In some cases, using 0.7M Tris buffer produced the desired N-terminal amino acid hydrolysis, aminoguanidine intermediate, and relatively low amounts of hydantoin product.

TABLE 5
Assessment of NTAA Elimination in Peptides
Treated with Hydrazine in Various Buffers

| | | % | % | % |
|---|---|---|---|---|
| Buffer ([Eff. M]) | pH₀ | Elimination | HydzFunct | Hydantoin |
| NaPhos (0.07M) | 6.0 | 44 | 19 | 24 |
| MOPS (0.07M) | 7.6 | 35 | 25 | 32 |
| NEMA (0.07M) | 8.0 | 50 | 26 | 24 |
| NEMA (0.14M) | 8.0 | 71 | 17 | 12 |
| TEAA (0.07M) | 8.5 | 57 | 24 | 19 |
| Kphos (0.07M) | 8.0 | 10 | 14 | 76 |
| PBS (0.11M) | 7.4 | 10 | 15 | 75 |
| CAPSO (0.07M) | 10.3 | 32 | 33 | 35 |
| CBc (0.07M) | 10.5 | 3 | 4 | 93 |
| Borate (0.07M) | 8.5 | 36 | 30 | 34 |
| HEPES (0.07M) | 8.0 | 42 | 31 | 27 |
| NaPhos (0.07M) | 7.0 | 30 | 26 | 44 |
| Tris (0.07M) | 7.6 | 64 | 19 | 17 |
| TEAA (0.35M) | 8.5 | 72 | 18 | 10 |
| Tris (0.7M) | 8.0 | 90 | 6 | 4 |

### Example 4: DNA treatment with Diheteronucleophiles and Diheterocyclic Methaneimines

The DNA sequence as set forth in SEQ ID NO:171 (TTT/i5OCTdU/TTUCGTAGTCCGCGACACTAGTAAGCCGGTATATCAACTGAGTG]) (1 μmol), was dissolved in 1 mL of water. Four tubes were prepared and the DNA was treated either water as control or with various hydrazines as follows:

Condition 1: 5 μL of the solution of DNA was combined with 45 μL water and heated at 40° C. for 1 h.
Condition 2: 5 μL of the solution of DNA was combined with 35 μL water and 10 μL of hydrazine hydrate (50% aqueous), and the mixture was heated at 40° C. for 1 h.
Condition 3: 5 μL of the solution of DNA was combined with 35 μL Tris buffer (1M) and 10 of hydrazine hydrate (50% aqueous), and the mixture was heated at 40 C for 1 h.
Condition 4: 5 μL of the solution of DNA was combined with 35 μL water and 10 μL of hydrazine hydrochloride (50% aqueous), and the mixture was heated at 40° C. for 1 h. The mixtures for Conditions 1-4 were then lyophilized overnight and analyzed by mass. FIGS. 53A, 53B, 53C, and 53D shows the mass analysis of the DNA with the sequence in SEQ ID NO:171 subjected to Conditions 1, 2, 3, and 4, respectively. Intact DNA was observed after various hydrazine treatments. The DNA sequence as set forth in SEQ ID NO:171 (1 μmol) was dissolved in 1 mL of water. 10 μL of the solution of DNA was combined with 10 μL bis-(4-trifluoromethylpyrazole)methanimine (150 mM, DMSO) and 80 μL N-ethylmorpholine buffer (0.2M, pH=8.0) and the mixture heated at 40° C. for 1 h. The mixtures were then lyophilized overnight and analyzed by mass. Intact DNA was observed after treatment with bis-(4-trifluoromethylpyrazole)methanimine (FIG. 54).

### Example 5: DNA Encoding Assay with N-Terminal Amino Acid (NTAA) Functionalization and Elimination Using an Exemplary Diheterocyclic Methanimine

This example demonstrates a ProteoCode assay including modification (e.g., functionalization) and elimination of the N-terminal amino acid (NTAA) of peptides treated with diheterocyclic methanimine. Binding of a binding agent to the modified NTAA and encoding by transferring information from a coding tag associated with the binding agent to a recording tag associated with the peptide, thereby generating an extended recording tag, was also performed as shown in FIG. 55A. Binding and encoding was performed using a pool of binding agents (phenylalanine (F) and leucine (L) binders) that recognize the modified NTAA (“mod”).

TABLE 6
Assay Peptides

| SEQ | |
|---|---|
| ID NO | Sequence |
| 152 | YAEALAESAFSGVARGDVRGGK(N3) |
| 153 | AEALAESAFSGVARGDVRGGK(N3) |
| 154 | EALAESAFSGVARGDVRGGK(N3) |
| 155 | ALAESAFSGVARGDVRGGK(N3) |
| 156 | LAESAFSGVARGDVRGGK(N3) |
| 157 | AESAFSGVARGDVRGGK(N3) |
| 158 | ESAFSGVARGDVRGGK(N3) |
| 159 | SAFSGVARGDVRGGK(N3) |
| 160 | AFSGVARGDVRGGK(N3) |
| 161 | FSGVARGDVRGGK(N3) |
| 162 | SGVARGDVRGGK(N3) |
| 163 | LAGELAGELAGEIRGDVRGGK(N3) |
| 164 | ELAGELAGELAGEIRGDVRGGK(N3) |
| 165 | GELAGELAGELAGEIRGDVRGGK(N3) |
| 166 | AGELAGELAGELAGEIRGDVRGGK(N3) |
| 167 | FAFAGVAMPRGAEDVRGGK(N3) |
| 172 | FLAEIRGDVRGGK(N3) |
| 173 | dimethyl-AESAESASRFSGVAMPGAEDDVVGSGSK(N3) |

Peptides labelled with a DNA recording tag were immobilized on a substrate (peptide sequences as set forth in SEQ ID NOs: 152-167, 172-173). Up to four cycles of elimination followed by binding and encoding was performed. For example, the peptides were treated with an exemplary diheterocyclic methanimine as the reagent for functionalization of the NTAA. For functionalization treatment, the assay beads were incubated with 150 μL of 15 mM of di-(4-trifluoromethyl-pyrazo-1-yl)methanimine, 200 mM MOPS, pH7.6, 50% DMA at 40° C. for 30 minutes. The beads were washed 3× with 200 μL of PBST. Following functionalization, the assay beads were subjected to treatment with 150 μL of 7% hydrazine hydrochloride in PBS, pH 7.0 at 40° C. for 30 min. After 3×PBST washes, the elimination treatment was performed by incubating the assay beads with 150 of 1 M ammonium phosphate, pH 6.0 at 95° C. for 30 min. The beads were then washed 3× with 200 μL of PBST. The first cycle of binding F and L-binder to the functionalized NTAA (4-trifluoromethylpyrazol-1-yl carboamidinyl)-peptide) and encoding was performed before any hydrazine treatment and elimination treatment (F-encoding, top panel of FIG. 55B; L-encoding, bottom panel of FIG. 55B). F and L-binder binding/encoding for subsequent cycles as indicated was performed after functionalization after either one, two, three, or four cycles of elimination.

After completion of the binding, encoding and described functionalization and elimination cycle(s), the extended recording tags were capped with an adapter sequence, subjected to PCR amplification, and analyzed by next-generation sequencing (NGS). FIG. 55B shows chemistry cycle-dependent encoding efficiency with the mod-F-binder and mod-L binder detection for peptides with the 5 residues of the N-terminal end indicated. Data on nine F and L containing peptides, in which either the F or L residue is stepped through the first 5 positions of the peptide, is shown. As each successive residue was eliminated, an N-terminal modified F or L residue was exposed on one of the peptides on the bead and detected by the corresponding mod-F or mod-L binder with concomitant DNA encoding. As shown, functionalization and binding of the modified NTAA was observed as indicated by elevated encoding levels. It was also observed that elimination was achieved as each binder detected the corresponding modified residue in the appropriate cycle after elimination of other residues that exposed the F or L residue. In summary, an increase in F-binder and L-binder encoding after functionalization (NTF) was observed and elimination (NTE) was detected, demonstrating the use of the exemplary diheterocyclic methanimine in the encoding assay for elimination of the NTAA and as a modification recognized by the shown exemplary binding agents.

### Example 6: Cleavage of N-Terminal Proline Residues from Surface-Anchored Peptides by Proline Iminopeptidase (PIP)

This example describes the assessment of N-terminal proline cleavage from surface anchored peptides using an exemplary amino acid cleaving enzyme, proline iminopeptidase (PIP; e.g., as classified in MEROPS family S33.001 or S33.008, or UniProt accession P46547 or P42786).

In general, the tested method included conjugating N-terminal proline peptides with an azide functional group to DBCO modified agarose beads, and treating surface anchored peptides with PIP to eliminate the proline amino acid residue. To analyze the completion of the PIP cleavage, the resulting peptides were further cleaved off the surface using trypsin and analyzed by LCMS.

To anchor the peptides to the surface, 1 mM azido peptide was treated with DBCO beads in 100 mM HEPES pH 7.5 at 60° C. overnight. After the reaction, the beads were washed three times with 100 mM NaOH, followed by three times PBST. The beads were resuspended in PBST. Exemplary azido peptides tested are set forth in SEQ ID NO: 174-190, wherein proline is in the N-terminal P1 position and K(N3) is an azido lysine. The surface anchored N-terminal proline peptides were treated with 4 μM PIP in 50 mM HEPES, pH 8. The mixture was heated at 25° C. for 22 hours. After reaction, the beads were washed with 50 mM HEPES pH 8 and resuspended in 100 μL 50 mM HEPES pH 8. The beads were digested with 0.4 ug sequencing grade trypsin at 37° C. for 1 hour. The supernatant of the trypsin digestion mixture containing peptide fragments were injected into an LCMS for analysis.

To analyze the LCMS data, raw mass counts corresponding to peptide fragment containing residues in the P2-P6 positions and peptide fragments containing residues in the P7-p10 positions were determined. For example, in the peptide provided in SEQ ID NO: 174, PAAEIRGDVRGGK(N3), the bolded portion and underlined portion represents the two peptide fragments analyzed. The ratio of the two fragments (Rₑₓₚ) were determined and compared to the standard (R_{std}) to determine the cleavage yield. As shown in Table 7, cleavage of N-terminal proline from the peptide fragment containing residues in the P2-P6 positions was observed as determined by the cleavage yield of N-terminal proline peptides described. In some cases, particular amino acids can be cleaved using an enzyme in addition to treatment with a chemical reagent (e.g. diheterocyclic methanimine). In some cases, the enzyme can be a functional homolog of PIP or fragment thereof.

TABLE 7
Assessment of N-terminal Proline Cleavage from Surface Anchored Peptides Using PIP

| | | Mass Count of | Mass Count of | | | |
|---|---|---|---|---|---|---|
| | SEQ | P2-P6 | P7-P10 | | | |
| Peptide | ID NO | Fragment | Fragment | Rₑₓₚ | R_{std} | Yield |
| PAAEIRGDVRGGK(N3) | 174 | 23779940 | 11143378 | 2.134 | 2.971 | 72% |
| PDAEIRGDVRGGK(N3) | 175 | 14638015 | 15288232 | 0.957 | 2.362 | 41% |
| PEAEIRGDVRGGK(N3) | 176 | 4675120 | 8008920 | 0.584 | 2.592 | 23% |
| PFAEIRGDVRGGK(N3) | 177 | 16734749 | dd31729 | 3.776 | 4.774 | 79% |
| PGAEIRGDVRGGK(N3) | 178 | 15941555 | 8081419 | 1.973 | 2.052 | 96% |
| PHAEIRGDVRGGK(N3) | 179 | 8778106 | 8424680 | 1.042 | 1.501 | 69% |
| PIAEIRGDVRGGK(N3) | 180 | 251557dd | 7282587 | 3.454 | 4.768 | 72% |
| PLAEIRGDVRGGK(N3) | 181 | 37433968 | 9049335 | 4.137 | 5.122 | 81% |
| PMAEIRGDVRGGK(N3) | 182 | 14806672 | 8276881 | 1.789 | 2.948 | 61% |
| PNAEIRGDVRGGK(N3) | 183 | 17536534 | 10512404 | 1.668 | 2.372 | 70% |
| PPAEIRGDVRGGK(N3) | 184 | 421224 | 2701155 | 0.156 | 3.845 | 10% |
| PQAEIRGDVRGGK(N3) | 185 | 10068328 | 10676044 | 0.943 | 0.559 | 40% |
| PSAEIRGDVRGGK(N3) | 186 | 14114769 | 9595561 | 1.471 | 2.236 | 66% |
| PTAEIRGDVRGGK(N3) | 187 | 16300255 | 11236549 | 1.451 | 2.804 | 52% |
| PVAEIRGDVRGGK(N3) | 188 | 19959460 | 7658112 | 2.606 | 4.187 | 62% |
| PWAEIRGDVRGGK(N3) | 189 | 40663948 | 22372022 | 1.818 | 3.239 | 56% |
| PYAEIRGDVRGGK(N3) | 190 | 33885980 | 15252256 | 2.222 | 3.022 | 74% |

### Example 7: Cleavage of N-Terminal Pyroglutamate from Surface-Anchored Peptides by Pyroglutamate Aminopeptidase (pGAP)

This example describes the assessment of N-terminal pyroglutamate cleavage from surface anchored peptides using an exemplary enzyme, pyroglutamate aminopeptidase (pGAP, UniProtKB accession number: A0A5C0XQC7).

In some cases, a peptide with a P2 glutamine can undergo the elimination step when treated with a diheterocyclic methanimine. During this step, the P1 amino acid is eliminated and newly formed N-terminal glutamine may cyclize to form pyroglutamate. In one example, pyroglutamate may form under the elimination reaction condition with 1 M ammonium phosphate pH 6.0 at 95° C. for 30 min. Because of the cyclic structure of pyroglutamate, in some cases, it may be desirable to remove pyroglutamate from the N-terminus using an enzymatic approach, such as by treating with pGAP.

To assess the activity of pGAP cleavage, peptides with an azide functional group were conjugated to DBCO modified agarose beads as described in Example 6, and the surface anchored N-terminal pyroglutamate peptides were treated with pGAP enzyme to eliminate the pyroglutamate amino acid residue. To analyze the completion of the pGAP cleavage, the resulting peptides were further cleaved off the surface using trypsin and analyzed by LCMS.

The cleavage of a pyroglutamate from the N-terminal pyroglutamate peptide was tested on the exemplary peptide sequences set forth in SEQ ID NOS: 191-207, where pyrogluatamate (pQ) is in the N-terminal P1 position. The surface anchored N-terminal pyroglutamate peptides were treated with 250 uU pfu pGAP in 1×pGAP buffer (50 mM sodium phosphate buffer pH 7.0, 10 mM DTT, 1 mM EDTA) at 80° C. for 2 hours. The beads were then washed on a filter plate with 50 mM HEPES pH 8 and resuspended in 100 μL 50 mM HEPES pH 8. The beads were digested with 0.4 ug sequencing grade trypsin at 37° C. for 1 hour. For analysis, the supernatant of the trypsin digestion mixture was injected into an LCMS. The data was analyzed using the method substantially as described in Example 6 by analyzing raw mass counts corresponding to peptide fragment containing residues in the P2-P6 positions and peptide fragments containing residues in the P7-P10 positions. For example, in the peptide provided in SEQ ID NO: 191, pQAAEIRGDVRGGK(N3), the bolded portion and underlined portion represents the two peptide fragments analyzed. Cleavage of N-terminal pyrogluatamate from the peptide fragments containing residues in the P2-P6 positions was observed as determined cleavage yield of N-terminal pyroglutamate peptides, as shown in Table 8.

TABLE 8
Assessment of Pyroglutamate Cleavage from Surface Anchored Peptides using pGAP

| | SEQ ID | Mass Count of | Mass Count of | | | |
|---|---|---|---|---|---|---|
| Peptide | NO | P2-P6 Fragment | P7-P10 Fragment | Rₑₓₚ | R_{std} | Yield |
| pQAAEIRGDVRGGK(N3) | 191 | 21559426 | 8779610 | 2.456 | 3.822 | 64% |
| pQDAEIRGDVRGGK(N3) | 192 | 20893582 | 10995079 | 1.900 | 3.246 | 74% |
| pQEAEIRGDVRGGK(N3) | 193 | 18083158 | 9699281 | 1.864 | 6.569 | 57% |
| pQFAEIRGDVRGGK(N3) | 194 | 27940712 | 6117624 | 4.567 | 3.060 | 70% |
| pQGAEIRGDVRGGK(N3) | 195 | 11058410 | 7127089 | 1.552 | 7.125 | 51% |
| pQHAEIRGDVRGGK(N3) | 196 | 13358820 | 8412278 | 1.588 | 5.994 | 77% |
| pQIAEIRGDVRGGK(N3) | 197 | 42224848 | 8435801 | 5.005 | 2.709 | 80% |
| pQLAEIRGDVRGGK(N3) | 198 | 20582000 | 3933664 | 5.232 | 3.731 | 73% |
| pQMAEIRGDVRGGK(N3) | 199 | 21582238 | 8010404 | 2.694 | 2.564 | 69% |
| pQNAEIRGDVRGGK(N3) | 200 | 20639178 | 11810859 | 1.747 | 2.057 | 63% |
| pQPAEIRGDVRGGK(N3) | 201 | 1741945 | 12904922 | 0.135 | 6.228 | 2% |
| pQQAEIRGDVRGGK(N3) | 202 | 7265370 | 5596064 | 1.298 | 3.928 | 80% |
| pQSAEIRGDVRGGK(N3) | 203 | 11152438 | 4632035 | 2.408 | 2.775 | 89% |
| pQTAEIRGDVRGGK(N3) | 204 | 23616410 | 9504565 | 2.485 | 0.692 | 73% |
| pQVAEIRGDVRGGK(N3) | 205 | 10918932 | 2408361 | 4.534 | 3.421 | 85% |
| pQWAEIRGDVRGGK(N3) | 206 | 32504282 | 11890270 | 2.734 | 5.356 | 73% |
| pQYAEIRGDVRGGK(N3) | 207 | 26991286 | 8686854 | 3.107 | 3.531 | 88% |

Homologs of pGAP enzymes from organisms other than Pyrococcus furiosus were also explored. For example, pGAPs from Pseudomonas fluorescens (UniProtKB accession number: A0A1B3DC66), Grimontia hollisae (UniProtKB accession number: A0A377J8L7), Streptomyces albidoflavus (UniProtKB accession number: A0A4R8P3K1), and Ollimonas pratensis (UniProtKB accession number: A0A127R4R6) were expressed in E. coli. and purified using nickel resin columns. The surface anchored N-terminal pyroglutamate peptides were treated with 1 μM pGAP from various organisms in 1×pGAP buffer at 40° C. for 2 hours. The beads were then digested and analyzed as described above. Cleavage yield of N-terminal pyroglutamates by different pGAPs were listed below in Table 9. In some cases, pGAP or a functional homolog or fragment thereof can be used to treat polypeptides.

TABLE 9
N-terminal pyrogluatamate cleavage yield by pGAPs from different organisms

| | SEQ | P. | G. | S. | O. |
|---|---|---|---|---|---|
| Peptide | ID NO | fluorescens | hollisae | albidoflavus | pratensis |
| pQAAEIRGDVRGGK(N3) | 191 | 73% | 95% | 91% | 88% |
| pQDAEIRGDVRGGK(N3) | 192 | 74% | 52% | 78% | 79% |
| pQEAEIRGDVRGGK(N3) | 193 | 69% | 77% | 80% | 81% |
| pQFAEIRGDVRGGK(N3) | 194 | 73% | 81% | 100% | 92% |
| pQGAEIRGDVRGGK(N3) | 195 | 93% | 94% | 100% | 100% |
| pQHAEIRGDVRGGK(N3) | 196 | 100% | 98% | 100% | 100% |
| pQIAEIRGDVRGGK(N3) | 197 | 88% | 69% | 90% | 79% |
| pQLAEIRGDVRGGK(N3) | 198 | 100% | 87% | 100% | 100% |
| pQMAEIRGDVRGGK(N3) | 199 | 85% | 73% | 93% | 81% |
| pQNAEIRGDVRGGK(N3) | 200 | 99% | 79% | 100% | 100% |
| pQPAEIRGDVRGGK(N3) | 201 | 4% | 4% | 4% | 4% |
| pQQAEIRGDVRGGK(N3) | 202 | 100% | 100% | 100% | 100% |
| pQSAEIRGDVRGGK(N3) | 203 | 100% | 100% | 100% | 100% |
| pQTAEIRGDVRGGK(N3) | 204 | 78% | 85% | 100% | 85% |
| pQVAEIRGDVRGGK(N3) | 205 | 78% | 88% | 100% | 83% |
| pQWAEIRGDVRGGK(N3) | 206 | 70% | 72% | 90% | 71% |
| pQYAEIRGDVRGGK(N3) | 207 | 86% | 93% | 100% | 89% |

The present disclosure is not intended to be limited in scope to the particular disclosed embodiments, which are provided to illustrate various aspects of the invention. Various modifications to the compositions and methods described will become apparent from the description and teachings herein. Such variations may be practiced without departing from the true scope and spirit of the disclosure with ordinary skill, and are intended to fall within the scope of the present invention. These and other changes can be made to the embodiments in light of the above-detailed description and the level of skill of the ordinary practitioner. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the examples.

### REFERENCES

Harlow, Ed, and David Lane. Using Antibodies. Cold Spring Harbor, New York: Cold Spring Harbor Laboratory Press, 1999.
Hennessy B T, Lu Y, Gonzalez-Angulo A M, et al. A Technical Assessment of the Utility of Reverse Phase Protein Arrays for the Study of the Functional Proteome in Non-microdissected Human Breast Cancers. Clinical proteomics. 2010; 6(4):129-151.
Davidson, G. R., S. D. Armstrong and R. J. Beynon (2011). “Positional proteomics at the N-terminus as a means of proteome simplification.” Methods Mol Biol 753: 229-242.
Zhang, L., Luo, S., and Zhang, B. (2016). The use of lectin microarray for assessing glycosylation of therapeutic proteins. mAbs 8, 524-535.
Akbani, R., K. F. Becker, N. Carragher, T. Goldstein, L. de Koning, U. Korf, L. Liotta, G. B. Mills, S. S. Nishizuka, M. Pawlak, E. F. Petricoin, 3rd, H. B. Pollard, B. Serrels and J. Zhu (2014). “Realizing the promise of reverse phase protein arrays for clinical, translational, and basic research: a workshop report: the RPPA (Reverse Phase Protein Array) society.” Mol Cell Proteomics 13(7): 1625-1643.
Amini, S., D. Pushkarev, L. Christiansen, E. Kostem, T. Royce, C. Turk, N. Pignatelli, A. Adey, J. O. Kitzman, K. Vijayan, M. Ronaghi, J. Shendure, K. L. Gunderson and F. J. Steemers (2014). “Haplotype-resolved whole-genome sequencing by contiguity-preserving transposition and combinatorial indexing.” Nat Genet 46(12): 1343-1349.
Assadi, M., J. Lamerz, T. Jarutat, A. Farfsing, H. Paul, B. Gierke, E. Breitinger, M. F. Templin, L. Essioux, S. Arbogast, M. Venturi, M. Pawlak, H. Langen and T. Schindler (2013). “Multiple protein analysis of formalin-fixed and paraffin-embedded tissue samples with reverse phase protein arrays.” Mol Cell Proteomics 12(9): 2615-2622.
Bailey, J. M. and J. E. Shively (1990). “Carboxy-terminal sequencing: formation and hydrolysis of C-terminal peptidylthiohydantoins.” Biochemistry 29(12): 3145-3156.
Bandara, H. M., D. P. Kennedy, E. Akin, C. D. Incarvito and S. C. Burdette (2009). “Photoinduced release of Zn²⁺ with ZinCleav-1: a nitrobenzyl-based caged complex.” Inorg Chem 48(17): 8445-8455.
Bandara, H. M., T. P. Walsh and S. C. Burdette (2011). “A Second-generation photocage for Zn²⁺ inspired by TPEN: characterization and insight into the uncaging quantum yields of ZinCleav chelators.” Chemistry 17(14): 3932-3941.
Basle, E., N. Joubert and M. Pucheault (2010). “Protein chemical modification on endogenous amino acids.” Chem Biol 17(3): 213-227.
Bilgicer, B., S. W. Thomas, 3rd, B. F. Shaw, G. K. Kaufman, V. M. Krishnamurthy, L. A. Estroff, J. Yang and G. M. Whitesides (2009). “A non-chromatographic method for the purification of a bivalently active monoclonal IgG antibody from biological fluids.” J Am Chem Soc 131(26): 9361-9367.
Bochman, M. L., K. Paeschke and V. A. Zakian (2012). “DNA secondary structures: stability and function of G-quadruplex structures.” Nat Rev Genet 13(11): 770-780.
Borgo, B. and J. J. Havranek (2014). “Motif-directed redesign of enzyme specificity.” Protein Sci 23(3): 312-320.
Brouzes, E., M. Medkova, N. Savenelli, D. Marran, M. Twardowski, J. B. Hutchison, J. M. Rothberg, D. R. Link, N. Perrimon and M. L. Samuels (2009). “Droplet microfluidic technology for single-cell high-throughput screening.” Proc Natl Acad Sci USA 106(34): 14195-14200.
Brudno, Y., M. E. Birnbaum, R. E. Kleiner and D. R. Liu (2010). “An in vitro translation,
selection and amplification system for peptide nucleic acids.” Nat Chem Biol 6(2): 148-155.
Calcagno, S. and C. D. Klein (2016). “N-Terminal methionine processing by the zinc-activated Plasmodium falciparum methionine aminopeptidase 1b.” Appl Microbiol Biotechnol.
Cao, Y., G. K. Nguyen, J. P. Tam and C. F. Liu (2015). “Butelase-mediated synthesis of protein thioesters and its application for tandem chemoenzymatic ligation.” Chem Commun (Camb) 51(97): 17289-17292.
Carty, R. P. and C. H. Hirs (1968). “Modification of bovine pancreatic ribonuclease A with 4-sulfonyloxy-2-nitrofluorobenzene. Isolation and identification of modified proteins.” J Biol Chem 243(20): 5244-5253.
Chan, A. I., L. M. McGregor and D. R. Liu (2015). “Novel selection methods for DNA-encoded chemical libraries.” Curr Opin Chem Biol 26: 55-61.
Chang, L., D. M. Rissin, D. R. Fournier, T. Piech, P. P. Patel, D. H. Wilson and D. C. Duffy (2012). “Single molecule enzyme-linked immunosorbent assays: theoretical considerations.” J Immunol Methods 378(1-2): 102-115.
Chang, Y. Y. and C. H. Hsu (2015). “Structural basis for substrate-specific acetylation of Nalpha-acetyltransferase Ard1 from Sulfolobus solfataricus.” Sci Rep 5: 8673.
Christoforou, A., C. M. Mulvey, L. M. Breckels, A. Geladaki, T. Hurrell, P. C. Hayward, T. Naake, L. Gatto, R. Viner, A. Martinez Arias and K. S. Lilley (2016). “A draft map of the mouse pluripotent stem cell spatial proteome.” Nat Commun 7: 8992.
Creighton, C. J. and S. Huang (2015). “Reverse phase protein arrays in signaling pathways: a data integration perspective.” Drug Des Devel Ther 9: 3519-3527.
Crosetto, N., M. Bienko and A. van Oudenaarden (2015). “Spatially resolved transcriptomics and beyond.” Nat Rev Genet 16(1): 57-66.
Cusanovich, D. A., R. Daza, A. Adey, H. A. Pliner, L. Christiansen, K. L. Gunderson, F. J. Steemers, C. Trapnell and J. Shendure (2015). “Multiplex single-cell profiling of chromatin accessibility by combinatorial cellular indexing.” Science 348(6237): 910-914.
Derrington, I. M., T. Z. Butler, M. D. Collins, E. Manrao, M. Pavlenok, M. Niederweis and J. H. Gundlach (2010). “Nanopore DNA sequencing with MspA.” Proc Natl Acad Sci USA 107(37): 16060-16065.
El-Sagheer, A. H., V. V. Cheong and T. Brown (2011). “Rapid chemical ligation of oligonucleotides by the Diels-Alder reaction.” Org Biomol Chem 9(1): 232-235.
El-Sagheer, A. H., A. P. Sanzone, R. Gao, A. Tavassoli and T. Brown (2011). “Biocompatible artificial DNA linker that is read through by DNA polymerases and is functional in Escherichia coli.” Proc Natl Acad Sci USA 108(28): 11338-11343.
Emili, A., M. McLaughlin, K. Zagorovsky, J. B. Olsen, W. C. W. Chan and S. S. Sidhu (2017). Protein Sequencing Method and Reagents. USPTO. USA, The Governing Council of University of Toronto. U.S. Pat. No. 9,566,335 B1.
Erde, J., R. R. Loo and J. A. Loo (2014). “Enhanced FASP (eFASP) to increase proteome coverage and sample recovery for quantitative proteomic experiments.” J Proteome Res 13(4): 1885-1895.
Farries, T. C., A. Harris, A. D. Auffret and A. Aitken (1991). “Removal of N-acetyl groups from blocked peptides with acylpeptide hydrolase. Stabilization of the enzyme and its application to protein sequencing.” Eur J Biochem 196(3): 679-685.
Feist, P. and A. B. Hummon (2015). “Proteomic challenges: sample preparation techniques for microgram-quantity protein analysis from biological samples.” Int J Mol Sci 16(2): 3537-3563.
Friedmann, D. R. and R. Marmorstein (2013). “Structure and mechanism of non-histone protein acetyltransferase enzymes.” FEBS J 280(22): 5570-5581.
Frokjaer, S. and D. E. Otzen (2005). “Protein drug stability: a formulation challenge.” Nat Rev Drug Discov 4(4): 298-306.
Fujii, Y., M. Kaneko, M. Neyazaki, T. Nogi, Y. Kato and J. Takagi (2014). “PA tag: a versatile protein tagging system using a super high affinity antibody against a dodecapeptide derived from human podoplanin.” Protein Expr Purif 95: 240-247.
Gebauer, M. and A. Skerra (2012). “Anticalins small engineered binding proteins based on the lipocalin scaffold.” Methods Enzymol 503: 157-188.
Gerry, N. P., N. E. Witowski, J. Day, R. P. Hammer, G. Barany and F. Barany (1999). “Universal DNA microarray method for multiplex detection of low abundance point mutations.” J Mol Biol 292(2): 251-262.
Gogliettino, M., M. Balestrieri, E. Cocca, S. Mucerino, M. Rossi, M. Petrillo, E. Mazzella and G. Palmieri (2012). “Identification and characterisation of a novel acylpeptide hydrolase from Sulfolobus solfataricus: structural and functional insights.” PLoS One 7(5): e37921.
Gogliettino, M., A. Riccio, M. Balestrieri, E. Cocca, A. Facchiano, T. M. D'Arco, C. Tesoro, M. Rossi and G. Palmieri (2014). “A novel class of bifunctional acylpeptide hydrolases—potential role in the antioxidant defense systems of the Antarctic fish Trematomus bernacchii.” FEBS J 281(1): 401-415.
Granvogl, B., M. Ploscher and L. A. Eichacker (2007). “Sample preparation by in-gel digestion for mass spectrometry-based proteomics.” Anal Bioanal Chem 389(4): 991-1002.
Gu, L., C. Li, J. Aach, D. E. Hill, M. Vidal and G. M. Church (2014). “Multiplex single-molecule interaction profiling of DNA-barcoded proteins.” Nature 515(7528): 554-557.
Gunderson, K. L., X. C. Huang, M. S. Morris, R. J. Lipshutz, D. J. Lockhart and M. S. Chee (1998). “Mutation detection by ligation to complete n-mer DNA arrays.” Genome Res 8(11): 1142-1153.
Gunderson, K. L., F. J. Steemers, J. S. Fisher and R. Rigatti (2016). Methods and Compositions for Analyzing Cellular Components. WIPO, Illumina, Inc.
Gunderson, K. L., F. J. Steemers, J. S. Fisher and R. Rigatti (2016). Methods and compositions for analyzing cellular components, Illumina, Inc.
Guo, H., W. Liu, Z. Ju, P. Tamboli, E. Jonasch, G. B. Mills, Y. Lu, B. T. Hennessy and D. Tsavachidou (2012). “An efficient procedure for protein extraction from formalin-fixed, paraffin-embedded tissues for reverse phase protein arrays.” Proteome Sci 10(1): 56.
Hamada, Y. (2016). “A novel N-terminal degradation reaction of peptides via N-amidination.” Bioorg Med Chem Lett 26(7): 1690-1695.
Hermanson, G. (2013). Bioconjugation Techniques, Academic Press.
Hernandez-Moreno, A. V., F. Villasenor, E. Medina-Rivero, N. O. Perez, L. F. Flores-Ortiz, G. Saab-Rincon and G. Luna-Barcenas (2014). “Kinetics and conformational stability studies of recombinant leucine aminopeptidase.” Int J Biol Macromol 64: 306-312.
Hori, M., H. Fukano and Y. Suzuki (2007). “Uniform amplification of multiple DNAs by emulsion PCR.” Biochem Biophys Res Commun 352(2): 323-328.
Horisawa, K. (2014). “Specific and quantitative labeling of biomolecules using click chemistry.” Front Physiol 5: 457.
Hoshika, S., F. Chen, N. A. Leal and S. A. Benner (2010). “Artificial genetic systems: self-avoiding DNA in PCR and multiplexed PCR.” Angew Chem Int Ed Engl 49(32): 5554-5557.
Hughes, A. J., D. P. Spelke, Z. Xu, C. C. Kang, D. V. Schaffer and A. E. Herr (2014). “Single-cell western blotting.” Nat Methods 11(7): 749-755.
Hughes, C. S., S. Foehr, D. A. Garfield, E. E. Furlong, L. M. Steinmetz and J. Krijgsveld (2014). “Ultrasensitive proteome analysis using paramagnetic bead technology.” Mol Syst Biol 10: 757.
Hughes, T. V., et al., J. Org. Chem. 63, 401-402 (1998).
Kang, C. C., K. A. Yamauchi, J. Vlassakis, E. Sinkala, T. A. Duncombe and A. E. Herr (2016). “Single cell-resolution western blotting.” Nat Protoc 11(8): 1508-1530.
Kang, T. S., L. Wang, C. N. Sarkissian, A. Gamez, C. R. Scriver and R. C. Stevens (2010). “Converting an injectable protein therapeutic into an oral form: phenylalanine ammonia lyase for phenylketonuria.” Mol Genet Metab 99(1): 4-9.
Katritzky, et al., J. Org. Chem. 65, 8080-8082 (2000).
Katritzky, A. R. and B. V. Rogovoy (2005). “Recent developments in guanylating agents.” ARKIVOC iv (Issue in Honor of Prof. Nikolai Zefirov): 49-87.
Klein, A. M., L. Mazutis, I. Akartuna, N. Tallapragada, A. Veres, V. Li, L. Peshkin, D. A. Weitz and M. W. Kirschner (2015). “Droplet barcoding for single-cell transcriptomics applied to embryonic stem cells.” Cell 161(5): 1187-1201.
Knall, A. C., M. Hollauf and C. Slugovc (2014). “Kinetic studies of inverse electron demand Diels-Alder reactions (iEDDA) of norbornenes and 3,6-dipyridin-2-yl-1,2,4,5-tetrazine.” Tetrahedron Lett 55(34): 4763-4766.
Kozlov, I. A., E. R. Thomsen, S. E. Munchel, P. Villegas, P. Capek, A. J. Gower, S. J. Pond, E. Chudin and M. S. Chee (2012). “A highly scalable peptide-based assay system for proteomics.” PLoS One 7(6): e37441.
Le, Z. G., Z. C. Chen, Y. Hu and Q. G. Zheng (2005). “Organic Reactions in Ionic Liquids: Ionic Liquid-promoted Efficient Synthesis of Disubstituted and Trisubstituted Thioureas Derivatives.” Chinese Chemical Letters 16(2): 201-204.
Lesch, V., A. Heuer, V. A. Tatsis, C. Holm and J. Smiatek (2015). “Peptides in the presence of aqueous ionic liquids: tunable co-solutes as denaturants or protectants?” Phys Chem Chem Phys 17(39): 26049-26053.
Li, G., Y. Liu, Y. Liu, L. Chen, S. Wu, Y. Liu and X. Li (2013). “Photoaffinity labeling of small-molecule-binding proteins by DNA-templated chemistry.” Angew Chem Int Ed Engl 52(36): 9544-9549.
Litovchick, A., M. A. Clark and A. D. Keefe (2014). “Universal strategies for the DNA-encoding of libraries of small molecules using the chemical ligation of oligonucleotide tags.” Artif DNA PNA XNA 5(1): e27896.
Liu, R., J. E. Barrick, J. W. Szostak and R. W. Roberts (2000). “Optimized synthesis of RNA-protein fusions for in vitro protein selection.” Methods Enzymol 318: 268-293.
Liu, Y. and S. Liang (2001). “Chemical carboxyl-terminal sequence analysis of peptides and proteins using tribenzylsilyl isothiocyanate.” J Protein Chem 20(7): 535-541.
Lundblad, R. L. (2014). Chemical reagents for protein modification. Boca Raton, CRC Press, Taylor & Francis Group.
Mashaghi, S. and A. M. van Oijen (2015). “External control of reactions in microdroplets.” Sci Rep 5: 11837.
McCormick, R. M. (1989). “A solid-phase extraction procedure for DNA purification.” Anal Biochem 181(1): 66-74.
Mendoza, V. L. and R. W. Vachet (2009). “Probing protein structure by amino acid-specific covalent labeling and mass spectrometry.” Mass Spectrom Rev 28(5): 785-815.
Mikami, T., T. Takao, K. Yanagi and H. Nakazawa (2012). “N (alpha) Selective Acetylation of Peptides.” Mass Spectrom (Tokyo) 1(2): A0010.
Moghaddam, M. J., L. de Campo, N. Kirby and C. J. Drummond (2012). “Chelating DTPA amphiphiles: ion-tunable self-assembly structures and gadolinium complexes.” Phys Chem Chem Phys 14(37): 12854-12862.
Mukherjee, S., M. Ura, R. J. Hoey and A. A. Kossiakoff (2015). “A New Versatile Immobilization Tag Based on the Ultra High Affinity and Reversibility of the Calmodulin-Calmodulin Binding Peptide Interaction.” J Mol Biol 427(16): 2707-2725.
Namimatsu, S., M. Ghazizadeh and Y. Sugisaki (2005). “Reversing the effects of formalin fixation with citraconic anhydride and heat: a universal antigen retrieval method.” J Histochem Cytochem 53(1): 3-11.
Nguyen, G. K., Y. Cao, W. Wang, C. F. Liu and J. P. Tam (2015). “Site-Specific N-Terminal Labeling of Peptides and Proteins using Butelase 1 and Thiodepsipeptide.” Angew Chem Int Ed Engl 54(52): 15694-15698.
Nguyen, G. K., S. Wang, Y. Qiu, X. Hemu, Y. Lian and J. P. Tam (2014). “Butelase 1 is an Asx-specific ligase enabling peptide macrocyclization and synthesis.” Nat Chem Biol 10(9): 732-738.
Nirantar, S. R. and F. J. Ghadessy (2011). “Compartmentalized linkage of genes encoding interacting protein pairs.” Proteomics 11(7): 1335-1339.
Nishizuka, S. S. and G. B. Mills (2016). “New era of integrated cancer biomarker discovery using reverse-phase protein arrays.” Drug Metab Pharmacokinet 31(1): 35-45.
Ohkubo, A., R. Kasuya, K. Sakamoto, K. Miyata, H. Taguchi, H. Nagasawa, T. Tsukahara, T. Watanobe, Y. Maki, K. Seio and M. Sekine (2008). “‘Protected DNA Probes’ capable of strong hybridization without removal of base protecting groups.” Nucleic Acids Res 36(6): 1952-1964.
Ojha, B., A. K. Singh, M. D. Adhikari, A. Ramesh and G. Das (2010). “2-Alkylmalonic acid: amphiphilic chelator and a potent inhibitor of metalloenzyme.” J Phys Chem B 114(33): 10835-10842.
Peng, X., H. Li and M. Seidman (2010). “A Template-Mediated Click-Click Reaction: PNA-DNA, PNA-PNA (or Peptide) Ligation, and Single Nucleotide Discrimination.” European J Org Chem 2010(22): 4194-4197.
Perbandt, M., O. Bruns, M. Vallazza, T. Lamla, C. Betzel and V. A. Erdmann (2007). “High resolution structure of streptavidin in complex with a novel high affinity peptide tag mimicking the biotin binding motif” Proteins 67(4): 1147-1153.
Rauth, S., D. Hinz, M. Borger, M. Uhrig, M. Mayhaus, M. Riemenschneider and A. Skerra (2016). “High-affinity Anticalins with aggregation-blocking activity directed against the Alzheimer beta-amyloid peptide.” Biochem J 473(11): 1563-1578.
Ray, A. and B. Norden (2000). “Peptide nucleic acid (PNA): its medical and biotechnical applications and promise for the future.” FASEB J 14(9): 1041-1060.
Ren, et al., J. Label Compd. Radiopharm. 53, 239-268 (2010).
Riley, N. M., A. S. Hebert and J. J. Coon (2016). “Proteomics Moves into the Fast Lane.” Cell Syst 2(3): 142-143.
Roloff, A., S. Ficht, C. Dose and O. Seitz (2014). “DNA-templated native chemical ligation of functionalized peptide nucleic acids: a versatile tool for single base-specific detection of nucleic acids.” Methods Mol Biol 1050: 131-141.
Roloff, A. and O. Seitz (2013). “The role of reactivity in DNA templated native chemical PNA ligation during PCR.” Bioorg Med Chem 21(12): 3458-3464.
Sakurai, K., T. M. Snyder and D. R. Liu (2005). “DNA-templated functional group transformations enable sequence-programmed synthesis using small-molecule reagents.” J Am Chem Soc 127(6): 1660-1661.
Schneider, K. and B. T. Chait (1995). “Increased stability of nucleic acids containing 7-deaza-guanosine and 7-deaza-adenosine may enable rapid DNA sequencing by matrix-assisted laser desorption mass spectrometry.” Nucleic Acids Res 23(9): 1570-1575.
Selvaraj, R. and J. M. Fox (2013). “trans-Cyclooctene—a stable, voracious dienophile for bioorthogonal labeling.” Curr Opin Chem Biol 17(5): 753-760.
Sharma, A. K., A. D. Kent and J. M. Heemstra (2012). “Enzyme-linked small-molecule detection using split aptamer ligation.” Anal Chem 84(14): 6104-6109.
Shembekar, N., C. Chaipan, R. Utharala and C. A. Merten (2016). “Droplet-based microfluidics in drug discovery, transcriptomics and high-throughput molecular genetics.” Lab Chip 16(8): 1314-1331.
Shenoy, N. R., J. E. Shively and J. M. Bailey (1993). “Studies in C-terminal sequencing: new reagents for the synthesis of peptidylthiohydantoins.” J Protein Chem 12(2): 195-205.
Shim, J. U., R. T. Ranasinghe, C. A. Smith, S. M. Ibrahim, F. Hollfelder, W. T. Huck, D. Klenerman and C. Abell (2013). “Ultrarapid generation of femtoliter microfluidic droplets for single-molecule-counting immunoassays.” ACS Nano 7(7): 5955-5964.
Shim, J. W., Q. Tan and L. Q. Gu (2009). “Single-molecule detection of folding and unfolding of the G-quadruplex aptamer in a nanopore nanocavity.” Nucleic Acids Res 37(3): 972-982.
Sidoli, S., Z. F. Yuan, S. Lin, K. Karch, X. Wang, N. Bhanu, A. M. Arnaudo, L. M. Britton, X. J. Cao, M. Gonzales-Cope, Y. Han, S. Liu, R. C. Molden, S. Wein, L. Afjehi-Sadat and B. A. Garcia (2015). “Drawbacks in the use of unconventional hydrophobic anhydrides for histone derivatization in bottom-up proteomics PTM analysis.” Proteomics 15(9): 1459-1469.
Sletten, E. M. and C. R. Bertozzi (2009). “Bioorthogonal chemistry: fishing for selectivity in a sea of functionality.” Angew Chem Int Ed Engl 48(38): 6974-6998.
Spencer, S. J., M. V. Tamminen, S. P. Preheim, M. T. Guo, A. W. Briggs, I. L. Brito, A. W. D, L. K. Pitkanen, F. Vigneault, M. P. Juhani Virta and E. J. Alm (2016). “Massively parallel sequencing of single cells by epicPCR links functional genes with phylogenetic markers.” ISME J 10(2): 427-436.
Spicer, C. D. and B. G. Davis (2014). “Selective chemical protein modification.” Nat Commun 5: 4740.
Spiropulos, N. G. and J. M. Heemstra (2012). “Templating effect in DNA proximity ligation enables use of non-bioorthogonal chemistry in biological fluids.” Artif DNA PNA XNA 3(3): 123-128.
Switzar, L., M. Giera and W. M. Niessen (2013). “Protein digestion: an overview of the available techniques and recent developments.” J Proteome Res 12(3): 1067-1077.
Tamminen, M. V. and M. P. Virta (2015). “Single gene-based distinction of individual microbial genomes from a mixed population of microbial cells.” Front Microbiol 6: 195.
Tessler, L. (2011). Digital Protein Analysis: Technologies for Protein Diagnostics and Proteomics through Single-Molecule Detection. Ph.D., WASHINGTON UNIVERSITY IN ST. LOUIS.
Tyson, J. and J. A. Armour (2012). “Determination of haplotypes at structurally complex regions using emulsion haplotype fusion PCR.” BMC Genomics 13: 693.
Vauquelin, G. and S. J. Charlton (2013). “Exploring avidity: understanding the potential gains in functional affinity and target residence time of bivalent and heterobivalent ligands.” Br J Pharmacol 168(8): 1771-1785.
Veggiani, G., T. Nakamura, M. D. Brenner, R. V. Gayet, J. Yan, C. V. Robinson and M. Howarth (2016). “Programmable polyproteams built using twin peptide superglues.” Proc Natl Acad Sci USA 113(5): 1202-1207.
Wang, D., S. Fang and R. M. Wohlhueter (2009). “N-terminal derivatization of peptides with isothiocyanate analogues promoting Edman-type cleavage and enhancing sensitivity in electrospray ionization tandem mass spectrometry analysis.” Anal Chem 81(5): 1893-1900.
Williams, B. A. and J. C. Chaput (2010). “Synthesis of peptide-oligonucleotide conjugates using a heterobifunctional crosslinker.” Curr Protoc Nucleic Acid Chem Chapter 4: Unit4 41.
Wu, H. and N. K. Devaraj (2016). “Inverse Electron-Demand Diels-Alder Bioorthogonal Reactions.” Top Curr Chem (J) 374(1): 3.
Xiong, A. S., R. H. Peng, J. Zhuang, F. Gao, Y. Li, Z. M. Cheng and Q. H. Yao (2008). “Chemical gene synthesis: strategies, softwares, error corrections, and applications.” FEMS Microbiol Rev 32(3): 522-540.
Yao, Y., M. Docter, J. van Ginkel, D. de Ridder and C. Joo (2015). “Single-molecule protein sequencing through fingerprinting: computational assessment.” Phys Biol 12(5): 055003.
Zakeri, B., J. O. Fierer, E. Celik, E. C. Chittock, U. Schwarz-Linek, V. T. Moy and M. Howarth
(2012). “Peptide tag forming a rapid covalent bond to a protein, through engineering a bacterial adhesin.” Proc Natl Acad Sci USA 109(12): E690-697.
Zhang, L., K. Zhang, S. Rauf, D. Dong, Y. Liu and J. Li (2016). “Single-Molecule Analysis of Human Telomere Sequence Interactions with G-quadruplex Ligand.” Anal Chem 88(8): 4533-4540.
Zhou, H., Z. Ning, A. E. Starr, M. Abu-Farha and D. Figeys (2012). “Advancements in top-down proteomics.” Anal Chem 84(2): 720-734.
Zilionis, R., J. Nainys, A. Veres, V. Savova, D. Zemmour, A. M. Klein and L. Mazutis (2017). “Single-cell barcoding and sequencing using droplet microfluidics.” Nat Protoc 12(1): 44-73.
Bachor et al., Mol. Divers. 2013, 17, 605-611.
Bader et al., Arch Occup Environ Healt, 1994, 65(6), 411-414.
Barrett et al., Tetrahedron Lett., 1985, 26(36), 4375-4378.
Bentley et al., Biochem. J. 1973(135), 507-511.
Bentley et al., Biochem. J, 1976(153), 137-138.
Bhattacharjree et al., J. Chem. Sci. 2016, 128(6):875-881.
Borgo et al., Protein Science. 2015, 24(4), 571-579.
Buckingham et al., J. Am. Chem. Soc. 1970, 92(19), 5571-5579.
Chi et al., 2015, Chem. Eur. J. 2015, 21, 10369-10378.
Fang et al., Peptide Science, 2010, 96 (1), 97-102.
Hamada, Y., Bioog. Med. Chem. Lett. 2016, 26, 1690-1695.
Huo et al., J. Am. Chem. Soc. 2007, 139, 9819-9822
Katritzky et al., Arkivoc. 2005, iv, 49-87.
Krishna et al., Protein Science. 1992, 1(5), 582-589.
Kwon et al., Org. Lett. 2014, 16, 6048-6051.
Martin et al., Organometallics. 2006, 34, 1787-1801.
Musiol et al., Org. Lett., 2001, 3 (15), 2341-2344.
Proulx et al., Peptide Science, 2016, 106(5), 726-736.
Rydberg et al., Chem. Res. Toxicol., 2002, 15(4), 570-581.
Sutton et al, Acc. Chem. Res. 1987, 20(10), 357-364.
Tam et al., 2007, J. Am. Chem. Soc. 2007, 129, 12670-12671.
Tian et al., J. Am. Chem. Soc., 2016, 138(43), pp. 14234-14237.
Tornqvist et al., Anal. Biochem. 1986, 154, 255-266
Vigneron et al., Proc. Natl. Acad. Sci. 1996, 93, 9682-9686.
Wu et al., J. Am. Chem. Soc. 2016, 138(44), 14554-14557
Xu et al., Organometallics. 2015, 34, 1787-1801.
Yong et al., J. Org. Chem. 1997, 62, 1540-1542.
Zhang et al., Org. Lett., 2001, 3 (15), 2341-2344.
Basten, D. E., A. P. Moers, A. J. Ooyen and P. J. Schaap (2005). “Characterisation of Aspergillus niger prolyl aminopeptidase.” Mol Genet Genomics 272(6): 673-679.
Bolumar, T., Y. Sanz, M. C. Aristoy and F. Toldra (2003). “Purification and properties of an arginyl aminopeptidase from Debaryomyces hansenii.” Int J Food Microbiol 86(1-2): 141-151.
Chanalia, P., D. Gandhi, P. Attri and S. Dhanda (2018). “Extraction, purification and characterization of low molecular weight Proline iminopeptidase from probiotic L. plantarum for meat tenderization.” Int J Biol Macromol 109: 651-663.
Kitazono, A., T. Yoshimoto and D. Tsuru (1992). “Cloning, sequencing, and high expression of the proline iminopeptidase gene from Bacillus coagulans.” J Bacteriol 174(24): 7919-7925.
Nakajima, Y., K. Ito, M. Sakata, Y. Xu, K. Nakashima, F. Matsubara, S. Hatakeyama and T. Yoshimoto (2006). “Unusual extra space at the active site and high activity for acetylated hydroxyproline of prolyl aminopeptidase from Serratia marcescens.” J Bacteriol 188(4): 1599-1606.
WO2011/126903
WO 2012/101654
WO 2006/17409
EP2862856

1. A method to cleave an N-terminal amino acid residue from a peptidic compound of Formula (I) 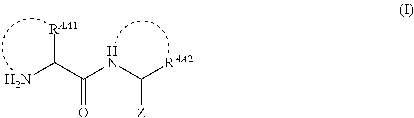
wherein the method comprises:
(1) converting the peptidic compound to a guanidinyl derivative of Formula (II): 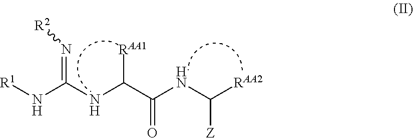
or a tautomer thereof; and
(2) contacting the guanidinyl derivative with a suitable medium to produce a compound of Formula (III) 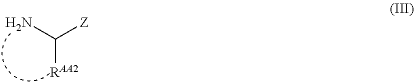
wherein:
R¹ is R⁶, NHR³, —NHC(O)—R³, or —NH—SO₂—R³
R² is H or R⁴;
R³ is H or R⁶, wherein R⁶ is an optionally substituted group selected from phenyl, 5-membered heteroaryl, 6-membered heteroaryl, C₁₋₃ haloalkyl, and C₁₋₆ alkyl,
wherein optional substituents of the optionally substituted group are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
and wherein two R′ or two R″ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;
R^{AA1} and R^{AA2} are each independently selected amino acid side chains;
and the dashed semi-circle connecting R^{AA1} and/or R^{AA2} to the nearest N atom indicates that R^{AA1} and/or R^{AA2} can optionally cyclize onto the designated N atom; and
Z is —COOH, CONH₂, or an amino acid or a polypeptide that is optionally attached to a carrier or solid support.

2. The method of claim 1, wherein Z is a polypeptide.

3. The method of claim 1, wherein Z is a polypeptide attached to a solid support

4-5. (canceled)

6. The method of claim 2, wherein the polypeptide is attached to a nucleic acid that is optionally covalently joined to a solid support.

7-12. (canceled)

13. The method of claim 5, wherein the suitable medium for step (2) has pH between about 5 and 9, and optionally includes a hydroxide, carbonate, phosphate, sulfate or amine

14. (canceled)

15. The method of claim 5, wherein the medium comprises a diheteronucleophile.

16. The method of claim 5, wherein R² is H and R¹ is NH₂.

17. The method of claim 5, wherein contacting the guanidinyl derivative with the suitable medium at step (2) occurs at temperature between 40° C. and 95° C.

18. (canceled)

19. The method of claim 1, wherein the compound of Formula (I) is of the formula (IA): 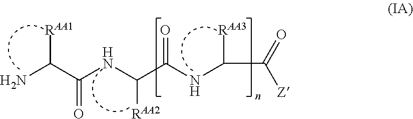
and the compound of Formula (III) is a compound of the formula (IIIA): 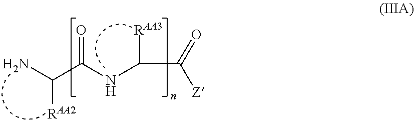
where n is an integer from 1 to 1000;
R^{AA1} and R^{AA2} are as defined in claim 1;
the dashed semi-circle connecting R^{AA1} and R^{AA2} and R^{AA3} to the adjacent N atom indicates that R^{AA1} and/or R^{AA2} and/or R^{AA3} can optionally cyclize onto the designated adjacent N atom; and
each R^{AA3} is independently selected from amino acid side chains, including natural and non-natural amino acids;
and Z′ is OH or NH₂, or Z′ is O or N that is attached to a carrier or solid support.

20. The method of claim 1, wherein the guanidinyl derivative of Formula (II) is produced by converting the peptidic compound of Formula (I) to a compound of the formula (IV): 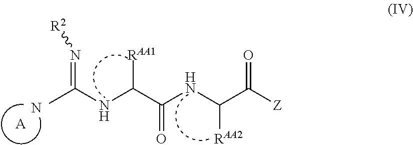
wherein ring A is a 5-6 membered heteroaryl ring containing up to three N atoms as ring members, optionally fused to an additional 5-6 membered heteroaryl or phenyl ring, and wherein the 5-6 membered heteroaryl ring and optional additional 5-6 membered heteroaryl or phenyl ring are each optionally substituted with up to four groups selected from C₁₋₄ alkyl, C₁₋₄ alkoxy, —OH, halo, C₁₋₄ haloalkyl, NO₂, COOR, CONR₂, —SO₂R*, and —NR₂;
wherein each R is independently selected from H and C₁₋₃ alkyl, optionally substituted with OH, OR*, —NH₂, and —NR*₂; and
each R* is C₁₋₃ alkyl, optionally substituted with OH, C₁₋₂ alkoxy, —NH₂, or CN;
or a salt thereof;
wherein two R or two R* on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;
the dashed semi-circle connecting R^{AA1} and R^{AA2} to the nearest N atom indicates that R^{AA1} and/or R^{AA2} optionally cyclize onto the designated N atom;
then contacting this compound with a diheteronucleophile, optionally in the presence of a buffer, to produce the compound of Formula (II).

21. The method of claim 20, wherein the peptidic compound of Formula (I) is converted to a compound of Formula (IV) by contacting the compound of Formula (I) with a compound of the formula: 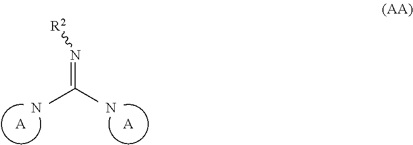
wherein:
R² is H or R⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, B(OR)₂, Bpin (boranyl pinacolate), phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, and CN;
to form the compound of Formula (IV).

22. The method of claim 21, wherein ring A is selected from: 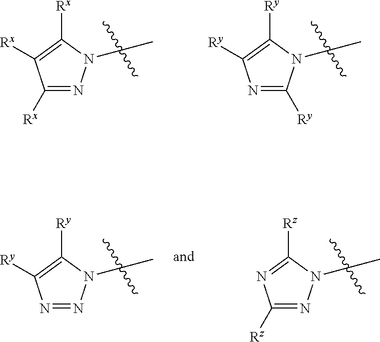
wherein:
each R^{x}, R^{y} and R^{z} is independently selected from H, halo, C₁₋₂ alkyl, C₁₋₂ haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂ haloalkyl, NO₂, SO₂(C₁₋₂ alkyl), COOR^{#}, and C(O)N(R^{#})₂,
and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;
wherein each R^{#} is independently H or C₁₋₂ alkyl; and wherein two R^{#} on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;
or a salt thereof.

23-28. (canceled)

29. The method of claim 20, wherein the suitable medium in step (2) comprises a diheteronucleophile that is selected from:

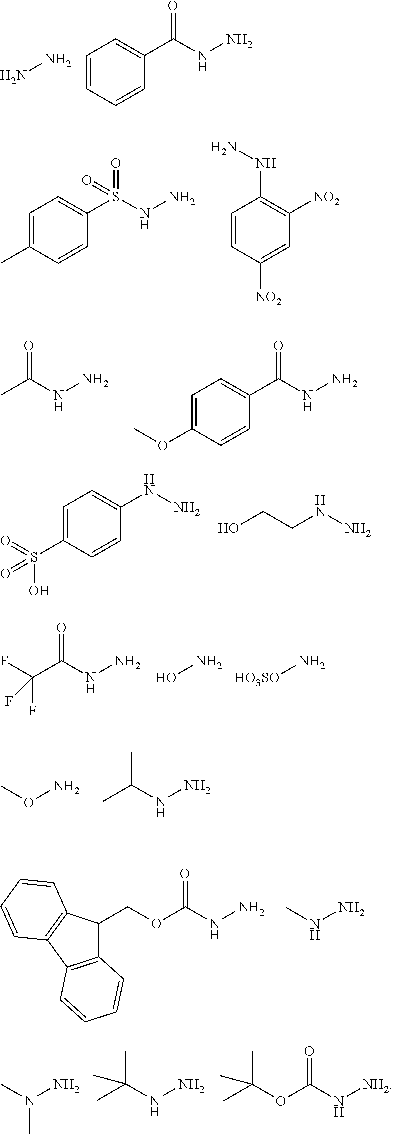

30-31. (canceled)

32. A compound of the Formula: 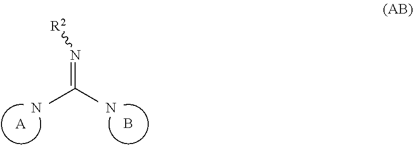
wherein:
R² is H or R⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
ring A and ring B are each independently a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
with the proviso that Ring A and Ring B are not both unsubstituted imidazole and that Ring A and Ring B are not both unsubstituted benzotriazole;
or a salt thereof.

33-35. (canceled)

36. The compound of claim 32, wherein Ring A and Ring B are selected from: 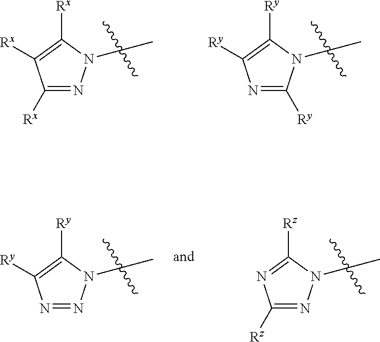
wherein:
each R^{x}, R^{y} and R^{z} is independently selected from H, halo, C₁₋₂ alkyl, C₁₋₂haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂haloalkyl, NO₂, SO₂(C₁₋₂ alkyl), COOR^{#}, and C(O)N(R¹⁹⁰)₂,
and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;
wherein each R^{#} is independently H or C₁₋₂ alkyl; and wherein two R^{#} on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;
or a salt thereof.

37-38. (canceled)

39. A compound of Formula (II): 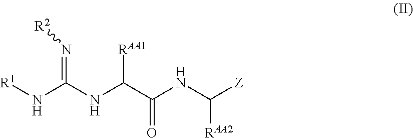
or a tautomer thereof,
wherein:
R¹ is R⁶, NHR³, —NHC(O)—R³, or —NH—SO₂—R³;
R² is H or R⁴;
R³ is H or R6, wherein R6 is an optionally substituted group selected from phenyl, 5-membered heteroaryl, 6-membered heteroaryl, C₁₋₃ haloalkyl, and C₁₋₆ alkyl,
wherein optional substituents of the optionally substituted group are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
wherein two R′ or two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
R^{AA1} and R^{AA2} are each independently selected from H and C₁₋₆ alkyl optionally substituted with one or two groups independently selected from —OR⁵, —N(R⁵)₂, —SR⁵, —SeR⁵, —COOR⁵, CON(R⁵)₂, —NR⁵—C(═NR⁵)—N(R⁵)₂, phenyl, imidazolyl, and indolyl, where phenyl, imidazolyl and indolyl are each optionally substituted with halo, C₁₋₃ alkyl, C₁₋₃ haloalkyl, —OH, C₁₋₃ alkoxy, CN, COOR⁵, or CON(R⁵)₂;
each R⁵ is independently selected from H and C₁₋₂ alkyl;
and Z is —COOH, CONH₂, or an amino acid or polypeptide that is optionally attached to a carrier or surface; or a salt thereof.

40-42. (canceled)

43. The compound of claim 39, wherein Z is a polypeptide attached to a solid support

44-48. (canceled)

49. A compound of Formula (IV): 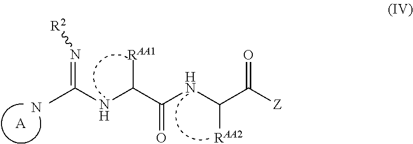
wherein: R² is H or R⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
wherein two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
R^{AA1} and R^{AA2} are each independently selected amino acid side chains;
and the dashed semi-circle connecting R^{AA1} and/or R^{AA2} to the nearest N atom indicates that R^{AA1} and/or R^{AA2} can optionally cyclize onto the designated N atom; and
Z is —COOH, CONH₂, or an amino acid or a polypeptide that is optionally attached to a carrier or solid support;
or a salt thereof.

50-52. (canceled)

53. The compound of claim 49, wherein Z is an amino acid or polypeptide that is attached to a solid support.

54-60. (canceled)

61. A method to identify the N-terminal amino acid residue of a peptidic compound of the Formula (I): 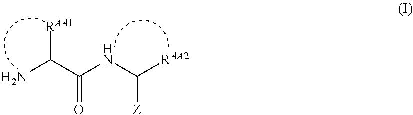
wherein the method comprises:
(1) converting the compound of Formula (I) to a guanidinyl derivative of Formula (II) or a tautomer thereof: 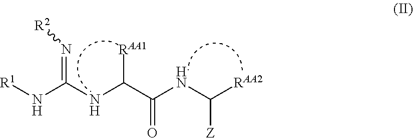
wherein:
R¹ is R⁶, NHR³, —NHC(O)—R³, or —NH—SO₂—R³
R² is H or R⁴;
R³ is H or R⁶, wherein R⁶ is an optionally substituted group selected from phenyl, 5-membered heteroaryl, 6-membered heteroaryl, C₁₋₃ haloalkyl, and C₁₋₆ alkyl,
wherein optional substituents of the optionally substituted group are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
wherein two R′ or two R″ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
R^{AA1} and R^{AA2} are each independently selected amino acid side chains;
and the dashed semi-circle connecting R^{AA1} and/or R^{AA2} to the nearest N atom indicates that R^{AA1} and/or R^{AA2} can optionally cyclize onto the designated N atom; and
and Z is —COOH, CONH₂, or an amino acid or polypeptide that is optionally attached to a carrier or surface;
(2) contacting the guanidinyl derivative with a suitable medium to induce elimination of the modified N-terminal amino acid and produce at least one cleavage product selected from: 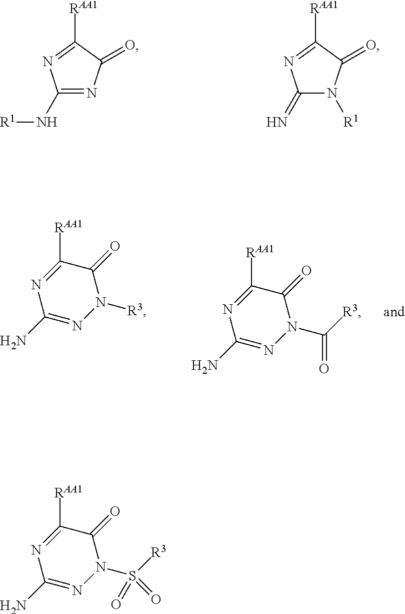
(wherein R¹ is NHR³, —NHC(O) R³, or —NH—SO₂—R³, respectively) or a tautomer thereof; and
determining the structure or identity of the at least one cleavage product to identify the N-terminal amino acid of the compound of Formula (I).

62. The method of claim 61, wherein R^{AA1} and R^{AA2} are each independently selected from H and C₁₋₆ alkyl optionally substituted with one or two groups independently selected from —OW, —N(R⁵)₂, —SR⁵, —SeR⁵, —COOR⁵, CON(R⁵)₂, —NR⁵—C(═NR⁵)—N(R⁵)₂, phenyl, imidazolyl, and indolyl, where phenyl, imidazolyl and indolyl are each optionally substituted with halo, C₁₋₃ alkyl, C₁₋₃ haloalkyl, —OH, C₁₋₃ alkoxy, CN, COOR⁵, or CON(R⁵)₂; and
each R⁵ is independently selected from H and C₁₋₂ alkyl.

63-67. (canceled)

68. The method of claim 61, wherein Z is an amino acid or polypeptide that is attached to a solid support.

69-73. (canceled)

74. A method for analyzing a polypeptide, comprising the steps of:
(a) providing the polypeptide optionally associated directly or indirectly with a recording tag;
(b) functionalizing the N-terminal amino acid (NTAA) of the polypeptide with a chemical reagent, wherein the chemical reagent is either:
(b1) a compound of Formula (AA): 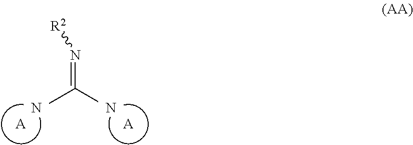
wherein:
R² is H or R⁴;
R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
or
(b2) a compound of the formula R³—NCS;
wherein R³ is H or an optionally substituted group selected from phenyl, 5-membered heteroaryl, 6-membered heteroaryl, C₁₋₃ haloalkyl, and C₁₋₆ alkyl,
wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;
wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
to provide an initial NTAA functionalized polypeptide;
optionally treating the initial NTAA functionalized polypeptide with an amine of Formula R²—NH₂ or with a diheteronucleophile to form a secondary NTAA functionalized polypeptide;
and optionally treating the initial NTAA functionalized polypeptide or the secondary NTAA functionalized polypeptide with a suitable medium to eliminate the NTAA and form an N-terminally truncated polypeptide;
(c) contacting the polypeptide with a first binding agent comprising a first binding portion capable of binding to the polypeptide, or to the initial NTAA functionalized polypeptide, or to the secondary NTAA functionalized polypeptide, or to the N-terminally truncated polypeptide; and either
(c1) a first coding tag with identifying information regarding the first binding agent, or
(c2) a first detectable label;
(d) (d1) transferring the information of the first coding tag to the recording tag to generate an extended recording tag and analyzing the extended recording tag, or
(d2) detecting the first detectable label.

75. The method of claim 74, further comprising repeating steps (b) through (d) to determine the sequence of at least a part of the polypeptide.

76-214. (canceled)


SEQUENCE LISTING (207 sequences)

SEQ ID NO: 1 - DNA, 15 bp, Artificial Sequence
  oligonucleotide barcode BC_1
atgtctagcatgccg

SEQ ID NO: 2 - DNA, 15 bp, Artificial Sequence
  oligonucleotide barcode BC_2
ccgtgtcatgtggaa

SEQ ID NO: 3 - DNA, 15 bp, Artificial Sequence
  oligonucleotide barcode BC_3
taagccggtatatca

SEQ ID NO: 4 - DNA, 15 bp, Artificial Sequence
  oligonucleotide barcode BC_4
ttcgatatgacggaa

SEQ ID NO: 5 - DNA, 15 bp, Artificial Sequence
  oligonucleotide barcode BC_5
cgtatacgcgttagg

SEQ ID NO: 6 - DNA, 15 bp, Artificial Sequence
  oligonucleotide barcode BC_6
aactgccgagattcc

SEQ ID NO: 7 - DNA, 15 bp, Artificial Sequence
  oligonucleotide barcode BC_7
tgatcttagctgtgc

SEQ ID NO: 8 - DNA, 15 bp, Artificial Sequence
  oligonucleotide barcode BC_8
gagtcggtaccttga

SEQ ID NO: 9 - DNA, 15 bp, Artificial Sequence
  oligonucleotide barcode BC_9
ccgcttgtgatctgg

SEQ ID NO: 10 - DNA, 15 bp, Artificial Sequence
  oligonucleotide barcode BC_10
agatagcgtaccgga

SEQ ID NO: 11 - DNA, 15 bp, Artificial Sequence
  oligonucleotide barcode BC_11
tccaggctcatcatc

SEQ ID NO: 12 - DNA, 15 bp, Artificial Sequence
  oligonucleotide barcode BC_12
gagtactagagccaa

SEQ ID NO: 13 - DNA, 15 bp, Artificial Sequence
  oligonucleotide barcode BC_13
gagcgtcaataacgg

SEQ ID NO: 14 - DNA, 15 bp, Artificial Sequence
  oligonucleotide barcode BC_14
gcggtatctacactg

SEQ ID NO: 15 - DNA, 15 bp, Artificial Sequence
  oligonucleotide barcode BC_15
cttctccgaagagaa

SEQ ID NO: 16 - DNA, 15 bp, Artificial Sequence
  oligonucleotide barcode BC_16
tgaagcctgtgttaa

SEQ ID NO: 17 - DNA, 15 bp, Artificial Sequence
  oligonucleotide barcode BC_17
ctggatggttgtcga

SEQ ID NO: 18 - DNA, 15 bp, Artificial Sequence
  oligonucleotide barcode BC_18
actgcacggttccaa

SEQ ID NO: 19 - DNA, 15 bp, Artificial Sequence
  oligonucleotide barcode BC_19
cgagagatggtcctt

SEQ ID NO: 20 - DNA, 15 bp, Artificial Sequence
  oligonucleotide barcode BC_20
tcttgagagacaaga

SEQ ID NO: 21 - DNA, 15 bp, Artificial Sequence
  oligonucleotide barcode BC_21
aattcgcactgtgtt

SEQ ID NO: 22 - DNA, 15 bp, Artificial Sequence
  oligonucleotide barcode BC_22
gtagtgccgctaaga

SEQ ID NO: 23 - DNA, 15 bp, Artificial Sequence
  oligonucleotide barcode BC_23
cctatagcacaatcc

SEQ ID NO: 24 - DNA, 15 bp, Artificial Sequence
  oligonucleotide barcode BC_24
atcaccgaggttgga

SEQ ID NO: 25 - DNA, 15 bp, Artificial Sequence
  oligonucleotide barcode BC_25
gattcaacggagaag

SEQ ID NO: 26 - DNA, 15 bp, Artificial Sequence
  oligonucleotide barcode BC_26
acgaacctcgcacca

SEQ ID NO: 27 - DNA, 15 bp, Artificial Sequence
  oligonucleotide barcode BC_27
aggacttcaagaaga

SEQ ID NO: 28 - DNA, 15 bp, Artificial Sequence
  oligonucleotide barcode BC_28
ggttgaatcctcgca

SEQ ID NO: 29 - DNA, 15 bp, Artificial Sequence
  oligonucleotide barcode BC_29
aaccaacctctagcg

SEQ ID NO: 30 - DNA, 15 bp, Artificial Sequence
  oligonucleotide barcode BC_30
acgcgaatatctaac

SEQ ID NO: 31 - DNA, 15 bp, Artificial Sequence
  oligonucleotide barcode BC_31
gttgagaattacacc

SEQ ID NO: 32 - DNA, 15 bp, Artificial Sequence
  oligonucleotide barcode BC_32
ctctctctgtgaacc

SEQ ID NO: 33 - DNA, 15 bp, Artificial Sequence
  oligonucleotide barcode BC_33
gccatcagtaagaga

SEQ ID NO: 34 - DNA, 15 bp, Artificial Sequence
  oligonucleotide barcode BC_34
gcaacgtgaattgag

SEQ ID NO: 35 - DNA, 15 bp, Artificial Sequence
  oligonucleotide barcode BC_35
ctaagtagagccaca

SEQ ID NO: 36 - DNA, 15 bp, Artificial Sequence
  oligonucleotide barcode BC_36
tgtctgttggaagcg

SEQ ID NO: 37 - DNA, 15 bp, Artificial Sequence
  oligonucleotide barcode BC_37
ttaatagacagcgcg

SEQ ID NO: 38 - DNA, 15 bp, Artificial Sequence
  oligonucleotide barcode BC_38
cgacgctctaacaag

SEQ ID NO: 39 - DNA, 15 bp, Artificial Sequence
  oligonucleotide barcode BC_39
catggcttattgaga

SEQ ID NO: 40 - DNA, 15 bp, Artificial Sequence
  oligonucleotide barcode BC_40
actaggtatggccgg

SEQ ID NO: 41 - DNA, 15 bp, Artificial Sequence
  oligonucleotide barcode BC_41
gtcctcgtctatcct

SEQ ID NO: 42 - DNA, 15 bp, Artificial Sequence
  oligonucleotide barcode BC_42
taggattccgttacc

SEQ ID NO: 43 - DNA, 15 bp, Artificial Sequence
  oligonucleotide barcode BC_43
tctgaccaccggaag

SEQ ID NO: 44 - DNA, 15 bp, Artificial Sequence
  oligonucleotide barcode BC_44
agagtcacctcgtgg

SEQ ID NO: 45 - DNA, 15 bp, Artificial Sequence
  oligonucleotide barcode BC_45
ctgatgtagtcgaag

SEQ ID NO: 46 - DNA, 15 bp, Artificial Sequence
  oligonucleotide barcode BC_46
gtcggttgcggatag

SEQ ID NO: 47 - DNA, 15 bp, Artificial Sequence
  oligonucleotide barcode BC_47
tcctcctcctaagaa

SEQ ID NO: 48 - DNA, 15 bp, Artificial Sequence
  oligonucleotide barcode BC_48
attcggtccacttca

SEQ ID NO: 49 - DNA, 15 bp, Artificial Sequence
  oligonucleotide barcode BC_49
ccttacaggtctgcg

SEQ ID NO: 50 - DNA, 15 bp, Artificial Sequence
  oligonucleotide barcode BC_50
gatcattggccaatt

SEQ ID NO: 51 - DNA, 15 bp, Artificial Sequence
  oligonucleotide barcode BC_51
ttcaaggctgagttg

SEQ ID NO: 52 - DNA, 15 bp, Artificial Sequence
  oligonucleotide barcode BC_52
tggctcgattgaatc

SEQ ID NO: 53 - DNA, 15 bp, Artificial Sequence
  oligonucleotide barcode BC_53
gtaagccatccgctc

SEQ ID NO: 54 - DNA, 15 bp, Artificial Sequence
  oligonucleotide barcode BC_54
acacatgcgtagaca

SEQ ID NO: 55 - DNA, 15 bp, Artificial Sequence
  oligonucleotide barcode BC_55
tgctatggattcaag

SEQ ID NO: 56 - DNA, 15 bp, Artificial Sequence
  oligonucleotide barcode BC_56
ccacgaggcttagtt

SEQ ID NO: 57 - DNA, 15 bp, Artificial Sequence
  oligonucleotide barcode BC_57
ggccaactaaggtgc

SEQ ID NO: 58 - DNA, 15 bp, Artificial Sequence
  oligonucleotide barcode BC_58
gcacctattcgacaa

SEQ ID NO: 59 - DNA, 15 bp, Artificial Sequence
  oligonucleotide barcode BC_59
tggacacgatcggct

SEQ ID NO: 60 - DNA, 15 bp, Artificial Sequence
  oligonucleotide barcode BC_60
ctataattccaacgg

SEQ ID NO: 61 - DNA, 15 bp, Artificial Sequence
  oligonucleotide barcode BC_61
aacgtggttagtaag

SEQ ID NO: 62 - DNA, 15 bp, Artificial Sequence
  oligonucleotide barcode BC_62
caaggaacgagtggc

SEQ ID NO: 63 - DNA, 15 bp, Artificial Sequence
  oligonucleotide barcode BC_63
caccagaacggaaga

SEQ ID NO: 64 - DNA, 15 bp, Artificial Sequence
  oligonucleotide barcode BC_64
cgtacggtcaagcaa

SEQ ID NO: 65 - DNA, 15 bp, Artificial Sequence
  oligonucleotide barcode BC_65
tcggtgacaggctaa

SEQ ID NO: 66 - DNA, 15 bp, Artificial Sequence
  oligonucleotide barcode BC_1 REV
cggcatgctagacat

SEQ ID NO: 67 - DNA, 15 bp, Artificial Sequence
  oligonucleotide barcode BC_2  REV
ttccacatgacacgg

SEQ ID NO: 68 - DNA, 15 bp, Artificial Sequence
  oligonucleotide barcode BC_3  REV
tgatataccggctta

SEQ ID NO: 69 - DNA, 15 bp, Artificial Sequence
  oligonucleotide barcode BC_4  REV
ttccgtcatatcgaa

SEQ ID NO: 70 - DNA, 15 bp, Artificial Sequence
  oligonucleotide barcode BC_5  REV
cctaacgcgtatacg

SEQ ID NO: 71 - DNA, 15 bp, Artificial Sequence
  oligonucleotide barcode BC_6  REV
ggaatctcggcagtt

SEQ ID NO: 72 - DNA, 15 bp, Artificial Sequence
  oligonucleotide barcode BC_7  REV
gcacagctaagatca

SEQ ID NO: 73 - DNA, 15 bp, Artificial Sequence
  oligonucleotide barcode BC_8  REV
tcaaggtaccgactc

SEQ ID NO: 74 - DNA, 15 bp, Artificial Sequence
  oligonucleotide barcode BC_9  REV
ccagatcacaagcgg

SEQ ID NO: 75 - DNA, 15 bp, Artificial Sequence
  oligonucleotide barcode BC_10  REV
tccggtacgctatct

SEQ ID NO: 76 - DNA, 15 bp, Artificial Sequence
  oligonucleotide barcode BC_11  REV
gatgatgagcctgga

SEQ ID NO: 77 - DNA, 15 bp, Artificial Sequence
  oligonucleotide barcode BC_12  REV
ttggctctagtactc

SEQ ID NO: 78 - DNA, 15 bp, Artificial Sequence
  oligonucleotide barcode BC_13  REV
ccgttattgacgctc

SEQ ID NO: 79 - DNA, 15 bp, Artificial Sequence
  oligonucleotide barcode BC_14  REV
cagtgtagataccgc

SEQ ID NO: 80 - DNA, 15 bp, Artificial Sequence
  oligonucleotide barcode BC_15  REV
ttctcttcggagaag

SEQ ID NO: 81 - DNA, 15 bp, Artificial Sequence
  oligonucleotide barcode BC_16  REV
ttaacacaggcttca

SEQ ID NO: 82 - DNA, 15 bp, Artificial Sequence
  oligonucleotide barcode BC_17  REV
tcgacaaccatccag

SEQ ID NO: 83 - DNA, 15 bp, Artificial Sequence
  oligonucleotide barcode BC_18  REV
ttggaaccgtgcagt

SEQ ID NO: 84 - DNA, 15 bp, Artificial Sequence
  oligonucleotide barcode BC_19 REV
aaggaccatctctcg

SEQ ID NO: 85 - DNA, 15 bp, Artificial Sequence
  oligonucleotide barcode BC_20 REV
tcttgtctctcaaga

SEQ ID NO: 86 - DNA, 15 bp, Artificial Sequence
  oligonucleotide barcode BC_21 REV
aacacagtgcgaatt

SEQ ID NO: 87 - DNA, 15 bp, Artificial Sequence
  oligonucleotide barcode BC_22 REV
tcttagcggcactac

SEQ ID NO: 88 - DNA, 15 bp, Artificial Sequence
  oligonucleotide barcode BC_23 REV
ggattgtgctatagg

SEQ ID NO: 89 - DNA, 15 bp, Artificial Sequence
  oligonucleotide barcode BC_24 REV
tccaacctcggtgat

SEQ ID NO: 90 - DNA, 15 bp, Artificial Sequence
  oligonucleotide barcode BC_25 REV
cttctccgttgaatc

SEQ ID NO: 91 - DNA, 15 bp, Artificial Sequence
  oligonucleotide barcode BC_26 REV
tggtgcgaggttcgt

SEQ ID NO: 92 - DNA, 15 bp, Artificial Sequence
  oligonucleotide barcode BC_27 REV
tcttcttgaagtcct

SEQ ID NO: 93 - DNA, 15 bp, Artificial Sequence
  oligonucleotide barcode BC_28 REV
tgcgaggattcaacc

SEQ ID NO: 94 - DNA, 15 bp, Artificial Sequence
  oligonucleotide barcode BC_29 REV
cgctagaggttggtt

SEQ ID NO: 95 - DNA, 15 bp, Artificial Sequence
  oligonucleotide barcode BC_30 REV
gttagatattcgcgt

SEQ ID NO: 96 - DNA, 15 bp, Artificial Sequence
  oligonucleotide barcode BC_31 REV
ggtgtaattctcaac

SEQ ID NO: 97 - DNA, 15 bp, Artificial Sequence
  oligonucleotide barcode BC_32 REV
ggttcacagagagag

SEQ ID NO: 98 - DNA, 15 bp, Artificial Sequence
  oligonucleotide barcode BC_33 REV
tctcttactgatggc

SEQ ID NO: 99 - DNA, 15 bp, Artificial Sequence
  oligonucleotide barcode BC_34 REV
ctcaattcacgttgc

SEQ ID NO: 100 - DNA, 15 bp, Artificial Sequence
  oligonucleotide barcode BC_35 REV
tgtggctctacttag

SEQ ID NO: 101 - DNA, 15 bp, Artificial Sequence
  oligonucleotide barcode BC_36 REV
cgcttccaacagaca

SEQ ID NO: 102 - DNA, 15 bp, Artificial Sequence
  oligonucleotide barcode BC_37 REV
cgcgctgtctattaa

SEQ ID NO: 103 - DNA, 15 bp, Artificial Sequence
  oligonucleotide barcode BC_38 REV
cttgttagagcgtcg

SEQ ID NO: 104 - DNA, 15 bp, Artificial Sequence
  oligonucleotide barcode BC_39 REV
tctcaataagccatg

SEQ ID NO: 105 - DNA, 15 bp, Artificial Sequence
  oligonucleotide barcode BC_40 REV
ccggccatacctagt

SEQ ID NO: 106 - DNA, 15 bp, Artificial Sequence
  oligonucleotide barcode BC_41 REV
aggatagacgaggac

SEQ ID NO: 107 - DNA, 15 bp, Artificial Sequence
  oligonucleotide barcode BC_42 REV
ggtaacggaatccta

SEQ ID NO: 108 - DNA, 15 bp, Artificial Sequence
  oligonucleotide barcode BC_43 REV
cttccggtggtcaga

SEQ ID NO: 109 - DNA, 15 bp, Artificial Sequence
  oligonucleotide barcode BC_44 REV
ccacgaggtgactct

SEQ ID NO: 110 - DNA, 15 bp, Artificial Sequence
  oligonucleotide barcode BC_45 REV
cttcgactacatcag

SEQ ID NO: 111 - DNA, 15 bp, Artificial Sequence
  oligonucleotide barcode BC_46 REV
ctatccgcaaccgac

SEQ ID NO: 112 - DNA, 15 bp, Artificial Sequence
  oligonucleotide barcode BC_47 REV
ttcttaggaggagga

SEQ ID NO: 113 - DNA, 15 bp, Artificial Sequence
  oligonucleotide barcode BC_48 REV
tgaagtggaccgaat

SEQ ID NO: 114 - DNA, 15 bp, Artificial Sequence
  oligonucleotide barcode BC_49 REV
cgcagacctgtaagg

SEQ ID NO: 115 - DNA, 15 bp, Artificial Sequence
  oligonucleotide barcode BC_50 REV
aattggccaatgatc

SEQ ID NO: 116 - DNA, 15 bp, Artificial Sequence
  oligonucleotide barcode BC_51 REV
caactcagccttgaa

SEQ ID NO: 117 - DNA, 15 bp, Artificial Sequence
  oligonucleotide barcode BC_52 REV
gattcaatcgagcca

SEQ ID NO: 118 - DNA, 15 bp, Artificial Sequence
  oligonucleotide barcode BC_53 REV
gagcggatggcttac

SEQ ID NO: 119 - DNA, 15 bp, Artificial Sequence
  oligonucleotide barcode BC_54 REV
tgtctacgcatgtgt

SEQ ID NO: 120 - DNA, 15 bp, Artificial Sequence
  oligonucleotide barcode BC_55 REV
cttgaatccatagca

SEQ ID NO: 121 - DNA, 15 bp, Artificial Sequence
  oligonucleotide barcode BC_56 REV
aactaagcctcgtgg

SEQ ID NO: 122 - DNA, 15 bp, Artificial Sequence
  oligonucleotide barcode BC_57 REV
gcaccttagttggcc

SEQ ID NO: 123 - DNA, 15 bp, Artificial Sequence
  oligonucleotide barcode BC_58 REV
ttgtcgaataggtgc

SEQ ID NO: 124 - DNA, 15 bp, Artificial Sequence
  oligonucleotide barcode BC_59 REV
agccgatcgtgtcca

SEQ ID NO: 125 - DNA, 15 bp, Artificial Sequence
  oligonucleotide barcode BC_60 REV
ccgttggaattatag

SEQ ID NO: 126 - DNA, 15 bp, Artificial Sequence
  oligonucleotide barcode BC_61 REV
cttactaaccacgtt

SEQ ID NO: 127 - DNA, 15 bp, Artificial Sequence
  oligonucleotide barcode BC_62 REV
gccactcgttccttg

SEQ ID NO: 128 - DNA, 15 bp, Artificial Sequence
  oligonucleotide barcode BC_63 REV
tcttccgttctggtg

SEQ ID NO: 129 - DNA, 15 bp, Artificial Sequence
  oligonucleotide barcode BC_64 REV
ttgcttgaccgtacg

SEQ ID NO: 130 - DNA, 15 bp, Artificial Sequence
  oligonucleotide barcode BC_65 REV
ttagcctgtcaccga

SEQ ID NO: 131 - protein, 16 aa, Artificial Sequence
  synthetic peptide
  MOD_RES (1)..(1): formyl-Methionine
MDVEAWLGARVPLVET

SEQ ID NO: 132 - protein, 10 aa, Artificial Sequence
  synthetic peptide
TENLYFQNHV

SEQ ID NO: 133 - DNA, 20 bp, Artificial Sequence
  oligonucleotide primer
aatgatacggcgaccaccga

SEQ ID NO: 134 - DNA, 24 bp, Artificial Sequence
  oligonucleotide primer
caagcagaagacggcatacgagat

SEQ ID NO: 135 - DNA, 5 bp, Artificial Sequence
  oligonucleotide
  misc_feature (1)..(5): n = A,T,C or G
nnnnn

SEQ ID NO: 136 - DNA, 4 bp, Artificial Sequence
  oligonucleotide
  misc_feature (1)..(4): n = A, T, C or G
nnnn

SEQ ID NO: 137 - DNA, 10 bp, Artificial Sequence
  Exemplary compartment tag
  misc_feature (1)..(10): n = A, T, C or G
nnnnnnnnnn

SEQ ID NO: 138 - protein, 6 aa, Artificial Sequence
  LIGASE PEPTIDE MOTIF
CGSNVH

SEQ ID NO: 139 - protein, 6 aa, Artificial Sequence
  LIGASE PEPTIDE MOTIF
  MISC_FEATURE (1)..(1): xaa = any amino acid
XCGSHV

SEQ ID NO: 140 - protein, 5 aa, Artificial Sequence
  Linker
GGGGS

SEQ ID NO: 141 - protein, 7 aa, Artificial Sequence
  TEV protease consensus sequence
ENLYFQS

SEQ ID NO: 142 - protein, 7 aa, Artificial Sequence
  Assay Peptide
GRFSGIY

SEQ ID NO: 143 - protein, 5 aa, Artificial Sequence
  Assay Peptide
AALAY

SEQ ID NO: 144 - protein, 8 aa, Artificial Sequence
  Assay Peptide
  MISC_FEATURE (8)..(8): C-terminal lysine with an azide substitution on the side chain
FGAALAWK

SEQ ID NO: 145 - protein, 7 aa, Artificial Sequence
  Assay Peptide
WTQIFGA

SEQ ID NO: 146 - protein, 6 aa, Artificial Sequence
  Peptide after elimination
TQIFGA

SEQ ID NO: 147 - protein, 7 aa, Artificial Sequence
  Peptide after elimination
  MISC_FEATURE (7)..(7): C-terminal lysine with an azide substitution on the side chain
GAALAWK

SEQ ID NO: 148 - protein, 4 aa, Artificial Sequence
  Peptide after elimination
ALAY

SEQ ID NO: 149 - protein, 6 aa, Artificial Sequence
  Peptide after elimination
RFSGIY

SEQ ID NO: 150 - protein, 8 aa, Artificial Sequence
  Assay Peptide
  MISC_FEATURE (8)..(8): C-terminal lysine with an azide substitution on the side chain
FHAALAWK

SEQ ID NO: 151 - protein, 7 aa, Artificial Sequence
  Peptide after elimination
  MISC_FEATURE (7)..(7): C-terminal lysine with an azide substitution on the side chain
HAALAWK

SEQ ID NO: 152 - protein, 22 aa, Artificial Sequence
  Assay Peptide
  MISC_FEATURE (22)..(22): C-terminal lysine with an azide substitution on the side chain
YAEALAESAFSGVARGDVRGGK

SEQ ID NO: 153 - protein, 21 aa, Artificial Sequence
  Assay Peptide
  MISC_FEATURE (21)..(21): C-terminal lysine with an azide substitution on the side chain
AEALAESAFSGVARGDVRGGK

SEQ ID NO: 154 - protein, 20 aa, Artificial Sequence
  Assay Peptide
  MISC_FEATURE (20)..(20): C-terminal lysine with an azide substitution on the side chain
EALAESAFSGVARGDVRGGK

SEQ ID NO: 155 - protein, 19 aa, Artificial Sequence
  Assay Peptide
  MISC_FEATURE (19)..(19): C-terminal lysine with an azide substitution on the side chain
ALAESAFSGVARGDVRGGK

SEQ ID NO: 156 - protein, 18 aa, Artificial Sequence
  Assay Peptide
  MISC_FEATURE (18)..(18): C-terminal lysine with an azide substitution on the side chain
LAESAFSGVARGDVRGGK

SEQ ID NO: 157 - protein, 17 aa, Artificial Sequence
  Assay Peptide
  MISC_FEATURE (17)..(17): C-terminal lysine with an azide substitution on the side chain
AESAFSGVARGDVRGGK

SEQ ID NO: 158 - protein, 16 aa, Artificial Sequence
  Assay Peptide
  MISC_FEATURE (16)..(16): C-terminal lysine with an azide substitution on the side chain
ESAFSGVARGDVRGGK

SEQ ID NO: 159 - protein, 15 aa, Artificial Sequence
  Assay Peptide
  MISC_FEATURE (15)..(15): C-terminal lysine with an azide substitution on the side chain
SAFSGVARGDVRGGK

SEQ ID NO: 160 - protein, 14 aa, Artificial Sequence
  Assay Peptide
  MISC_FEATURE (14)..(14): C-terminal lysine with an azide substitution on the side chain
AFSGVARGDVRGGK

SEQ ID NO: 161 - protein, 13 aa, Artificial Sequence
  Assay Peptide
  MISC_FEATURE (13)..(13): C-terminal lysine with an azide substitution on the side chain
FSGVARGDVRGGK

SEQ ID NO: 162 - protein, 12 aa, Artificial Sequence
  Assay Peptide
  MISC_FEATURE (12)..(12): C-terminal lysine with an azide substitution on the side chain
SGVARGDVRGGK

SEQ ID NO: 163 - protein, 21 aa, Artificial Sequence
  Assay Peptide
  MISC_FEATURE (21)..(21): C-terminal lysine with an azide substitution on the side chain
LAGELAGELAGEIRGDVRGGK

SEQ ID NO: 164 - protein, 22 aa, Artificial Sequence
  Assay Peptide
  MISC_FEATURE (22)..(22): C-terminal lysine with an azide substitution on the side chain
ELAGELAGELAGEIRGDVRGGK

SEQ ID NO: 165 - protein, 23 aa, Artificial Sequence
  Assay Peptide
  MISC_FEATURE (23)..(23): C-terminal lysine with an azide substitution on the side chain
GELAGELAGELAGEIRGDVRGGK

SEQ ID NO: 166 - protein, 24 aa, Artificial Sequence
  Assay Peptide
  MISC_FEATURE (24)..(24): C-terminal lysine with an azide substitution on the side chain
AGELAGELAGELAGEIRGDVRGGK

SEQ ID NO: 167 - protein, 19 aa, Artificial Sequence
  Assay Peptide
  MISC_FEATURE (19)..(19): C-terminal lysine with an azide substitution on the side chain
FAFAGVAMPRGAEDVRGGK

SEQ ID NO: 168 - DNA, 734 bp, Artificial Sequence
  extended recording tag construct
aatcacggtacaagtcactcatccgtacgctatctgagaatcgtccagatccggcatgctagtatctggtgcagactacgattgttacagatcactcagatgatgagcacagaaaatcgtcgaatcttccatcaccatcgaacagttacgattaatgtagtccgcacaatcgaatgtctaacatgccgaatcccggacgtctccagcttctaaaccaacagtagtcgcacaaatcattgtacggtacaagatctaacgagagatgatcggatctgaccactttaaacactgattacgcagactacgattacgatttaagaatcctcgtccggtacaatcatagtccgcacaatcaaccgtgtcatgtgaagatcagatcgatctcgaatagcgtaccagacagtgatcttgcaaatcgtaatgtgtccgcgccaatcgatagccatgaatcccagtcgatctcccgcttgtgatctggcgatcgccttgtaccgtcgtacgatttgagatcacctcgttaactcaagctaaagatcgtccggatcgctttataaacatctgattgcgcggtacgattatcgtagtccgcacatatcgaacctgttgaagatccggatcgtctctccaggctcatcatccgagtgatccttgcaaataatcatgtccgcaccatcaggtgtctaacgcttgccggatccgaatcgatctctccaggctcatcatcgaagtgatgt

SEQ ID NO: 169 - protein, 10 aa, Artificial Sequence
  synthetic peptide
CPVQLWVDST

SEQ ID NO: 170 - protein, 10 aa, Artificial Sequence
  synthetic peptide
  MISC_FEATURE (3)..(3): xaa = any amino acid
  MISC_FEATURE (5)..(5): xaa = any amino acid
  MISC_FEATURE (7)..(7): xaa = any amino acid
  MISC_FEATURE (9)..(9): xaa = any amino acid
CPXQXWXDXT

SEQ ID NO: 171 - DNA, 48 bp, Artificial Sequence
  synthetic oligonucleotide
  misc_feature (4)..(4): n = an internal 5-Octadiynyl dU
tttnttucgtagtccgcgacactagtaagccggtatatcaactgagtg

SEQ ID NO: 172 - protein, 13 aa, Artificial Sequence
  Assay Peptide
  MISC_FEATURE (13)..(13): C-terminal lysine with an azide substitution on the side chain
FLAEIRGDVRGGK

SEQ ID NO: 173 - protein, 28 aa, Artificial Sequence
  Assay Peptide
  MOD_RES (1)..(1): N-terminal dismethylated alanine
  MISC_FEATURE (28)..(28): C-terminal lysine with an azide substitution on the side chain
AESAESASRFSGVAMPGAEDDVVGSGSK

SEQ ID NO: 174 - protein, 13 aa, Artificial Sequence
  synthetic peptide
  MISC_FEATURE (13)..(13): C-terminal lysine with an azide substitution on the side chain
PAAEIRGDVRGGK

SEQ ID NO: 175 - protein, 13 aa, Artificial Sequence
  synthetic peptide
  MISC_FEATURE (13)..(13): C-terminal lysine with an azide substitution on the side chain
PDAEIRGDVRGGK

SEQ ID NO: 176 - protein, 13 aa, Artificial Sequence
  synthetic peptide
  MISC_FEATURE (13)..(13): C-terminal lysine with an azide substitution on the side chain
PEAEIRGDVRGGK

SEQ ID NO: 177 - protein, 13 aa, Artificial Sequence
  synthetic peptide
  MISC_FEATURE (13)..(13): C-terminal lysine with an azide substitution on the side chain
PFAEIRGDVRGGK

SEQ ID NO: 178 - protein, 13 aa, Artificial Sequence
  synthetic peptide
  MISC_FEATURE (13)..(13): C-terminal lysine with an azide substitution on the side chain
PGAEIRGDVRGGK

SEQ ID NO: 179 - protein, 13 aa, Artificial Sequence
  synthetic peptide
  MISC_FEATURE (13)..(13): C-terminal lysine with an azide substitution on the side chain
PHAEIRGDVRGGK

SEQ ID NO: 180 - protein, 13 aa, Artificial Sequence
  synthetic peptide
  MISC_FEATURE (13)..(13): C-terminal lysine with an azide substitution on the side chain
PIAEIRGDVRGGK

SEQ ID NO: 181 - protein, 13 aa, Artificial Sequence
  synthetic peptide
  MISC_FEATURE (13)..(13): C-terminal lysine with an azide substitution on the side chain
PLAEIRGDVRGGK

SEQ ID NO: 182 - protein, 13 aa, Artificial Sequence
  synthetic peptide
  MISC_FEATURE (13)..(13): C-terminal lysine with an azide substitution on the side chain
PMAEIRGDVRGGK

SEQ ID NO: 183 - protein, 13 aa, Artificial Sequence
  synthetic peptide
  MISC_FEATURE (13)..(13): C-terminal lysine with an azide substitution on the side chain
PNAEIRGDVRGGK

SEQ ID NO: 184 - protein, 13 aa, Artificial Sequence
  synthetic peptide
  MISC_FEATURE (13)..(13): C-terminal lysine with an azide substitution on the side chain
PPAEIRGDVRGGK

SEQ ID NO: 185 - protein, 13 aa, Artificial Sequence
  synthetic peptide
  MISC_FEATURE (13)..(13): C-terminal lysine with an azide substitution on the side chain
PQAEIRGDVRGGK

SEQ ID NO: 186 - protein, 13 aa, Artificial Sequence
  synthetic peptide
  MISC_FEATURE (13)..(13): C-terminal lysine with an azide substitution on the side chain
PSAEIRGDVRGGK

SEQ ID NO: 187 - protein, 13 aa, Artificial Sequence
  synthetic peptide
  MISC_FEATURE (13)..(13): C-terminal lysine with an azide substitution on the side chain
PTAEIRGDVRGGK

SEQ ID NO: 188 - protein, 13 aa, Artificial Sequence
  synthetic peptide
  MISC_FEATURE (13)..(13): C-terminal lysine with an azide substitution on the side chain
PVAEIRGDVRGGK

SEQ ID NO: 189 - protein, 13 aa, Artificial Sequence
  synthetic peptide
  MISC_FEATURE (13)..(13): C-terminal lysine with an azide substitution on the side chain
PWAEIRGDVRGGK

SEQ ID NO: 190 - protein, 13 aa, Artificial Sequence
  synthetic peptide
  MISC_FEATURE (13)..(13): C-terminal lysine with an azide substitution on the side chain
PYAEIRGDVRGGK

SEQ ID NO: 191 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QAAEIRGDVRGGK

SEQ ID NO: 192 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QDAEIRGDVRGGK

SEQ ID NO: 193 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QEAEIRGDVRGGK

SEQ ID NO: 194 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QFAEIRGDVRGGK

SEQ ID NO: 195 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QGAEIRGDVRGGK

SEQ ID NO: 196 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QHAEIRGDVRGGK

SEQ ID NO: 197 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QIAEIRGDVRGGK

SEQ ID NO: 198 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QLAEIRGDVRGGK

SEQ ID NO: 199 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QMAEIRGDVRGGK

SEQ ID NO: 200 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QNAEIRGDVRGGK

SEQ ID NO: 201 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QPAEIRGDVRGGK

SEQ ID NO: 202 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QQAEIRGDVRGGK

SEQ ID NO: 203 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QSAEIRGDVRGGK

SEQ ID NO: 204 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QTAEIRGDVRGGK

SEQ ID NO: 205 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QVAEIRGDVRGGK

SEQ ID NO: 206 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QWAEIRGDVRGGK

SEQ ID NO: 207 - protein, 13 aa, Artificial Sequence
  synthetic peptide
  MOD_RES (1)..(1): N-terminal pyrrolidone carboxylic acid
  MISC_FEATURE (13)..(13): C-terminal lysine with an azide substitution on the side chain
QYAEIRGDVRGGK